SEQUENCE LISTING (529 sequences)

SEQ ID NO: 1 - protein, 10 aa, synthetic construct
  REGION 1..10: note = AS63930 CDR1
GYTYSGNYMA

SEQ ID NO: 2 - protein, 10 aa, synthetic construct
  REGION 1..10: note = AS63932 CDR1
GYTYGSTFMG

SEQ ID NO: 3 - protein, 10 aa, synthetic construct
  REGION 1..10: note = AS63951 CDR1
RDIYGNNCMA

SEQ ID NO: 4 - protein, 10 aa, synthetic construct
  REGION 1..10: note = AS63984 CDR1
GYTYSSNFMG

SEQ ID NO: 5 - protein, 7 aa, synthetic construct
  REGION 1..7: note = AS63987 CDR1
GYRNCMA

SEQ ID NO: 6 - protein, 10 aa, synthetic construct
  REGION 1..10: note = AS63997 CDR1
FSGYGVSTMA

SEQ ID NO: 7 - protein, 10 aa, synthetic construct
  REGION 1..10: note = AS64047 CDR1
QYVYRWDLMG

SEQ ID NO: 8 - protein, 10 aa, synthetic construct
  REGION 1..10: note = AS64052 CDR1
GYTYRSNFMG

SEQ ID NO: 9 - protein, 11 aa, synthetic construct
  REGION 1..11: note = AS64062 CDR1
RSPYSSSRCMG

SEQ ID NO: 10 - protein, 10 aa, synthetic construct
  REGION 1..10: note = AS64072 CDR1
GYSYYINLMA

SEQ ID NO: 11 - protein, 10 aa, synthetic construct
  REGION 1..10: note = AS64097 CDR1
GYTYSRNCMG

SEQ ID NO: 12 - protein, 10 aa, synthetic construct
  REGION 1..10: note = AS64114 CDR1
GNTYSTNYMG

SEQ ID NO: 13 - protein, 10 aa, synthetic construct
  REGION 1..10: note = AS64123 CDR1
GYTYTSNWLG

SEQ ID NO: 14 - protein, 10 aa, synthetic construct
  REGION 1..10: note = AS64130 CDR1
GYTYRSNFMG

SEQ ID NO: 15 - protein, 10 aa, synthetic construct
  REGION 1..10: note = AS64137 CDR1
GSTYSTNFMG

SEQ ID NO: 16 - protein, 10 aa, synthetic construct
  REGION 1..10: note = AS64142 CDR1
GFTFDRNAMR

SEQ ID NO: 17 - protein, 10 aa, synthetic construct
  REGION 1..10: note = AS64154 CDR1
GYTYRYLYMG

SEQ ID NO: 18 - protein, 10 aa, synthetic construct
  REGION 1..10: note = AS64160 CDR1
VYTSSSYCMG

SEQ ID NO: 19 - protein, 10 aa, synthetic construct
  REGION 1..10: note = AS64228 CDR1
GVSYNRCSMG

SEQ ID NO: 20 - protein, 10 aa, synthetic construct
  REGION 1..10: note = AS64300 CDR1
GDIYNLMSMA

SEQ ID NO: 21 - protein, 10 aa, synthetic construct
  REGION 1..10: note = AS64380 CDR1
GNTYSSNYMG

SEQ ID NO: 22 - protein, 10 aa, synthetic construct
  REGION 1..10: note = AS64395 CDR1
GSTYSTNFMG

SEQ ID NO: 23 - protein, 10 aa, synthetic construct
  REGION 1..10: note = AS64443 CDR1
GYTDSSVYIG

SEQ ID NO: 24 - protein, 10 aa, synthetic construct
  REGION 1..10: note = AS64511 CDR1
RATYSTNYIS

SEQ ID NO: 25 - protein, 10 aa, synthetic construct
  REGION 1..10: note = AS64536 CDR1
RYTDNFVYMG

SEQ ID NO: 26 - protein, 10 aa, synthetic construct
  REGION 1..10: note = AS64597 CDR1
GYTYRVNFMG

SEQ ID NO: 27 - protein, 9 aa, synthetic construct
  REGION 1..9: note = AS64617 CDR1
GYTDRCSMA

SEQ ID NO: 28 - protein, 12 aa, synthetic construct
  REGION 1..12: note = AS64634 CDR1
GYSFRGDFMCMG

SEQ ID NO: 29 - protein, 10 aa, synthetic construct
  REGION 1..10: note = AS69498 CDR1
GNTYSSNYMA

SEQ ID NO: 30 - protein, 10 aa, synthetic construct
  REGION 1..10: note = AS69500 CDR1
RYTYSSACMG

SEQ ID NO: 31 - protein, 10 aa, synthetic construct
  REGION 1..10: note = AS69527 CDR1
RYTFSSTCMA

SEQ ID NO: 32 - protein, 10 aa, synthetic construct
  REGION 1..10: note = AS68280 CDR1
GSTYSSNYLG

SEQ ID NO: 33 - protein, 10 aa, synthetic construct
  REGION 1..10: note = AS68355 CDR1
GYTYSGVCMG

SEQ ID NO: 34 - protein, 10 aa, synthetic construct
  REGION 1..10: note = AS69443 CDR1
GFTFDDSDMA

SEQ ID NO: 35 - protein, 10 aa, synthetic construct
  REGION 1..10: note = AS75376 CDR1
GYTYSSHSMG

SEQ ID NO: 36 - protein, 10 aa, synthetic construct
  REGION 1..10: note = AS75387 CDR1
GYPYSSPCMA

SEQ ID NO: 37 - protein, 10 aa, synthetic construct
  REGION 1..10: note = AS75695 CDR1
GYTVSAYCMG

SEQ ID NO: 38 - protein, 10 aa, synthetic construct
  REGION 1..10: note = AS76169 CDR1
GYIYSSFCMG

SEQ ID NO: 39 - protein, 10 aa, synthetic construct
  REGION 1..10: note = AS63931 CDR1
FSGYGVSTMA

SEQ ID NO: 40 - protein, 10 aa, synthetic construct
  REGION 1..10: note = AS63937 CDR1
GSTISSRPMA

SEQ ID NO: 41 - protein, 10 aa, synthetic construct
  REGION 1..10: note = AS63948 CDR1
GYTYRYLYMG

SEQ ID NO: 42 - protein, 10 aa, synthetic construct
  REGION 1..10: note = AS63956 CDR1
GFTYSNCCMR

SEQ ID NO: 43 - protein, 10 aa, synthetic construct
  REGION 1..10: note = AS63965 CDR1
GYSSGSCRMG

SEQ ID NO: 44 - protein, 10 aa, synthetic construct
  REGION 1..10: note = AS63993 CDR1
GFTFDDLVMA

SEQ ID NO: 45 - protein, 10 aa, synthetic construct
  REGION 1..10: note = AS63999 CDR1
GYTYSSNWMG

SEQ ID NO: 46 - protein, 10 aa, synthetic construct
  REGION 1..10: note = AS64006 CDR1
GYTGDTTYIG

SEQ ID NO: 47 - protein, 10 aa, synthetic construct
  REGION 1..10: note = AS64057 CDR1
GFTFDRNAMR

SEQ ID NO: 48 - protein, 10 aa, synthetic construct
  REGION 1..10: note = AS64060 CDR1
GSTYCTYRMS

SEQ ID NO: 49 - protein, 10 aa, synthetic construct
  REGION 1..10: note = AS64071 CDR1
GNTYRLNSMG

SEQ ID NO: 50 - protein, 10 aa, synthetic construct
  REGION 1..10: note = AS64093 CDR1
RYIYGNNCMA

SEQ ID NO: 51 - protein, 10 aa, synthetic construct
  REGION 1..10: note = AS64118 CDR1
GYTYSACRMA

SEQ ID NO: 52 - protein, 10 aa, synthetic construct
  REGION 1..10: note = AS64120 CDR1
RYIYGNNCMA

SEQ ID NO: 53 - protein, 10 aa, synthetic construct
  REGION 1..10: note = AS64124 CDR1
TYTPSNNYMG

SEQ ID NO: 54 - protein, 10 aa, synthetic construct
  REGION 1..10: note = AS64135 CDR1
TSTYCRYYMR

SEQ ID NO: 55 - protein, 10 aa, synthetic construct
  REGION 1..10: note = AS64163 CDR1
GYRYRWNCMA

SEQ ID NO: 56 - protein, 10 aa, synthetic construct
  REGION 1..10: note = AS64182 CDR1
GQTSRYLYMG

SEQ ID NO: 57 - protein, 10 aa, synthetic construct
  REGION 1..10: note = AS64183 CDR1
GHTYSANCMA

SEQ ID NO: 58 - protein, 10 aa, synthetic construct
  REGION 1..10: note = AS64207 CDR1
GYTYSSNFMG

SEQ ID NO: 59 - protein, 10 aa, synthetic construct
  REGION 1..10: note = AS64276 CDR1
GYTGSSRCMA

SEQ ID NO: 60 - protein, 10 aa, synthetic construct
  REGION 1..10: note = AS64336 CDR1
GRTYSSCSMG

SEQ ID NO: 61 - protein, 7 aa, synthetic construct
  REGION 1..7: note = AS64346 CDR1
GYTYFMG

SEQ ID NO: 62 - protein, 10 aa, synthetic construct
  REGION 1..10: note = AS64420 CDR1
GDTSRSVWMG

SEQ ID NO: 63 - protein, 10 aa, synthetic construct
  REGION 1..10: note = AS64473 CDR1
GYTYRYLYMA

SEQ ID NO: 64 - protein, 10 aa, synthetic construct
  REGION 1..10: note = AS64475 CDR1
GYTWSRNWMG

SEQ ID NO: 65 - protein, 10 aa, synthetic construct
  REGION 1..10: note = AS64513 CDR1
DYPYIDNCMG

SEQ ID NO: 66 - protein, 10 aa, synthetic construct
  REGION 1..10: note = AS64562 CDR1
GYTARRDFMA

SEQ ID NO: 67 - protein, 10 aa, synthetic construct
  REGION 1..10: note = AS64583 CDR1
GFTIAVYTMG

SEQ ID NO: 68 - protein, 10 aa, synthetic construct
  REGION 1..10: note = AS64594 CDR1
GYTYNSNYMG

SEQ ID NO: 69 - protein, 10 aa, synthetic construct
  REGION 1..10: note = AS64605 CDR1
RYPYSSICMG

SEQ ID NO: 70 - protein, 10 aa, synthetic construct
  REGION 1..10: note = AS64606 CDR1
GYTSRSNYMG

SEQ ID NO: 71 - protein, 10 aa, synthetic construct
  REGION 1..10: note = AS68121 CDR1
GYTYSRNCMG

SEQ ID NO: 72 - protein, 10 aa, synthetic construct
  REGION 1..10: note = AS68170 CDR1
GYTYRSNCMG

SEQ ID NO: 73 - protein, 10 aa, synthetic construct
  REGION 1..10: note = AS63964 CDR1
GYTYSYNNMG

SEQ ID NO: 74 - protein, 8 aa, synthetic construct
  REGION 1..8: note = AS64116 CDR1
GYIYSCVG

SEQ ID NO: 75 - protein, 13 aa, synthetic construct
  REGION 1..13: note = AS68270 CDR1
GYPSSTYYMLSMA

SEQ ID NO: 76 - protein, 10 aa, synthetic construct
  REGION 1..10: note = AS68320 CDR1
GYTYNTNYMG

SEQ ID NO: 77 - protein, 10 aa, synthetic construct
  REGION 1..10: note = AS68351 CDR1
GDTFRAYYMN

SEQ ID NO: 78 - protein, 10 aa, synthetic construct
  REGION 1..10: note = AS75378 CDR1
GNTRSTTYMG

SEQ ID NO: 79 - protein, 10 aa, synthetic construct
  REGION 1..10: note = AS75383 CDR1
GYTFSSYCLG

SEQ ID NO: 80 - protein, 11 aa, synthetic construct
  REGION 1..11: note = AS75751 CDR1
GYFYNTYYFMG

SEQ ID NO: 81 - protein, 10 aa, synthetic construct
  REGION 1..10: note = AS76422 CDR1
GYTFAGNCLG

SEQ ID NO: 82 - protein, 20 aa, synthetic construct
  REGION 1..20: note = AS63930 CDR2
VVYNIDGGGRFTTYADSVKG

SEQ ID NO: 83 - protein, 17 aa, synthetic construct
  REGION 1..17: note = AS63932 CDR2
VIYTGGGSTWYASSVKG

SEQ ID NO: 84 - protein, 17 aa, synthetic construct
  REGION 1..17: note = AS63951 CDR2
SIYPAGGRPYYADSVKG

SEQ ID NO: 85 - protein, 17 aa, synthetic construct
  REGION 1..17: note = AS63984 CDR2
TIVSGGGTTYYADSVRG

SEQ ID NO: 86 - protein, 17 aa, synthetic construct
  REGION 1..17: note = AS63987 CDR2
VIYTPSGITDYASSVKG

SEQ ID NO: 87 - protein, 17 aa, synthetic construct
  REGION 1..17: note = AS63997 CDR2
AITVGSGNTYYADSVKG

SEQ ID NO: 88 - protein, 17 aa, synthetic construct
  REGION 1..17: note = AS64047 CDR2
AVYTGDGITYYADSVKG

SEQ ID NO: 89 - protein, 17 aa, synthetic construct
  REGION 1..17: note = AS64052 CDR2
TIHSGVATTYYADSVKG

SEQ ID NO: 90 - protein, 17 aa, synthetic construct
  REGION 1..17: note = AS64062 CDR2
ALYTGGGSTSYADSVKG

SEQ ID NO: 91 - protein, 17 aa, synthetic construct
  REGION 1..17: note = AS64072 CDR2
AHGPVSGTAYYTDSVKG

SEQ ID NO: 92 - protein, 17 aa, synthetic construct
  REGION 1..17: note = AS64097 CDR2
AINTGGGSTYYADSLEG

SEQ ID NO: 93 - protein, 17 aa, synthetic construct
  REGION 1..17: note = AS64114 CDR2
VIYTRGGHTYYVDSVRG

SEQ ID NO: 94 - protein, 17 aa, synthetic construct
  REGION 1..17: note = AS64123 CDR2
IIYTGSGSTHYRSSVKG

SEQ ID NO: 95 - protein, 17 aa, synthetic construct
  REGION 1..17: note = AS64130 CDR2
TIDSRGTITYYADSVKG

SEQ ID NO: 96 - protein, 17 aa, synthetic construct
  REGION 1..17: note = AS64137 CDR2
TLVTWVERTAYADSVKG

SEQ ID NO: 97 - protein, 17 aa, synthetic construct
  REGION 1..17: note = AS64142 CDR2
CIDWTGANIAYADSVKG

SEQ ID NO: 98 - protein, 17 aa, synthetic construct
  REGION 1..17: note = AS64154 CDR2
CIYTGSGSTGYADSVKG

SEQ ID NO: 99 - protein, 17 aa, synthetic construct
  REGION 1..17: note = AS64160 CDR2
AMCFGGLVTHYADSVKG

SEQ ID NO: 100 - protein, 16 aa, synthetic construct
  REGION 1..16: note = AS64228 CDR2
RIQPGGNTYYADSVKG

SEQ ID NO: 101 - protein, 17 aa, synthetic construct
  REGION 1..17: note = AS64300 CDR2
YINTIIGNTYYTDSVKG

SEQ ID NO: 102 - protein, 17 aa, synthetic construct
  REGION 1..17: note = AS64380 CDR2
VIYTRGGHTYYVDSVRG

SEQ ID NO: 103 - protein, 17 aa, synthetic construct
  REGION 1..17: note = AS64395 CDR2
TLVTWAERTAYADSVKG

SEQ ID NO: 104 - protein, 17 aa, synthetic construct
  REGION 1..17: note = AS64443 CDR2
IIYTGGESTHYRSSVKG

SEQ ID NO: 105 - protein, 17 aa, synthetic construct
  REGION 1..17: note = AS64511 CDR2
TITTGDGETAYADSVKG

SEQ ID NO: 106 - protein, 17 aa, synthetic construct
  REGION 1..17: note = AS64536 CDR2
LIYPGGGSTYYASSVKG

SEQ ID NO: 107 - protein, 17 aa, synthetic construct
  REGION 1..17: note = AS64597 CDR2
TIDSGVGTTYYADSVKG

SEQ ID NO: 108 - protein, 16 aa, synthetic construct
  REGION 1..16: note = AS64617 CDR2
RISTSGFTNYAASVKG

SEQ ID NO: 109 - protein, 17 aa, synthetic construct
  REGION 1..17: note = AS64634 CDR2
VFYPGGGSTNYADSAKG

SEQ ID NO: 110 - protein, 17 aa, synthetic construct
  REGION 1..17: note = AS69498 CDR2
VIYTRGGHTYYIDSVRG

SEQ ID NO: 111 - protein, 18 aa, synthetic construct
  REGION 1..18: note = AS69500 CDR2
SIFTGTGGSTYYADSVKG

SEQ ID NO: 112 - protein, 17 aa, synthetic construct
  REGION 1..17: note = AS69527 CDR2
AIYTDDGSTWYADSVKG

SEQ ID NO: 113 - protein, 17 aa, synthetic construct
  REGION 1..17: note = AS68280 CDR2
AISTGDGATAYADSVKG

SEQ ID NO: 114 - protein, 16 aa, synthetic construct
  REGION 1..16: note = AS68355 CDR2
AIDSDGSTSYADSVKG

SEQ ID NO: 115 - protein, 16 aa, synthetic construct
  REGION 1..16: note = AS69443 CDR2
TISSDGSTYYADSVKG

SEQ ID NO: 116 - protein, 17 aa, synthetic construct
  REGION 1..17: note = AS75376 CDR2
VIYTGDGSTYYADSVKG

SEQ ID NO: 117 - protein, 17 aa, synthetic construct
  REGION 1..17: note = AS75387 CDR2
VAYTGGDIQYLTDSVKG

SEQ ID NO: 118 - protein, 16 aa, synthetic construct
  REGION 1..16: note = AS75695 CDR2
FIDAGGATIYADPVKG

SEQ ID NO: 119 - protein, 16 aa, synthetic construct
  REGION 1..16: note = AS76169 CDR2
YIRDNIMTSYTDSVKG

SEQ ID NO: 120 - protein, 17 aa, synthetic construct
  REGION 1..17: note = AS63931 CDR2
AITVGSGNTYYADSVTG

SEQ ID NO: 121 - protein, 17 aa, synthetic construct
  REGION 1..17: note = AS63937 CDR2
CIHTGLGRTYYADSVKG

SEQ ID NO: 122 - protein, 17 aa, synthetic construct
  REGION 1..17: note = AS63948 CDR2
CIYTGSGSTGYADSVKG

SEQ ID NO: 123 - protein, 16 aa, synthetic construct
  REGION 1..16: note = AS63956 CDR2
LINSSGGTYYADSVRG

SEQ ID NO: 124 - protein, 16 aa, synthetic construct
  REGION 1..16: note = AS63965 CDR2
KVISDGTTVYADSVKG

SEQ ID NO: 125 - protein, 16 aa, synthetic construct
  REGION 1..16: note = AS63993 CDR2
LVATAGNSVYADSVKG

SEQ ID NO: 126 - protein, 17 aa, synthetic construct
  REGION 1..17: note = AS63999 CDR2
IIYTGGISTHYRSSVKG

SEQ ID NO: 127 - protein, 17 aa, synthetic construct
  REGION 1..17: note = AS64006 CDR2
LIYTSGTSEYYADSVKG

SEQ ID NO: 128 - protein, 17 aa, synthetic construct
  REGION 1..17: note = AS64057 CDR2
CISWTGANIAYADSVKG

SEQ ID NO: 129 - protein, 16 aa, synthetic construct
  REGION 1..16: note = AS64060 CDR2
VIDSGGSTSYADSVKG

SEQ ID NO: 130 - protein, 17 aa, synthetic construct
  REGION 1..17: note = AS64071 CDR2
FIVMIRGTTYYGASVKG

SEQ ID NO: 131 - protein, 17 aa, synthetic construct
  REGION 1..17: note = AS64093 CDR2
SIYPAGGRTYYADSVKG

SEQ ID NO: 132 - protein, 16 aa, synthetic construct
  REGION 1..16: note = AS64118 CDR2
FINSAGSTYYADSVKG

SEQ ID NO: 133 - protein, 17 aa, synthetic construct
  REGION 1..17: note = AS64120 CDR2
SIYPAGGRPYYADSVKG

SEQ ID NO: 134 - protein, 17 aa, synthetic construct
  REGION 1..17: note = AS64124 CDR2
AIATIGGTTRYADSVKG

SEQ ID NO: 135 - protein, 16 aa, synthetic construct
  REGION 1..16: note = AS64135 CDR2
AMQPDGTTSYSDSVKG

SEQ ID NO: 136 - protein, 17 aa, synthetic construct
  REGION 1..17: note = AS64163 CDR2
AISTGSGSTYYAGSVKG

SEQ ID NO: 137 - protein, 17 aa, synthetic construct
  REGION 1..17: note = AS64182 CDR2
CIYTGSGSTGYADSVKG

SEQ ID NO: 138 - protein, 17 aa, synthetic construct
  REGION 1..17: note = AS64183 CDR2
SVYTDDDSTMYTDSVKG

SEQ ID NO: 139 - protein, 17 aa, synthetic construct
  REGION 1..17: note = AS64207 CDR2
TIVSGGGTTYYADSVRG

SEQ ID NO: 140 - protein, 17 aa, synthetic construct
  REGION 1..17: note = AS64276 CDR2
QIFTGRGTTGYADSVKG

SEQ ID NO: 141 - protein, 16 aa, synthetic construct
  REGION 1..16: note = AS64336 CDR2
HIFSDGSRYYADSVKG

SEQ ID NO: 142 - protein, 20 aa, synthetic construct
  REGION 1..20: note = AS64346 CDR2
TIGTGDIFNGAAYYVDSVKG

SEQ ID NO: 143 - protein, 17 aa, synthetic construct
  REGION 1..17: note = AS64420 CDR2
TISTAGGSTWYTDSVKG

SEQ ID NO: 144 - protein, 17 aa, synthetic construct
  REGION 1..17: note = AS64473 CDR2
CIYTGSGTTGYADSVKG

SEQ ID NO: 145 - protein, 17 aa, synthetic construct
  REGION 1..17: note = AS64475 CDR2
TITISGGSTWYADSVKG

SEQ ID NO: 146 - protein, 17 aa, synthetic construct
  REGION 1..17: note = AS64513 CDR2
AACTGGGSTYYADSVKG

SEQ ID NO: 147 - protein, 17 aa, synthetic construct
  REGION 1..17: note = AS64562 CDR2
VIHTGGDTTYYADSVKG

SEQ ID NO: 148 - protein, 17 aa, synthetic construct
  REGION 1..17: note = AS64583 CDR2
CTSWAGGRTYTADSVKG

SEQ ID NO: 149 - protein, 17 aa, synthetic construct
  REGION 1..17: note = AS64594 CDR2
LIYTGGGSTYYADSVKG

SEQ ID NO: 150 - protein, 17 aa, synthetic construct
  REGION 1..17: note = AS64605 CDR2
RIYTGTGSTWYTDSVKG

SEQ ID NO: 151 - protein, 20 aa, synthetic construct
  REGION 1..20: note = AS64606 CDR2
AFYLIYTRGGSTYYASSVKG

SEQ ID NO: 152 - protein, 17 aa, synthetic construct
  REGION 1..17: note = AS68121 CDR2
TDYIRFGRTYYADSVKG

SEQ ID NO: 153 - protein, 18 aa, synthetic construct
  REGION 1..18: note = AS68170 CDR2
TIYTGGGRNLYYADSVKG

SEQ ID NO: 154 - protein, 16 aa, synthetic construct
  REGION 1..16: note = AS63964 CDR2
AISGGRFTAYADSVKG

SEQ ID NO: 155 - protein, 17 aa, synthetic construct
  REGION 1..17: note = AS64116 CDR2
GISTGGGGTVYADSVKG

SEQ ID NO: 156 - protein, 17 aa, synthetic construct
  REGION 1..17: note = AS68270 CDR2
AITSGTGSTSYADSVKD

SEQ ID NO: 157 - protein, 17 aa, synthetic construct
  REGION 1..17: note = AS68320 CDR2
AIYRHSGNTAYADSVKG

SEQ ID NO: 158 - protein, 17 aa, synthetic construct
  REGION 1..17: note = AS68351 CDR2
GISASGGRTSYADSVKG

SEQ ID NO: 159 - protein, 17 aa, synthetic construct
  REGION 1..17: note = AS75378 CDR2
IVYTGGRDTYYAASVKG

SEQ ID NO: 160 - protein, 16 aa, synthetic construct
  REGION 1..16: note = AS75383 CDR2
TFNNRGVANYHDSVKG

SEQ ID NO: 161 - protein, 16 aa, synthetic construct
  REGION 1..16: note = AS75751 CDR2
AIDTDGRTSYADSVKG

SEQ ID NO: 162 - protein, 16 aa, synthetic construct
  REGION 1..16: note = AS76422 CDR2
TYNNFGVANYADSVKG

SEQ ID NO: 163 - protein, 17 aa, synthetic construct
  REGION 1..17: note = AS63930 CDR3
EVADPTWGSRDQRRYKY

SEQ ID NO: 164 - protein, 9 aa, synthetic construct
  REGION 1..9: note = AS63932 CDR3
RYGSGNVNY

SEQ ID NO: 165 - protein, 21 aa, synthetic construct
  REGION 1..21: note = AS63951 CDR3
RSFSIAVCATRSGITRSNFAY

SEQ ID NO: 166 - protein, 20 aa, synthetic construct
  REGION 1..20: note = AS63984 CDR3
GGPVTNAPRWYPLRPPGYNY

SEQ ID NO: 167 - protein, 16 aa, synthetic construct
  REGION 1..16: note = AS63987 CDR3
DRPFVCNIANMRRSSN

SEQ ID NO: 168 - protein, 16 aa, synthetic construct
  REGION 1..16: note = AS63997 CDR3
GYLSGGSWDVPGRYNY

SEQ ID NO: 169 - protein, 16 aa, synthetic construct
  REGION 1..16: note = AS64047 CDR3
GFVSGGRWNQSYRYKY

SEQ ID NO: 170 - protein, 20 aa, synthetic construct
  REGION 1..20: note = AS64052 CDR3
GGPPANADRWYPLRPPGYNY

SEQ ID NO: 171 - protein, 17 aa, synthetic construct
  REGION 1..17: note = AS64062 CDR3
VVPRGGSCRLDERGYYH

SEQ ID NO: 172 - protein, 14 aa, synthetic construct
  REGION 1..14: note = AS64072 CDR3
ETTMGWAHERGYRY

SEQ ID NO: 173 - protein, 18 aa, synthetic construct
  REGION 1..18: note = AS64097 CDR3
GPDLGGSWCRPVERAFTY

SEQ ID NO: 174 - protein, 16 aa, synthetic construct
  REGION 1..16: note = AS64114 CDR3
ASRHRLRLNNPRDYDY

SEQ ID NO: 175 - protein, 7 aa, synthetic construct
  REGION 1..7: note = AS64123 CDR3
RFSEYNY

SEQ ID NO: 176 - protein, 20 aa, synthetic construct
  REGION 1..20: note = AS64130 CDR3
GGPRTNDDRWYPLRPPGYNY

SEQ ID NO: 177 - protein, 17 aa, synthetic construct
  REGION 1..17: note = AS64137 CDR3
AAASTDVRLLDPGDFAY

SEQ ID NO: 178 - protein, 17 aa, synthetic construct
  REGION 1..17: note = AS64142 CDR3
DTTSGYCSGFWSTSRYS

SEQ ID NO: 179 - protein, 17 aa, synthetic construct
  REGION 1..17: note = AS64154 CDR3
SSPRWGGTCRRWSQYNY

SEQ ID NO: 180 - protein, 19 aa, synthetic construct
  REGION 1..19: note = AS64160 CDR3
DFGRDKNYLRPLLPHAYNY

SEQ ID NO: 181 - protein, 9 aa, synthetic construct
  REGION 1..9: note = AS64228 CDR3
LCWRENVNY

SEQ ID NO: 182 - protein, 15 aa, synthetic construct
  REGION 1..15: note = AS64300 CDR3
FNYGGAWYEERSYKY

SEQ ID NO: 183 - protein, 16 aa, synthetic construct
  REGION 1..16: note = AS64380 CDR3
SSRHRLGLNNPRDYDY

SEQ ID NO: 184 - protein, 16 aa, synthetic construct
  REGION 1..16: note = AS64395 CDR3
AASTAVRLLDPGDFAY

SEQ ID NO: 185 - protein, 7 aa, synthetic construct
  REGION 1..7: note = AS64443 CDR3
RFPAVTY

SEQ ID NO: 186 - protein, 16 aa, synthetic construct
  REGION 1..16: note = AS64511 CDR3
NLRIGGDWFDGRDFRA

SEQ ID NO: 187 - protein, 16 aa, synthetic construct
  REGION 1..16: note = AS64536 CDR3
KWGLGGGGLKSDTYMY

SEQ ID NO: 188 - protein, 20 aa, synthetic construct
  REGION 1..20: note = AS64597 CDR3
GGPPTDGDRWYPLRPPGYNY

SEQ ID NO: 189 - protein, 10 aa, synthetic construct
  REGION 1..10: note = AS64617 CDR3
IVGRTCSLNY

SEQ ID NO: 190 - protein, 16 aa, synthetic construct
  REGION 1..16: note = AS64634 CDR3
RRWVSGTCYWDSDFHY

SEQ ID NO: 191 - protein, 16 aa, synthetic construct
  REGION 1..16: note = AS69498 CDR3
SSRHRLRLSDPRDYDY

SEQ ID NO: 192 - protein, 17 aa, synthetic construct
  REGION 1..17: note = AS69500 CDR3
RAFQVGYCYLRTDVYNY

SEQ ID NO: 193 - protein, 16 aa, synthetic construct
  REGION 1..16: note = AS69527 CDR3
RRWACPRVGSWHEFAY

SEQ ID NO: 194 - protein, 16 aa, synthetic construct
  REGION 1..16: note = AS68280 CDR3
ARGRFIDWTKATQYDY

SEQ ID NO: 195 - protein, 20 aa, synthetic construct
  REGION 1..20: note = AS68355 CDR3
AIVGGFNAYCSGGYVLDFGA

SEQ ID NO: 196 - protein, 24 aa, synthetic construct
  REGION 1..24: note = AS69443 CDR3
DFLTGFYYSDSPHPAPCSASDFGY

SEQ ID NO: 197 - protein, 17 aa, synthetic construct
  REGION 1..17: note = AS75376 CDR3
DPNPDYMLPFRPSRRSW

SEQ ID NO: 198 - protein, 14 aa, synthetic construct
  REGION 1..14: note = AS75387 CDR3
DLRLPRAGGCAYSY

SEQ ID NO: 199 - protein, 13 aa, synthetic construct
  REGION 1..13: note = AS75695 CDR3
DRRGRVRRCEYNA

SEQ ID NO: 200 - protein, 17 aa, synthetic construct
  REGION 1..17: note = AS76169 CDR3
DRGGYANSCAVAARYDY

SEQ ID NO: 201 - protein, 16 aa, synthetic construct
  REGION 1..16: note = AS63931 CDR3
GWLSGGSWHVPGRYNY

SEQ ID NO: 202 - protein, 14 aa, synthetic construct
  REGION 1..14: note = AS63937 CDR3
DSRRPCMVAAGYTY

SEQ ID NO: 203 - protein, 17 aa, synthetic construct
  REGION 1..17: note = AS63948 CDR3
ASPRWGGTCRRWSEYNY

SEQ ID NO: 204 - protein, 15 aa, synthetic construct
  REGION 1..15: note = AS63956 CDR3
YQAKYCSGPCAPPTD

SEQ ID NO: 205 - protein, 13 aa, synthetic construct
  REGION 1..13: note = AS63965 CDR3
WCREYPGGILNNG

SEQ ID NO: 206 - protein, 10 aa, synthetic construct
  REGION 1..10: note = AS63993 CDR3
RTDSEHAFKF

SEQ ID NO: 207 - protein, 7 aa, synthetic construct
  REGION 1..7: note = AS63999 CDR3
RYTDYNY

SEQ ID NO: 208 - protein, 7 aa, synthetic construct
  REGION 1..7: note = AS64006 CDR3
RSRTMMY

SEQ ID NO: 209 - protein, 17 aa, synthetic construct
  REGION 1..17: note = AS64057 CDR3
DTTSGSCSGFWSTSRYY

SEQ ID NO: 210 - protein, 14 aa, synthetic construct
  REGION 1..14: note = AS64060 CDR3
DPTIGCPQTYRYNY

SEQ ID NO: 211 - protein, 16 aa, synthetic construct
  REGION 1..16: note = AS64071 CDR3
STKDQFYVFNPIGYDS

SEQ ID NO: 212 - protein, 21 aa, synthetic construct
  REGION 1..21: note = AS64093 CDR3
RSFSIGVCATQSGITWSNFAY

SEQ ID NO: 213 - protein, 10 aa, synthetic construct
  REGION 1..10: note = AS64118 CDR3
TWDSSCRFQY

SEQ ID NO: 214 - protein, 21 aa, synthetic construct
  REGION 1..21: note = AS64120 CDR3
RSFSIADCATQSGITRSNFAY

SEQ ID NO: 215 - protein, 19 aa, synthetic construct
  REGION 1..19: note = AS64124 CDR3
GRPYSLPLPLPLESGAYRY

SEQ ID NO: 216 - protein, 13 aa, synthetic construct
  REGION 1..13: note = AS64135 CDR3
DPMGGSRTPCTSA

SEQ ID NO: 217 - protein, 17 aa, synthetic construct
  REGION 1..17: note = AS64163 CDR3
DPSVCPGGMWYSKEYRY

SEQ ID NO: 218 - protein, 17 aa, synthetic construct
  REGION 1..17: note = AS64182 CDR3
SSPHWGGTCRRWSEYKY

SEQ ID NO: 219 - protein, 13 aa, synthetic construct
  REGION 1..13: note = AS64183 CDR3
DLSGGPAGCGYTH

SEQ ID NO: 220 - protein, 20 aa, synthetic construct
  REGION 1..20: note = AS64207 CDR3
GGPPTNGAKWYPLRPPGYNY

SEQ ID NO: 221 - protein, 13 aa, synthetic construct
  REGION 1..13: note = AS64276 CDR3
SLGPGRGACGYNY

SEQ ID NO: 222 - protein, 13 aa, synthetic construct
  REGION 1..13: note = AS64336 CDR3
RTGWAPRCAVPGY

SEQ ID NO: 223 - protein, 18 aa, synthetic construct
  REGION 1..18: note = AS64346 CDR3
VQSKSSNYVLRDASTYNY

SEQ ID NO: 224 - protein, 7 aa, synthetic construct
  REGION 1..7: note = AS64420 CDR3
RSRYATY

SEQ ID NO: 225 - protein, 17 aa, synthetic construct
  REGION 1..17: note = AS64473 CDR3
SSPQWGGTCRRWSEYNY

SEQ ID NO: 226 - protein, 7 aa, synthetic construct
  REGION 1..7: note = AS64475 CDR3
RDTARTY

SEQ ID NO: 227 - protein, 18 aa, synthetic construct
  REGION 1..18: note = AS64513 CDR3
GYYSGSGPGYLLPWRYNY

SEQ ID NO: 228 - protein, 22 aa, synthetic construct
  REGION 1..22: note = AS64562 CDR3
GFRPRGGGYTGDVLAQAAAYNY

SEQ ID NO: 229 - protein, 17 aa, synthetic construct
  REGION 1..17: note = AS64583 CDR3
KAHPDCSGDWSPSGYEY

SEQ ID NO: 230 - protein, 7 aa, synthetic construct
  REGION 1..7: note = AS64594 CDR3
RTQTRNY

SEQ ID NO: 231 - protein, 22 aa, synthetic construct
  REGION 1..22: note = AS64605 CDR3
RSNSYSYSSCDYGPLTRGGYNF

SEQ ID NO: 232 - protein, 7 aa, synthetic construct
  REGION 1..7: note = AS64606 CDR3
RLDEKMY

SEQ ID NO: 233 - protein, 21 aa, synthetic construct
  REGION 1..21: note = AS68121 CDR3
DPGSRTDDSCGTSYNKGNFGY

SEQ ID NO: 234 - protein, 20 aa, synthetic construct
  REGION 1..20: note = AS68170 CDR3
ASDVAVGVNSCGGRTAGFDA

SEQ ID NO: 235 - protein, 17 aa, synthetic construct
  REGION 1..17: note = AS63964 CDR3
EVVDPTWGSRDQRRYKY

SEQ ID NO: 236 - protein, 17 aa, synthetic construct
  REGION 1..17: note = AS64116 CDR3
DRWNSFANCGAWGRYTY

SEQ ID NO: 237 - protein, 18 aa, synthetic construct
  REGION 1..18: note = AS68270 CDR3
ASGWIVPSRSLTANLYRY

SEQ ID NO: 238 - protein, 16 aa, synthetic construct
  REGION 1..16: note = AS68320 CDR3
GRAGPWALMRPTEFGY

SEQ ID NO: 239 - protein, 14 aa, synthetic construct
  REGION 1..14: note = AS68351 CDR3
GAVRLSTSSVRDSS

SEQ ID NO: 240 - protein, 7 aa, synthetic construct
  REGION 1..7: note = AS75378 CDR3
RSYEYTY

SEQ ID NO: 241 - protein, 16 aa, synthetic construct
  REGION 1..16: note = AS75383 CDR3
DRRYGRQWYQPCEWNT

SEQ ID NO: 242 - protein, 20 aa, synthetic construct
  REGION 1..20: note = AS75751 CDR3
GFGYMNVIQALNGMRQNPDY

SEQ ID NO: 243 - protein, 16 aa, synthetic construct
  REGION 1..16: note = AS76422 CDR3
DRRDGRRWSQPCEWNT

SEQ ID NO: 244 - protein, 10 aa, synthetic construct
  REGION 1..10: note = AS64380VH4 CDR1
GNTYSSNYMG

SEQ ID NO: 245 - protein, 10 aa, synthetic construct
  REGION 1..10: note = AS64380VH5 CDR1
GNTYSSNYMG

SEQ ID NO: 246 - protein, 10 aa, synthetic construct
  REGION 1..10: note = AS64380VH6 CDR1
GNTYSSNYMG

SEQ ID NO: 247 - protein, 10 aa, synthetic construct
  REGION 1..10: note = AS64380VH7 CDR1
GNTYSSNYMG

SEQ ID NO: 248 - protein, 10 aa, synthetic construct
  REGION 1..10: note = AS64511VH4 CDR1
RATYSTNYIS

SEQ ID NO: 249 - protein, 10 aa, synthetic construct
  REGION 1..10: note = AS64511VH5 CDR1
RATYSTNYIS

SEQ ID NO: 250 - protein, 10 aa, synthetic construct
  REGION 1..10: note = AS64511VH6 CDR1
RATYSTNYIS

SEQ ID NO: 251 - protein, 10 aa, synthetic construct
  REGION 1..10: note = AS63997VH4 CDR1
FSGYGVSTMA

SEQ ID NO: 252 - protein, 10 aa, synthetic construct
  REGION 1..10: note = AS63997VH5 CDR1
FSGYGVSTMA

SEQ ID NO: 253 - protein, 10 aa, synthetic construct
  REGION 1..10: note = AS63997VH6 CDR1
FSGYGVSTMA

SEQ ID NO: 254 - protein, 17 aa, synthetic construct
  REGION 1..17: note = AS64380VH4 CDR2
VIYTRGGHTYYVDSVRG

SEQ ID NO: 255 - protein, 17 aa, synthetic construct
  REGION 1..17: note = AS64380VH5 CDR2
VIYTRGGHTYYVDSVRG

SEQ ID NO: 256 - protein, 17 aa, synthetic construct
  REGION 1..17: note = AS64380VH6 CDR2
VIYTRGGHTYYVDSVRG

SEQ ID NO: 257 - protein, 17 aa, synthetic construct
  REGION 1..17: note = AS64380VH7 CDR2
VIYTRGGHTYYVDSVRG

SEQ ID NO: 258 - protein, 17 aa, synthetic construct
  REGION 1..17: note = AS64511VH4 CDR2
TITTGDGETAYADSVKG

SEQ ID NO: 259 - protein, 17 aa, synthetic construct
  REGION 1..17: note = AS64511VH5 CDR2
TITTGDGETAYADSVKG

SEQ ID NO: 260 - protein, 17 aa, synthetic construct
  REGION 1..17: note = AS64511VH6 CDR2
TITTGDGETAYADSVKG

SEQ ID NO: 261 - protein, 17 aa, synthetic construct
  REGION 1..17: note = AS63997VH4 CDR2
AITVGSGNTYYADSVKG

SEQ ID NO: 262 - protein, 17 aa, synthetic construct
  REGION 1..17: note = AS63997VH5 CDR2
AITVGSGNTYYADSVKG

SEQ ID NO: 263 - protein, 17 aa, synthetic construct
  REGION 1..17: note = AS63997VH6 CDR2
AITVGSGNTYYADSVKG

SEQ ID NO: 264 - protein, 16 aa, synthetic construct
  REGION 1..16: note = AS64380VH4 CDR3
SSRHRLGLNNPRDYDY

SEQ ID NO: 265 - protein, 16 aa, synthetic construct
  REGION 1..16: note = AS64380VH5 CDR3
SSRHRLGLNNPRDYDY

SEQ ID NO: 266 - protein, 16 aa, synthetic construct
  REGION 1..16: note = AS64380VH6 CDR3
SSRHRLGLNNPRDYDY

SEQ ID NO: 267 - protein, 16 aa, synthetic construct
  REGION 1..16: note = AS64380VH7 CDR3
SSRHRLGLNNPRDYDY

SEQ ID NO: 268 - protein, 16 aa, synthetic construct
  REGION 1..16: note = AS64511VH4 CDR3
NLRIGGDWFDGRDFRA

SEQ ID NO: 269 - protein, 16 aa, synthetic construct
  REGION 1..16: note = AS64511VH5 CDR3
NLRIGGDWFDGRDFRA

SEQ ID NO: 270 - protein, 16 aa, synthetic construct
  REGION 1..16: note = AS64511VH6 CDR3
NLRIGGDWFDGRDFRA

SEQ ID NO: 271 - protein, 16 aa, synthetic construct
  REGION 1..16: note = AS63997VH4 CDR3
GYLSGGSWDVPGRYNY

SEQ ID NO: 272 - protein, 16 aa, synthetic construct
  REGION 1..16: note = AS63997VH5 CDR3
GYLSGGSWDVPGRYNY

SEQ ID NO: 273 - protein, 16 aa, synthetic construct
  REGION 1..16: note = AS63997VH6 CDR3
GYLSGGSWDVPGRYNY

SEQ ID NO: 274 - protein, 129 aa, synthetic construct
  REGION 1..129: note = Anti-DLL3 camel sdAb amino acid sequence AS63930
EVQLAESGGGSVQAGGSLRLSCAASGYTYSGNYMAWFRQAPGNEREGVAVVYNIDGGGRFTTYADSVKGRFTISRGNDKNTVYLQMNSLKPEDSGMYYCAAEVADPTWGSRDQRRYKYWGQGTQVTVSS

SEQ ID NO: 275 - protein, 118 aa, synthetic construct
  REGION 1..118: note = Anti-DLL3 camel sdAb amino acid sequence AS63932
QVQLEESGGGSVQAGGSLRLSCVASGYTYGSTFMGWFRQNPGKEREGVAVIYTGGGSTWYASSVKGRFTISQDNAKNTVYLQMNSLKPEDTAMYYCAARYGSGNVNYWGQGTQVTVSS

SEQ ID NO: 276 - protein, 130 aa, synthetic construct
  REGION 1..130: note = Anti-DLL3 camel sdAb amino acid sequence AS63951
QVHLMESGGGSVQAGGSLRLACETSRDIYGNNCMAWFRQAPGKEREGVASIYPAGGRPYYADSVKGRFTISQDNAKNTVYLQMDSLKPEDTAMYYCAARSFSIAVCATRSGITRSNFAYWGQGTQVTVSS

SEQ ID NO: 277 - protein, 129 aa, synthetic construct
  REGION 1..129: note = Anti-DLL3 camel sdAb amino acid sequence AS63984
QVKLVESGGGSVQAGGSLRLSCAASGYTYSSNFMGWFRQAPGKEREGVATIVSGGGTTYYADSVRGRFTISQDNAKNTVYLQMNSLKPEDTAMYYCAAGGPVTNAPRWYPLRPPGYNYWGQGTQVTVSS

SEQ ID NO: 278 - protein, 122 aa, synthetic construct
  REGION 1..122: note = Anti-DLL3 camel sdAb amino acid sequence AS63987
QVRLVESGGGSVQAGGSLRLSCEATGYRNCMAWFRQAPGKEREGVAVIYTPSGITDYASSVKGRFTISQNNARNTQYLQMNSLKPEDTAMYYCAADRPFVCNIANMRRSSNWGRGTQVTVSS

SEQ ID NO: 279 - protein, 125 aa, synthetic construct
  REGION 1..125: note = Anti-DLL3 camel sdAb amino acid sequence AS63997
QVRLVESGGGSVQAGGSLRLSCAGSFSGYGVSTMAWFRQAPGKEREGVAAITVGSGNTYYADSVKGRFTISRDNAKRTVFLQMNSLKPEDTAMYYCAVGYLSGGSWDVPGRYNYWGQGTQVTVSS

SEQ ID NO: 280 - protein, 125 aa, synthetic construct
  REGION 1..125: note = Anti-DLL3 camel sdAb amino acid sequence AS64047
QVHLVESGGGSVQAGGSLRLSCAASQYVYRWDLMGWFRQAPGKEREAVAAVYTGDGITYYADSVKGRFSISQDNAKNTVYLQMNSLKPEDTGMYFCAAGFVSGGRWNQSYRYKYWGQGTQVTVSS

SEQ ID NO: 281 - protein, 129 aa, synthetic construct
  REGION 1..129: note = Anti-DLL3 camel sdAb amino acid sequence AS64052
QVHLMESGGGSVQAGGSLRLSCAASGYTYRSNFMGWFRQAPGKEREGIATIHSGVATTYYADSVKGRFTISQDNAKNTVYLQMNSLKPEDTAMYYCAAGGPPANADRWYPLRPPGYNYWGQGTQVTVSS

SEQ ID NO: 282 - protein, 127 aa, synthetic construct
  REGION 1..127: note = Anti-DLL3 camel sdAb amino acid sequence AS64062
QVRLVESGGGSVQVGGSLRLSCAASRSPYSSSRCMGWFRQAPGKEREGVAALYTGGGSTSYADSVKGRFTISQDNAKNTVYLQMNSLKPEDTAMYYCAAVVPRGGSCRLDERGYYHWGQGTQVTVSS

SEQ ID NO: 283 - protein, 123 aa, synthetic construct
  REGION 1..123: note = Anti-DLL3 camel sdAb amino acid sequence AS64072
QVQLVESGGGSVQAGGFLRLSCALSGYSYYINLMAWFRQAPGKEREAVAAHGPVSGTAYYTDSVKGRFTISRDPGKNTMYLQMFSLQPEDTALYYCAAETTMGWAHERGYRYWGQGTQVTVSS

SEQ ID NO: 284 - protein, 127 aa, synthetic construct
  REGION 1..127: note = Anti-DLL3 camel sdAb amino acid sequence AS64097
QVHLMESGGGSVQAGGSLRLSCEASGYTYSRNCMGWFRQAPGKEREGVAAINTGGGSTYYADSLEGRFTISQDNAKNTMYLQMNSLKPEDTAMYYCAAGPDLGGSWCRPVERAFTYWGQGTQVTVSS

SEQ ID NO: 285 - protein, 125 aa, synthetic construct
  REGION 1..125: note = Anti-DLL3 camel sdAb amino acid sequence AS64114
QVQLQESGGGSVQAGGSLTLSCEASGNTYSTNYMGWFRQAPGKEREEVAVIYTRGGHTYYVDSVRGRFTISQDNAKNTVYLQMNSLKPEDTAMYYCAAASRHRLRLNNPRDYDYWGQGTQVTVSS

SEQ ID NO: 286 - protein, 116 aa, synthetic construct
  REGION 1..116: note = Anti-DLL3 camel sdAb amino acid sequence AS64123
QVQLAESGGGSVQAGGSLRLSCAASGYTYTSNWLGWFRQAPGKEREEVAIIYTGSGSTHYRSSVKGRFTISQDNAKNTVYLQMNSLKPEDTAMYYCAARFSEYNYWGQGTQVTVSS

SEQ ID NO: 287 - protein, 129 aa, synthetic construct
  REGION 1..129: note = Anti-DLL3 camel sdAb amino acid sequence AS64130
EVQLAESGGGSVQAGGSLRLSCAASGYTYRSNFMGWFRQAPAKEREGVATIDSRGTITYYADSVKGRFTISQDNEKNTVYLQMNSLKPEDTAMYYCAAGGPRTNDDRWYPLRPPGYNYWGQGTQVTVSS

SEQ ID NO: 288 - protein, 126 aa, synthetic construct
  REGION 1..126: note = Anti-DLL3 camel sdAb amino acid sequence AS64137
QVRLVESGGGSVQAGGSLRLSCAASGSTYSTNFMGWFRQAPGKEREGVATLVTWVERTAYADSVKGRFTISQDRAKNTVYLQMNSLKPEDTAMYYCAAAAASTDVRLLDPGDFAYWGQGTQVTVSS

SEQ ID NO: 289 - protein, 126 aa, synthetic construct
  REGION 1..126: note = Anti-DLL3 camel sdAb amino acid sequence AS64142
QVHLMESGGGLVQTGGSLRLSCTASGFTFDRNAMRWYRQAPGKEREGVSCIDWTGANIAYADSVKGRFTISRDNAKNTLYLQMNSLKPEDTGMYYCAADTTSGYCSGFWSTSRYSWGQGTQVTVSS

SEQ ID NO: 290 - protein, 126 aa, synthetic construct
  REGION 1..126: note = Anti-DLL3 camel sdAb amino acid sequence AS64154
QVQLKESGGGSVQAGGSLRLSCTASGYTYRYLYMGWFRQTPGKEREGVACIYTGSGSTGYADSVKGRFTISQDNAKNTVYLQMNNLKPEDTAMYYCAASSPRWGGTCRRWSQYNYWGQGTQVTVSS

SEQ ID NO: 291 - protein, 128 aa, synthetic construct
  REGION 1..128: note = Anti-DLL3 camel sdAb amino acid sequence AS64160
EVQLVESGGGSVQAGGSLRLSCAASVYTSSSYCMGWFRQAPGKEREGVAAMCFGGLVTHYADSVKGRFTISQDNAKNTVYLQMNSLKPEDTAMYYCAADFGRDKNYLRPLLPHAYNYWGQGTQVTVSS

SEQ ID NO: 292 - protein, 117 aa, synthetic construct
  REGION 1..117: note = Anti-DLL3 camel sdAb amino acid sequence AS64228
QVQLKESGGGSIQAGGSLRLSCAASGVSYNRCSMGWYRQAPGKGRELVSRIQPGGNTYYADSVKGRFTVSQDNAKNTVSLQMNSLKPEDTAMYYCNALCWRENVNYWGQGTQVTVSS

SEQ ID NO: 293 - protein, 124 aa, synthetic construct
  REGION 1..124: note = Anti-DLL3 camel sdAb amino acid sequence AS64300
QVHLVESGGGSVQTGGSLRLSCAVSGDIYNLMSMAWFRRAPGKEREGVAYINTIIGNTYYTDSVKGRFTISRDNSKNTLYLQMNNLKPEDTAMYYCAAFNYGGAWYEERSYKYWGQGTQVTVSS

SEQ ID NO: 294 - protein, 125 aa, synthetic construct
  REGION 1..125: note = Anti-DLL3 camel sdAb amino acid sequence AS64380
EVQLVESGGGSVQAGGSLTLSCEASGNTYSSNYMGWFRQAPGKEREEVAVIYTRGGHTYYVDSVRGRFTISQDNAKNTVYLQMNSLKPEDTAMYYCAASSRHRLGLNNPRDYDYWGQGTQVTVSS

SEQ ID NO: 295 - protein, 125 aa, synthetic construct
  REGION 1..125: note = Anti-DLL3 camel sdAb amino acid sequence AS64395
QVRLVESGGGSVQAGGSLRLSCAASGSTYSTNFMGWFRQAPGKEREGVATLVTWAERTAYADSVKGRFTISQDRAKNTVYLQMNGLKPEDTAMYYCAAAASTAVRLLDPGDFAYWGQGTQVTVSS

SEQ ID NO: 296 - protein, 116 aa, synthetic construct
  REGION 1..116: note = Anti-DLL3 camel sdAb amino acid sequence AS64443
QVHLVESGGGSVQAGGSLRLSCAASGYTDSSVYIGWFRQAPGKEREEVAIIYTGGESTHYRSSVKGRFTVSQDNAKNTLYLQMNSLKPEDTAMYYCAARFPAVTYWGQGTQVTVSS

SEQ ID NO: 297 - protein, 125 aa, synthetic construct
  REGION 1..125: note = Anti-DLL3 camel sdAb amino acid sequence AS64511
QVQLVESGGGSVQAGGSLRLSCAASRATYSTNYISWFRQAPGKEREAVATITTGDGETAYADSVKGRFTISRDNAKNTVYLQMNSLKPEDTAMYYCAANLRIGGDWFDGRDFRAWGQGTQVTVSS

SEQ ID NO: 298 - protein, 125 aa, synthetic construct
  REGION 1..125: note = Anti-DLL3 camel sdAb amino acid sequence AS64536
QVKLVESGGGSVQAGGSLRLSCAASRYTDNFVYMGWFRQAPGKEREGVALIYPGGGSTYYASSVKGRFTISQDNAKGTVHLQMNNLKPEDTAMYYCAAKWGLGGGGLKSDTYMYWGQGTQVTVSS

SEQ ID NO: 299 - protein, 129 aa, synthetic construct
  REGION 1..129: note = Anti-DLL3 camel sdAb amino acid sequence AS64597
QVHLVESGGGSVQAGGSLRLSCAASGYTYRVNFMGWFRQTPGKEREGVATIDSGVGTTYYADSVKGRFTISHNNAKNTIYLQMNSLKPEDTAMYYCAAGGPPTDGDRWYPLRPPGYNYWGQGTQVTVSS

SEQ ID NO: 300 - protein, 117 aa, synthetic construct
  REGION 1..117: note = Anti-DLL3 camel sdAb amino acid sequence AS64617
QVQLVESGGGSVQAGGSLRLSCAASGYTDRCSMAWYRQAPGKERELVSRISTSGFTNYAASVKGRFTISQDNAKNTVYLQMNSLNPGDTGMYYCAIIVGRTCSLNYWGNGILVTVSS

SEQ ID NO: 301 - protein, 127 aa, synthetic construct
  REGION 1..127: note = Anti-DLL3 camel sdAb amino acid sequence AS64634
QVRLVESGGGSVQAGGSLRLSCAASGYSFRGDFMCMGWFRQTPGKGREGVAVFYPGGGSTNYADSAKGRFTISQDNAKNTMYLQMNTLKPEDTAMYYCAARRWVSGTCYWDSDFHYWGQGTQVTVSS

SEQ ID NO: 302 - protein, 125 aa, synthetic construct
  REGION 1..125: note = Anti-DLL3 camel sdAb amino acid sequence AS69498
QVQLQESGGGSVQAGGSLRLSCEASGNTYSSNYMAWFRQAPGKEREEVAVIYTRGGHTYYIDSVRGRFTISQDNAKNTVYLQMNSLKPEDTAMYYCAASSRHRLRLSDPRDYDYWGQGTQVTVSS

SEQ ID NO: 303 - protein, 127 aa, synthetic construct
  REGION 1..127: note = Anti-DLL3 camel sdAb amino acid sequence AS69500
QVRLVESGGGSVQAGGSLRLSCAADRYTYSSACMGWFRQAPGKEREGVASIFTGTGGSTYYADSVKGRFTISQDNAKNTVYLQMNSLKPEDTAIYYCAARAFQVGYCYLRTDVYNYWGQGTQVTVSS

SEQ ID NO: 304 - protein, 125 aa, synthetic construct
  REGION 1..125: note = Anti-DLL3 camel sdAb amino acid sequence AS69527
EVQLAESGGGSVQAGGSLRLSCVASRYTFSSTCMAWFRQAPGKEREEVAAIYTDDGSTWYADSVKGRFTISRDNAKNTVYLQMNSLKPEDTAMYYCAARRWACPRVGSWHEFAYWGQGTQVTVSS

SEQ ID NO: 305 - protein, 125 aa, synthetic construct
  REGION 1..125: note = Anti-DLL3 camel sdAb amino acid sequence AS68280
QVQLVESGGGSVHPGGSLRLSCAASGSTYSSNYLGWFRQAPGKGRDWVAAISTGDGATAYADSVKGRFTISQDNAKNTVYLQMNSLKLEDSAMYYCAAARGRFIDWTKATQYDYWGQGTQVTVSS

SEQ ID NO: 306 - protein, 128 aa, synthetic construct
  REGION 1..128: note = Anti-DLL3 camel sdAb amino acid sequence AS68355
QMQLVESGGDSVQAGGSLRLSCAASGYTYSGVCMGWFRQAPGKEREGVAAIDSDGSTSYADSVKGRFTISKDNAKNTLYLQMNSLKPEDTAMYYCAAAIVGGFNAYCSGGYVLDFGAWGQGTQVTVSS

SEQ ID NO: 307 - protein, 132 aa, synthetic construct
  REGION 1..132: note = Anti-DLL3 camel sdAb amino acid sequence AS69443
EVQLAESGGGSVQAGGSLRLSCSASGFTFDDSDMAWYRQAPGDGCDLVSTISSDGSTYYADSVKGRFTISQDNAKNTVYLQMHSLKPEDTAVYYCAADFLTGFYYSDSPHPAPCSASDFGYWGQGTQVTVSS

SEQ ID NO: 308 - protein, 126 aa, synthetic construct
  REGION 1..126: note = Anti-DLL3 camel sdAb amino acid sequence AS75376
QVQLKESGGGSVQAGGSLRLSCAASGYTYSSHSMGWFRQAPGKEREGVAVIYTGDGSTYYADSVKGRFTISQDNAKNTVYLQMNSLKPEDTAMYYCAADPNPDYMLPFRPSRRSWWGQGTQVTVSS

SEQ ID NO: 309 - protein, 123 aa, synthetic construct
  REGION 1..123: note = Anti-DLL3 camel sdAb amino acid sequence AS75387
QVHLVESGGGSVQAGGSLRLSCAASGYPYSSPCMAWFRQAPGKEREGVLVAYTGGDIQYLTDSVKGRFTISRDNAKNTVYLQMNSLKPEDTAMYYCAADLRLPRAGGCAYSYWGQGTQVTVSS

SEQ ID NO: 310 - protein, 121 aa, synthetic construct
  REGION 1..121: note = Anti-DLL3 camel sdAb amino acid sequence AS75695
QVRLVESGGGSVQAGGSLRLSCVASGYTVSAYCMGWFRQVLGKGRERIAFIDAGGATIYADPVKGRFTISKDNAKNTLYLQMNSLKPEDTAMYYCVADRRGRVRRCEYNAWGQGTQVTVSS

SEQ ID NO: 311 - protein, 125 aa, synthetic construct
  REGION 1..125: note = Anti-DLL3 camel sdAb amino acid sequence AS76169
QVHLMESGGGSVQAGGSLRLSCAASGYIYSSFCMGWFRQAPGKEREVVAYIRDNIMTSYTDSVKGRFTISKDNAKRTLYLQMNGLKPEDTGMYYCAVDRGGYANSCAVAARYDYWGRGTQVTVSS

SEQ ID NO: 312 - protein, 125 aa, synthetic construct
  REGION 1..125: note = Anti-DLL3 camel sdAb amino acid sequence AS63931
EVQLAESGGGSVQAGGSLRLSCAGSFSGYGVSTMAWFRQAPGKEREGVAAITVGSGNTYYADSVTGRFTISRDNAKRTVYLQMNSLKPEDTAMYYCAAGWLSGGSWHVPGRYNYWGQGTQVTVSS

SEQ ID NO: 313 - protein, 123 aa, synthetic construct
  REGION 1..123: note = Anti-DLL3 camel sdAb amino acid sequence AS63937
QVKLVESGGGSVQAGGSLRLSCAASGSTISSRPMAWFRQAPGKEREGVACIHTGLGRTYYADSVKGRFTISQDNAKNTVYLQVNSLKPEDTAMYYCAADSRRPCMVAAGYTYWGQGTQVTVSS

SEQ ID NO: 314 - protein, 126 aa, synthetic construct
  REGION 1..126: note = Anti-DLL3 camel sdAb amino acid sequence AS63948
QVQLVESGGGSVQAGGSLRLSCTASGYTYRYLYMGWFRQTPGKEREGVACIYTGSGSTGYADSVKGRFTISQDNAENTVYLQMNSLKPEDTAMYYCAAASPRWGGTCRRWSEYNYWGQGTQVTVSS

SEQ ID NO: 315 - protein, 123 aa, synthetic construct
  REGION 1..123: note = Anti-DLL3 camel sdAb amino acid sequence AS63956
QVHLVESGGGLVQAGGSLRLSCAASGFTYSNCCMRWYRQAPGKARELVSLINSSGGTYYADSVRGRFTISKDNAKNTLYLQMNSLKPEDTAMYYCAAYQAKYCSGPCAPPTDWGQGTQVTVSS

SEQ ID NO: 316 - protein, 121 aa, synthetic construct
  REGION 1..121: note = Anti-DLL3 camel sdAb amino acid sequence AS63965
QVQLVESGGGSVQAGGSLRLSCVASGYSSGSCRMGWYRQAPGKERELVSKVISDGTTVYADSVKGRFTLSQGNAKNTVYLQMSSLLPEDTAMYYCNAWCREYPGGILNNGWGQGTQVTVSS

SEQ ID NO: 317 - protein, 118 aa, synthetic construct
  REGION 1..118: note = Anti-DLL3 camel sdAb amino acid sequence AS63993
QVKLVESGGGLVQAGGSLRLSCTVSGFTFDDLVMAWFRQAPGKERQLVSLVATAGNSVYADSVKGRFTLSRDNAHSTAYLQMNGLKPEDTAMYYCAARTDSEHAFKFWGQGTQVTVSS

SEQ ID NO: 318 - protein, 116 aa, synthetic construct
  REGION 1..116: note = Anti-DLL3 camel sdAb amino acid sequence AS63999
EVQLVESGGGSVQAGGSLRLSCAASGYTYSSNWMGWFRQAPGKEREEVAIIYTGGISTHYRSSVKGRFTISQDNAKNTVYLQMNSLKPEDTAMYYCAARYTDYNYWGQGTQVTVSS

SEQ ID NO: 319 - protein, 116 aa, synthetic construct
  REGION 1..116: note = Anti-DLL3 camel sdAb amino acid sequence AS64006
QVHLVESGGGSVQAGGSLRLSCEVSGYTGDTTYIGWFRQAPGKEREGVALIYTSGTSEYYADSVKGRFIISRDNAKNTVYLQMNSLKPEDTAMYYCGARSRTMMYWGQGTQVTVSS

SEQ ID NO: 320 - protein, 126 aa, synthetic construct
  REGION 1..126: note = Anti-DLL3 camel sdAb amino acid sequence AS64057
QVQLEESGGGLVQTGGSLRLSCTASGFTFDRNAMRWYRQAPGKEREGVSCISWTGANIAYADSVKGRFTISRDNAKNTLYLQMNSLKPEDTGMYYCAADTTSGSCSGFWSTSRYYWGQGTQVTVSS

SEQ ID NO: 321 - protein, 122 aa, synthetic construct
  REGION 1..122: note = Anti-DLL3 camel sdAb amino acid sequence AS64060
QVKLVESGGGSVQAGGSLRLSCAASGSTYCTYRMSWFRQAPGKEREFVAVIDSGGSTSYADSVKGRFTISRDNAKNTVYLQMNSLKPEDTAMYYCKTDPTIGCPQTYRYNYWGQGTQVTVSS

SEQ ID NO: 322 - protein, 125 aa, synthetic construct
  REGION 1..125: note = Anti-DLL3 camel sdAb amino acid sequence AS64071
QVHLMESGGGSVQAGGSLRLSCVASGNTYRLNSMGWFRQAPGKEREGVAFIVMIRGTTYYGASVKGRFTISQDNAQTTVYLQMSSLKPEDTAMYYCAASTKDQFYVFNPIGYDSWGQGTQVTVSS

SEQ ID NO: 323 - protein, 130 aa, synthetic construct
  REGION 1..130: note = Anti-DLL3 camel sdAb amino acid sequence AS64093
QVHLVESGGGSVQAGGSLRLSCATSRYIYGNNCMAWFRQAPGKEREGVASIYPAGGRTYYADSVKGRFTISQDNAKNTVYLQIDSLKPEDTAMYYCAARSFSIGVCATQSGITWSNFAYWGQGTQVTVSS

SEQ ID NO: 324 - protein, 117 aa, synthetic construct
  REGION 1..117: note = Anti-DLL3 camel sdAb amino acid sequence AS64118
QVQLAESGGGSVQAGGSLRLSCAASGYTYSACRMAWYRQAPGKERELVSFINSAGSTYYADSVKGRFAISRDNAKTTVYLQMNALKAEDTAIYYCNTWDSSCRFQYWGQGTQVTVSS

SEQ ID NO: 325 - protein, 130 aa, synthetic construct
  REGION 1..130: note = Anti-DLL3 camel sdAb amino acid sequence AS64120
QVRLVESGGGSVQAGGSLRLSCETSRYIYGNNCMAWFRQAPGKEREGVASIYPAGGRPYYADSVKGRFTISQDNAKNTVYLQMDSLKPEDTAMYYCAARSFSIADCATQSGITRSNFAYWGQGTQVTVSS

SEQ ID NO: 326 - protein, 128 aa, synthetic construct
  REGION 1..128: note = Anti-DLL3 camel sdAb amino acid sequence AS64124
QVKLVESGGGSVQTGGSLRLSCAVSTYTPSNNYMGWFRQAPGKEREGVAAIATIGGTTRYADSVKGRFTISQDGAKNTIYLQMNGLKPEDTAMYYCAAGRPYSLPLPLPLESGAYRYWGQGTQVTVSS

SEQ ID NO: 327 - protein, 121 aa, synthetic construct
  REGION 1..121: note = Anti-DLL3 camel sdAb amino acid sequence AS64135
QVKLVESGGGSVQAGGSLRLSCVASTSTYCRYYMRWYRQAPGKEREFVSAMQPDGTTSYSDSVKGRFTMSQDRANNMLYLQMNSLRPEDTAMYYCKRDPMGGSRTPCTSAWGQGTQVTVSS

SEQ ID NO: 328 - protein, 125 aa, synthetic construct
  REGION 1..125: note = Anti-DLL3 camel sdAb amino acid sequence AS64163
QVRLVESGGGSVQAGGSLRLSCAVSGYRYRWNCMAWFRQAPGKEREGVAAISTGSGSTYYAGSVKGRFTISQDNAKNMYLQMNSLKPEDTAMYYCAADPSVCPGGMWYSKEYRYWGQGTQVTVSS

SEQ ID NO: 329 - protein, 126 aa, synthetic construct
  REGION 1..126: note = Anti-DLL3 camel sdAb amino acid sequence AS64182
QVHLMESGGGSVQAGGSLRLSCTASGQTSRYLYMGWFRQTPGKEREGVACIYTGSGSTGYADSVKGRFTISQDNAKNTVYLQTNSLKPEDTAMYYCAASSPHWGGTCRRWSEYKYWGQGTQVTVSS

SEQ ID NO: 330 - protein, 122 aa, synthetic construct
  REGION 1..122: note = Anti-DLL3 camel sdAb amino acid sequence AS64183
QVHLVESGGGSVQAGGSLRLSCAASGHTYSANCMAWFRRAPGKEREWVASVYTDDDSTMYTDSVKGRFTIFQDNAKNTVYLQMNSLKPEDTGMYICAADLSGGPAGCGYTHWGQGTQVTVSS

SEQ ID NO: 331 - protein, 129 aa, synthetic construct
  REGION 1..129: note = Anti-DLL3 camel sdAb amino acid sequence AS64207
EVQLVESGGGSVQAGGSLRLSCAASGYTYSSNFMGWFRQAPGKEREGVATIVSGGGTTYYADSVRGRFTISQDNAKNTVYLQMNSLKPEDTAMYYCAAGGPPTNGAKWYPLRPPGYNYWGQGTQVTVSS

SEQ ID NO: 332 - protein, 122 aa, synthetic construct
  REGION 1..122: note = Anti-DLL3 camel sdAb amino acid sequence AS64276
QVHLMESGGGSVQAGGSLSLSCVVSGYTGSSRCMAWFRQAPGKEREAVAQIFTGRGTTGYADSVKGRFTISQDNAKNTVYLRMNSLRPEDTAIYYCAASLGPGRGACGYNYWGQGTQVTVSS

SEQ ID NO: 333 - protein, 121 aa, synthetic construct
  REGION 1..121: note = Anti-DLL3 camel sdAb amino acid sequence AS64336
QVQLVESGGGSVQAGGSLRLSCTTSGRTYSSCSMGWYRQAPGKERELVSHIFSDGSRYYADSVKGRFTISQDNAKNTVYLQMNSLKPEDTAMYYCNRRTGWAPRCAVPGYWGQGTQVTVSS

SEQ ID NO: 334 - protein, 127 aa, synthetic construct
  REGION 1..127: note = Anti-DLL3 camel sdAb amino acid sequence AS64346
QVHLVESGGGSVQAGGSLRLSCAASGYTYFMGWFRQAPQKEREWVATIGTGDIFNGAAYYVDSVKGRFAISQDNAKNTVYLQMNSLKPEDTAVYVCAAVQSKSSNYVLRDASTYNYWGQGTQVTVSS

SEQ ID NO: 335 - protein, 116 aa, synthetic construct
  REGION 1..116: note = Anti-DLL3 camel sdAb amino acid sequence AS64420
EVQLVESGGGSVQAEGSLRLSCAASGDTSRSVWMGWARQVPGKEREVVATISTAGGSTWYTDSVKGRFTISQDNAKNTVYLQMNSLKPEDTAIYYCAARSRYATYWGQGTQVTVSS

SEQ ID NO: 336 - protein, 126 aa, synthetic construct
  REGION 1..126: note = Anti-DLL3 camel sdAb amino acid sequence AS64473
QVRLVESGGGSVQAGGSLRLSCTASGYTYRYLYMAWFRQTPGKEREGVACIYTGSGTTGYADSVKGRFTISQDNAKNTVYLQMNSLNAEDTAMYYCAASSPQWGGTCRRWSEYNYWGQGTQVTVSS

SEQ ID NO: 337 - protein, 116 aa, synthetic construct
  REGION 1..116: note = Anti-DLL3 camel sdAb amino acid sequence AS64475
QVQLQESGGGSVQAGGSLRLSCAASGYTWSRNWMGWFRQAPGKEREGFATITISGGSTWYADSVKGRFTISLDNAGNTVYLQMNSLKPEDTAMYYCAARDTARTYWGQGTQVTVSS

SEQ ID NO: 338 - protein, 127 aa, synthetic construct
  REGION 1..127: note = Anti-DLL3 camel sdAb amino acid sequence AS64513
EVQLVESGGGSVQAGGSLRLSCVASDYPYIDNCMGWFRQGPGKEREGVAAACTGGGSTYYADSVKGRFTISRDNAKNTVYLQMNSLKPEDTDVYYCATGYYSGSGPGYLLPWRYNYWGQGTQVTVSS

SEQ ID NO: 339 - protein, 131 aa, synthetic construct
  REGION 1..131: note = Anti-DLL3 camel sdAb amino acid sequence AS64562
EVQLVESGGGSVQAGGSLRLSCAASGYTARRDFMAWFRQVPGKEREGVAVIHTGGDTTYYADSVKGRFTISRDNAQNIMNLQMNSLKPEDTAMYYCAAGFRPRGGGYTGDVLAQAAAYNYWGQGTQVTVSS

SEQ ID NO: 340 - protein, 126 aa, synthetic construct
  REGION 1..126: note = Anti-DLL3 camel sdAb amino acid sequence AS64583
EVQLAESGGGLVQAGGSLRLSCTASGFTIAVYTMGWFRQAPGKEREGISCTSWAGGRTYTADSVKGRFTISRDNAKNTLYLQMNSLKPEDTAMYYCAAKAHPDCSGDWSPSGYEYWGQGTQVTVSS

SEQ ID NO: 341 - protein, 116 aa, synthetic construct
  REGION 1..116: note = Anti-DLL3 camel sdAb amino acid sequence AS64594
QVHLVESGGGSVQAGGSLRLSCAASGYTYNSNYMGWFRQAPGKEREGVALIYTGGGSTYYADSVKGRFTISRDNAKNTVYLQMNSLKPEDTAMYYCSVRTQTRNYWGQGTQVTVSS

SEQ ID NO: 342 - protein, 131 aa, synthetic construct
  REGION 1..131: note = Anti-DLL3 camel sdAb amino acid sequence AS64605
QVKLVESGGGSVQAGGSLRLSCAVSRYPYSSICMGWFRQAPGKESEGVARIYTGTGSTWYTDSVKGRFTIARDNAQNTVYLQMNSLKPEDTAMYYCAARSNSYSYSSCDYGPLTRGGYNFWGQGTQVTVSS

SEQ ID NO: 343 - protein, 115 aa, synthetic construct
  REGION 1..115: note = Anti-DLL3 camel sdAb amino acid sequence AS64606
EVQLAESGGGSVQAGGSLRLSCAVSGYTSRSNYMGWFRQAPGKEREGVALIYTRGGSTYYASSVKGRFTISQDSAKKTYLQMNSVKPEDTAMYYCALRLDEKMYWGQGTQVTVSS

SEQ ID NO: 344 - protein, 133 aa, synthetic construct
  REGION 1..133: note = Anti-DLL3 camel sdAb amino acid sequence AS68121
EVQLAESGGGSVQAGGSLRLSCDASGYTYSRNCMGWFRQAPGKEREGVAAFYTDYIRFGRTYYADSVKGRFTIFQDNAKNTVYLQMNSLKPEDTAMYYCAADPGSRTDDSCGTSYNKGNFGYWGQGTQVTVSS

SEQ ID NO: 345 - protein, 130 aa, synthetic construct
  REGION 1..130: note = Anti-DLL3 camel sdAb amino acid sequence AS68170
QVQLVESGGGSVQAGGSLRLSCTASGYTYRSNCMGWFRQAPGKEREGVATIYTGGGRNLYYADSVKGRFTISRDNAKNTLYLQMNSLKPEDSARYYCAAASDVAVGVNSCGGRTAGFDAWGQGTQVTVSS

SEQ ID NO: 346 - protein, 125 aa, synthetic construct
  REGION 1..125: note = Anti-DLL3 camel sdAb amino acid sequence AS63964
QVRLVESGGGSVQAGGSLRLSCSASGYTYSYNNMGWFRQAPGNEREGVAAISGGRFTAYADSVKGRFTISRDNAENTLYLQMNNLKPEDTGMYYCAAEVVDPTWGSRDQRRYKYWGQGTQVTVSS

SEQ ID NO: 347 - protein, 124 aa, synthetic construct
  REGION 1..124: note = Anti-DLL3 camel sdAb amino acid sequence AS64116
QVKLVESGGGSVQAGGSLRLSCAASGYIYSCVGWFRQAPGKEREGVAGISTGGGGTVYADSVKGQFTISRDNAKNTVYLQMDSLKPEDTAMYYCAADRWNSFANCGAWGRYTYWGQGTQVTVSS

SEQ ID NO: 348 - protein, 130 aa, synthetic construct
  REGION 1..130: note = Anti-DLL3 camel sdAb amino acid sequence AS68270
QVQLAESGGGSVQAGGSLRLSCVASGYPSSTYYMLSMAWFRQAPGKEREGVAAITSGTGSTSYADSVKDRFTISKDYANNTLYLHINNLKPEDTAMYYCAAASGWIVPSRSLTANLYRYWGQGTQVTVSS

SEQ ID NO: 349 - protein, 125 aa, synthetic construct
  REGION 1..125: note = Anti-DLL3 camel sdAb amino acid sequence AS68320
QVHLVESGGDSVQAGGSLRLSCAASGYTYNTNYMGWFRQAPGKEREGVAAIYRHSGNTAYADSVKGRFTISQDYAKNTVYLQMNSLKPEDTAMYYCAAGRAGPWALMRPTEFGYWGQGTQVTVSS

SEQ ID NO: 350 - protein, 123 aa, synthetic construct
  REGION 1..123: note = Anti-DLL3 camel sdAb amino acid sequence AS68351
QVQLEESGGGLVQPGGSLRLSCAASGDTFRAYYMNWVRQAPGKGFEWVSGISASGGRTSYADSVKGRFTISRDNAKNTLYLQLNSLSTEDTGMYYCVKGAVRLSTSSVRDSSWGQGTQVTVSS

SEQ ID NO: 351 - protein, 116 aa, synthetic construct
  REGION 1..116: note = Anti-DLL3 camel sdAb amino acid sequence AS75378
QVQLEESGGGSVQAGGSLRLSCVVSGNTRSTTYMGWFRQAPGKEREGVAIVYTGGRDTYYAASVKGRFTISQDNAKTTIYLQMNSLEPEDTAMYYCAARSYEYTYWGRGTQVTVSS

SEQ ID NO: 352 - protein, 124 aa, synthetic construct
  REGION 1..124: note = Anti-DLL3 camel sdAb amino acid sequence AS75383
EVQLAESGGGSVQAGGSLRLSCVASGYTFSSYCLGWFRQAPGKQRQGVATFNNRGVANYHDSVKGRFTASVDNAKNTLLLQMNSLEPDDTAMYYCAADRRYGRQWYQPCEWNTWGQGTQVTVSS

SEQ ID NO: 353 - protein, 129 aa, synthetic construct
  REGION 1..129: note = Anti-DLL3 camel sdAb amino acid sequence AS75751
QVRLVESGGGSVQAGGSLRLSCVASGYFYNTYYFMGWFRQAPGKEREGVAAIDTDGRTSYADSVKGRFTISKDNAKNTLYLQMNSLKPEDTAMYYCAAGFGYMNVIQALNGMRQNPDYWGQGTQVTVSS

SEQ ID NO: 354 - protein, 124 aa, synthetic construct
  REGION 1..124: note = Anti-DLL3 camel sdAb amino acid sequence AS76422
QVKLVESGGGSVQAGGSLRLSCAASGYTFAGNCLGWFRQAPGKGREGVVTYNNFGVANYADSVKGRFTVSQDNAKNTLLLQMNSLEPEDTAMYYCAADRRDGRRWSQPCEWNTWGQGTQVTVSS

SEQ ID NO: 355 - protein, 125 aa, synthetic construct
  REGION 1..125: note = Anti-DLL3 humanized sdAb amino acid sequence AS64380VH4
EVQLVESGGGLVQPGGSLRLSCAASGNTYSSNYMGWFRQAPGKGLEEVAVIYTRGGHTYYVDSVRGRFTISRDNAKNSLYLQMNSLRAEDTAVYYCAASSRHRLGLNNPRDYDYWGQGTLVTVSS

SEQ ID NO: 356 - protein, 125 aa, synthetic construct
  REGION 1..125: note = Anti-DLL3 humanized sdAb amino acid sequence AS64380VH5
EVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSS

SEQ ID NO: 357 - protein, 125 aa, synthetic construct
  REGION 1..125: note = Anti-DLL3 humanized sdAb amino acid sequence AS64380VH6
EVQLVESGGGLVQPGGSLRLSCAASGNTYSSNYMGWFRQAPGKGLEEVAVIYTRGGHTYYVDSVRGRFTISQDNAKNSVYLQMNSLRAEDTAMYYCAASSRHRLGLNNPRDYDYWGQGTLVTVSS

SEQ ID NO: 358 - protein, 125 aa, synthetic construct
  REGION 1..125: note = Anti-DLL3 humanized sdAb amino acid sequence AS64380VH7
EVQLVESGGGLVQPGGSLRLSCAASGNTYSSNYMGWFRQAPGKGREEVAVIYTRGGHTYYVDSVRGRFTISQDNAKNSVYLQMNSLRAEDTAMYYCAASSRHRLGLNNPRDYDYWGQGTLVTVSS

SEQ ID NO: 359 - protein, 125 aa, synthetic construct
  REGION 1..125: note = Anti-DLL3 humanized sdAb amino acid sequence AS64511VH4
EVQLVESGGGLVQPGGSLRLSCAASRATYSTNYISWFRQAPGKGLEAVATITTGDGETAYADSVKGRFTISRDNAKNSLYLQMNSLRAEDTAVYYCAANLRIGGDWFDGRDFRAWGQGTLVTVSS

SEQ ID NO: 360 - protein, 125 aa, synthetic construct
  REGION 1..125: note = Anti-DLL3 humanized sdAb amino acid sequence AS64511VH5
EVQLVESGGGLVQPGGSLRLSCAASRATYSTNYISWFRQAPGKGLEAVATITTGDGETAYADSVKGRFTISRDNAKNSVYLQMNSLRAEDTAMYYCAANLRIGGDWFDGRDFRAWGQGTLVTVSS

SEQ ID NO: 361 - protein, 125 aa, synthetic construct
  REGION 1..125: note = Anti-DLL3 humanized sdAb amino acid sequence AS64511VH6
EVQLVESGGGLVQPGGSLRLSCAASRATYSTNYISWFRQAPGKGREAVATITTGDGETAYADSVKGRFTISRDNAKNSVYLQMNSLRAEDTAMYYCAANLRIGGDWFDGRDFRAWGQGTLVTVSS

SEQ ID NO: 362 - protein, 125 aa, synthetic construct
  REGION 1..125: note = Anti-DLL3 humanized sdAb amino acid sequence AS63931VH4
QVQLVESGGGVVQPGGSLRLSCAGSFSGYGVSTMAWFRQAPGKGLEGVAAITVGSGNTYYADSVTGRFTISRDNSKNTLYLQMNSLRAEDTAVYYCAAGWLSGGSWHVPGRYNYWGQGTLVTVSS

SEQ ID NO: 363 - protein, 125 aa, synthetic construct
  REGION 1..125: note = Anti-DLL3 humanized sdAb amino acid sequence AS63931VH5
QVQLVESGGGVVQPGGSLRLSCAGSFSGYGVSTMAWFRQAPGKGLEGVAAITVGSGNTYYADSVTGRFTISRDNSKNTVYLQMNSLRAEDTAMYYCAAGWLSGGSWHVPGRYNYWGQGTLVTVSS

SEQ ID NO: 364 - protein, 125 aa, synthetic construct
  REGION 1..125: note = Anti-DLL3 humanized sdAb amino acid sequence AS63931VH6
QVQLVESGGGVVQPGGSLRLSCAGSFSGYGVSTMAWFRQAPGKGREGVAAITVGSGNTYYADSVTGRFTISRDNSKNTVYLQMNSLRAEDTAMYYCAAGWLSGGSWHVPGRYNYWGQGTLVTVSS

SEQ ID NO: 365 - protein, 125 aa, synthetic construct
  REGION 1..125: note = Anti-DLL3 humanized sdAb amino acid sequence AS63997VH4
QVQLVESGGGVVQPGGSLRLSCAASFSGYGVSTMAWFRQAPGKGLEGVAAITVGSGNTYYADSVKGRFTISRDNSKNTLYLQMNSLRAEDTAVYYCAVGYLSGGSWDVPGRYNYWGQGTLVTVSS

SEQ ID NO: 366 - protein, 125 aa, synthetic construct
  REGION 1..125: note = Anti-DLL3 humanized sdAb amino acid sequence AS63997VH5
QVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSS

SEQ ID NO: 367 - protein, 125 aa, synthetic construct
  REGION 1..125: note = Anti-DLL3 humanized sdAb amino acid sequence AS63997VH6
QVQLVESGGGVVQPGGSLRLSCAASFSGYGVSTMAWFRQAPGKGREGVAAITVGSGNTYYADSVKGRFTISRDNSKNTVYLQMNSLRAEDTAMYYCAVGYLSGGSWDVPGRYNYWGQGTLVTVSS

SEQ ID NO: 368 - DNA, 387 bp, synthetic construct
  source 1..387: mol_type = other DNA
gaggtgcaactggcggagtctgggggaggatcggtgcaggctggagggtctctgagactctcctgtgcagcctctggatacacctacagtggcaactatatggcctggttccgccaggctccagggaacgagcgcgagggggtcgcagttgtttataatattgacggtggcggtcgtttcactacctatgccgactccgtgaagggccgattcaccatctcccgaggcaacgacaagaacacggtgtatctgcaaatgaacagcctgaaacctgaggatagtggcatgtactactgtgcggcagaggtagctgatccgacctgggggtcgcgtgaccaaagacgatataagtactggggccaggggacccaggtcaccgtctcctca

SEQ ID NO: 369 - DNA, 354 bp, synthetic construct
  source 1..354: mol_type = other DNA
caggtgcaattggaggagtctgggggaggctcggtgcaggctggagggtctctgagactctcctgtgtagcctctggatacacctacggtagcaccttcatgggctggttccgccagaatccagggaaggagcgcgagggggtcgcagttatttatactggtggtggtagtacatggtatgccagctccgtgaagggccgattcaccatctcccaggacaacgccaagaacacggtgtatctgcaaatgaacagcctgaaacctgaggacactgccatgtactactgtgcggcgcgttacgggtcgggaaacgttaactactggggccaggggacccaggtcaccgtctcctca

SEQ ID NO: 370 - DNA, 390 bp, synthetic construct
  source 1..390: mol_type = other DNA
caggtgcacctgatggagtctgggggaggctcggtgcaggctggagggtctctgagactcgcctgtgaaacctctagagacatctacggtaacaactgcatggcctggttccgccaggctccagggaaggagcgcgagggagtcgcgtctatttatcctgctggtggtcgcccgtactatgccgactccgtgaagggccgattcaccatctcccaagacaacgccaagaacacggtgtatctgcaaatggacagcctgaaacctgaggacacggccatgtactactgcgcggcacgctctttttcgatagcagtttgcgcgacgcgctctggtattaccaggtctaattttgcttactggggccaggggacccaggtcaccgtctcctca

SEQ ID NO: 371 - DNA, 387 bp, synthetic construct
  source 1..387: mol_type = other DNA
caggtgaagttggtggagtctgggggaggctcggtgcaggctggagggtctctgagactctcctgtgcagcctctggatacacctacagtagcaacttcatgggctggttccgccaggctccagggaaggagcgcgagggggtcgcaactattgtttctggtggtggtaccacatactatgccgactccgtgaggggccgattcaccatctcccaagacaacgccaagaacacggtgtatctgcaaatgaacagcctgaaacctgaggacactgccatgtactactgtgcggcagggggccccgttacgaacgcacctagatggtaccccctccgacctcctggttataactactggggccaggggacccaggtcaccgtctcctca

SEQ ID NO: 372 - DNA, 366 bp, synthetic construct
  source 1..366: mol_type = other DNA
caggtgaggttggtggagtctgggggaggctcggtgcaggctggagggtctctgagactctcctgtgaagccactggatatagaaactgcatggcctggttccgccaagctccagggaaggaacgcgagggggtcgcagttatttatactcctagtggtatcacggactatgcaagctccgtgaagggccgattcaccatctcccaaaacaacgccaggaacacgcagtatctgcaaatgaacagcctgaaacctgaggacactgccatgtactactgtgcggcagatcgaccctttgtttgtaatatagcgaatatgagaaggtcctccaactggggccgggggacccaggtcaccgtctcctca

SEQ ID NO: 373 - DNA, 375 bp, synthetic construct
  source 1..375: mol_type = other DNA
caggtgaggttggtggagtctgggggaggctcggtgcaggctggagggtctctgagactctcctgtgcaggctctttctctggatacggcgtcagtaccatggcctggttccgccaggctccagggaaggagcgcgagggggtcgcagctattacagttggtagtggaaacacatactatgccgactccgtgaagggccgattcaccatctcccgagacaacgccaagaggacggtgtttttgcaaatgaacagcctgaaacctgaggacactgccatgtactactgtgcggtcggatacttgtcgggtggtagttgggacgttcccggaaggtataactactggggccaggggacccaggtcaccgtctcctca

SEQ ID NO: 374 - DNA, 375 bp, synthetic construct
  source 1..375: mol_type = other DNA
caggtgcacctggtggagtctgggggaggctcggtgcaggctggagggtctctgagactctcctgtgcagcctctcagtacgtttacaggtgggacctcatgggctggttccgccaggctccagggaaggagcgcgaggcggtcgctgctgtttatactggtgatggtattacatactatgccgactccgtgaagggccgattcagcatttcccaagacaacgccaagaacacggtgtatctgcaaatgaacagcctgaaacctgaggacactggcatgtacttctgtgcggcaggcttcgtctctggtggtagatggaaccagtcatatcgttataaatactggggccaggggacccaggtcaccgtctcctca

SEQ ID NO: 375 - DNA, 387 bp, synthetic construct
  source 1..387: mol_type = other DNA
caggtgcacctgatggagtctgggggaggctcggtgcaggctggagggtctctgagactctcctgtgcagcctctggatacacctaccgcagcaacttcatgggctggttccgccaggctccagggaaggagcgcgaggggatcgcaactattcattctggtgtggctaccacatactatgccgactccgtgaagggccgattcaccatctcccaagacaacgccaagaacacggtgtatctgcaaatgaacagcctgaaacctgaggacactgccatgtactactgtgcggcagggggcccccctgcgaacgctgatagatggtaccccctccgacctcctggttataactactggggccaggggacccaggtcaccgtctcctca

SEQ ID NO: 376 - DNA, 381 bp, synthetic construct
  source 1..381: mol_type = other DNA
caggtgaggttggtggagtctgggggaggctcggtgcaggttggagggtctctgagactctcctgtgcagcctctagatctccctacagtagcagtaggtgcatggggtggttccgccaggctccagggaaggagcgcgagggggtcgcagctctttatactggtggtggtagcacatcctatgccgactccgtgaagggccgattcaccatctcccaagacaacgccaagaatacggtgtatctgcaaatgaacagcctgaaacctgaggacactgccatgtactactgtgcggcagttgtccctaggggtggtagctgccgtcttgatgaaagagggtattaccactggggccaggggacccaggtcaccgtctcctca

SEQ ID NO: 377 - DNA, 369 bp, synthetic construct
  source 1..369: mol_type = other DNA
caggtgcagctggtggagtctgggggaggctcggtgcaggccggagggtttctgagactctcctgtgcactctctggatacagctactatattaacttgatggcgtggttccgtcaggctccagggaaggagcgcgaggcagtcgcagctcatggtcctgtgagtgggacagcatactataccgactccgtgaagggccgattcaccatctcccgagaccccggcaagaacacgatgtatcttcaaatgtttagcctgcaaccggaggacactgccctctactactgtgcggcggaaacgactatgggttgggcccacgaacgcgggtataggtactggggccaggggacccaggtcaccgtctcctca

SEQ ID NO: 378 - DNA, 381 bp, synthetic construct
  source 1..381: mol_type = other DNA
caggtgcacctgatggagtctgggggaggctcggtgcaggctggagggtctctgagactctcctgtgaagcctctggatacacctacagtcgcaactgcatgggctggttccgccaggctccagggaaggagcgcgagggggtcgcagctattaacactggtggtggtagcacatattatgccgactcccttgagggccgattcaccatctcccaagacaacgccaagaatactatgtatctgcaaatgaacagcctgaaacctgaggacactgccatgtactactgtgcggcaggtcccgatctcggtggtagctggtgtcggcccgttgagcgggcttttacgtactggggccaggggacccaggtcaccgtctcctca

SEQ ID NO: 379 - DNA, 375 bp, synthetic construct
  source 1..375: mol_type = other DNA
caggtgcaactgcaggagtctggggggggctcggtgcaggctggagggtctctgacactctcctgtgaagcctctggaaacacctacagtactaattacatgggctggttccgccaggctccagggaaggagcgcgaagaggtcgcggttatttacactcgtggtggtcacacatactatgtcgactccgtgaggggccgattcaccatctcccaagacaacgccaagaacacggtgtatctgcaaatgaacagcctgaaacctgaggacactgccatgtactactgtgcggcagcttcacgacatagactccgtttaaataacccacgggactatgactactggggccaggggacccaggtcaccgtctcctca

SEQ ID NO: 380 - DNA, 348 bp, synthetic construct
  source 1..348: mol_type = other DNA
caggtgcaactggcggagtctgggggaggctcggtgcaggctggagggtctctgagactctcctgtgcagcctctggatacacttatacgagcaactggctgggctggttccgccaggctccagggaaggagcgcgaggaggtcgcaattatttatactggtagtggtagtacacactatcgcagctccgtgaagggccgattcaccatctcccaagacaacgccaagaacacggtgtatctgcaaatgaacagcctgaaacctgaggacactgccatgtactactgtgcggcgcgtttctcagagtataattactggggccaggggacccaggtcaccgtctcctca

SEQ ID NO: 381 - DNA, 387 bp, synthetic construct
  source 1..387: mol_type = other DNA
gaggtgcaactggcggagtctgggggaggctcggtgcaggctggagggtctctgagactctcctgtgcagcctctggatacacctaccgtagcaacttcatgggctggttccgccaggctccggcgaaggagcgcgagggggtcgcaactattgattctcgtggtactatcacatactatgccgactccgtgaagggccgattcaccatctcccaagacaacgagaagaacacggtgtatctgcaaatgaacagcctgaaacctgaggacactgccatgtactactgtgcggcagggggcccccgcacgaacgatgatagatggtaccccctccgacctcctggttataactactggggccaggggacccaggtcaccgtctcctca

SEQ ID NO: 382 - DNA, 378 bp, synthetic construct
  source 1..378: mol_type = other DNA
caggtgaggttagtggagtctgggggaggctcggtgcaggctggagggtctctgagactctcctgtgctgcctctggatccacctacagtacaaacttcatgggctggttccgccaggctccagggaaggagcgcgagggggtcgcaacgctggttacttgggttgaacgcacagcctatgccgactccgtgaagggccgattcaccatctcccaagaccgcgccaagaacacggtgtatctacaaatgaacagcctgaaacctgaggacactgccatgtactactgtgcggcagccgccgcttccactgatgtacgtctcctcgacccgggggactttgcttactggggccaggggacccaggtcaccgtctcctca

SEQ ID NO: 383 - DNA, 378 bp, synthetic construct
  source 1..378: mol_type = other DNA
caggtgcacctgatggagtctgggggaggcttggtgcagacaggggggtctctgagactctcctgtacagcctctggattcacttttgatcgtaatgccatgcgctggtaccgccaggctccagggaaggagcgcgagggggtctcatgtattgattggacgggtgcaaatattgcctatgcagactccgtgaagggccgattcaccatctccagagacaacgccaagaacacgctgtatctgcaaatgaacagcctgaaacctgaggacacgggcatgtattactgtgcggcagatacgacgtcggggtattgttcaggcttttggtctacgagccggtactcatggggccaggggacccaggtcaccgtctcctca

SEQ ID NO: 384 - DNA, 378 bp, synthetic construct
  source 1..378: mol_type = other DNA
caggtgcaattgaaggagtctgggggaggctcggtgcaggctggagggtctctgagactctcctgtacagcctctggatacacctacagatacctctacatgggctggttccgccagactccagggaaggagcgcgagggggtcgcatgtatttatactggtagtggtagcacagggtatgccgactccgtgaagggccgattcaccatctcccaagacaacgccaagaacacggtgtatctgcaaatgaacaacctgaaacctgaggacactgccatgtactactgtgcggcaagttcgccccggtggggcggtacctgtcgacgctggtctcagtataactactggggccaggggacccaggtcaccgtctcctca

SEQ ID NO: 385 - DNA, 384 bp, synthetic construct
  source 1..384: mol_type = other DNA
gaggtgcagctggtggagtctgggggaggctcggtgcaggctggagggtctctgagactctcctgtgcggcctctgtatacaccagcagtagctactgcatgggctggttccgccaggctccagggaaggagcgcgagggggtcgcagctatgtgttttggtggtcttgtcacacactatgccgactccgtgaagggccgattcaccatctcccaagacaatgccaagaacacggtgtatctgcaaatgaacagcctgaaacctgaggacactgccatgtactactgtgcggcagatttcggcagggataaaaactatttacgaccgttactgccccatgcatataactactggggccaagggacccaggtcaccgtctcctca

SEQ ID NO: 386 - DNA, 351 bp, synthetic construct
  source 1..351: mol_type = other DNA
caggtgcaattgaaggagtctgggggaggttcgatccaggctggagggtctctgagactctcctgtgcagcctctggagtctcctacaataggtgcagtatgggctggtaccgccaggctccagggaaggggcgcgagttggtctcacgtattcagccgggtggtaatacatactatgcagactccgtgaagggccgattcaccgtctcccaagacaacgccaagaacacagtatctctacaaatgaacagcctgaaacctgaggacacggccatgtattactgtaacgcactgtgctggcgggagaatgttaactactggggccaggggacccaggtcaccgtctcctca

SEQ ID NO: 387 - DNA, 372 bp, synthetic construct
  source 1..372: mol_type = other DNA
caggtgcacctggtggagtctgggggaggctcggtgcagactggaggatctctgagactctcctgtgcagtctctggagacatctataacctcatgtcgatggcctggttccgccgggctccagggaaggagcgcgagggggtcgcatatattaatactattattggtaacacatactatactgactccgtgaagggccgattcaccatctcccgcgataactccaagaacactttgtatctgcaaatgaacaacctgaaacctgaggacacagccatgtactactgtgcggcgttcaattacggaggtgcctggtacgaggaacgcagctataaatactggggccaggggacccaggtcaccgtctcctca

SEQ ID NO: 388 - DNA, 375 bp, synthetic construct
  source 1..375: mol_type = other DNA
gaggtgcagctggtggagtctggggggggctcggtgcaggctggagggtctctgacactctcctgtgaagcctctggaaacacctacagtagtaattacatgggctggttccgccaggctccagggaaggagcgcgaggaggtcgcggttatttacactcgtggtggtcacacatactatgtcgactccgtgaggggccgattcaccatctcccaagacaacgccaagaacacggtgtatctgcaaatgaacagcctgaaacctgaggacactgccatgtactactgtgcggcgtcttcacgacatagactcggtttaaataacccacgggactatgactactggggccaggggacccaggtcaccgtctcctca

SEQ ID NO: 389 - DNA, 375 bp, synthetic construct
  source 1..375: mol_type = other DNA
caggtgaggttggtggagtctgggggaggctcggtgcaggctggagggtctctgagactctcctgtgctgcctctggatccacctacagtacaaacttcatgggctggttccgccaggctccagggaaggagcgcgagggggtcgcaacgcttgttacttgggctgaacgcacagcctatgccgactccgtgaagggccgattcaccatctcccaagaccgcgccaagaacacggtgtatctacaaatgaacggcctgaaacctgaggacactgccatgtactactgtgcggcagccgcttccactgctgtacgtctcctcgacccgggggactttgcttactggggccaggggacccaggtcaccgtctcctca

SEQ ID NO: 390 - DNA, 348 bp, synthetic construct
  source 1..348: mol_type = other DNA
caggtgcacctggtggagtctgggggaggctcggtgcaggctggagggtctctgagactctcctgtgcagcctctggatataccgacagtagcgtctacataggctggttccgccaggctccagggaaggagcgcgaggaggtcgcgattatttatactggtggtgaaagcacacactatcgcagctccgtgaagggccgattcaccgtctcccaagacaacgccaagaacacgctgtatctgcaaatgaacagcctgaaacctgaggacacggccatgtattactgtgcagcacgattcccagctgttacctactggggccaggggacccaggtcaccgtctcctca

SEQ ID NO: 391 - DNA, 375 bp, synthetic construct
  source 1..375: mol_type = other DNA
caggtgcagctggtggagtctgggggaggctcggtgcaggctggagggtctctgagactctcctgtgcagcctctagagccacctacagtaccaactacataagctggttccgccaggctccagggaaggagcgcgaggcggtcgcaacaattactactggtgatggtgagacagcgtatgccgactccgtgaagggccgattcaccatctcccgagacaacgccaagaacacggtctatctgcaaatgaacagcctgaaacctgaggacactgccatgtactactgtgcggcgaacttgcgaatcggtggcgactggttcgacggacgcgattttcgtgcctggggccaggggacccaggtcaccgtctcctca

SEQ ID NO: 392 - DNA, 375 bp, synthetic construct
  source 1..375: mol_type = other DNA
caggtgaagttggtggagtctgggggaggctcggtgcaggctggagggtctctgagactctcctgtgcagcctctagatacaccgacaatttcgtgtacatgggctggttccgccaggctccagggaaggagcgcgagggggtcgcactgatttatcctggtggtggtagcacctactatgcctcctccgtgaagggccgattcaccatctcccaagacaacgccaagggcacggtgcatctgcaaatgaacaacctgaaacctgaggacactgccatgtactactgtgcggcaaaatgggggctgggcggggggggcctgaaatcagatacgtatatgtactggggccaggggacccaggtcaccgtctcctca

SEQ ID NO: 393 - DNA, 387 bp, synthetic construct
  source 1..387: mol_type = other DNA
caggtgcacctggtggagtctgggggaggctcggtgcaggctggagggtctctgagactctcctgtgcagcctctggatacacctaccgtgtcaacttcatgggctggttccgccagactccagggaaggagcgcgagggggtcgcaactattgattctggtgtgggtaccacatactatgccgactccgtgaagggccgattcaccatctcccataacaacgccaagaacacgatttatctgcaaatgaacagcctgaaacctgaggacactgccatgtactactgtgcggcagggggcccccctacggacggtgatagatggtaccccctccgacctcctggttataactattggggccaggggacccaggtcaccgtctcctca

SEQ ID NO: 394 - DNA, 351 bp, synthetic construct
  source 1..351: mol_type = other DNA
caggtgcagctggtggagtctgggggaggctcggtgcaggctggagggtctctgagactctcctgtgcagcctctggatacactgatagatgcagcatggcctggtaccgccaggctccagggaaggagcgcgagttggtctcgcgtattagcacgagcggtttcacaaactacgcagcctccgtgaagggccgattcaccatctcccaagacaacgccaagaacacggtgtatctgcaaatgaacagcctgaaccccggggacacgggcatgtattactgtgccataatcgtaggacgtacttgtagtttgaactactggggcaacggcatcctggtcaccgtctcctca

SEQ ID NO: 395 - DNA, 381 bp, synthetic construct
  source 1..381: mol_type = other DNA
caggtgaggttggtggagtctgggggaggctcggtgcaggctggagggtctctgagactctcctgtgcagcctccggatacagttttagaggcgactttatgtgtatgggctggttccgccagactccagggaaggggcgcgagggggtcgcagttttttatcctggtggcggcagcacaaactatgccgactccgcgaagggccgattcaccatctcccaagacaacgccaagaacacgatgtatctgcaaatgaacaccctgaaacctgaggacactgccatgtactactgtgcggctcgacggtgggtcagtggtacctgctactgggatagtgactttcattactggggccaggggacccaggtcaccgtctcctca

SEQ ID NO: 396 - DNA, 375 bp, synthetic construct
  source 1..375: mol_type = other DNA
cagatgcagctggtggagtctgggggaggctcggtgcaggctggagggtctctgagactctcctgtgcagcctctggaaacacctacagtaccaattacatggcgtggttccgccaggctccagggaaggagcgcgaggaggtcgcggttatttacactcgtggtggtcacacctactatatcgactccgtgaggggccgattcaccatctcccaagacaacgccaagaacacggtgtatctgcaaataaacagcctgaaacctgaggacactgccatgtactactgtgcggcgtcttcacgaattagactccattgaatcgacccacgggactatcacgactggggccaggggacccaggtcaccgtctcctca

SEQ ID NO: 397 - DNA, 381 bp, synthetic construct
  source 1..381: mol_type = other DNA
caggtgaggttagtggagtctgggggaggctcggtgcaggctggagggtctctgagactctcctgtgcagccgacagatacacctacagtagcgcctgcatgggctggttccgccaggctccagggaaggagcgcgagggggtcgcctctatttttactggtactggtggtagcacatactatgccgactccgtgaagggccgattcaccatctcccaagacaacgccaagaacacggtgtatctgcaaatgaacagcctgaaacctgaggacactgccatatactactgtgcggcaagggccttccaggtcggttactgctacctgcgaaccgatgtgtataactactggggccaggggacccaggtcaccgtctcctca

SEQ ID NO: 398 - DNA, 375 bp, synthetic construct
  source 1..375: mol_type = other DNA
gaggtgcagctggcggagtctgggggaggctcggtccaggctggagggtctctgagactctcctgtgtagcctctagatacaccttcagtagcacctgcatggcctggttccgccaggctccagggaaggagcgcgaggaggtcgcagctatttatactgatgatggtagcacatggtatgccgactccgtgaagggccgattcaccatctccagagacaacgccaagaacacggtgtatctgcaaatgaacagcctgaaacctgaggacactgctatgtactactgtgcggcacgtaggtgggcgtgccccagggttggtagctggcatgagttcgcctactggggccaggggacccaggtcaccgtctcctca

SEQ ID NO: 399 - DNA, 375 bp, synthetic construct
  source 1..375: mol_type = other DNA
caggtgcaactggtggagtctgggggaggctcggtgcaccctggagggtctctgagactctcctgtgcagcctctggatccacctacagttccaactacctcggctggttccgccaggctccaggaaaggggcgcgactgggttgcggctattagcactggtgacggtgccacagcctatgccgactccgtgaagggccgattcaccatctcccaagacaacgccaagaacacggtgtatctgcaaatgaacagcttgaaacttgaggacagtgccatgtactactgtgcggcggctcgcggcagatttatcgattggacaaaggcaacccagtatgactactggggccaggggacccaggtcaccgtctcctca

SEQ ID NO: 400 - DNA, 384 bp, synthetic construct
  source 1..384: mol_type = other DNA
cagatgcagctggtggagtctgggggagactcggtgcaggctggagggtctctgagactctcctgtgcagcctctggatacacctacagcggcgtctgcatgggctggttccgccaggctccagggaaggagcgcgagggggtcgcagctattgatagtgatggtagcacaagctacgcagactccgtgaagggccgattcaccatctccaaagacaacgccaagaacactctgtatctgcaaatgaacagcctgaaacctgaggacactgccatgtactactgtgcggcagccattgtcgggggttttaatgcatattgtagtggtggttatgttctggactttggtgcctggggccaggggacccaggtcaccgtctcctca

SEQ ID NO: 401 - DNA, 396 bp, synthetic construct
  source 1..396: mol_type = other DNA
gaggtgcagctggcggagtctgggggaggctcggtgcaggctggagggtctctgagactctcctgttcagcctctggtttcacttttgatgattctgacatggcctggtaccgccaggctccaggggatgggtgcgacttggtctcaactattagtagtgatggtagcacatactatgcagactccgtgaagggccgattcaccatctcccaagacaacgccaagaacacggtgtatctgcaaatgcacagcctgaaacctgaggacacggccgtgtattactgtgcggcagatttcctcaccggcttttactatagcgactccccccatccggccccttgttctgcatccgactttggttactggggccaggggacccaggtcaccgtctcctca

SEQ ID NO: 402 - DNA, 378 bp, synthetic construct
  source 1..378: mol_type = other DNA
caggtgcaattgaaggagtctgggggaggctcggtgcaggctggagggtctctgagactctcctgtgcagcctctggatacacctacagtagccactccatgggctggttccgccaggctccagggaaggagcgcgagggggtcgcagttatttatactggtgatggtagcacatactatgccgactccgtgaagggccgattcaccatctcccaagacaacgccaagaacacggtgtatctgcaaatgaacagcctgaaacctgaggacactgccatgtactactgtgcggcagatccgaaccccgattatatgcttccgtttcggccgtcccgtaggtcgtggtggggccaggggacccaggtcaccgtctcctca

SEQ ID NO: 403 - DNA, 369 bp, synthetic construct
  source 1..369: mol_type = other DNA
caggtgcacctggtggagtctgggggaggctcggtgcaggctggagggtctctgagactctcctgtgcagcctctggatacccctacagtagcccctgcatggcctggtttcgccaggctccagggaaggagcgcgagggggttttagttgcttatactggtggggacattcaataccttaccgactccgtgaagggccgattcaccatctcccgagacaacgccaagaacacggtgtatctgcaaatgaacagcctgaaacctgaggacactgccatgtactactgtgcggcggatctgcgattacctcgtgccggcggttgtgcgtatagctactggggccaggggacccaggtcaccgtctcctca

SEQ ID NO: 404 - DNA, 363 bp, synthetic construct
  source 1..363: mol_type = other DNA
caggtgaggttggtggagtctgggggaggctcggtgcaggctggagggtctctgagactctcctgtgtagcctctggatacaccgtcagtgcctactgtatgggctggttccgccaggttctagggaaggggcgcgagaggatcgcatttatcgatgccgggggtgctacgatttacgcagaccccgtgaagggccgattcaccatctccaaagacaacgccaagaacactctgtatctgcaaatgaacagcctgaaacctgaggacactgccatgtactactgtgttgcagatcgccgggggcgggtacgtcggtgcgagtataacgcctggggccaggggacccaggtcaccgtctcctca

SEQ ID NO: 405 - DNA, 375 bp, synthetic construct
  source 1..375: mol_type = other DNA
caggtgcacctgatggagtctgggggaggctcggtgcaggctggagggtctctgagactctcctgtgcagcctctggatacatttacagtagcttctgcatgggctggttccgccaggctccagggaaggagcgcgaggtggtcgcgtatattcgcgataatattatgacaagttacacagactccgtgaagggccgattcaccatctccaaagacaacgccaagagaactctgtatctacaaatgaacggcctgaaacctgaagatactggcatgtactactgtgcggtagaccgggggggatacgctaatagttgcgcggtagcggcccggtatgattactggggccgggggacccaggtcaccgtctcctca

SEQ ID NO: 406 - DNA, 375 bp, synthetic construct
  source 1..375: mol_type = other DNA
gaggtgcagctggcggagtctgggggaggctcggtgcaggctggggggtctctgagactctcctgtgcaggctctttctctggatacggcgtcagtaccatggcctggttccgccaggctccagggaaggagcgcgagggggtcgcagctattactgttggtagtggaaacacatactatgccgactccgtgacgggccgattcaccatctcccgagacaacgccaagaggacggtgtatttgcaaatgaacagcctgaaacctgaggacactgccatgtactactgtgcggccggatggttgtcgggtggtagttggcacgttcccggcaggtataactactggggccaggggacccaggtcaccgtctcctca

SEQ ID NO: 407 - DNA, 369 bp, synthetic construct
  source 1..369: mol_type = other DNA
caggtgaagttagtggagtctgggggaggctcggtgcaggctggagggtctctgagactctcctgtgcagcctctggatccaccatcagtagtcgcccgatggcctggttccgccaggctccagggaaggagcgcgagggggtcgcgtgtatacatactggtcttggtagaacatactatgccgactccgtgaagggccgattcaccatctcccaagacaacgccaagaacacggtgtatctgcaagtgaacagcctgaaacctgaggacactgccatgtactactgtgcggcagactcgcggcggccgtgtatggtagccgcagggtatacctactggggccaggggacccaggtcaccgtctcctca

SEQ ID NO: 408 - DNA, 378 bp, synthetic construct
  source 1..378: mol_type = other DNA
caggtgcagctggtggagtctgggggaggctcggtgcaggctggagggtctctgagactctcctgtacagcctctggatacacctatagatacctctacatgggctggttccgccagactccagggaaggagcgcgagggggtcgcatgtatttatactggtagtggtagcacagggtatgccgactccgtgaagggccgattcaccatctcccaagacaacgccgagaacacggtgtatctgcaaatgaacagcctgaaacctgaggacactgccatgtactactgtgcggcagcttcgccccggtggggtggtacctgtcgacggtggtccgagtataactactggggccaggggacccaggtcaccgtctcctca

SEQ ID NO: 409 - DNA, 369 bp, synthetic construct
  source 1..369: mol_type = other DNA
caggtgcacctggtggagtctgggggaggcttggtgcaggctggagggtctctgagactctcctgtgcagcctccggattcacctacagtaactgctgcatgaggtggtaccgccaggctccagggaaggcgcgcgagttggtctcattaattaatagtagtggtggcacatactatgcagactctgtgaggggccgattcaccatctccaaagacaacgccaagaacacgctgtatctgcaaatgaacagcctgaaacctgaggacacggccatgtattactgtgcggcttaccaagccaagtactgttcaggcccttgcgcccccccaactgactggggccaggggacccaggtcaccgtctcctca

SEQ ID NO: 410 - DNA, 363 bp, synthetic construct
  source 1..363: mol_type = other DNA
caggtgcagctggtggagtctgggggaggctcggtgcaggctggagggtctctgagactctcctgtgtagcctctggatacagcagcggtagttgtcgcatgggctggtaccgccaggctccagggaaggagcgcgagttggtttcaaaggttattagtgatggtactacagtctatgcagactccgtgaagggccgattcaccctctcccaaggaaacgccaagaacacggtgtatctgcaaatgagtagcctgttacctgaggacacggccatgtattactgtaatgcatggtgtagggagtatcccggggggatcctgaataacggctggggccaggggacccaggtcaccgtctcctca

SEQ ID NO: 411 - DNA, 354 bp, synthetic construct
  source 1..354: mol_type = other DNA
caggtgaagttggtggagtctggggggggcttggtgcaggcaggggggtctctgagactctcctgtacagtttctggattcactttcgatgacctcgtcatggcctggttccgccaggctccagggaaggagcgccaacttgtctcgttggttgcgactgctggtaatagcgtctatgcagactccgtgaagggccgattcacactctccagagacaacgcccacagcacggcgtatctgcaaatgaacggcctgaaacctgaggacacggccatgtattactgtgcggcacgtaccgattctgagcatgcgtttaagttctggggtcaggggacccaggtcaccgtctcctca

SEQ ID NO: 412 - DNA, 348 bp, synthetic construct
  source 1..348: mol_type = other DNA
gaggtgcaactggtggagtctgggggaggctcggtgcaggctggagggtctctgagactctcctgtgcagcctctggatacacttacagtagcaactggatgggctggttccgccaggctccagggaaggagcgcgaggaggtcgcaattatttatactggtggtattagtacacactatcgcagctccgtgaagggccgattcaccatctcccaagacaacgccaagaacacggtgtatctgcaaatgaacagcctgaaacctgaggacactgccatgtactactgtgcggcgcgttatacagactataactactggggccaggggacccaggtcaccgtctcctca

SEQ ID NO: 413 - DNA, 348 bp, synthetic construct
  source 1..348: mol_type = other DNA
caggtgcacctggtggagtctgggggaggctcggtgcaggctggagggtctctgagactctcctgtgaagtctctggatacaccggcgatacgacttacataggctggttccgccaggctccagggaaggagcgcgagggggtcgcacttatttatactagtggtactagcgagtactacgccgactccgtgaagggccgattcatcatctcccgagacaacgccaagaacacggtgtatttacaaatgaacagcctgaaacctgaggacactgccatgtactactgtggcgcacggagccgcacgatgatgtactggggccaggggacccaggtcaccgtctcctca

SEQ ID NO: 414 - DNA, 378 bp, synthetic construct
  source 1..378: mol_type = other DNA
caggtgcaattggaggagtctgggggaggcttggtgcagacaggggggtctctgagactctcctgtacagcctctggattcacttttgatcgtaatgccatgcgctggtaccgccaggctccagggaaggagcgcgagggggtctcatgtattagttggacgggtgcaaatattgcctatgcagactccgtgaagggccgattcaccatctccagagacaacgccaagaacacgctgtatctgcaaatgaacagcctgaaacctgaggacacgggcatgtattactgtgcggcagatacgacgtcggggtcttgttcaggcttttggtctacgagccggtactactggggccaggggacccaggtcaccgtctcctca

SEQ ID NO: 415 - DNA, 366 bp, synthetic construct
  source 1..366: mol_type = other DNA
caggtgaagttagtggagtctgggggaggctcggtgcaggctggagggtctctgagactctcctgtgcagcctctggatccacctactgtacctaccgtatgagctggttccgccaggctccagggaaggagcgcgagttcgtcgcagttattgatagtggcggtagcacaagctacgcagactccgtgaagggccgattcaccatctcccgagacaacgccaagaacacggtgtatctgcaaatgaacagcctgaaacctgaggacacggccatgtattactgtaaaacagatccaaccatcggctgcccccagacatataggtataactactggggccaggggacccaggtcaccgtctcctca

SEQ ID NO: 416 - DNA, 375 bp, synthetic construct
  source 1..375: mol_type = other DNA
caggtgcacctgatggagtctgggggaggctcggtgcaggctggagggtctctgagactctcctgtgtagcctctggaaacacttacaggctcaactctatgggctggttccgccaggctccagggaaggagcgcgagggggtcgcatttattgttatgattagaggtaccacatactatggcgcctccgtaaagggccgattcaccatctcccaagacaacgcccagaccacggtgtatctgcaaatgagcagcctgaaaccggaggacactgccatgtactactgtgcggcatccactaaggaccagttttatgtatttaatcctattgggtatgactcttggggccaggggacccaggtcaccgtctcctca

SEQ ID NO: 417 - DNA, 390 bp, synthetic construct
  source 1..390: mol_type = other DNA
caggtgcacctggtggagtctgggggaggctcggtgcaggctggagggtctctgagactctcctgtgcaacctctagatacatctacggtaacaactgcatggcctggttccgccaggctccagggaaggagcgcgagggagtcgcgtctatttatcctgctggtggtcgcacgtactatgccgactccgtgaagggccgattcaccatctcccaagacaacgccaagaacacggtgtatctgcaaattgacagcctgaaacctgaggacacggccatgtactactgcgcggcacgctctttttcgataggagtttgcgcgacgcagtctggtattacctggtctaattttgcttactggggccaggggacccaggtcaccgtctcctca

SEQ ID NO: 418 - DNA, 351 bp, synthetic construct
  source 1..351: mol_type = other DNA
caggtgcaactggcggagtctgggggaggctcggtgcaggctggagggtctctgagactctcctgtgcagcctctggatacacctacagtgcctgtagaatggcctggtaccgccaggctcccgggaaggagcgcgagttggtttcatttattaatagtgctggtagcacatactatgccgactccgtgaagggccgattcgccatctcccgagacaacgccaagacaacggtgtatctacaaatgaacgccctgaaagctgaggacacggccatatattactgtaacacatgggatagtagctgccgctttcagtactggggccaggggacccaggtcaccgtctcctca

SEQ ID NO: 419 - DNA, 404 bp, synthetic construct
  source 1..404: mol_type = other DNA
caggtgaggttggtggagtctgggggaggctcggtgcaggctggagggtctctgagactctcctgtgaaacctctagatacatctacggtaacaactgcatggcctggttccgccaggctccagggaaggagcgcgagggagtcgcgtctatttatcctgctggtggtcgcccgtactatgccgactccgtgaagggccgattcaccatctcccaaagacaacgccaagaacacggtgtatctgcaaaatggacagccctgaaaacctgaggacacggcccatgtactactgcgcgggcacgcttcttttttcgatagcaaattgcgccaacgcaatcctggtattaaccagggccaaattttggcttacgggggccaggggacccaggtcacccttctcctca

SEQ ID NO: 420 - DNA, 384 bp, synthetic construct
  source 1..384: mol_type = other DNA
caggtgaagttggtggagtctgggggaggctcggtgcagactggagggtctctgagactctcctgtgcagtctctacgtacacccccagtaacaactacatgggctggttccgccaggctccagggaaggagcgcgagggcgtcgcggctatcgctactattggtggtaccacacgttatgccgactccgtgaagggccgattcaccatctcccaagacggcgccaagaacacgatatatctgcaaatgaacggcctgaaaccggaggacactgccatgtactactgtgcggccgggcggccatactcattacccttacccttacccttggaaagcggtgcgtatcgctactggggccaggggacccaggtcaccgtctcctca

SEQ ID NO: 421 - DNA, 363 bp, synthetic construct
  source 1..363: mol_type = other DNA
caggtgaagttggtggagtctgggggaggctcggtgcaggctggagggtctctgagactctcctgtgtagcctctacatcaacctactgtaggtactacatgcgctggtaccggcaggctccagggaaagagcgcgagttcgtctcagcgatgcaacccgatggtacgacaagctactcagactccgtgaagggccgattcaccatgtcccaagacagagccaacaatatgttgtatctgcaaatgaacagcctgaggcctgaggacacggccatgtattactgtaaaagagatccaatggggggttcaaggaccccgtgcacctccgcctggggccaggggacccaggtcaccgtctcctca

SEQ ID NO: 422 - DNA, 375 bp, synthetic construct
  source 1..375: mol_type = other DNA
caggtgaggttggtggagtctgggggaggctcggtgcaggctggagggtctctgagactctcctgtgcagtctctggatacagatatagatggaactgcatggcctggttccgccaggctccagggaaggagcgcgagggggtcgcagctatttctactggaagcggaagcacatactatgccggctccgtgaagggccgattcaccatctcccaagacaacgccaagaacatgtatctgcaaatgaacagcctgaaacctgaggacactgccatgtactactgtgcggcagatccttcggtttgccccggtggtatgtggtactccaaagagtataggtactggggccaggggacccaggtcaccgtctcctca

SEQ ID NO: 423 - DNA, 378 bp, synthetic construct
  source 1..378: mol_type = other DNA
caggtgcacctgatggagtctgggggaggctcggtgcaggctggagggtctctgagactctcctgcacagcctctggacagacctccagatacctctacatgggctggtttcgccagactccagggaaggagcgcgagggggtcgcatgtatttatactggtagtggtagcacagggtatgccgactccgtgaagggccgattcaccatctcccaagacaacgccaagaacacggtgtatctgcaaacgaatagcctgaaacctgaggacactgccatgtactactgtgcggcaagttcgccccattggggcggtacctgtcgacgctggtccgagtataagtactggggccaggggacccaggtcaccgtctcctca

SEQ ID NO: 424 - DNA, 366 bp, synthetic construct
  source 1..366: mol_type = other DNA
caggtgcacctggtggagtctgggggaggctcggtgcaggctggagggtctctgagactctcctgtgcagcctctggacacacctacagtgccaactgcatggcctggttccgccgggccccagggaaggagcgcgagtgggtcgcgtcggtttatactgatgatgatagcacaatgtataccgactccgtgaagggccgattcaccatcttccaagacaacgccaagaacacggtgtatctgcaaatgaacagcctgaaacctgaggacactggcatgtacatctgtgcggcagatttaagcggaggaccggccggttgtgggtatacccactggggccaggggacccaggtcaccgtctcctca

SEQ ID NO: 425 - DNA, 387 bp, synthetic construct
  source 1..387: mol_type = other DNA
gaggtgcagctggtggagtctgggggaggctcggtgcaggctggagggtctctgagactctcctgtgcagcctctggatacacctacagtagcaacttcatgggctggttccgccaggctccagggaaggagcgcgagggggtcgcaactattgtttctggtggtggtaccacatactatgccgactccgtgaggggccgattcaccatctcccaagacaacgccaagaacacggtgtatctgcaaatgaacagcctgaaacctgaggacactgccatgtactactgtgcggcagggggcccccctacgaacggtgctaagtggtaccccctccgacctcctggttataactactggggccaggggacccaggtcaccgtctcctca

SEQ ID NO: 426 - DNA, 366 bp, synthetic construct
  source 1..366: mol_type = other DNA
caggtgcacctgatggagtctgggggaggctcggtgcaggctggagggtctctgagcctctcctgtgtagtctctggatacaccggcagtagccgctgtatggcctggttccgccaggctccagggaaggagcgcgaggcggtcgcacaaatttttactggtcgtggtaccacaggctatgccgactccgtgaagggccgattcactatttcccaagacaacgccaagaacacggtgtatctgcgaatgaacagtctgagacctgaggacactgccatttactactgtgcggcgagtctcggcccgggacgcggagcctgtgggtataactactggggccaggggacccaggtcaccgtctcctca

SEQ ID NO: 427 - DNA, 363 bp, synthetic construct
  source 1..363: mol_type = other DNA
caggtgcagctggtggagtctgggggaggctcggtgcaggctggagggtctctaagactctcctgtacaacctctggacgcacctacagtagctgcagcatgggctggtaccgccaggctccagggaaggagcgcgagttggtctcacatatttttagtgatggtagcagatactatgcagactccgtgaagggccgattcaccatctcccaagacaacgccaagaacacggtgtatctgcaaatgaacagcctgaaacctgaggacacggccatgtattactgtaaccgccgtacgggttgggcaccaaggtgcgctgttcccggttactggggccaggggacccaggtcaccgtctcctca

SEQ ID NO: 428 - DNA, 381 bp, synthetic construct
  source 1..381: mol_type = other DNA
caggtgcacctggtggagtctgggggaggctcggtgcaggctggagggtctctgagactctcctgtgcagcctctggatacacctatttcatgggctggttccgccaggctccacagaaggagcgcgaatgggtcgcgactattggtactggtgatattttcaatggcgctgcttactatgtcgactccgtgaagggccgattcgccatctcccaagacaacgccaagaacacggtgtatctgcaaatgaacagcctgaaacctgaagacactgccgtgtacgtctgtgcggcagttcaatcgaaatcctcaaactacgtgttgagagacgcatctacctacaactactggggccaggggacccaggtcaccgtctcctca

SEQ ID NO: 429 - DNA, 348 bp, synthetic construct
  source 1..348: mol_type = other DNA
gaggtgcagctggtggagtctgggggaggctcggtgcaggctgaagggtctctgagactctcctgtgcagcctctggagacaccagtagaagcgtctggatgggctgggcccgccaggttccagggaaagagcgcgaggtggtcgcaaccattagtactgccggtggtagtacatggtataccgactccgtgaagggccgattcaccatctcccaagacaacgccaagaacacggtgtacctgcaaatgaacagcctgaaacctgaggacactgccatatactattgtgcggccagaagcagatatgctacctactggggccaggggacccaggtcaccgtctcctca

SEQ ID NO: 430 - DNA, 378 bp, synthetic construct
  source 1..378: mol_type = other DNA
caggtgaggttggtggagtctgggggaggctcggtgcaggctggagggtctctgagactctcctgtacagcctctggatacacctacagatacctctacatggcctggttccgccagactccagggaaggagcgcgagggggtcgcatgtatttatactggtagtggtaccacagggtatgccgactccgtgaagggccgattcaccatctcccaagacaacgccaagaatacggtgtatctgcaaatgaacagcctgaacgctgaggacactgccatgtactactgtgcggcaagttcgccccagtggggcggtacctgtcgacgctggtccgagtataactactggggccaggggacccaggtcaccgtctcctca

SEQ ID NO: 431 - DNA, 348 bp, synthetic construct
  source 1..348: mol_type = other DNA
caggtgcaactgcaggagtctgggggaggctcggtgcaggctggagggtctctgagactctcctgtgcagcctctggatacacctggagtcgcaactggatgggctggttccgccaggctccagggaaggagcgcgaggggttcgcaactattacaattagtggtggtagcacatggtatgccgactccgtgaagggccgattcaccatctccctagacaacgccgggaacacggtgtatctgcaaatgaacagcctgaaacctgaggacactgccatgtactactgtgcggcgcgggataccgcgcggacctactggggccagggtacccaggtcaccgtctcctca

SEQ ID NO: 432 - DNA, 381 bp, synthetic construct
  source 1..381: mol_type = other DNA
gaggtgcagctggtggaatctgggggaggctcggtgcaggctggagggtctctgagactctcctgtgtagcctctgactacccctacatagacaactgcatgggctggttccgccagggtccagggaaggagcgcgagggggtcgcagctgcgtgtactggtggtggtagcacatattatgccgactccgtgaagggccgattcaccatctcccgagacaacgccaagaacacggtgtatctgcaaatgaacagcctgaaacctgaggacactgacgtgtactactgtgcgacaggctactatagcggctctggtccggggtatttactcccatggaggtataactactggggccaggggacccaggtcaccgtctcctca

SEQ ID NO: 433 - DNA, 393 bp, synthetic construct
  source 1..393: mol_type = other DNA
gaggtgcaactggtggaatctgggggaggctcggtgcaggctggagggtctctgagactctcctgtgcagcctctggatacaccgctaggcgcgacttcatggcctggttccgccaggttccagggaaggagcgcgagggggtcgcagtcattcatactggtggtgacaccacatactatgccgactccgtgaagggccgattcaccatctcccgcgacaacgcccagaacataatgaatctgcaaatgaacagccttaaacctgaggacactgccatgtactactgtgcggcaggtttccgtccgcgtggtggaggatacacgggtgacgtcttggcccaggctgcggcatacaactactggggccaggggacccaggtcaccgtctcctca

SEQ ID NO: 434 - DNA, 378 bp, synthetic construct
  source 1..378: mol_type = other DNA
gaggtgcagctggcggagtctgggggaggcttggtgcaggcaggggggtctctgagactctcctgtacagcctctggattcactattgctgtttataccatgggctggttccgccaggctccagggaaggagcgcgagggaatctcatgtactagctgggctggtggtcgcacatacactgcagactccgtgaagggccgattcaccatctccagagacaacgccaagaacacgctgtatctgcaaatgaacagcctgaaacctgaggacacggccatgtattactgtgcggcaaaggcacatcccgactgttcaggggattggtccccatctgggtatgaatactggggccaggggacccaggtcaccgtctcctca

SEQ ID NO: 435 - DNA, 348 bp, synthetic construct
  source 1..348: mol_type = other DNA
caggtgcacctggtggagtctgggggaggctcggtgcaggctggagggtctctgagactctcctgtgcagcctctggatacacctacaatagcaactacatgggctggttccgccaggctccagggaaggagcgcgagggggtcgcacttatttatactggtggtggtagcacatattatgccgactccgtgaagggccgattcaccatctcccgagacaacgccaaaaacacggtgtatctgcaaatgaacagcctgaaacctgaggacactgccatgtactactgttcggtaagaacgcagacgcgtaactactggggccaggggacccaggtcaccgtctcctca

SEQ ID NO: 436 - DNA, 393 bp, synthetic construct
  source 1..393: mol_type = other DNA
caggtgaagttggtggagtctgggggaggctcggtgcaggctggagggtctctgagactctcctgtgcagtttctagatatccctacagcagcatctgcatgggctggttccgccaggctccagggaaggagagcgagggtgtcgcacgtatttatactggtactggtagtacatggtataccgactccgtgaagggccgattcaccatcgcccgagacaacgcccagaacacggtgtatctgcaaatgaacagcctgaaacctgaggacactgccatgtactactgtgcggcgcgtagcaattcatattcatattcaagttgtgattacggccccctcacgaggggggggtataacttctggggccaggggacccaggtcaccgtctcctca

SEQ ID NO: 437 - DNA, 345 bp, synthetic construct
  source 1..345: mol_type = other DNA
gaggtgcagctggcggagtctgggggaggatcggtgcaggctggagggtctctgagactctcctgtgcagtctctggatacaccagccgtagcaattacatgggctggttccgccaggctccagggaaggagcgcgagggggtcgcgctaatttatactcgtggtggtagcacatactatgcctcctccgtgaagggccggttcaccatctcccaagacagtgccaagaaaacgtatctgcaaatgaacagtgtgaaaccggaggacactgccatgtactactgtgctttgcgccttgatgagaagatgtactggggccaggggacccaggtcaccgtctcctca

SEQ ID NO: 438 - DNA, 399 bp, synthetic construct
  source 1..399: mol_type = other DNA
gaggtgcagctggcggagtctgggggaggctcggtgcaggctggagggtctctgagactctcctgtgatgcctctggatacacctacagccgcaactgcatgggctggttccgccaggctccagggaaggagcgcgagggggtcgcagcgttctatactgattatattcgttttgggcgcacatattatgccgactccgtgaagggccgattcaccatcttccaagacaacgccaagaacacggtgtatctgcaaatgaacagcctgaaacctgaggacactgccatgtactactgtgcggcagatcctgggagtcgtacagacgatagttgtggtacctcatacaacaaagggaattttggttactggggccaggggacccaggtcaccgtctcctca

SEQ ID NO: 439 - DNA, 390 bp, synthetic construct
  source 1..390: mol_type = other DNA
caggtgcagctggtggagtctgggggaggctcggtgcaggctggagggtcgctgagactctcctgtacagcctctggatacacctacagaagcaactgtatgggctggttccgccaggctccagggaaggagcgcgagggggtcgcaacaatctatactggtggtggtcgtaatctatactatgccgactccgtgaagggccgattcaccatctcccgagacaacgccaagaacaccctgtacctgcaaatgaacagcctgaagcctgaggactctgccaggtactactgtgcggccgcgagtgacgtggcagttggtgttaattcctgcgggggaaggactgcggggtttgacgcctggggccaggggacccaggtcaccgtctcctca

SEQ ID NO: 440 - DNA, 375 bp, synthetic construct
  source 1..375: mol_type = other DNA
caggtgaggttagtggagtctgggggaggatcggtacaggctggagggtctctgagactctcctgctcagcctctggatacacctacagttacaacaatatgggctggttccgccaggctccagggaacgagcgcgagggggtcgcagctattagtggtggtcgtttcaccgcctatgccgactccgtgaagggccgattcaccatctcccgagacaacgccgagaacacgctgtatctgcaaatgaacaacctgaaacctgaggacactgggatgtactactgtgcggcagaggtagttgatccgacctgggggtcgcgtgaccaaagacgatataagtactggggccaggggacccaggtcaccgtctcctca

SEQ ID NO: 441 - DNA, 372 bp, synthetic construct
  source 1..372: mol_type = other DNA
caggtgaagttggtggagtctgggggaggctcggtgcaggctggagggtctctgagactctcgtgtgcagcctctggatacatctacagttgcgtgggctggttccgccaggctccagggaaggagcgcgagggggtcgcaggtattagtactggtggtggtggcacagtctatgccgactccgtgaagggccaattcaccatctcccgagacaacgccaagaacacggtgtacctgcaaatggacagcctgaaacctgaggacactgccatgtactactgtgcggcagatcgatggaattcattcgctaattgcggtgcctggggaaggtatacctactggggccaggggacccaggtcaccgtctcctca

SEQ ID NO: 442 - DNA, 390 bp, synthetic construct
  source 1..390: mol_type = other DNA
caggtgcaactggcggagtctgggggaggctcggtgcaggctggagggtctctgagactctcctgtgtagcctctggatacccctcttccacctactacatgctcagcatggcgtggttccgccaggctccagggaaggagcgcgagggggtcgccgctattactagcggtactgggagcacaagctacgcagactccgtgaaggaccgattcaccatctccaaagactacgccaacaacactctgtatctgcacataaacaacctgaaacctgaggacactgccatgtactactgtgcggcagcctcaggttggatcgttcctagtaggtccctgaccgccaacctatataggtattggggccaggggacccaggtcaccgtctcctca

SEQ ID NO: 443 - DNA, 375 bp, synthetic construct
  source 1..375: mol_type = other DNA
caggtgcacctggtggagtctgggggagactcggtgcaggctggagggtccctgagactctcctgtgcagcctctggatacacctacaataccaactacatgggctggttccgccaggctccagggaaggagcgcgagggggtcgcagctatttatagacatagtggtaacacagcctatgccgactccgtgaagggccgattcaccatctcccaagactacgccaagaacaccgtgtatctgcaaatgaacagcctgaaacctgaggacactgccatgtactactgtgcggcagggcgcgctggtccctgggccctgatgcgcccgactgagtttggttactggggccaggggacccaggtcaccgtctcctca

SEQ ID NO: 444 - DNA, 369 bp, synthetic construct
  source 1..369: mol_type = other DNA
caggtgcaattggaggagtctgggggaggcttggtgcagcctggggggtctctgagactctcctgtgcggcctctggagacacatttcgtgcctattacatgaactgggtccgccaggctccagggaagggattcgagtgggtctcaggtattagcgccagtggcggccgtacgtcatacgcagactccgtgaagggccgattcaccatctccagagacaacgccaaaaacacgctgtatctgcaattgaacagcctgagcactgaggacacgggcatgtattattgtgtaaagggagctgtccgtctctcgacatcgtcagtacgggattcgtcctggggccaggggacccaggtcaccgtctcctca

SEQ ID NO: 445 - DNA, 348 bp, synthetic construct
  source 1..348: mol_type = other DNA
caggtgcaattggaggagtctgggggaggctcggtgcaggctggagggtctctgagactctcctgtgtagtctctggaaacacccgcagtaccacgtacatgggctggttccgccaggctccagggaaggagcgcgagggggtcgcaatagtttatactggtggtcgtgacacatactatgccgcctccgtgaagggccgattcaccatctcccaagacaacgccaagacaacgatctatctgcaaatgaacagtctggaacctgaggacactgccatgtactactgtgcggcacgctcatatgagtatacctactggggtcgggggacccaggtcaccgtctcctca

SEQ ID NO: 446 - DNA, 372 bp, synthetic construct
  source 1..372: mol_type = other DNA
gaggtgcaactggcggagtctgggggaggctcggtgcaggctggagggtctctgagactctcctgtgtagcctctggatacaccttcagtagctactgcttgggctggttccgccaggctccagggaagcagcgccagggggtcgcaacgtttaataatagaggtgtcgcaaactaccacgattccgtgaagggccgattcaccgcctccgtagacaacgccaagaacactctgcttctgcaaatgaacagcctggaacctgacgacacggccatgtactactgtgcggcggatcgccggtacggtcgtcagtggtatcagccttgcgagtggaacacctggggccaggggacccaggtcaccgtctcctca

SEQ ID NO: 447 - DNA, 387 bp, synthetic construct
  source 1..387: mol_type = other DNA
caggtgaggttggtggagtctgggggaggctcggtgcaggctggagggtctctgagactctcctgtgtagcctctggatacttctacaatacctactactttatgggctggttccgccaggctccagggaaggagcgcgagggggtcgcagctattgatactgatggtagaacaagttacgcagactccgtgaagggccgattcaccatctccaaagacaacgccaagaacactctgtatctgcaaatgaacagcctgaaacctgaggacactgccatgtactactgtgcggcaggttttggctatatgaatgttattcaggctcttaatggcatgagacagaatcccgactactggggccaggggacccaggtcaccgtctcctca

SEQ ID NO: 448 - DNA, 372 bp, synthetic construct
  source 1..372: mol_type = other DNA
caggtgaagttggtggagtctgggggaggctcggtgcaggctggagggtctctgagactctcctgtgcagcctctggatacaccttcgctggcaactgcttgggctggtttcgccaggctccagggaaggggcgcgagggggtcgtaacgtacaataacttcggtgtcgccaactacgccgattccgtgaagggccgattcaccgtctcccaagacaacgccaagaacactctgcttctgcaaatgaacagcctggaacctgaggacactgccatgtactactgtgcggcggaccgccgggacggtcgtcgctggtctcagccttgcgagtggaatacctggggccaggggacccaggtcaccgtctcctca

SEQ ID NO: 449 - DNA, 375 bp, synthetic construct
  source 1..375: mol_type = other DNA
gaggtgcagctggtggagagcggaggaggactggtgcagccaggaggcagcctgaggctgtcctgcgcagcatctggaaacacctacagctccaattatatgggatggttcaggcaggcacctggcaagggactggaggaggtggccgtgatctacaccaggggaggacacacatactatgtggactccgtgcggggacggttcaccatcagcagggataacgccaagaacagcctgtatctgcagatgaactctctgagagccgaggacacagccgtgtactattgtgcagcatctagcaggcacaggctgggcctgaacaatccaagggactacgattattggggccagggcaccctggtgacagtgtcctct

SEQ ID NO: 450 - DNA, 375 bp, synthetic construct
  source 1..375: mol_type = other DNA
gaggtgcagctggtggagagcggaggaggactggtgcagccaggaggcagcctgaggctgtcctgcgcagcatctggaaacacctacagctccaattatatgggatggttcaggcaggcacctggcaagggcctggaggaggtggccgtgatctacaccagaggcggccacacatactatgtggactccgtgcggggacggttcaccatcagccaggataacgccaagaacagcctgtatctgcagatgaactctctgagggccgaggacacagccgtgtactattgtgcagcatctagcaggcacaggctgggcctgaacaatccaagggactacgattattggggccagggcaccctggtgacagtgtcctct

SEQ ID NO: 451 - DNA, 375 bp, synthetic construct
  source 1..375: mol_type = other DNA
gaggtgcagctggtggagagcggaggaggactggtgcagccaggaggcagcctgaggctgtcctgcgcagcatctggaaacacctacagctccaattatatgggatggttcaggcaggcacctggcaagggcctggaggaggtggccgtgatctacaccagaggcggccacacatactatgtggactccgtgcggggacggttcaccatcagccaggataacgccaagaacagcgtgtatctgcagatgaactctctgagggccgaggacacagccatgtactattgtgcagcatctagcaggcacaggctgggcctgaacaatccaagggactacgattattggggccagggcaccctggtgacagtgtcctct

SEQ ID NO: 452 - DNA, 375 bp, synthetic construct
  source 1..375: mol_type = other DNA
gaggtgcagctggtggagagcggaggaggactggtgcagccaggaggcagcctgaggctgtcctgcgcagcatctggaaacacctacagctccaattatatgggatggttcaggcaggcacctggcaagggaagagaggaggtggccgtgatctacaccaggggaggacacacatactatgtggactccgtgcggggacggttcaccatcagccaggataacgccaagaacagcgtgtatctgcagatgaactctctgagggccgaggacacagccatgtactattgtgcagcatctagcaggcacaggctgggcctgaacaatccaagggactacgattattggggccagggcaccctggtgacagtgtcctct

SEQ ID NO: 453 - DNA, 375 bp, synthetic construct
  source 1..375: mol_type = other DNA
gaggtgcagctggtggagtccggaggaggactggtgcagccaggaggctctctgaggctgagctgcgcagcatccagagcaacctactctacaaactatatcagctggttcaggcaggcacctggcaagggactggaggcagtggcaaccatcaccacaggcgatggcgagacagcctacgccgactctgtgaagggcaggtttaccatctcccgcgataacgccaagaacagcctgtatctgcagatgaacagcctgagggccgaggacacagccgtgtactattgtgcagcaaatctgaggatcggaggcgactggttcgatggaagggactttagagcatggggacagggaaccctggtgacagtgagctcc

SEQ ID NO: 454 - DNA, 375 bp, synthetic construct
  source 1..375: mol_type = other DNA
gaggtgcagctggtggagtccggaggaggactggtgcagccaggaggctctctgaggctgagctgcgcagcatccagagcaacctactctacaaactatatcagctggttcaggcaggcacctggcaagggactggaggcagtggcaaccatcaccacaggcgatggcgagacagcctacgccgactctgtgaagggcaggtttaccatctcccgcgataacgccaagaacagcgtgtatctgcagatgaacagcctgagggccgaggacacagccatgtactattgtgcagcaaatctgaggatcggaggcgactggttcgatggaagggactttagagcatggggacagggaaccctggtgacagtgagctcc

SEQ ID NO: 455 - DNA, 375 bp, synthetic construct
  source 1..375: mol_type = other DNA
gaggtgcagctggtggagtccggaggaggactggtgcagccaggaggctctctgaggctgagctgcgcagcatccagagcaacctactctacaaactatatcagctggttcaggcaggcacctggcaagggaagggaggcagtggccaccatcaccacaggcgatggcgagacagcctacgccgactctgtgaagggcaggtttaccatctcccgcgataacgccaagaacagcgtgtatctgcagatgaacagcctgcgggccgaggacacagccatgtactattgtgcagcaaatctgaggatcggaggcgactggttcgatggaagggactttagagcatggggacagggaaccctggtgacagtgagctcc

SEQ ID NO: 456 - DNA, 375 bp, synthetic construct
  source 1..375: mol_type = other DNA
caggtgcagctggtggagagcggaggaggagtggtgcagccaggaggcagcctgaggctgtcctgcgcaggctctttcagcggatacggcgtgtccaccatggcatggtttaggcaggcacctggcaagggactggagggagtggcagcaatcaccgtgggatccggaaacacatactatgccgactctgtgaccggccggttcacaatctctagagataacagcaagaataccctgtatctgcagatgaacagcctgcgggccgaggacacagccgtgtactattgtgcagcaggatggctgtccggaggatcttggcacgtgcccggcaggtacaactattggggccagggcaccctggtgacagtgagctcc

SEQ ID NO: 457 - DNA, 375 bp, synthetic construct
  source 1..375: mol_type = other DNA
caggtgcagctggtggagagcggaggaggagtggtgcagccaggaggcagcctgaggctgtcctgcgcaggctctttcagcggatacggcgtgtccaccatggcatggtttaggcaggcacctggcaagggactggagggagtggcagcaatcaccgtgggatccggaaacacatactatgccgactctgtgaccggccggttcacaatctctagagataacagcaagaataccgtgtatctgcagatgaacagcctgcgggccgaggacacagccatgtactattgtgcagcaggatggctgtccggaggatcttggcacgtgcccggcaggtacaactattggggccagggcaccctggtgacagtgagctcc

SEQ ID NO: 458 - DNA, 375 bp, synthetic construct
  source 1..375: mol_type = other DNA
caggtgcagctggtggagagcggaggaggagtggtgcagccaggaggcagcctgaggctgtcctgcgcaggctctttcagcggatacggcgtgtccaccatggcctggtttaggcaggcacctggcaagggaagggagggagtggcagcaatcaccgtgggatccggaaacacatactatgccgactctgtgaccggccggttcacaatctctagagataacagcaagaataccgtgtatctgcagatgaacagcctgcgggccgaggacacagccatgtactattgtgcagcaggatggctgtccggaggatcttggcacgtgcccggcaggtacaactattggggccagggcaccctggtgacagtgagctcc

SEQ ID NO: 459 - DNA, 375 bp, synthetic construct
  source 1..375: mol_type = other DNA
caggtgcagctggtggagagcggaggaggagtggtgcagccaggaggcagcctgaggctgtcctgcgcagcctctttcagcggatacggcgtgtccaccatggcatggtttaggcaggcacctggcaagggactggagggagtggcagcaatcaccgtgggatccggaaacacatactatgccgactctgtgaagggccggttcaccatctctagagataacagcaagaatacactgtacctgcagatgaacagcctgcgggccgaggacacagccgtgtactattgtgccgtgggctatctgtccggaggatcttgggatgtgccaggaaggtacaactattggggccagggcaccctggtgacagtgagctcc

SEQ ID NO: 460 - DNA, 375 bp, synthetic construct
  source 1..375: mol_type = other DNA
caggtgcagctggtggagagcggaggaggagtggtgcagccaggaggcagcctgaggctgtcctgcgcagcctctttcagcggatacggcgtgtccaccatggcatggtttaggcaggcacctggcaagggactggagggagtggcagcaatcaccgtgggatccggaaacacatactatgccgactctgtgaagggccggttcaccatctctagagataacagcaagaatacagtgtacctgcagatgaacagcctgcgggccgaggacacagccatgtactattgtgccgtgggctatctgtccggaggatcttgggatgtgccaggaaggtacaactattggggccagggcaccctggtgacagtgagctcc

SEQ ID NO: 461 - DNA, 375 bp, synthetic construct
  source 1..375: mol_type = other DNA
caggtgcagctggtggagagcggaggaggagtggtgcagccaggaggcagcctgaggctgtcctgcgcagcctctttcagcggatacggcgtgtccaccatggcctggtttaggcaggcacctggcaagggaagggagggagtggcagcaatcaccgtgggatccggaaacacatactatgccgactctgtgaagggccggttcaccatctctagagataacagcaagaatacagtgtacctgcagatgaacagcctgcgggccgaggacacagccatgtactattgtgccgtgggctatctgtccggaggatcttgggatgtgccaggaaggtacaactattggggccagggcaccctggtgacagtgagctcc

SEQ ID NO: 462 - protein, 15 aa, synthetic construct
GGGGSGGGGSGGGGS

SEQ ID NO: 463 - protein, 10 aa, synthetic construct
GGGGSGGGGS

SEQ ID NO: 464 - protein, 5 aa, synthetic construct
GGGGS

SEQ ID NO: 465 - protein, 21 aa, synthetic construct
MALPVTALLLPLALLLHAARP

SEQ ID NO: 466 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 467 - protein, 24 aa, synthetic construct
IYIWAPLAGTCGVLLLSLVITLYC

SEQ ID NO: 468 - protein, 42 aa, synthetic construct
KRGRKKLLYIFKQPFMRPVQTTQEEDGCSCRFPEEEEGGCEL

SEQ ID NO: 469 - protein, 41 aa, synthetic construct
RSKRSRLLHSDYMNMTPRRPGPTRKHYQPYAPPRDFAAYRS

SEQ ID NO: 470 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 471 - protein, 25 aa, synthetic construct
GSGVKQTLNFDLLKLAGDVESNPGP

SEQ ID NO: 472 - protein, 22 aa, synthetic construct
GSGATNFSLLKQAGDVEENPGP

SEQ ID NO: 473 - protein, 257 aa, synthetic construct
MALPVTALLLPLALLLHAARPAIQLTQSPSSLSASVGDRVTITCRASENIYYNLAWYQQKPGKAPKLLIYTANSLEDVPSRFSGSGSGTDFTLTISSLQPEDFATYFCKQAYDVPPTFGGGTKLEIKGGGGSGGGGSGGGGSQVQLVQSGAEVKKPGASVKVSCKASGYTFTRYWIHWIRQAPGQGLEWMGYINPTVYTEFNQNFKDRVTMTRDTSTSTVYMELSSLRSEDTAVYYCARGGSNFFDYWQGTTVTVSS

SEQ ID NO: 474 - protein, 27 aa, synthetic construct
FWVLVVVGGVLACYSLLVTVAFIIFWV

SEQ ID NO: 475 - protein, 39 aa, synthetic construct
IEVMYPPPYLDNEKSNGTIIHVKGKHLCPSPLFPGPSKP

SEQ ID NO: 476 - protein, 369 aa, synthetic construct
MALPVTALLLPLALLLHAARPQVRLVESGGGSVQAGGSLRLSCAGSFSGYGVSTMAWFRQAPGKEREGVAAITVGSGNTYYADSVKGRFTISRDNAKRTVFLQMNSLKPEDTAMYYCAVGYLSGGSWDVPGRYNYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 477 - protein, 369 aa, synthetic construct
MALPVTALLLPLALLLHAARPEVQLVESGGGSVQAGGSLTLSCEASGNTYSSNYMGWFRQAPGKEREEVAVIYTRGGHTYYVDSVRGRFTISQDNAKNTVYLQMNSLKPEDTAMYYCAASSRHRLGLNNPRDYDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 478 - protein, 369 aa, synthetic construct
MALPVTALLLPLALLLHAARPQVQLVESGGGSVQAGGSLRLSCAASRATYSTNYISWFRQAPGKEREAVATITTGDGETAYADSVKGRFTISRDNAKNTVYLQMNSLKPEDTAMYYCAANLRIGGDWFDGRDFRAWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 479 - protein, 361 aa, synthetic construct
MALPVTALLLPLALLLHAARPQVQLVESGGGSVQAGGSLRLSCAASGYTDRCSMAWYRQAPGKERELVSRISTSGFTNYAASVKGRFTISQDNAKNTVYLQMNSLNPGDTGMYYCAIIVGRTCSLNYWGNGILVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 480 - protein, 376 aa, synthetic construct
MALPVTALLLPLALLLHAARPEVQLAESGGGSVQAGGSLRLSCSASGFTFDDSDMAWYRQAPGDGCDLVSTISSDGSTYYADSVKGRFTISQDNAKNTVYLQMHSLKPEDTAVYYCAADFLTGFYYSDSPHPAPCSASDFGYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 481 - protein, 369 aa, synthetic construct
MALPVTALLLPLALLLHAARPEVQLAESGGGSVQAGGSLRLSCAGSFSGYGVSTMAWFRQAPGKEREGVAAITVGSGNTYYADSVTGRFTISRDNAKRTVYLQMNSLKPEDTAMYYCAAGWLSGGSWHVPGRYNYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 482 - protein, 369 aa, synthetic construct
MALPVTALLLPLALLLHAARPQVHLVESGGGSVQAGGSLRLSCAASQYVYRWDLMGWFRQAPGKEREAVAAVYTGDGITYYADSVKGRFSISQDNAKNTVYLQMNSLKPEDTGMYFCAAGFVSGGRWNQSYRYKYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 483 - protein, 373 aa, synthetic construct
MALPVTALLLPLALLLHAARPQVHLMESGGGSVQAGGSLRLSCAASGYTYRSNFMGWFRQAPGKEREGIATIHSGVATTYYADSVKGRFTISQDNAKNTVYLQMNSLKPEDTAMYYCAAGGPPANADRWYPLRPPGYNYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 484 - protein, 371 aa, synthetic construct
MALPVTALLLPLALLLHAARPQVRLVESGGGSVQVGGSLRLSCAASRSPYSSSRCMGWFRQAPGKEREGVAALYTGGGSTSYADSVKGRFTISQDNAKNTVYLQMNSLKPEDTAMYYCAAVVPRGGSCRLDERGYYHWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 485 - protein, 369 aa, synthetic construct
MALPVTALLLPLALLLHAARPEVQLVESGGGLVQPGGSLRLSCAASGNTYSSNYMGWFRQAPGKGLEEVAVIYTRGGHTYYVDSVRGRFTISRDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 486 - protein, 369 aa, synthetic construct
MALPVTALLLPLALLLHAARPEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 487 - protein, 369 aa, synthetic construct
MALPVTALLLPLALLLHAARPEVQLVESGGGLVQPGGSLRLSCAASGNTYSSNYMGWFRQAPGKGLEEVAVIYTRGGHTYYVDSVRGRFTISQDNAKNSVYLQMNSLRAEDTAMYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 488 - protein, 369 aa, synthetic construct
MALPVTALLLPLALLLHAARPEVQLVESGGGLVQPGGSLRLSCAASGNTYSSNYMGWFRQAPGKGREEVAVIYTRGGHTYYVDSVRGRFTISQDNAKNSVYLQMNSLRAEDTAMYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 489 - protein, 369 aa, synthetic construct
MALPVTALLLPLALLLHAARPEVQLVESGGGLVQPGGSLRLSCAASRATYSTNYISWFRQAPGKGLEAVATITTGDGETAYADSVKGRFTISRDNAKNSLYLQMNSLRAEDTAVYYCAANLRIGGDWFDGRDFRAWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 490 - protein, 369 aa, synthetic construct
MALPVTALLLPLALLLHAARPEVQLVESGGGLVQPGGSLRLSCAASRATYSTNYISWFRQAPGKGLEAVATITTGDGETAYADSVKGRFTISRDNAKNSVYLQMNSLRAEDTAMYYCAANLRIGGDWFDGRDFRAWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 491 - protein, 369 aa, synthetic construct
MALPVTALLLPLALLLHAARPEVQLVESGGGLVQPGGSLRLSCAASRATYSTNYISWFRQAPGKGREAVATITTGDGETAYADSVKGRFTISRDNAKNSVYLQMNSLRAEDTAMYYCAANLRIGGDWFDGRDFRAWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 492 - protein, 369 aa, synthetic construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTLYLQMNSLRAEDTAVYYCAVGYLSGGSWDVPGRYNYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 493 - protein, 369 aa, synthetic construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 494 - protein, 369 aa, synthetic construct
MALPVTALLLPLALLLHAARPQVQLVESGGGVVQPGGSLRLSCAASFSGYGVSTMAWFRQAPGKGREGVAAITVGSGNTYYADSVKGRFTISRDNSKNTVYLQMNSLRAEDTAMYYCAVGYLSGGSWDVPGRYNYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 495 - protein, 11 aa, synthetic construct
RASQSVSSAVA

SEQ ID NO: 496 - protein, 7 aa, synthetic construct
SASSLYS

SEQ ID NO: 497 - protein, 9 aa, synthetic construct
QQASWSPIT

SEQ ID NO: 498 - protein, 10 aa, synthetic construct
GFNISSSYMH

SEQ ID NO: 499 - protein, 17 aa, synthetic construct
YIYPSYGYTSYADSVKG

SEQ ID NO: 500 - protein, 10 aa, synthetic construct
GGYYYHGMDY

SEQ ID NO: 501 - protein, 11 aa, synthetic construct
RASQSVSSAVA

SEQ ID NO: 502 - protein, 7 aa, synthetic construct
SASSLYS

SEQ ID NO: 503 - protein, 10 aa, synthetic construct
QQHYAPSLIT

SEQ ID NO: 504 - protein, 10 aa, synthetic construct
GFNISSYSMH

SEQ ID NO: 505 - protein, 17 aa, synthetic construct
YISSYYGYTYYADSVKG

SEQ ID NO: 506 - protein, 9 aa, synthetic construct
YSYYYGMDY

SEQ ID NO: 507 - protein, 107 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQASWSPITFGQGTKVEIK

SEQ ID NO: 508 - protein, 119 aa, synthetic construct
EVQLVESGGGLVQPGGSLRLSCAASGFNISSSYMHWVRQAPGKGLEWVAYIYPSYGYTSYADSVKGRFTISADTSKNTAYLQMNSLRAEDTAVYYCARGGYYYHGMDYWGQGTLVTVSS

SEQ ID NO: 509 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQHYAPSLITFGQGTKVEIK

SEQ ID NO: 510 - protein, 118 aa, synthetic construct
EVQLVESGGGLVQPGGSLRLSCAASGFNISSYSMHWVRQAPGKGLEWVAYISSYYGYTYYADSVKGRFTISADTSKNTAYLQMNSLRAEDTAVYYCARYSYYYGMDYWGQGTLVTVSS

SEQ ID NO: 511 - DNA, 321 bp, synthetic construct
  source 1..321: mol_type = other DNA
gacatccagatgacccagagcccgagcagcctgagcgcgagcgttggtgaccgtgttaccattacctgccgtgcgagccagagcgttagcagcgcggtggcgtggtaccagcaaaagccgggtaaagcgccgaagctgctgatctatagcgcgagcagcctgtatagcggcgttccgagccgtttcagcggtagccgtagcggcaccgactttaccctgaccattagcagcctgcagccggaagatttcgcaacttattactgtcagcaagcttcttggtctccgatcacgttcggacagggcaccaaagttgagattaaa

SEQ ID NO: 512 - DNA, 357 bp, synthetic construct
  source 1..357: mol_type = other DNA
gaggttcaactggtggagagcggtggtggtctggttcagccgggtggtagcctgcgtctgagctgcgcagcttctggcttcaacatctcttcttcttatatgcactgggtgcgtcaggcgccgggtaaaggcctggaatgggttgcatatatttatccttcttatggctatacttcttatgccgatagcgtcaagggccgtttcaccatcagcgcggataccagcaaaaacaccgcatacctgcaaatgaacagcctgcgtgcggaagataccgccgtctattattgtgctcgcggtggttactactaccatggtatggactactggggtcaaggcaccctggttaccgtgagcagc

SEQ ID NO: 513 - DNA, 324 bp, synthetic construct
  source 1..324: mol_type = other DNA
gacatccagatgacccagagcccgagcagcctgagcgcgagcgttggtgaccgtgttaccattacctgccgtgcgagccagagcgttagcagcgcggtggcgtggtaccagcaaaagccgggtaaagcgccgaagctgctgatctatagcgcgagcagcctgtatagcggcgttccgagccgtttcagcggtagccgtagcggcaccgactttaccctgaccattagcagcctgcagccggaagatttcgcaacttattactgtcagcaacattacgctccgtctctgatcacgttcggacagggcaccaaagttgagattaaa

SEQ ID NO: 514 - DNA, 354 bp, synthetic construct
  source 1..354: mol_type = other DNA
gaggttcaactggtggagagcggtggtggtctggttcagccgggtggtagcctgcgtctgagctgcgcagcttctggcttcaacatctcttcttattctatgcactgggtgcgtcaggcgccgggtaaaggcctggaatgggttgcatatatttcttcttattatggctatacttattatgccgatagcgtcaagggccgtttcaccatcagcgcggataccagcaaaaacaccgcatacctgcaaatgaacagcctgcgtgcggaagataccgccgtctattattgtgctcgctactcttactactacggtatggactactggggtcaaggcaccctggttaccgtgagcagc

SEQ ID NO: 515 - protein, 485 aa, synthetic construct
MALPVTALLLPLALLLHAARPDIQMTQSPSSLSASVGDRVTITCRASQSVSSAVAWYQQKPGKAPKLLIYSASSLYSGVPSRFSGSRSGTDFTLTISSLQPEDFATYYCQQASWSPITFGQGTKVEIKGGGGSGGGGSGGGGSEVQLVESGGGLVQPGGSLRLSCAASGFNISSSYMHWVRQAPGKGLEWVAYIYPSYGYTSYADSVKGRFTISADTSKNTAYLQMNSLRAEDTAVYYCARGGYYYHGMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 516 - protein, 485 aa, synthetic construct
MALPVTALLLPLALLLHAARPDIQMTQSPSSLSASVGDRVTITCRASQSVSSAVAWYQQKPGKAPKLLIYSASSLYSGVPSRFSGSRSGTDFTLTISSLQPEDFATYYCQQHYAPSLITFGQGTKVEIKGGGGSGGGGSGGGGSEVQLVESGGGLVQPGGSLRLSCAASGFNISSYSMHWVRQAPGKGLEWVAYISSYYGYTYYADSVKGRFTISADTSKNTAYLQMNSLRAEDTAVYYCARYSYYYGMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 517 - protein, 476 aa, synthetic construct
MALPVTALLLPLALLLHAARPQVQLQESGPGLVKPSETLSLTCTVSGDSISSYYWTWIRQPPGKGLEWIGYIYYSGTTNYNPSLKSRVTISVDTSKSQFSLKLSSVTAADTAVYYCASIAVRGFFFDYWGQGTLVTVSSGGGGSGGGGSGGGGSEIVLTQSPGTLSLSPGERATLSCRASQSVSSSYLAWYQQKPGQAPRLLIYGASTRATGIPDRFSGSGSGTDFTLTISRLEPEDFAVYYCQQYGTSPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 518 - protein, 509 aa, synthetic construct
MALPVTALLLPLALLLHAARPEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 519 - protein, 509 aa, synthetic construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 520 - protein, 509 aa, synthetic construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 521 - protein, 775 aa, synthetic construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQAAAPTTTPAPRPPTPAPTIASQPLSLRPEACRPAAGGAVHTRGLDFACDIYIWAPLAGTCGVLLLSLVITLYCNHRRIQ

SEQ ID NO: 522 - protein, 766 aa, synthetic construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 523 - protein, 244 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQAAAPTTTPAPRPPTPAPTIASQPLSLRPEACRPAAGGAVHTRGLDFACDIYIWAPLAGTCGVLLLSLVITLYCNHRRIQ

SEQ ID NO: 524 - protein, 235 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 525 - protein, 730 aa, synthetic construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 526 - protein, 730 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGATNFSLLKQAGDVEENPGPMALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 527 - protein, 729 aa, synthetic construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 528 - protein, 729 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGEGRGSLLTCGDVEENPGPMALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 529 - protein, 199 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

(19) United States
(12) Patent Application Publication
(10) Pub. No.: US 2026/0078166 A1
(43) Pub. Date: Mar. 19, 2026

(54) ANTI-DLL3 CHIMERIC ANTIGEN RECEPTORS AND USES THEREOF

(71) Applicant: Legend Biotech Ireland Limited, Dublin 18 (IE)

(72) Inventors: Tao Zhao, Nanjing (CN); Yuanyuan Peng, Nanjing (CN); An Tang, Nanjing (CN); Sujuan Wang, Nanjing (CN); Shuai Yang, Nanjing (CN); Wang Zhang, Nanjing (CN); Shu Wu, Nanjing (CN); Ruidong Hao, Shanghai (CN)

(21) Appl. No.: 19/308,077

(22) Filed: Aug. 22, 2025

Related U.S. Application Data
(62) Division of application No. 17/618,642, now Pat. No. 12,421,292.

(30) Foreign Application Priority Data
Jul. 17, 2019 (WO) PCT/CN2019/096360
May 15, 2020 (WO) PCT/CN2020/090587

(51) Int. Cl. C07K 14/705; A61K 38/00; A61K 39/00; A61K 40/11; A61K 40/31; A61K 40/42; A61P 35/00; C07K 14/71; C07K 14/725; C07K 16/18; C12N 5/0783

(52) U.S. Cl. CPC C07K 14/70521; A61K 40/11; A61K 40/31; A61K 40/4202; A61K 40/4229; A61P 35/00; C07K 14/7051; C07K 14/70596; C07K 14/71; C07K 16/18; C12N 5/0636; C12N 5/0638; C12N 5/0646; A61K 38/00; A61K 2039/505; A61K 2239/31; A61K 2239/38; C07K 2317/22; C07K 2317/569; C07K 2317/622

(57) ABSTRACT

Provided herein are anti-DLL3 chimeric antigen receptors (CARs), DLL3 binding proteins and uses of such CARs or DLL3 binding proteins in the treatment of DLL3 associated disorders, such as small cell lung cancer.

### CROSS REFERENCE TO RELATED APPLICATIONS

This application is a divisional application of U.S. application Ser. No. 17/618,642, filed on Dec. 13, 2021, which is a National Stage Application under 35 U.S.C. § 371 and claims the benefit of international Application No. PCT/CN2020/102717, filed on Jul. 17, 2020, which claims the benefit of international Application No. PCT/CN2020/090587, filed on May 15, 2020, and International Application No. PCT/CN2019/096360, filed on Jul. 17, 2019. The entire contents of the foregoing applications are incorporated herein by reference.

### SEQUENCE LISTING

This application contains a Sequence Listing that has been submitted electronically as an XML file named 51624-0010002_SL_ST26.xml. The XML file, created on Aug. 18, 2025, is 664,048 bytes in size. The material in the XML file is hereby incorporated by reference in its entirety.

### FIELD OF THE INVENTION

The present invention relates to chimeric antigen receptors (CARs) targeting DLL3 and binding proteins specific for DLL3. The present invention also concerns nucleic acid sequences encoding the CARs or the binding proteins, modified immune cells expressing the CARs, as well as their uses for treating DLL3 associated disorders.

### BACKGROUND

Advances in cellular immunotherapy have provided a promising approach for the treatment of various tumors. One such treatment involves genetic engineering of immune cells, particularly T cells, to express chimeric antigen receptors (CARs) on the cell surface. Chimeric antigen receptors are proteins which, in their usual format, graft the specificity of a monoclonal antibody (mAb) to the effector function of a T cell. Once the CARs are expressed in a T cell, the CAR modified T cell (CAR-T or CAR-T cell) acquires some properties, such as antigen specific recognition, antitumor reactivity and proliferation, and thus can act as “living drugs” to eradicate targeted tumor cells. In principal, any antigens (e.g., cell surface molecules) can be targeted by these CAR-T cells. CAR-T cell therapy can override tolerance to self-antigens and provide a treatment which is not reliant on the MHC status of a patient. Using T cells engineered to express chimeric antigen receptors targeting CD19, recent trials have demonstrated remarkable clinical responses in leukaemia and lymphoma patients.

CARs are expressed as transmembrane proteins, including an antigen-specific binding site, a transmembrane region, and a signaling cytoplasmic domain (e.g., a CD3ζ chain). The antigen-specific binding site is usually a monoclonal antibody-derived single chain variable fragment (scFv) consisting of a heavy and light chain joined by a flexible linker. Recently CAR constructs have incorporated additional cytoplasmic domains from co-stimulatory molecules such as CD28 or 4-1 BB to enhance T cell survival in vivo. Other genetic modifications have also been made to CARs, e.g. the addition of cytokine genes or genes to avoid immunosuppressive mechanisms at the tumor site.

DLL3 (delta-like ligand 3) protein has been found to be clinically associated with various proliferative disorders, including tumors exhibiting neuroendocrine features, such as small-cell lung cancer (SCLC). SCLC, originating from neuroendocrine progenitor cells, comprises approximately 15% of all lung cancers, and has one of the lowest 5-year survival rates at 6% (Alvarado-Luna et al., 2016, Transl Lung Cancer Res 5:26-38; Siegel et al., 2017, CA Cancer J Clin 67:7-30). This is because it is highly aggressive, with about two-thirds of patients having metastatic diseases at diagnosis, and is highly refractory to conventional treatment (e.g., platinum-based chemotherapy).

There is a need for improved therapeutic approaches to treat SCLC and other DLL3-expressing cancers.

### SUMMARY

In one aspect, the present disclosure provides a chimeric antigen receptor (CAR) targeting DLL3 (anti-DLL3 CAR). The anti-DLL3 CAR comprises a DLL3 binding domain, wherein the DLL3 binding domain comprises or is derived from a single domain antibody (sdAb) or a single chain variable fragment (scFv).

In some embodiments, the sdAb comprises a polypeptide that comprises a CDR1 comprising the amino acid sequence of any one of SEQ ID NOs: 1-81 or a variant thereof comprising up to about 3 amino acid substitutions in the CDR1, a CDR2 comprising the amino acid sequence of any one of SEQ ID NOs: 82-162 or a variant thereof comprising up to about 3 amino acid substitutions in the CDR2, and a CDR3 comprising the amino acid sequence of any one of SEQ ID NOs: 163-243 or a variant thereof comprising up to about 3 amino acid substitutions in the CDR3.

In some embodiments, the sdAb comprises a polypeptide that comprises a CDR1 comprising the amino acid sequence of any one of SEQ ID NOs: 1-81, a CDR2 comprising the amino acid sequence of any one of SEQ ID NOs: 82-162 and a CDR3 comprising the amino acid sequence of any one of SEQ ID NOs: 163-243, or a variant of the polypeptide comprising up to about 3 amino acid substitutions in the CDR1, CDR2 and CDR3.

In some embodiments, the sdAb comprises a polypeptide comprising any one of the following:
(1) a CDR1 comprising the amino acid sequence of SEQ ID NO: 6, or a variant thereof comprising up to about 3 amino acid substitutions; a CDR2 comprising the amino acid sequence of SEQ ID NO: 87, or a variant thereof comprising up to about 3 amino acid substitutions; and a CDR3 comprising the amino acid sequence of SEQ ID NO: 168, or a variant thereof comprising up to about 3 amino acid substitutions;
(2) a CDR1 comprising the amino acid sequence of SEQ ID NO: 21, or a variant thereof comprising up to about 3 amino acid substitutions; a CDR2 comprising the amino acid sequence of SEQ ID NO: 102, or a variant thereof comprising up to about 3 amino acid substitutions; and a CDR3 comprising the amino acid sequence of SEQ ID NO: 183, or a variant thereof comprising up to about 3 amino acid substitutions;
(3) a CDR1 comprising the amino acid sequence of SEQ ID NO: 24, or a variant thereof comprising up to about 3 amino acid substitutions; a CDR2 comprising the amino acid sequence of SEQ ID NO: 105, or a variant thereof comprising up to about 3 amino acid substitutions; and a CDR3 comprising the amino acid sequence of SEQ ID NO: 186, or a variant thereof comprising up to about 3 amino acid substitutions;
(4) a CDR1 comprising the amino acid sequence of SEQ ID NO: 27, or a variant thereof comprising up to about 3 amino acid substitutions; a CDR2 comprising the amino acid sequence of SEQ ID NO: 108, or a variant thereof comprising up to about 3 amino acid substitutions; and a CDR3 comprising the amino acid sequence of SEQ ID NO: 189, or a variant thereof comprising up to about 3 amino acid substitutions;
(5) a CDR1 comprising the amino acid sequence of SEQ ID NO: 34, or a variant thereof comprising up to about 3 amino acid substitutions; a CDR2 comprising the amino acid sequence of SEQ ID NO: 115, or a variant thereof comprising up to about 3 amino acid substitutions; and a CDR3 comprising the amino acid sequence of SEQ ID NO: 196, or a variant thereof comprising up to about 3 amino acid substitutions;
(6) a CDR1 comprising the amino acid sequence of SEQ ID NO: 39, or a variant thereof comprising up to about 3 amino acid substitutions; a CDR2 comprising the amino acid sequence of SEQ ID NO: 120, or a variant thereof comprising up to about 3 amino acid substitutions; and a CDR3 comprising the amino acid sequence of SEQ ID NO: 201, or a variant thereof comprising up to about 3 amino acid substitutions;
(7) a CDR1 comprising the amino acid sequence of SEQ ID NO: 7, or a variant thereof comprising up to about 3 amino acid substitutions; a CDR2 comprising the amino acid sequence of SEQ ID NO: 88, or a variant thereof comprising up to about 3 amino acid substitutions; and a CDR3 comprising the amino acid sequence of SEQ ID NO: 169, or a variant thereof comprising up to about 3 amino acid substitutions;
(8) a CDR1 comprising the amino acid sequence of SEQ ID NO: 8, or a variant thereof comprising up to about 3 amino acid substitutions; a CDR2 comprising the amino acid sequence of SEQ ID NO: 89, or a variant thereof comprising up to about 3 amino acid substitutions; and a CDR3 comprising the amino acid sequence of SEQ ID NO: 170, or a variant thereof comprising up to about 3 amino acid substitutions; or
(9) a CDR1 comprising the amino acid sequence of SEQ ID NO: 9, or a variant thereof comprising up to about 3 amino acid substitutions; a CDR2 comprising the amino acid sequence of SEQ ID NO: 90, or a variant thereof comprising up to about 3 amino acid substitutions; and a CDR3 comprising the amino acid sequence of SEQ ID NO: 171, or a variant thereof comprising up to about 3 amino acid substitutions.

In some embodiments, the sdAb comprises a polypeptide comprising any one of the following:
(1) a CDR1 comprising the amino acid sequence of SEQ ID NO: 6; a CDR2 comprising the amino acid sequence of SEQ ID NO: 87; and a CDR3 comprising the amino acid sequence of SEQ ID NO: 168;
(2) a CDR1 comprising the amino acid sequence of SEQ ID NO: 21; a CDR2 comprising the amino acid sequence of SEQ ID NO: 102; and a CDR3 comprising the amino acid sequence of SEQ ID NO: 183;
(3) a CDR1 comprising the amino acid sequence of SEQ ID NO: 24; a CDR2 comprising the amino acid sequence of SEQ ID NO: 105; and a CDR3 comprising the amino acid sequence of SEQ ID NO: 186;
(4) a CDR1 comprising the amino acid sequence of SEQ ID NO: 27; a CDR2 comprising the amino acid sequence of SEQ ID NO: 108; and a CDR3 comprising the amino acid sequence of SEQ ID NO: 189;
(5) a CDR1 comprising the amino acid sequence of SEQ ID NO: 34; a CDR 2 comprising the amino acid sequence of SEQ ID NO: 115; and a CDR 3 comprising the amino acid sequence of SEQ ID NO: 196;
(6) a CDR1 comprising the amino acid sequence of SEQ ID NO: 39; a CDR2 comprising the amino acid sequence of SEQ ID NO: 120; and a CDR 3 comprising the amino acid sequence of SEQ ID NO: 201;
(7) a CDR1 comprising the amino acid sequence of SEQ ID NO: 7; a CDR2 comprising the amino acid sequence of SEQ ID NO: 88; and a CDR 3 comprising the amino acid sequence of SEQ ID NO: 169;
(8) a CDR1 comprising the amino acid sequence of SEQ ID NO: 8; a CDR2 comprising the amino acid sequence of SEQ ID NO: 89; and a CDR 3 comprising the amino acid sequence of SEQ ID NO: 170; or
(9) a CDR1 comprising the amino acid sequence of SEQ ID NO: 9; a CDR 2 comprising the amino acid sequence of SEQ ID NO: 90; and a CDR 3 comprising the amino acid sequence of SEQ ID NO: 171.

In some embodiments, the sdAb is a camel sdAb raised against human or rhesus DLL3.

In some embodiments, the sdAb comprises an amino acid sequence having at least about 95% sequence identity to the amino acid sequence of any one of SEQ ID NOs: 274-354.

In some embodiments, the sdAb is humanized through CDR grafting.

In some embodiments, the humanized sdAb comprises an amino acid sequence having at least about 95% sequence identity to the amino acid sequence of any one of SEQ ID NOs: 355-367.

In some embodiments, the scFv comprises a heavy chain variable region (VH) and a light chain variable region (VL), wherein the VH domain of the scFv comprises a CDR1 set forth in SEQ ID NO: 498 or 504 or a variant thereof comprising up to about 3 amino acid substitutions in the CDR1, a CDR2 set forth in SEQ ID NO: 499 or 505 or a variant thereof comprising up to about 3 amino acid substitutions in the CDR2, and a CDR3 set forth in SEQ ID NO: 500 or 506 or a variant thereof comprising up to about 3 amino acid substitutions in the CDR3, and the VL domain of the scFv comprises a CDR1 set forth in SEQ ID NO: 495 or 501 or a variant thereof comprising up to about 3 amino acid substitutions in the CDR1, a CDR2 set forth in SEQ ID NO: 496 or 502 or a variant thereof comprising up to about 3 amino acid substitutions in the CDR2, and a CDR3 set forth in SEQ ID NO: 497 or 503 or a variant thereof comprising up to about 3 amino acid substitutions in the CDR3.

In some embodiments, the VH domain of the scFv comprises a CDR1 set forth in SEQ ID NO: 498, a CDR2 set forth in SEQ ID NO: 499, and a CDR3 set forth in SEQ ID NO: 500, and the VL domain of the scFv comprises a CDR1 set forth in SEQ ID NO: 495, a CDR2 set forth in SEQ ID NO: 496, and a CDR3 set forth in SEQ ID NO: 497; or the VH domain of the scFv comprises a CDR1 set forth in SEQ ID NO: 504, a CDR2 set forth in SEQ ID NO: 505, and a CDR3 set forth in SEQ ID NO: 506, and the VL domain of the scFv comprises a CDR1 set forth in SEQ ID NO: 501, a CDR2 set forth in SEQ ID NO: 502, and a CDR3 set forth in SEQ ID NO: 503.

In some embodiments, the VH domain of the scFv comprises an amino acid sequence having at least about 95% sequence identity to the amino acid sequence set forth in SEQ ID NO: 508, and the VL domain of the scFv comprises an amino acid sequence having at least about 95% sequence identity to the amino acid sequence set forth in SEQ ID NO: 507; or the VH domain of the scFv comprises an amino acid sequence having at least about 95% sequence identity to the amino acid sequence set forth in SEQ ID NO: 510, and the VL domain of the scFv comprises an amino acid sequence having at least about 95% sequence identity to the amino acid sequence set forth in SEQ ID NO: 509.

In some embodiments, the scFv is obtained from a synthetic human Fab phage library.

In some embodiments, the DLL3 is human or rhesus DLL3.

In some embodiments, the anti-DLL3 CAR comprises, from N-terminus to C-terminus, a signal peptide, the DLL3 binding domain, a hinge domain, a transmembrane domain, and an intracellular signaling domain.

In some embodiments, the intracellular signaling domain is derived from CD3ζ, FcRγ, FcRβ, CD3γ, CD3δ, CD3ε, CD5, CD22, CD79a, CD79b, or CD66d.

In some embodiments, the intracellular signaling domain further comprises an intracellular co-stimulatory sequence.

In some embodiments, the intracellular co-stimulatory sequence is derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR, LGALS9, DAP10, DAP12, CD83, ligands of CD83 and combinations thereof.

In some embodiments, the CAR comprises an amino acid sequence having at least about 95% sequence identity to an amino acid sequence selected from the group consisting of SEQ ID NOs: 476-484, SEQ ID NOs: 485-494 or SEQ ID NOs: 515-516.

In some embodiments, the DLL3 binding domain comprises two sdAbs linked to each other.

In some embodiments, each of the sdAbs independently comprises an amino acid sequence having at least about 95% sequence identity to SEQ ID NO:356 or SEQ ID NO:366.

In some embodiments, the CAR comprises an amino acid sequence having at least about 95% sequence identity to an amino acid sequence selected from the group consisting of SEQ ID NOs: 518-520.

In some embodiments, the CAR comprises an amino acid sequence of SEQ ID NO: 520.

In another aspect, the present disclosure provides a DLL3 binding protein comprising a single domain antibody (sdAb) moiety that specifically binds to DLL3, wherein the sdAb moiety comprises a polypeptide that comprises a CDR1 comprising the amino acid sequence of any one of SEQ ID NOs: 1-81 or a variant thereof comprising up to about 3 amino acid substitutions in the CDR1, a CDR2 comprising the amino acid sequence of any one of SEQ ID NOs: 82-162 or a variant thereof comprising up to about 3 amino acid substitutions in the CDR2, and a CDR3 comprising the amino acid sequence of any one of SEQ ID NOs: 163-243 or a variant thereof comprising up to about 3 amino acid substitutions in the CDR3.

In some embodiments, the sdAb moiety comprises a polypeptide that comprises a CDR1 comprising the amino acid sequence of any one of SEQ ID NOs: 1-81, a CDR2 comprising the amino acid sequence of any one of SEQ ID NOs: 82-162 and a CDR3 comprising the amino acid sequence of any one of SEQ ID NOs: 163-243, or a variant of the polypeptide comprising up to about 3 amino acid substitutions in the CDR1, CDR2 and CDR3.

In some embodiments, the sdAb moiety comprises a polypeptide comprising any one of the following:
(1) a CDR1 comprising the amino acid sequence of SEQ ID NO: 6, or a variant thereof comprising up to about 3 amino acid substitutions; a CDR 2 comprising the amino acid sequence of SEQ ID NO: 87, or a variant thereof comprising up to about 3 amino acid substitutions; and a CDR 3 comprising the amino acid sequence of SEQ ID NO: 168, or a variant thereof comprising up to about 3 amino acid substitutions;
(2) a CDR1 comprising the amino acid sequence of SEQ ID NO: 21, or a variant thereof comprising up to about 3 amino acid substitutions; a CDR2 comprising the amino acid sequence of SEQ ID NO: 102, or a variant thereof comprising up to about 3 amino acid substitutions; and a CDR3 comprising the amino acid sequence of SEQ ID NO: 183, or a variant thereof comprising up to about 3 amino acid substitutions;
(3) a CDR1 comprising the amino acid sequence of SEQ ID NO: 24, or a variant thereof comprising up to about 3 amino acid substitutions; a CDR2 comprising the amino acid sequence of SEQ ID NO: 105, or a variant thereof comprising up to about 3 amino acid substitutions; and a CDR3 comprising the amino acid sequence of SEQ ID NO: 186, or a variant thereof comprising up to about 3 amino acid substitutions;
(4) a CDR1 comprising the amino acid sequence of SEQ ID NO: 27, or a variant thereof comprising up to about 3 amino acid substitutions; a CDR2 comprising the amino acid sequence of SEQ ID NO: 108, or a variant thereof comprising up to about 3 amino acid substitutions; and a CDR3 comprising the amino acid sequence of SEQ ID NO: 189, or a variant thereof comprising up to about 3 amino acid substitutions;
(5) a CDR1 comprising the amino acid sequence of SEQ ID NO: 34, or a variant thereof comprising up to about 3 amino acid substitutions; a CDR2 comprising the amino acid sequence of SEQ ID NO: 115, or a variant thereof comprising up to about 3 amino acid substitutions; and a CDR3 comprising the amino acid sequence of SEQ ID NO: 196, or a variant thereof comprising up to about 3 amino acid substitutions;
(6) a CDR1 comprising the amino acid sequence of SEQ ID NO: 39, or a variant thereof comprising up to about 3 amino acid substitutions; a CDR2 comprising the amino acid sequence of SEQ ID NO: 120, or a variant thereof comprising up to about 3 amino acid substitutions; and a CDR 3 comprising the amino acid sequence of SEQ ID NO: 201, or a variant thereof comprising up to about 3 amino acid substitutions;
(7) a CDR1 comprising the amino acid sequence of SEQ ID NO: 7, or a variant thereof comprising up to about 3 amino acid substitutions; a CDR2 comprising the amino acid sequence of SEQ ID NO: 88, or a variant thereof comprising up to about 3 amino acid substitutions; and a CDR 3 comprising the amino acid sequence of SEQ ID NO: 169, or a variant thereof comprising up to about 3 amino acid substitutions;
(8) a CDR1 comprising the amino acid sequence of SEQ ID NO: 8, or a variant thereof comprising up to about 3 amino acid substitutions; a CDR 2 comprising the amino acid sequence of SEQ ID NO: 89, or a variant thereof comprising up to about 3 amino acid substitutions; and a CDR3 comprising the amino acid sequence of SEQ ID NO: 170, or a variant thereof comprising up to about 3 amino acid substitutions; or
(9) a CDR1 comprising the amino acid sequence of SEQ ID NO: 9, or a variant thereof comprising up to about 3 amino acid substitutions; a CDR2 comprising the amino acid sequence of SEQ ID NO: 90, or a variant thereof comprising up to about 3 amino acid substitutions; and a CDR 3 comprising the amino acid sequence of SEQ ID NO: 171, or a variant thereof comprising up to about 3 amino acid substitutions.

In some embodiments, the sdAb moiety comprises a polypeptide comprising any one of the following:
(1) a CDR1 comprising the amino acid sequence of SEQ ID NO: 6; a CDR2 comprising the amino acid sequence of SEQ ID NO: 87; and a CDR 3 comprising the amino acid sequence of SEQ ID NO: 168;
(2) a CDR1 comprising the amino acid sequence of SEQ ID NO: 21; a CDR 2 comprising the amino acid sequence of SEQ ID NO: 102; and a CDR 3 comprising the amino acid sequence of SEQ ID NO: 183;
(3) a CDR1 comprising the amino acid sequence of SEQ ID NO: 24; a CDR2 comprising the amino acid sequence of SEQ ID NO: 105; and a CDR 3 comprising the amino acid sequence of SEQ ID NO: 186;
(4) a CDR1 comprising the amino acid sequence of SEQ ID NO: 27; a CDR 2 comprising the amino acid sequence of SEQ ID NO: 108; and a CDR3 comprising the amino acid sequence of SEQ ID NO: 189;
(5) a CDR1 comprising the amino acid sequence of SEQ ID NO: 34; a CDR2 comprising the amino acid sequence of SEQ ID NO: 115; and a CDR3 comprising the amino acid sequence of SEQ ID NO: 196;
(6) a CDR1 comprising the amino acid sequence of SEQ ID NO: 39; a CDR2 comprising the amino acid sequence of SEQ ID NO: 120; and a CDR3 comprising the amino acid sequence of SEQ ID NO: 201;
(7) a CDR1 comprising the amino acid sequence of SEQ ID NO: 7; a CDR2 comprising the amino acid sequence of SEQ ID NO: 88; and a CDR3 comprising the amino acid sequence of SEQ ID NO: 169;
(8) a CDR1 comprising the amino acid sequence of SEQ ID NO: 8; a CDR2 comprising the amino acid sequence of SEQ ID NO: 89; and a CDR3 comprising the amino acid sequence of SEQ ID NO: 170; or
(9) a CDR1 comprising the amino acid sequence of SEQ ID NO: 9; a CDR2 comprising the amino acid sequence of SEQ ID NO: 90; and a CDR3 comprising the amino acid sequence of SEQ ID NO: 171.

In some embodiments, the sdAb moiety is a camel sdAb raised against human or rhesus DLL3.

In some embodiments, the sdAb moiety comprises an amino acid sequence having at least about 95% sequence identity to the amino acid sequence of any one of SEQ ID NOs: 274-354.

In some embodiments, the sdAb moiety is humanized through CDR grafting.

In some embodiments, the humanized sdAb comprises an amino acid sequence having at least about 95% sequence identity to the amino acid sequence of any one of SEQ ID NOS: 355-367.

In some embodiments, the DLL3 is human or rhesus DLL3.

In another aspect, the present disclosure provides a DLL3 binding protein comprising a single chain variable fragment (scFv) moiety that specifically binds to DLL3, wherein the scFv moiety comprises a heavy chain variable region (VH) and a light chain variable region (VL), wherein the VH domain of the scFv moiety comprises a CDR1 set forth in SEQ ID NO: 498 or 504 or a variant thereof comprising up to about 3 amino acid substitutions in the CDR1, a CDR2 set forth in SEQ ID NO: 499 or 505 or a variant thereof comprising up to about 3 amino acid substitutions in the CDR2, and a CDR3 set forth in SEQ ID NO: 500 or 506 or a variant thereof comprising up to about 3 amino acid substitutions in the CDR3, and the VL domain of the scFv moiety comprises a CDR1 set forth in SEQ ID NO: 495 or 501 or a variant thereof comprising up to about 3 amino acid substitutions in the CDR1, a CDR2 set forth in SEQ ID NO: 496 or 502 or a variant thereof comprising up to about 3 amino acid substitutions in the CDR2, and a CDR3 set forth in SEQ ID NO: 497 or 503 or a variant thereof comprising up to about 3 amino acid substitutions in the CDR3.

In some embodiments, the scFv moiety comprises a heavy chain variable region (VH) and a light chain variable region (VL), wherein the VH domain of the scFv moiety comprises a CDR1 set forth in SEQ ID NO: 498, a CDR2 set forth in SEQ ID NO: 499, and a CDR3 set forth in SEQ ID NO: 500, and the VL domain of the scFv moiety comprises a CDR1 set forth in SEQ ID NO: 495, a CDR2 set forth in SEQ ID NO: 496, and a CDR3 set forth in SEQ ID NO: 497; or the VH domain of the scFv moiety comprises a CDR1 set forth in SEQ ID NO: 504, a CDR2 set forth in SEQ ID NO: 505, and a CDR3 set forth in SEQ ID NO: 506, and the VL domain of the scFv moiety comprises a CDR1 set forth in SEQ ID NO: 501, a CDR2 set forth in SEQ ID NO: 502, and a CDR3 set forth in SEQ ID NO: 503.

In some embodiments, the VH domain of the scFv moiety comprises an amino acid sequence having at least about 95% sequence identity to the amino acid sequence set forth in SEQ ID NO: 508, and the VL domain of the scFv moiety comprises an amino acid sequence having at least about 95% sequence identity to the amino acid sequence set forth in SEQ ID NO: 507; or the VH domain of the scFv moiety comprises an amino acid sequence having at least about 95% sequence identity to the amino acid sequence set forth in SEQ ID NO: 510, and the VL domain of the scFv moiety comprises an amino acid sequence having at least about 95% sequence identity to the amino acid sequence set forth in SEQ ID NO: 509.

In some embodiments, the scFv moiety is obtained from a synthetic human Fab phage library.

In some embodiments, the DLL3 is human or rhesus DLL3.

In another aspect, the present disclosure provides an isolated nucleic acid molecule encoding an anti-DLL3 CAR or a DLL3 binding protein as described above.

In some embodiments, the isolated nucleic acid molecule comprises a polynucleotide sequence selected from the group consisting of SEQ ID NOs: 368-448 which encodes a camel single domain antibody (sdAb).

In some embodiments, the isolated nucleic acid molecule comprises a polynucleotide sequence selected from the group consisting of SEQ ID NOs: 449-461 which encodes a humanized camel sdAb.

In some embodiments, the isolated nucleic acid molecule comprises a polynucleotide sequence selected from the group consisting of SEQ ID NOs: 511-514 which encodes a VL or VH domain of a human scFv.

In some embodiments, the nucleic acid molecule further comprises a polynucleotide sequence encoding a Chimeric Switch Receptor (CSR) or a Dominant Negative Receptor (DNR).

In some embodiments, the nucleic acid molecule further comprises a polynucleotide sequence encoding a PD-1 Dominant Negative Receptor (PD-1 DNR), a PD-1 Chimeric Switch Receptor (PD-1 CSR), or a TGF-β Dominant Negative Receptor (TGF-β DNR).

In some embodiments, the PD-1 DNR comprises an amino acid sequence having at least about 95% sequence identity to SEQ ID NO: 523.

In some embodiments, the PD-1 CSR comprises an amino acid sequence having at least about 95% sequence identity to SEQ ID NO: 524.

In some embodiments, the TGF-β DNR comprises an amino acid sequence having at least about 95% sequence identity to SEQ ID NO: 529.

In some embodiments, the polynucleotide sequence encoding the PD-1 DNR, the PD-1 CSR or the TGF-β DNR is linked to the polynucleotide sequence encoding the CAR through a polynucleotide sequence encoding a 2A self-cleaving peptide.

In some embodiments, the 2A self-cleaving peptide is a T2A peptide or a P2A peptide.

In some embodiments, the nucleic acid molecule comprises, in the 5′ to 3′ direction, a polynucleotide sequence encoding the CAR, a polynucleotide sequence encoding the 2A self-cleaving peptide, and a polynucleotide sequence encoding the PD-1 DNR, the PD-1 CSR or the TGF-β DNR.

In some embodiments, the nucleic acid molecule encoding a peptide having at least about 95% sequence identity to SEQ ID NO: 521 or 522, or the nucleic acid molecule encoding a peptide having at least about 95% sequence identity to an amino acid sequence selected from the group consisting of SEQ ID NOs: 525-528.

In another aspect, the present disclosure provides an expression vector comprising an isolated nucleic acid molecule as described above.

In another aspect, the present disclosure provides an engineered immune cell comprising an isolated nucleic acid molecule as described above.

In some embodiments, the engineered immune cell is selected from the group consisting of a cytotoxic T cell, a helper T cell, a natural killer T cell, a γδ T cell, a NKT cell and a Nature Killer cell.

In another aspect, the present disclosure provides an engineered immune cell expressing an anti-DLL3 CAR as described above.

In some embodiments, the engineered immune cell also expresses a CSR or a DNR.

In some embodiments, the CSR is a PD-1 CSR, the DNR is a PD-1 DNR or a TGF-β DNR.

In some embodiments, the PD-1 DNR comprises an amino acid sequence having at least about 95% sequence identity to SEQ ID NO: 523.

In some embodiments, the PD-1 CSR comprises an amino acid sequence having at least about 95% sequence identity to SEQ ID NO: 524.

In some embodiments, the TGF-β DNR comprises an amino acid sequence having at least about 95% sequence identity to SEQ ID NO: 529.

In some embodiments, the CAR and the CSR, or the CAR and the DNR, are co-expressed through a 2A self-cleaving peptide.

In some embodiments, the 2A self-cleaving peptide is a T2A peptide or a P2A peptide.

In some embodiments, the engineered immune cell expresses the CAR and the PD-1 DNR, and is stimulated by a cell expressing DLL3 and PD-L1.

In some embodiments, the engineered immune cell expresses the CAR and the PD-1 CSR, and is stimulated by a cell expressing DLL3 and PD-L1.

In some embodiments, the engineered immune cell expresses the CAR and the TGF-β DNR, and is stimulated by a cell expressing DLL3 in the present of TGF-β.

In some embodiments, the engineered immune cell is selected from the group consisting of a cytotoxic T cell, a helper T cell, a natural killer T cell, a γδ T cell, a NKT cell and a Nature Killer cell.

In another aspect, the present disclosure provides a pharmaceutical composition comprising an anti-DLL3 CAR, an isolated DLL3 binding protein, an expression vector, or an engineered immune cell as described above, and a physiologically acceptable excipient.

In another aspect, the present disclosure provides a method for treating a DLL3 associated disorder in a subject, comprising administrating to the subject a therapeutically effective amount of engineered immune cells as described above or a therapeutically effective amount of a pharmaceutical composition as described above.

In another aspect, the present disclosure provides the use of an anti-DLL3 CAR, an isolated DLL3 binding protein, an expression vector, or an engineered immune cell as described above for the preparation of a medicament for treating a DLL3 associated disorder.

In another aspect, the present disclosure provides a medicament for use in treating a DLL3 associated disorder comprising an anti-DLL3 CAR, a DLL3 binding protein, an expression vector, or an engineered immune cell as described above.

In some embodiments, the DLL3 associated disorder is a cancer selected from the group consisting of lung cancer, melanoma, breast cancer, prostate cancer, colon cancer, renal cell carcinoma, ovarian cancer, neuroblastoma, rhabdomyosarcoma, leukemia and lymphoma.

In some embodiments, the cancer expresses DLL3 and PD-L1.

In some embodiments, the cancer has a higher expression level of TGF-β compared to corresponding normal tissue.

In some embodiments, the DLL3 associated disorder is small cell lung cancer.

### BRIEF DESCRIPTION OF THE DRAWINGS

FIG. 1 shows schematic representation of a VHH based CAR construct. Sequences which can be used in the construct are listed in SEQ ID NOs: 462-472, 474 and 475.

FIG. 2 shows the results of an in vitro cytotoxicity assay of T cells expressing exemplary monospecific CARs comprising various camel anti-DLL3 sdAbs against small cell lung cancer cell line SHP-77 with an E:T of 2:1 or 5:1. The results for the CARs are depicted in the order as in the legend shown on the right.

FIGS. 3A-3B show the results of cytokine release levels of T cells expressing exemplary monospecific CARs comprising various camel anti-DLL3 sdAbs, after co-incubated with DLL3-expressing tumor cell line SHP-77. IFN-release levels and TNF-□ release levels (with an E:T of 2:1 or 5:1) are showed in FIG. 3A and FIG. 3B, respectively. In each figure, the results for the CARs are depicted in the order as in the legend shown on the right.

FIG. 4 shows fold expansions of T cells expressing exemplary monospecific CARs comprising various camel anti-DLL3 sdAbs, after long-term stimulation with small cell lung cancer cell line SHP-77.

FIG. 5 shows the results of in vivo anti-tumor efficacy of the CAR-T cells expressing CARs with camel anti-DLL3 sdAbs in a SHP-77 tumor model. In this model, each mouse was infused with a dose of 1.0 million CAR-T cells.

FIGS. 6A-6D show the results of an in vitro cytotoxicity assay of T cells expressing exemplary monospecific CARs comprising various humanized camel anti-DLL3 sdAbs against small cell lung cancer cell line SHP-77 (FIG. 6A, 6B) and NCI-H82 (FIG. 6C, 6D).

FIGS. 7A-7B show the results of cytokine release levels of T cells expressing exemplary monospecific CARs comprising various humanized camel anti-DLL3 sdAbs after stimulated by SHP-77. IFN-γ release levels and TNF-α release levels (with an E:T of 3:1 or 10:1) are showed in FIG. 7A and FIG. 7B, respectively. In each figure, the results for the CARs are depicted in the order as in the legend shown on the right.

FIG. 8 shows fold expansions of T cells expressing exemplary monospecific CARs comprising various humanized camel anti-DLL3 sdAbs, after long-term stimulation of with small cell lung cancer cell line SHP-77.

FIGS. 9A-9J show the results of in vivo anti-tumor efficacy of the CAR-T cells expressing CARs with humanized camel anti-DLL3 sdAbs in a SHP-77 tumor model. In this model, each mouse was infused with a dose of 0.2 million CAR-T cells. The results of 9 groups are compared in FIG. 9A. The results of each mouse in each group are shown in FIG. 9B-9J, respectively.

FIGS. 10A-10C show schematic representation of tandem CAR (FIG. 10A) and armored CAR constructs (FIG. 10B, C)

FIGS. 11A-11Z show comparison results of tandem CAR-T cells and monospecific CAR-T cells in vitro functional activities by measuring short-term cytotoxicity (FIG. 11A-E,V) and cytokine release (FIG. 11F-K, W-X), and by long-term stimulation assays (FIG. 11L-U, Y-Z).

FIGS. 12A-12D show in vitro functional comparison results of PD-1 DNR or PD-1 CSR armored CAR-T cells and T3 targeting SHP-77 cells (FIG. 12A, B) and SHP-77/PD-L1, respectively (FIG. 12C, D).

FIGS. 13A-13I show that TGF-β-DNR enhances the in vitro and in vivo anti-tumor efficacy of DLL3 CAR-T cells. FIG. 13A shows schematic representation of the TGF-β-DNR armored CARs. FIG. 13B shows the positive ratio of CAR and TGF-β-DNR on respective CAR-T cells. In vitro anti-tumor efficacy was evaluated in short-term (FIG. 13C, D) and long-term stimulation assays (FIG. 13E-G). FIG. 13H shows in vivo anti-tumor efficacy. FIG. 13I shows the pharmacokinetics of CAR-T cells in the peripheral blood of SHP77 xenograft model after treatment.

### DETAILED DESCRIPTION

Unless defined otherwise, technical and scientific terms used herein have the same meaning as commonly understood by a person of ordinary skill in the art. Any methods, devices and materials similar or equivalent to those described herein can be used in the practice of this invention. The following definitions are provided to facilitate understanding of certain terms used frequently herein and are not meant to limit the scope of the present disclosure.

The articles “a” and “an” are used herein to refer to one or to more than one (i.e., to at least one) of the grammatical object of the article. By way of example, “an element” means one element or more than one element.

The term “binding protein” as used herein refers to a molecule or a portion of a molecule which binds a target molecule (e.g., DLL3). In some embodiments, the binding protein comprises an antibody. In some embodiments, the binding protein comprises an antigen-binding fragment of an antibody. In some embodiments, the binding protein can further comprise a small molecular weight component, such as a small-molecule drug or toxin. The binding protein can also be an antibody or an antigen-binding fragment thereof. In some embodiments, the binding protein comprises the ligand-binding domain of a receptor. In some embodiments, the binding protein comprises the extracellular domain of a transmembrane receptor. The binding protein can also be the ligand-binding domain of a receptor, or the extracellular domain of a transmembrane receptor. In some embodiments, the binding protein comprises a single domain antibody (sdAb) or a single chain variable fragment (scFv). In some embodiments, the binding protein can be an sdAb or a scFv. A DLL3 binding protein can be a DLL3 binding domain. In some embodiment, the DLL3 binding protein comprises an antibody or an antigen-binding fragment of an antibody which binds DLL3. In some embodiments, the DLL3 binding protein can be an antibody or an antigen-binding fragment of an antibody. In some embodiments, the DLL3 binding protein comprises a single domain antibody (sdAb) or a single chain variable fragment (scFv) which binds DLL3. In some embodiments, the DLL3 binding protein can be an sdAb or a scFv.

The term “antibody” generally refers to any immunoglobulin (lg) molecule comprised of four polypeptide chains, two heavy (H) chains and two light (L) chains, or any functional fragment thereof, which retains the essential epitope binding features of the Ig molecule. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1. FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass. In a broad meaning, the term “antibody” further refers to scFv or sdAb which is not derived from an immunoglobulin molecule with four polypeptide chains.

An antibody fragment is a portion of an antibody, for example as F(ab′)2, Fab, Fv, scFv, sdAb, and the like. Functional fragments of a full length antibody retain the target specificity of a full length antibody. Recombinant functional antibody fragments, such as scFv (single chain variable chain fragments), have therefore been used to develop therapeutics as an alternative to therapeutics based on mAbs. scFv fragments (˜25 kDa) consist of the two variable domains, VH and VL. Naturally, VH and VL domains are non-covalently associated via hydrophobic interaction and tend to dissociate. However, stable fragments can be engineered by linking the domains with a hydrophilic flexible linker to create a scFv.

As used herein, the term “single domain antibody” (sdAb) has its general meaning in the art and refers to the single heavy chain variable domain of antibodies of the type that can be found in Camelid mammals and are naturally devoid of light chains. Such single-domain antibody is also called VHH or “Nanobody”. The amino acid sequence and structure of a single-domain antibody can be considered to be comprised of four framework regions (FR1, FR2, FR3, and FR4), and three complementary determining regions (CDR1, CDR2, and CDR3). Accordingly, the single-domain antibody can be defined as an amino acid sequence with the general structure: FR1-CDR1-FR2-CDR2-FR3-CDR3-FR4, which is similar to variable domain VH or VL. The use of sdAbs as single antigen-binding proteins or as an antigen-binding domain in larger proteins or polypeptides offer a number of significant advantages over the use of conventional antibodies or antibody fragments (e.g., scFv). The advantages of sdAbs include: only a single domain is required to bind an antigen with high affinity and with high selectivity; sdAbs are highly stable to denaturing agents or conditions including heat, pH, and proteases; and sdAbs can access targets and epitopes not accessible to conventional antibodies. Typically, sdAbs are produced in camelids such as llamas, but can also be synthetically generated using techniques that are well known in the art.

As used herein, the term “humanized sdAb” means an sdAb that has had one or more amino acid residues in the amino acid sequence of the naturally occurring VHH sequence replaced by one or more of the amino acid residues that occur at the corresponding position in a VH domain from a conventional 4-chain antibody from a human. This can be performed by methods that are well known in the art. For example, the FRs of the sdAbs can be replaced by human variable FRs. Humanized sdAbs thus have less antigenicity when administrated into human bodies.

As used herein, the term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

“DLL3”, also known as “delta-like ligand 3”, is a transmembrane protein involved in Notch signaling pathway. The Notch signaling pathway, first identified in C. elegans and Drosophila and subsequently shown to be evolutionarily conserved from invertebrates to vertebrates, participates in a series of fundamental biological processes including normal embryonic development, adult tissue homeostasis, and stein cell maintenance. In Drosophila, Notch signaling is mediated primarily by one Notch receptor gene and two ligand genes, known as Serrate and Delta (Wharton et al., 1985; Rebay et al., 1991). In humans, there are four known Notch receptors and five DSL (Delta-Serrate LAG2) ligands—two homologs of Serrate, known as Jagged1 and Jagged 2, and three homologs of Delta, termed delta-like ligands or DLL1, DLL3 and DLL4. In humans, the DLL3 gene is located on chromosome 19q13, and consists of 8 exons spanning 9.5 kb. Alternate splicing within the last exon gives rise to two protein isoforms. Both share overall 100% identity across their extracellular domains and their transmembrane domains, differing only in that the longer isoform contains an extended cytoplasmic tail.

As used herein, the term “specifically bind” or “specific binding” or any synonym thereof refers to the ability of a polypeptide, such as a single domain antibody (sdAb), to specifically recognize and detectably bind, as assayed by standard in vitro assays, to a DLL3 molecule. For example, binding, as used herein, is measured by the capacity of an anti-DLL3 polypeptide of the invention to recognize a DLL3 molecule on the surface of a cell using well described antigen-antibody binding assays, flow cytometry, and other assays known to those of skill in the art.

As used herein, the term “expression vector” is a nucleic acid construct or sequence, generated recombinantly or synthetically, with specific nucleic acid elements that permit transcription and/or expression of another nucleic acid in a host cell. An expression vector can be part of a plasmid, virus, or nucleic acid fragment. In one example, an expression vector is a DNA vector, such as a plasmid, that comprises at least one promoter sequence and at least one terminator sequence (e.g., a polyadenylation sequence), and optionally an origin of replication (ori) sequence, and optionally a selection or selectable marker sequence. Optionally, the expression vector may further comprise at least one nucleotide coding sequence of interest that codes for at least one polypeptide, wherein the at least one promoter sequence is operably linked with the at least one coding sequence. The term “expression” includes any step involved in the production of the polypeptide including, but not limited to, transcription, post-transcriptional modification, translation, post-translational modification, and/or secretion.

The term “isolated” refers to a material that is substantially or essentially free from components that normally accompany it in its native state. The material can be a cell or a macromolecule such as a protein or nucleic acid. For example, an “isolated nucleic acid,” as used herein, refers to a polynucleotide, which has been purified from the sequences which flank it in a naturally-occurring state, e.g., a DNA fragment which has been removed from the sequences that are normally adjacent to the fragment. Alternatively, an “isolated antibody” or an “isolated polypeptide” and the like, as used herein, refer to in vitro isolation and/or purification of an antibody or polypeptide molecule from its natural cellular environment, and from association with other components of the cell.

“Humanized” forms of non-human (e.g., camelid) antibodies are chimeric antibodies that contain minimal sequence derived from non-human immunoglobulin. In some embodiments, a humanized antibody is a human immunoglobulin (recipient antibody) in which residues from an hypervariable region (HVR) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

“sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The dissociation constant (KD or Kd) is used as an indicator showing affinity of antibodies to antigens. For example, easy analysis is possible by the Scatchard method using antibodies marked with a variety of marker agents, as well as by using BiacoreX (made by Amersham Biosciences), which is an over-the-counter, measuring kit, or similar kit, according to the user's manual and experiment operation method attached with the kit. The KD value that can be derived using these methods is expressed in units of M (Mols).

“Chimeric Switch Receptor (CSR)” as used herein refers to a receptor which is created to reverse the outcomes of its original signaling pathway in order to confer an immune cell (e.g., a CAR T cell) with a desired activity, such as, the ability to overcome the immunosuppressive tumor microenvironment and to allow it to have greater in vivo persistence.

In some embodiments, a CSR can exploit the inhibitory molecules expressed by a cancer cell to further stimulate the CAR T cell. In a non-limiting example, a CAR T cell can be engineered to express a CSR composed of the extracellular ligand binding domain of the human inhibitory receptor programmed cell death protein 1 (PD-1) fused to the transmembrane and cytoplasmic co-stimulatory signaling domains of CD28. When the CAR T cell is administrated into a subject with a cancer expressing DLL3 and programmed cell death ligand 1 (PD-L1), the expressed CAR can bind to the DLL3 and the expressed switch receptor can bind to PD-L1. The nature of the PD-1/CD28 chimeric switch receptor fusion protein prevents the normal PD1/PD-L1-mediated T-cell suppression and, instead, promotes signaling through the CD28 domain, which results in the stimulation of the CAR T cell. Thus, exchanging the transmembrane and intracellular domain of PD-1 with that of CD28 converts PD-L1 into a co-stimulation ligand of the CAR T cell. This will induce enhanced toxicity against PD-L1-expressing cancer cells. In other embodiments, a CSR can also be used to inhibit the effects of a CAR T cell on unintended target cells.

“Dominant Negative Receptor (DNR)” as used herein refers to a receptor which is able to bind its ligand but will not induce a signaling cascade inside the cell. A DNR usually has an intact ligand binding region but is missing an intracellular enzymatic region. It may be a mutated form of a full length receptor or a truncated form of the receptor. Following CAR T cell immunotherapy, some cancers, especially solid caners may upregulate inhibitory ligands that bind to inhibitory receptors on CAR T cells. This adaptive resistance compromises the efficacy of chimeric CAR T cell therapies. Some cancers, particularly solid cancer, are known to secrete transforming growth factor β (TGF-β), creating an immunosuppressive milieu. TGF-β is known to induce or promote metastasis and to potently suppress the immune system. Therefore, in some embodiments, we use a truncated version of the TGF-β receptor TGFBRII as a TGF-β DNR to improve the antitumor performance of the CAR T cells disclosed herein. In some embodiments, the CAR and the TGF-β DNR are co-expressed on a T cell's surface by using a 2A self-cleaving peptide. In some embodiments, the CAR and the TGF-β DNR are separately expressed on a T cell's surface by using two expression vectors. We find TGF-β DNR, when introduced into the anti-DLL3 CAR T cells disclosed herein, is able to enhance the cytotoxicity of the CAR T cells against some DLL3 positive cancer cells, such as SCLC cells. Similarly, in some embodiments, we use a truncated version of the PD-1 receptor as a PD-1 DNR to improve the antitumor performance of the CAR T cells disclosed herein.

As used herein, “treatment” or “treating.” includes any beneficial or desirable effect on the symptoms or pathology of a disease or pathological condition, and may include even minimal reductions in one or more measurable markers of the disease or condition being treated, e.g., cancer, autoimmune disease, immune disorder, etc. Treatment can optionally involve delaying of the progression of the disease or condition. “Treatment” does not necessarily indicate complete eradication or cure of the disease or condition, or associated symptoms thereof.

Some aspects of the present invention relate to a DLL3 binding protein, which has binding specificity for human or rhesus DLL3 protein.

In some embodiments, the DLL3 binding protein comprising a single domain antibody (“sdAb”) moiety that specifically binds to DLL3, wherein the sdAb moiety comprises a polypeptide that comprises a CDR1 comprising the amino acid sequence of any one of SEQ ID NOs: 1-81 or a variant thereof comprising up to about 3 amino acid substitutions in the CDR1, a CDR2 comprising the amino acid sequence of any one of SEQ ID NOs: 82-162 or a variant thereof comprising up to about 3 amino acid substitutions in the CDR2, and a CDR3 comprising the amino acid sequence of any one of SEQ ID NOs: 163-243 or a variant thereof comprising up to about 3 amino acid substitutions in the CDR3.

In some embodiments, the sdAb comprises a polypeptide that comprises a CDR1 comprising the amino acid sequence of any one of SEQ ID NOs: 1-81, a CDR2 comprising the amino acid sequence of any one of SEQ ID NOs: 82-162; and a CDR3 comprising the amino acid sequence of any one of SEQ ID NOs: 163-243, or a variant of the polypeptide comprising up to about 3 amino acid substitutions in the CDR regions. In some embodiments, the anti-DLL3 antibody is or comprises a single domain antibody (sdAb), which is produced from camel after been immunized with human or rhesus DLL3 proteins. In some embodiments, the sdAb comprises a CDR set (i.e., a combination of CDR1, CDR2, and CDR3) as listed in each row of Table 1.

In some embodiments, the sdAb comprises a polypeptide comprising any one of the following:
(1) a CDR1 comprising the amino acid sequence of SEQ ID NO: 6, or a variant thereof comprising up to about 3 amino acid substitutions; a CDR 2 comprising the amino acid sequence of SEQ ID NO: 87, or a variant thereof comprising up to about 3 amino acid substitutions; and a CDR 3 comprising the amino acid sequence of SEQ ID NO: 168, or a variant thereof comprising up to about 3 amino acid substitutions;
(2) a CDR1 comprising the amino acid sequence of SEQ ID NO: 21, or a variant thereof comprising up to about 3 amino acid substitutions; a CDR2 comprising the amino acid sequence of SEQ ID NO: 102, or a variant thereof comprising up to about 3 amino acid substitutions; and a CDR3 comprising the amino acid sequence of SEQ ID NO: 183, or a variant thereof comprising up to about 3 amino acid substitutions;
(3) a CDR1 comprising the amino acid sequence of SEQ ID NO: 24, or a variant thereof comprising up to about 3 amino acid substitutions; a CDR2 comprising the amino acid sequence of SEQ ID NO: 105, or a variant thereof comprising up to about 3 amino acid substitutions; and a CDR3 comprising the amino acid sequence of SEQ ID NO: 186, or a variant thereof comprising up to about 3 amino acid substitutions;
(4) a CDR1 comprising the amino acid sequence of SEQ ID NO: 27, or a variant thereof comprising up to about 3 amino acid substitutions; a CDR2 comprising the amino acid sequence of SEQ ID NO: 108, or a variant thereof comprising up to about 3 amino acid substitutions; and a CDR3 comprising the amino acid sequence of SEQ ID NO: 189, or a variant thereof comprising up to about 3 amino acid substitutions;
(5) a CDR1 comprising the amino acid sequence of SEQ ID NO: 34, or a variant thereof comprising up to about 3 amino acid substitutions; a CDR2 comprising the amino acid sequence of SEQ ID NO: 115, or a variant thereof comprising up to about 3 amino acid substitutions; and a CDR3 comprising the amino acid sequence of SEQ ID NO: 196, or a variant thereof comprising up to about 3 amino acid substitutions;
(6) a CDR1 comprising the amino acid sequence of SEQ ID NO: 39, or a variant thereof comprising up to about 3 amino acid substitutions; a CDR2 comprising the amino acid sequence of SEQ ID NO: 120, or a variant thereof comprising up to about 3 amino acid substitutions; and a CDR 3 comprising the amino acid sequence of SEQ ID NO: 201, or a variant thereof comprising up to about 3 amino acid substitutions;
(7) a CDR1 comprising the amino acid sequence of SEQ ID NO: 7, or a variant thereof comprising up to about 3 amino acid substitutions; a CDR2 comprising the amino acid sequence of SEQ ID NO: 88, or a variant thereof comprising up to about 3 amino acid substitutions; and a CDR 3 comprising the amino acid sequence of SEQ ID NO: 169, or a variant thereof comprising up to about 3 amino acid substitutions;
(8) a CDR1 comprising the amino acid sequence of SEQ ID NO: 8, or a variant thereof comprising up to about 3 amino acid substitutions; a CDR 2 comprising the amino acid sequence of SEQ ID NO: 89, or a variant thereof comprising up to about 3 amino acid substitutions; and a CDR3 comprising the amino acid sequence of SEQ ID NO: 170, or a variant thereof comprising up to about 3 amino acid substitutions; or
(9) a CDR1 comprising the amino acid sequence of SEQ ID NO: 9, or a variant thereof comprising up to about 3 amino acid substitutions; a CDR2 comprising the amino acid sequence of SEQ ID NO: 90, or a variant thereof comprising up to about 3 amino acid substitutions; and a CDR 3 comprising the amino acid sequence of SEQ ID NO: 171, or a variant thereof comprising up to about 3 amino acid substitutions.

In some embodiments, the sdAb comprises a polypeptide comprising any one of the following:
(1) a CDR1 comprising the amino acid sequence of SEQ ID NO: 6; a CDR2 comprising the amino acid sequence of SEQ ID NO: 87; and a CDR 3 comprising the amino acid sequence of SEQ ID NO: 168;
(2) a CDR1 comprising the amino acid sequence of SEQ ID NO: 21; a CDR 2 comprising the amino acid sequence of SEQ ID NO: 102; and a CDR 3 comprising the amino acid sequence of SEQ ID NO: 183;
(3) a CDR1 comprising the amino acid sequence of SEQ ID NO: 24; a CDR2 comprising the amino acid sequence of SEQ ID NO: 105; and a CDR 3 comprising the amino acid sequence of SEQ ID NO: 186;
(4) a CDR1 comprising the amino acid sequence of SEQ ID NO: 27; a CDR 2 comprising the amino acid sequence of SEQ ID NO: 108; and a CDR3 comprising the amino acid sequence of SEQ ID NO: 189;
(5) a CDR1 comprising the amino acid sequence of SEQ ID NO: 34; a CDR2 comprising the amino acid sequence of SEQ ID NO: 115; and a CDR3 comprising the amino acid sequence of SEQ ID NO: 196;
(6) a CDR1 comprising the amino acid sequence of SEQ ID NO: 39; a CDR2 comprising the amino acid sequence of SEQ ID NO: 120; and a CDR3 comprising the amino acid sequence of SEQ ID NO: 201;
(7) a CDR1 comprising the amino acid sequence of SEQ ID NO: 7; a CDR2 comprising the amino acid sequence of SEQ ID NO: 88; and a CDR3 comprising the amino acid sequence of SEQ ID NO: 169;
(8) a CDR1 comprising the amino acid sequence of SEQ ID NO: 8; a CDR2 comprising the amino acid sequence of SEQ ID NO: 89; and a CDR3 comprising the amino acid sequence of SEQ ID NO: 170; or
(9) a CDR1 comprising the amino acid sequence of SEQ ID NO: 9; a CDR2 comprising the amino acid sequence of SEQ ID NO: 90; and a CDR3 comprising the amino acid sequence of SEQ ID NO: 171.

In some embodiments, the sdAb comprises an amino acid sequence having at least about 95% (e.g., about 96%, 97%, 98%, 99% or 100%) sequence identity to the amino acid sequence of any one of SEQ ID NOs: 274-354. In some embodiments, the sdAb comprises the amino acid sequence of any one of SEQ ID NOs: 274-354. In other embodiments, the sdAb is humanized, and comprises an amino acid sequence having at least about 95% (e.g., about 96%, 97%, 98%, 99% or 100%) sequence identity to the amino acid sequence of any one of SEQ ID NOs: 355-367. In some embodiments, the sdAb comprises the amino acid sequence of any one of SEQ ID NOs: 355-367. A humanized antibody can be produced using a variety of techniques known in the art, including but not limited to, CDR-grafting (see, e.g., U.S. Pat. Nos. 5,225,539, 5,530,101, and 5,585,089), veneering or resurfacing (see, e.g., European Patent Nos. EP 592,106 and EP 519,596), and chain shuffling (see, e.g., U.S. Pat. No. 5,565,332). Generally, during humanization, the CDR residues of a receptor antibody (e.g., a human antibody) are replaced with CDR residues of a donor antibody (e.g., a rodent antibody), to retain the antigen-binding specificity while minimizing the in vivo immunogenicity. Often, framework residues in the framework regions will also be substituted with the corresponding residue from the donor antibody to alter, for example improve, antigen binding. These framework substitutions, e.g., conservative substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323).

In some cases, the sdAb can be fused with a human IgG hinge fragment and a Fc fragment to form a heave chain antibody (HCAb). In some cases, the sdAb can be fused with another sdAb or scFv which is specific for an antigen other than DLL3 to form a bispecific antibody. In some cases, the sdAb can be fused with two or more sdAbs or scFvs which are specific for an antigen other than DLL3 to form a multispecific antibody. In other cases, the sdAb can be chemically modified to carry a drug molecule. Thus the anti-DLL3 sdAb can be used in vivo to lead the drug molecule to a DLL3-expressing cell.

In some embodiments, the DLL3 binding protein is or comprises a single chain variable fragment (scFv) that specifically bind DLL3. In some cases, the scFv is isolated from a synthetic human Fab or scFv phage library through repeated rounds of phage panning, with each round of panning involving the processes of binding, removal of nonspecific phages, and the elution and amplification of bound phages for the next round. In some embodiments, the DLL3 binding protein comprising a scFv moiety that specifically binds to DLL3, wherein the scFv comprises a heavy chain variable region (VH) and a light chain variable region (VL), wherein the VH domain of the scFv comprises CDR1 set forth in SEQ ID NO: 498 or 504, CDR2 set forth in SEQ ID NO: 499 or 505, and CDR3 set forth in SEQ ID NO: 500 or 506, and the VL domain of the scFv comprises CDR1 set forth in SEQ ID NO: 495 or 501, CDR2 set forth in SEQ ID NO: 496 or 502, and CDR3 set forth in SEQ ID NO: 497 or 503. In some embodiments, the DLL3 binding protein comprising scFv moiety that specifically binds to DLL3, wherein the scFv comprises a heavy chain variable region (VH) and a light chain variable region (VL), wherein the VH domain of the scFv comprises CDR1 set forth in SEQ ID NO: 498, CDR2 set forth in SEQ ID NO: 499, and CDR3 set forth in SEQ ID NO: 500, and the VL domain of the scFv comprises CDR1 set forth in SEQ ID NO: 495, CDR2 set forth in SEQ ID NO: 496, and CDR3 set forth in SEQ ID NO: 497; or the VH domain of the scFv comprises CDR1 set forth in SEQ ID NO: 504, CDR2 set forth in SEQ ID NO: 505, and CDR3 set forth in SEQ ID NO: 506, and the VL domain of the scFv comprises CDR1 set forth in SEQ ID NO: 501, CDR2 set forth in SEQ ID NO: 502, and CDR3 set forth in SEQ ID NO: 503.

In some cases, the scFv can be fused with another sdAb or scFv which is specific for an antigen other than DLL3 to form a bispecific antibody. In some cases, the scFv can be fused with two or more sdAbs or scFvs which are specific for an antigen other than DLL3 to form a multispecific antibody. In other cases, the scFv can be chemically modified to carry a drug molecule. Thus the anti-DLL3 scFv can be used in vivo to lead the drug molecule to a DLL3-expressing cell.

Some aspects of the present invention relate to a CAR or a CAR-T cell comprising a DLL3 binding domain (anti-DLL3 CAR or anti-DLL3 CAR-T cells).

A CAR of the present invention comprises an extracellular domain, a transmembrane domain and an intracellular domain. In some embodiments, the CAR further includes a signal peptide at N-terminus, and a hinge region between the extracellular domain and the transmembrane domain. The extracellular domain includes a target-specific binding element (also referred to as an antigen recognition domain or antigen binding domain). The intracellular domain, or otherwise the cytoplasmic domain, often includes one or more co-stimulatory signaling domains and a CD3 (chain portion. The co-stimulatory signaling domain refers to a portion of the CAR including the intracellular domain of a co-stimulatory molecule.

Antigen recognition or antigen targeting by a CAR molecule most commonly involves the use of an antibody or antibody fragment. In accordance with the present invention, the antigen binding domain is an antibody or antibody fragment that specifically binds to DLL3. Preferably, the antigen binding domain of the CAR of the invention is an anti-DLL3 scFv or sdAb as mentioned above.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of), for example, the alpha, beta or zeta chain of the T-cell receptor, CD8α chain.

The intracellular signaling domain of the CAR of the invention is responsible for activation of at least one of the normal effector functions of an immune cell. The term “effector function” refers to a specialized function of a cell. Effector function of a T cell, for example, may be cytolytic activity or helper activity including the secretion of cytokines. Thus, the term “intracellular domain” refers to the portion of a protein which transduces the effector function signal and directs the cell to perform a specialized function. While usually the entire cytoplasmic domain can be employed, in many cases it is not necessary to use the entire chain. To the extent that a truncated portion of a cytoplasmic domain is used, such truncated portion may be used in place of an intact chain as long as it transduces the effector function signal. The term intracellular domain is thus meant to include any truncated portion of a cytoplasmic domain sufficient to transduce the effector function signal. Preferred examples of cytoplasmic domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability. In some embodiments, the intracellular signaling domain is derived from CD3ζ, FcRγ, FcRβ, CD3γ, CD3δ, CD3ε, CD5, CD22, CD79a, CD79b, or CD66d.

Often, signals generated through the TCR alone are insufficient for full activation of the T cell. Accordingly, a secondary or co-stimulatory signal is used. Thus, T cell activation can be said to be mediated by two distinct classes of cytoplasmic signaling sequences: those that initiate antigen-dependent primary activation through the TCR (primary cytoplasmic signaling sequences) and those that act in an antigen-independent manner to provide a secondary or co-stimulatory signal (secondary cytoplasmic signaling sequences). The co-stimulatory signaling sequence refers to a portion of the CAR including the intracellular domain of a co-stimulatory molecule. A co-stimulatory molecule is a cell surface molecule other than an antigen receptor or their ligands that is required for an efficient response of lymphocytes to an antigen. Examples of such molecules include CD27, CD28, 4-1BB, OX40, CD40, PD-1, LFA-1, ICOS, CD2, CD7, LIGHT, NKG2C, B7-H3, TNFRSF9), TNFRSF4, TNFRSF8, CD40LG, ITGB2, KLRC2, TNFRSF18, TNFRSF14, HAVCR, LGALS9, DAP10, DAP12, CD83, ligands of CD83 and combinations thereof.

The hinge region between the extracellular domain and the transmembrane domain of the CAR generally means any oligo- or polypeptide that functions to link the transmembrane domain to the extracellular domain in the polypeptide chain. The hinge region may be up to 300 amino acids, preferably 2 to 100 amino acids and most preferably 2 to 10 amino acids.

In addition to the antigen binding domain, transmembrane domain, cytoplasmic domain, and hinge region, the CAR of the present invention can also include a signal peptide sequence linked to the N-terminus of the CAR. Signal peptide sequences exist at the N-terminus of many secretory proteins and membrane proteins, and typically have a length of 15 to 30 amino acids. Since many of the protein molecules mentioned above have signal peptide sequences, these signal peptides can be used as a signal peptide for the CAR of this invention.

In some embodiments, the CAR comprising a DLL3 binding domain, wherein the DLL3 binding domain comprises or is derived from a single domain antibody (sdAb) or a single chain variable fragment (scFv).

In some embodiments, the CAR comprising a DLL3 binding domain, wherein the DLL3 binding domain comprises or is derived from a single domain antibody (sdAb), wherein the sdAb comprises a CDR1 comprising the amino acid sequence of any one of SEQ ID NOs: 1-81 or a variant thereof comprising up to about 3 amino acid substitutions in the CDR1; a CDR2 comprising the amino acid sequence of any one of SEQ ID NOs: 82-162 or a variant thereof comprising up to about 3 amino acid substitutions in the CDR2; and a CDR3 comprising the amino acid sequence of any one of SEQ ID NOs: 163-243 or a variant thereof comprising up to about 3 amino acid substitutions in the CDR3. In some embodiments, the CAR comprising a DLL3 binding domain, wherein the DLL3 binding domain comprises or is derived from a single domain antibody (sdAb), wherein the sdAb comprises a CDR1 comprising the amino acid sequence of any one of SEQ ID NOs: 1-81; a CDR2 comprising the amino acid sequence of any one of SEQ ID NOs: 82-162; and a CDR3 comprising the amino acid sequence of any one of SEQ ID NOs: 163-243.

In some embodiments, the CAR comprising a DLL3 binding domain, wherein the DLL3 binding domain comprises an sdAb comprising any one of the following:
(1) a CDR1 comprising the amino acid sequence of SEQ ID NO: 6, or a variant thereof comprising up to about 3 amino acid substitutions; a CDR2 comprising the amino acid sequence of SEQ ID NO: 87, or a variant thereof comprising up to about 3 amino acid substitutions; and a CDR3 comprising the amino acid sequence of SEQ ID NO: 168, or a variant thereof comprising up to about 3 amino acid substitutions;
(2) a CDR1 comprising the amino acid sequence of SEQ ID NO: 21, or a variant thereof comprising up to about 3 amino acid substitutions; a CDR2 comprising the amino acid sequence of SEQ ID NO: 102, or a variant thereof comprising up to about 3 amino acid substitutions; and a CDR3 comprising the amino acid sequence of SEQ ID NO: 183, or a variant thereof comprising up to about 3 amino acid substitutions;
(3) a CDR1 comprising the amino acid sequence of SEQ ID NO: 24, or a variant thereof comprising up to about 3 amino acid substitutions; a CDR2 comprising the amino acid sequence of SEQ ID NO: 105, or a variant thereof comprising up to about 3 amino acid substitutions; and a CDR3 comprising the amino acid sequence of SEQ ID NO: 186, or a variant thereof comprising up to about 3 amino acid substitutions;
(4) a CDR1 comprising the amino acid sequence of SEQ ID NO: 27, or a variant thereof comprising up to about 3 amino acid substitutions; a CDR2 comprising the amino acid sequence of SEQ ID NO: 108, or a variant thereof comprising up to about 3 amino acid substitutions; and a CDR3 comprising the amino acid sequence of SEQ ID NO: 189, or a variant thereof comprising up to about 3 amino acid substitutions;
(5) a CDR1 comprising the amino acid sequence of SEQ ID NO: 34, or a variant thereof comprising up to about 3 amino acid substitutions; a CDR2 comprising the amino acid sequence of SEQ ID NO: 115, or a variant thereof comprising up to about 3 amino acid substitutions; and a CDR3 comprising the amino acid sequence of SEQ ID NO: 196, or a variant thereof comprising up to about 3 amino acid substitutions;
(6) a CDR1 comprising the amino acid sequence of SEQ ID NO: 39, or a variant thereof comprising up to about 3 amino acid substitutions; a CDR2 comprising the amino acid sequence of SEQ ID NO: 120, or a variant thereof comprising up to about 3 amino acid substitutions; and a CDR3 comprising the amino acid sequence of SEQ ID NO: 201, or a variant thereof comprising up to about 3 amino acid substitutions;
(7) a CDR1 comprising the amino acid sequence of SEQ ID NO: 7, or a variant thereof comprising up to about 3 amino acid substitutions; a CDR2 comprising the amino acid sequence of SEQ ID NO: 88, or a variant thereof comprising up to about 3 amino acid substitutions; and a CDR3 comprising the amino acid sequence of SEQ ID NO: 169, or a variant thereof comprising up to about 3 amino acid substitutions;
(8) a CDR1 comprising the amino acid sequence of SEQ ID NO: 8, or a variant thereof comprising up to about 3 amino acid substitutions; a CDR2 comprising the amino acid sequence of SEQ ID NO: 89, or a variant thereof comprising up to about 3 amino acid substitutions; and a CDR3 comprising the amino acid sequence of SEQ ID NO: 170, or a variant thereof comprising up to about 3 amino acid substitutions; or
(9) a CDR1 comprising the amino acid sequence of SEQ ID NO: 9, or a variant thereof comprising up to about 3 amino acid substitutions; a CDR2 comprising the amino acid sequence of SEQ ID NO: 90, or a variant thereof comprising up to about 3 amino acid substitutions; and a CDR3 comprising the amino acid sequence of SEQ ID NO: 171, or a variant thereof comprising up to about 3 amino acid substitutions.

In some embodiments, the CAR comprising a DLL3 binding domain, wherein the DLL3 binding domain comprises an sdAb comprising any one of the following:
(1) a CDR1 comprising the amino acid sequence of SEQ ID NO: 6; a CDR2 comprising the amino acid sequence of SEQ ID NO: 87; and a CDR3 comprising the amino acid sequence of SEQ ID NO: 168;
(2) a CDR1 comprising the amino acid sequence of SEQ ID NO: 21; a CDR2 comprising the amino acid sequence of SEQ ID NO: 102; and a CDR3 comprising the amino acid sequence of SEQ ID NO: 183;
(3) a CDR1 comprising the amino acid sequence of SEQ ID NO: 24; a CDR2 comprising the amino acid sequence of SEQ ID NO: 105; and a CDR3 comprising the amino acid sequence of SEQ ID NO: 186;
(4) a CDR1 comprising the amino acid sequence of SEQ ID NO: 27; a CDR2 comprising the amino acid sequence of SEQ ID NO: 108; and a CDR3 comprising the amino acid sequence of SEQ ID NO: 189;
(5) a CDR1 comprising the amino acid sequence of SEQ ID NO: 34; a CDR 2 comprising the amino acid sequence of SEQ ID NO: 115; and a CDR 3 comprising the amino acid sequence of SEQ ID NO: 196;
(6) a CDR1 comprising the amino acid sequence of SEQ ID NO: 39; a CDR2 comprising the amino acid sequence of SEQ ID NO: 120; and a CDR 3 comprising the amino acid sequence of SEQ ID NO: 201;
(7) a CDR1 comprising the amino acid sequence of SEQ ID NO: 7; a CDR2 comprising the amino acid sequence of SEQ ID NO: 88; and a CDR 3 comprising the amino acid sequence of SEQ ID NO: 169;
(8) a CDR1 comprising the amino acid sequence of SEQ ID NO: 8; a CDR2 comprising the amino acid sequence of SEQ ID NO: 89; and a CDR 3 comprising the amino acid sequence of SEQ ID NO: 170; or
(9) a CDR1 comprising the amino acid sequence of SEQ ID NO: 9; a CDR 2 comprising the amino acid sequence of SEQ ID NO: 90; and a CDR 3 comprising the amino acid sequence of SEQ ID NO: 171.

In some embodiments, the CAR comprising a DLL3 binding domain, wherein the DLL3 binding domain comprises a camel sdAb, wherein the sdAb comprises an amino acid sequence having at least about 95% (e.g. 96%, 97%, 98%, 99% or 100%) sequence identity to the amino acid sequence of any one of SEQ ID NOs: 274-354. In some embodiments, the CAR comprising a DLL3 binding domain, wherein the DLL3 binding domain comprises a camel sdAb comprising an amino acid of any one of SEQ ID NOs: 274-354. In some embodiments, the CAR comprising a DLL3 binding domain, wherein the DLL3 binding domain comprises a humanized sdAb, wherein the sdAb comprises an amino acid sequence having at least about 95% (e.g. 96%, 97%, 98%, 99% or 100%) sequence identity to the amino acid sequence of any one of SEQ ID NOs: 355-367. In some embodiments, the CAR comprising a DLL3 binding domain, wherein the DLL3 binding domain comprises a humanized sdAb comprising an amino acid sequence of any one of SEQ ID NOs: 355-367.

In some embodiments, the CAR comprising a DLL3 binding domain, wherein the DLL3 binding domain comprises or is derived from a single chain variable fragment (scFv), wherein the VH domain of the scFv comprises CDR1 set forth in SEQ ID NO: 498, CDR2 set forth in SEQ ID NO: 499, and CDR3 set forth in SEQ ID NO: 500, and the VL domain of the scFv comprises CDR1 set forth in SEQ ID NO: 495, CDR2 set forth in SEQ ID NO: 496, and CDR3 set forth in SEQ ID NO: 497; or the VH domain of the scFv comprises CDR1 set forth in SEQ ID NO: 504, CDR2 set forth in SEQ ID NO: 505, and CDR3 set forth in SEQ ID NO: 506, and the VL domain of the scFv comprises CDR1 set forth in SEQ ID NO: 501, CDR2 set forth in SEQ ID NO: 502, and CDR3 set forth in SEQ ID NO: 503. In some embodiments, the VH domain of the scFv comprises an amino acid sequence set forth in SEQ ID NO: 508, and the VL domain of the scFv comprises an amino acid sequence set forth in SEQ ID NO: 507; or the VH domain of the scFv comprises an amino acid sequence set forth in SEQ ID NO: 510, and the VL domain of the scFv comprises an amino acid sequence set forth in SEQ ID NO: 509.

In some embodiments, the CAR of the present invention comprises a camel sdAb provided herein as the DLL3 binding domain and comprises an amino acid sequence having at least about 95% (e.g. 96%, 97%, 98%, 99% or 100%) sequence identity to the amino acid sequence of any one of SEQ ID NOs: 476-484. In some embodiments, the CAR of the present invention comprises a camel sdAb provided herein as the DLL3 binding domain and has an amino acid sequence selected from the group consisting of SEQ ID NOs: 476-484. In some embodiments, the CAR of the present invention comprises a humanized sdAb provided herein as the DLL3 binding domain and comprises an amino acid sequence having at least about 95% (e.g. 96%, 97%, 98%, 99% or 100%) sequence identity to the amino acid sequence of any one of SEQ ID NOs: 485-494. In other embodiments, the CAR of the present invention comprises a humanized sdAb provided herein as the DLL3 binding domain and has an amino acid sequence selected from the group consisting of SEQ ID NOs: 485-494.

In some embodiments, the CAR of the present invention comprises a human scFv provided herein as the DLL3 binding domain and comprises an amino acid sequence having at least about 95% (e.g. 96%, 97%, 98%, 99% or 100%) sequence identity to the amino acid sequence of any one of SEQ ID NOs: 515-516. In some embodiments, the CAR of the present invention comprises a human scFv provided herein as the DLL3 binding domain and has an amino acid sequence selected from the group consisting of SEQ ID NOs: 515-516.

In some embodiments, the CAR of the present invention comprises, from N-terminus to C-terminus, a signal peptide, the DLL3 binding domain, a hinge region, a transmembrane domain, and a cytoplasmic signaling domain. In particular embodiments, the CAR of the present invention comprises, from N-terminus to C-terminus, a CD8α signal peptide as set forth in SEQ ID NO: 465, the DLL3 binding domain, a CD8α hinge domain as set forth in SEQ ID NO: 466, a CD8α transmembrane domain as set forth in SEQ ID NO: 467, a CD137 cytoplasmic domain as set forth in SEQ ID NO: 468, a CD28 cytoplasmic domain as set forth in SEQ ID NO: 469, and a CD3ζ cytoplasmic domain as set forth in SEQ ID NO: 470.

Some aspects of the present invention relate to an isolated nucleic acid molecule which encodes the sdAb, scFv, or CAR of the present invention. In some embodiments, the nucleic acid molecule encodes a camel sdAb and comprises a polynucleotide sequence selected from the group consisting of SEQ ID NOs: 368-448. In some embodiments, the nucleic acid molecule encodes a humanized sdAb and comprises a polynucleotide sequence selected from the group consisting of SEQ ID NOs: 449-461. In some embodiments, the nucleic acid molecule encodes a VH and a VL domain of a scFv, wherein the VH domain encoding sequence comprises a polynucleotide sequence of SEQ ID NOs: 512 or 514, the VL domain encoding sequence comprises a polynucleotide sequence of SEQ ID NOs: 511 or 513.

Some aspects of present application relate to an engineered immune cell, comprising any one of the CARs provided above, or any one of the isolated nucleic acids described above, or any one of the vectors described above. In some embodiments, the engineered immune cell is a cytotoxic T cell, a helper T cell, a natural killer T cell, a γδ T cell, a NKT cell and a Nature Killer cell. In some embodiments, the cells comprise an expression vector which carries an isolated nucleic acid molecule of the present invention. Genetically modifying a cell with an expression vector to express a polypeptide encoded by a portion of the nucleic acid molecule is a genetic technique well known in the art.

Some aspects of the present invention relate to uses of the DLL3 binding proteins, anti-DLL3 CARs, nucleic acid molecules or CAR-T cells of the present invention. In some embodiments, the CAR-T cells of the present invention are formulated as a pharmaceutical composition with a physiologically acceptable excipient. As used herein, “physiologically acceptable excipient” includes without limitation any adjuvant, carrier, diluent, preservative, dispersing agent, suspending agent, stabilizer, isotonic agent, solvent, surfactant, or emulsifier as being acceptable for use in humans or domestic animals. In some embodiments, the CAR-T cells of the present invention or the pharmaceutical composition comprising the same is used to treat a DLL3-related disorder in a subject. Accordingly, a method for treating a DLL3-related disorder is provided, which comprises administrating to a subject suffering from a DLL3 associated disorder a therapeutically effective amount of the CAR-T cells or the pharmaceutical composition of the present invention. A “therapeutically effective amount” of antibodies, CAR-T cells or a pharmaceutical composition may vary according to factors such as the disease state, age, sex, and weight of a subject (e.g., a patient). The term “therapeutically effective amount” may include an amount that is effective to “treat” a subject. When a therapeutic amount is indicated, the precise amount contemplated in particular embodiments, to be administered, can be determined by a physician in view of the condition of the subject. In some embodiments, the DLL3 associated disorder is a cancer expressing DLL3 as cell surface proteins, such as, melanoma, breast cancer, prostate cancer, colon cancer, renal cell carcinoma, ovarian cancer, neuroblastoma, rhabdomyosarcoma, leukemia and lymphoma. Preferably, the DLL3 associated disorder is a lung cancer, especially small cell lung cancer (SCLC).

### EXAMPLES

The examples described herein are not intended to represent that the experiments below are all or the only experiments performed. Efforts have been made to ensure accuracy with respect to numbers used (for example, amounts, temperature, etc.), but some experimental errors and deviations should be accounted for. Unless indicated otherwise, parts are parts by weight, molecular weight is weight average molecular weight, temperature is in degrees Centigrade, and pressure is at or near atmospheric.

### Example 1. Animal Immunization and Antibody Library Construction

This example demonstrated that the immunized camel showed good immune response towards human or rhesus DLL3 protein and the acquired immunized library showed superior quality.

### Animal Immunization

Immunogens comprising extracellular domain of human DLL3 protein (aa27-466) having an N-terminal FLAG tag (AdipoGen, AG-40B-0151) or/and DLL3-expressing plasmid or DLL3-expressing cells (CHO-K1/DLL3 or/and CHO-K1/EGF4) were mixed with adjuvant or PBS and injected to camels. Typically, the camels were immunized for 2-4 times with 1-week to 2-week intervals. After multiple rounds of immunization, immune reactions against the target antigen DLL3 were assessed by serum titration through both enzyme-linked immune sorbent assay (ELISA) and flow cytometric assay.

### Phage Display Library Construction

Total RNA was extracted from lymphocytes of immunized camel using TRIZOL® Reagent according to the manufacturer's protocol. cDNA was synthesized based on RNA template with an oligo(dT)20 primer using PRIMESCRIPT™ 1st Strand cDNA Synthesis Kit according to the manufacturer's protocol. V_{H}Hs were amplified from camel cDNA for generation of VHH phage library.

### Example 2. Generation of Anti-DLL3 Antibodies

Anti-DLL3 antibodies provided herein include single domain antibodies (sdAbs) generated from an immunized camel or human Fab isolated from synthetic human Fab library.

### Phage Display

A phage display library was constructed with the sdAbs obtained by immunization (Immunogens comprising extracellular domain of human DLL3 protein (aa27-466) having an N-terminal FLAG tag (AdipoGen, AG-40B-0151) or/and DLL3-expressing plasmid or DLL3-expressing cells (CHO-K1/DLL3 or/and CHO-K1/EGF4)). Another human Fab phage display library was synthesized. Both phage libraries were rescued and stored after filter sterilization at 4° C. for further use. Bound phages were isolated with the above-mentioned two phage libraries using protein-based panning as well as cell-based panning. At least one round of panning was carried for both protein- and cell-based panning approaches using both libraries until the percentage of DLL3-specific phage clones reached 30%. Output phages of each round were assessed for the number of total output clones, percentage of DLL3 positive clones by ELISA and sequence diversity of DLL3-specific clones. Based on these results the best panning outputs were selected for high-throughput screening.

### High-Throughput Screening

The selected output phages were used to infect exponentially growing E. coli cells. Double-strand DNA of the output phages was extracted. The sdAb/Fab insert were cut from the phagemid vector and inserted into an antibody fragment expression vector for high-throughput screening. The resulting plasmid was used to transform exponentially growing E. coli cells, which were subsequently plated and grown overnight at 37° C. Thousands of colonies were picked individually and grown in 96 deep well plates containing 1 mL 2×YT medium. The expression of antibody fragment was induced by adding 1 mM IPTG.

The sdAb/Fab proteins in the supernatant were analyzed for their ability to bind DLL3 ECD protein by ELISA and DLL3 expressing SHP-77 cell lines (American Type Culture Collection (ATCC)® CRL-2195™) and CHO-K1/human DLL3 (in-house generation) by FACS. All binders were sequenced. The redundant sequences were removed. All together, 81 camel sdAbs and 2 human Fab binders that bound both human and rhesus DLL3 proteins and cell lines were obtained. All these binders have unique amino acid sequences.

Some of these unique binders were subjected to further characterization by surface plasmon resonance (SPR) on a BIAcore T200 instrument (GE Healthcare). The experiment was carried out as follows: the crude sdAb/Fab proteins were captured through an affinity tag onto the sensorchip. High-concentration (100 nM) of human DLL3 flowed over the sensorchip surface, and were allowed to bind the antibody fragments for 300 s followed by injection of running buffer to allow the dissociation of the complex formed. On-rate (ka) and off-rate (kd) were roughly calculated based on one association and dissociation curve, and were used to estimate the equilibrium dissociation constant (KD). The binding affinities of some of these unique binders were shown in Table 7.

CDR sequences of anti-DLL3 camel sdAbs were listed in Table 1 and CDR sequences of anti-DLL3 human scFvs were listed in Table 2.

TABLE 1
Anti-DLL3 camel sdAb CDR sequences

| | | CDR1 | | CDR2 | | CDR3 |
|---|---|---|---|---|---|---|
| sdAb | ID | Sequence | ID | Sequence | ID | Sequence |
| AS63930 | 1 | GYTYS | 82 | VVYNI | 163 | EVADP |
| | | GNYMA | | DGGGR | | TWGSR |
| | | | | FTTYA | | DQRRY |
| | | | | DSVKG | | KY |
| AS63932 | 2 | GYTYG | 83 | VIYTG | 164 | RYGSG |
| | | STFMG | | GGSTW | | NVNY |
| | | | | YASSV | | |
| | | | | KG | | |
| AS63951 | 3 | RDIYG | 84 | SIYPA | 165 | RSFSI |
| | | NNCMA | | GGRPY | | AVCAT |
| | | | | YADSV | | RSGIT |
| | | | | KG | | RSNFA |
| | | | | | | Y |
| AS63984 | 4 | GYTYS | 85 | TIVSG | 166 | GGPVT |
| | | SNFMG | | GGTTY | | NAPRW |
| | | | | YADSV | | YPLRP |
| | | | | RG | | PGYNY |
| AS63987 | 5 | GYRNC | 86 | VIYTP | 167 | DRPFV |
| | | MA | | SGITD | | CNIAN |
| | | | | YASSV | | MRRSS |
| | | | | KG | | N |
| AS63997 | 6 | FSGYG | 87 | AITVG | 168 | GYLSG |
| | | VSTMA | | SGNTY | | GSWDV |
| | | | | YADSV | | PGRYN |
| | | | | KG | | Y |
| AS64047 | 7 | QYVYR | 88 | AVYTG | 169 | GFVSG |
| | | WDLMG | | DGITY | | GRWNQ |
| | | | | YADSV | | SYRYK |
| | | | | KG | | Y |
| AS64052 | 8 | GYTYR | 89 | TIHSG | 170 | GGPPA |
| | | SNFMG | | VATTY | | NADRW |
| | | | | YADSV | | YPLRP |
| | | | | KG | | PGYNY |
| AS64062 | 9 | RSPYS | 90 | ALYTG | 171 | VVPRG |
| | | SSRCM | | GGSTS | | GSCRL |
| | | G | | YADSV | | DERGY |
| | | | | KG | | YH |
| AS64072 | 10 | GYSYY | 91 | AHGP | 172 | ETTMG |
| | | INLMA | | VSGTA | | WAHER |
| | | | | YYTDS | | GYRY |
| | | | | VKG | | |
| AS64097 | 11 | GYTYS | 92 | AINTG | 173 | GPDLG |
| | | RNCMG | | GGSTY | | GSWCR |
| | | | | YADSL | | PVERA |
| | | | | EG | | FTY |
| AS64114 | 12 | GNTYS | 93 | VIYTR | 174 | ASRHR |
| | | TNYMG | | GGHTY | | LRLNN |
| | | | | YVDSV | | PRDYD |
| | | | | RG | | Y |
| AS64123 | 13 | GYTYT | 94 | IIYTG | 175 | RFSEY |
| | | SNWLG | | SGSTH | | NY |
| | | | | YRSSV | | |
| | | | | KG | | |
| AS64130 | 14 | GYTYR | 95 | TIDSR | 176 | GGPRT |
| | | SNFMG | | GTITY | | NDDRW |
| | | | | YADSV | | YPLRP |
| | | | | KG | | PGYNY |
| AS64137 | 15 | GSTYS | 96 | TLVTW | 177 | AAAST |
| | | TNFMG | | VERTA | | DVRLL |
| | | | | YADSV | | DPGDF |
| | | | | KG | | AY |
| AS64142 | 16 | GFTFD | 97 | CIDWT | 178 | DTTSG |
| | | RNAMR | | GANIA | | YCSGF |
| | | | | YADSV | | WSTSR |
| | | | | KG | | YS |
| AS64154 | 17 | GYTYR | 98 | CIYTG | 179 | SSPRW |
| | | YLYMG | | SGSTG | | GGTCR |
| | | | | YADSV | | RWSQY |
| | | | | KG | | NY |
| AS64160 | 18 | VYTSS | 99 | AMCFG | 180 | DFGRD |
| | | SYCMG | | GLVTH | | KNYLR |
| | | | | YADSV | | PLLPH |
| | | | | KG | | AYNY |
| AS64228 | 19 | GVSYN | 100 | RIQPG | 181 | LCWRE |
| | | RCSMG | | GNTYY | | NVNY |
| | | | | ADSVK | | |
| | | | | G | | |
| AS64300 | 20 | GDIYN | 101 | YINTI | 182 | FNYGG |
| | | LMSMA | | IGNTY | | AWYEE |
| | | | | YTDSV | | RSYKY |
| | | | | KG | | |
| AS64380 | 21 | GNTYS | 102 | VIYTR | 183 | SSRH |
| | | SNYMG | | GGHTY | | RLGLN |
| | | | | YVDSV | | NPRDY |
| | | | | RG | | DY |
| AS64395 | 22 | GSTYS | 103 | TLVTW | 184 | AASTA |
| | | TNFMG | | AERTA | | VRLLD |
| | | | | YADSV | | PGDFA |
| | | | | KG | | Y |
| AS64443 | 23 | GYTDS | 104 | IIYTG | 185 | RFPAV |
| | | SVYIG | | GESTH | | TY |
| | | | | YRSSV | | |
| | | | | KG | | |
| AS64511 | 24 | RATYS | 105 | TITTG | 186 | NLRIG |
| | | TNYIS | | DGETA | | GDWFD |
| | | | | YADSV | | GRDFR |
| | | | | KG | | A |
| AS64536 | 25 | RYTDN | 106 | LIYPG | 187 | KWGLG |
| | | FVYMG | | GGSTY | | GGGLK |
| | | | | YASSV | | SDTYM |
| | | | | KG | | Y |
| AS64597 | 26 | GYTYR | 107 | TIDSG | 188 | GGPPT |
| | | VNFMG | | VGTTY | | DGDRW |
| | | | | YADSV | | YPLRP |
| | | | | KG | | PGYNY |
| AS64617 | 27 | GYTDR | 108 | RISTS | 189 | IVGRT |
| | | CSMA | | GFTNY | | CSLNY |
| | | | | AASVK | | |
| | | | | G | | |
| AS64634 | 28 | GYSFR | 109 | VFYPG | 190 | RRWVS |
| | | GDFMC | | GGSTN | | GTCYW |
| | | MG | | YADSA | | DSDFH |
| | | | | KG | | Y |
| AS69498 | 29 | GNTYS | 110 | VIYTR | 191 | SSRHR |
| | | SNYMA | | GGHTY | | LRLSD |
| | | | | YIDSV | | PRDYD |
| | | | | RG | | Y |
| AS69500 | 30 | RYTYS | 111 | SIFTG | 192 | RAFQV |
| | | SACMG | | TGGST | | GYCYL |
| | | | | YYADS | | RTDVY |
| | | | | VKG | | NY |
| AS69527 | 31 | RYTFS | 112 | AIYTD | 193 | RRWAC |
| | | STCMA | | DGSTW | | PRVGS |
| | | | | YADSV | | WHEFA |
| | | | | KG | | Y |
| AS68280 | 32 | GSTYS | 113 | AISTG | 194 | ARGRF |
| | | SNYLG | | DGATA | | IDWTK |
| | | | | YADSV | | ATQYD |
| | | | | KG | | Y |
| AS68355 | 33 | GYTYS | 114 | AIDSD | 195 | AIVGG |
| | | GVCMG | | GSTSY | | FNAYC |
| | | | | ADSVK | | SGGYV |
| | | | | G | | LDFGA |
| AS69443 | 34 | GFTFD | 115 | TISSD | 196 | DFLTG |
| | | DSDMA | | GSTYY | | FYYSD |
| | | | | ADSVK | | SPHPA |
| | | | | G | | PCSAS |
| | | | | | | DFGY |
| AS75376 | 35 | GYTYS | 116 | VIYTG | 197 | DPNPD |
| | | SHSMG | | DGSTY | | YMLPF |
| | | | | YADSV | | RPSRR |
| | | | | KG | | SW |
| AS75387 | 36 | GYPY | 117 | VAYT | 198 | DLRL |
| | | SSPC | | GGDI | | PRAG |
| | | MA | | QYLT | | GCAY |
| | | | | DSVK | | SY |
| | | | | G | | |
| AS75695 | 37 | GYTV | 118 | FIDA | 199 | DRRG |
| | | SAYC | | GGAT | | RVRR |
| | | MG | | IYAD | | CEYN |
| | | | | PVKG | | A |
| AS76169 | 38 | GYIY | 119 | YIRD | 200 | DRGG |
| | | SSFC | | NIMT | | YANS |
| | | MG | | SYTD | | CAVA |
| | | | | SVKG | | ARYD |
| | | | | | | Y |
| AS63931 | 39 | FSGY | 120 | AITV | 201 | GWLS |
| | | GVST | | GSGN | | GGSW |
| | | MA | | TYYA | | HVPG |
| | | | | DSVT | | RYNY |
| | | | | G | | |
| AS63937 | 40 | GSTI | 121 | CIHT | 202 | DSRR |
| | | SSRP | | GLGR | | PCMV |
| | | MA | | TYYA | | AAGY |
| | | | | DSVK | | TY |
| | | | | G | | |
| AS63948 | 41 | GYTY | 122 | CIYT | 203 | ASPR |
| | | RYLY | | GSGS | | WGGT |
| | | MG | | TGYA | | CRRW |
| | | | | DSVK | | SEYN |
| | | | | G | | Y |
| AS63956 | 42 | GFTY | 123 | LINS | 204 | YQAK |
| | | SNCC | | SGGT | | YCSG |
| | | MR | | YYAD | | PCAP |
| | | | | SVRG | | PTD |
| AS63965 | 43 | GYSS | 124 | KVIS | 205 | WCRE |
| | | GSCR | | DGTT | | YPGG |
| | | MG | | VYAD | | ILNN |
| | | | | SVKG | | G |
| AS63993 | 44 | GFTF | 125 | LVAT | 206 | RTDS |
| | | DDLV | | AGNS | | EHAF |
| | | MA | | VYAD | | KF |
| | | | | SVKG | | |
| AS63999 | 45 | GYTY | 126 | IIYT | 207 | RYTD |
| | | SSNW | | GGIS | | YNY |
| | | MG | | THYR | | |
| | | | | SSVK | | |
| | | | | G | | |
| AS64006 | 46 | GYTG | 127 | LIYT | 208 | RSRT |
| | | DTTY | | SGTS | | MMY |
| | | IG | | EYYA | | |
| | | | | DSVK | | |
| | | | | G | | |
| AS64057 | 47 | GFTF | 128 | CISW | 209 | DTTS |
| | | DRNA | | TGAN | | GSCS |
| | | MR | | IAYA | | GFWS |
| | | | | DSVK | | TSRY |
| | | | | G | | Y |
| AS64060 | 48 | GSTY | 129 | VIDS | 210 | DPTI |
| | | CTYR | | GGST | | GCPQ |
| | | MS | | SYAD | | TYRY |
| | | | | SVKG | | NY |
| AS64071 | 49 | GNTY | 130 | FIVM | 211 | STKD |
| | | RLNS | | IRGT | | QFYV |
| | | MG | | TYYG | | FNPI |
| | | | | ASVK | | GYDS |
| | | | | G | | |
| AS64093 | 50 | RYIY | 131 | SIYP | 212 | RSFS |
| | | GNNC | | AGGR | | IGVC |
| | | MA | | TYYA | | ATQS |
| | | | | DSVK | | GITW |
| | | | | G | | SNFA |
| | | | | | | Y |
| AS64118 | 51 | GYTY | 132 | FINS | 213 | TWDS |
| | | SACR | | AGST | | SCRF |
| | | MA | | YYAD | | QY |
| | | | | SVKG | | |
| AS64120 | 52 | RYIY | 133 | SIYP | 214 | RSFS |
| | | GNNC | | AGGR | | IADC |
| | | MA | | PYYA | | ATQS |
| | | | | DSVK | | GITR |
| | | | | G | | SNFA |
| | | | | | | Y |
| AS64124 | 53 | TYTP | 134 | AIAT | 215 | GRPY |
| | | SNNY | | IGGT | | SLPL |
| | | MG | | TRYA | | PLPL |
| | | | | DSVK | | ESGA |
| | | | | G | | YRY |
| AS64135 | 54 | TSTY | 135 | AMQP | 216 | DPMG |
| | | CRYY | | DGTT | | GSRT |
| | | MR | | SYSD | | PCTS |
| | | | | SVKG | | A |
| AS64163 | 55 | GYRY | 136 | AIST | 217 | DPSV |
| | | RWNC | | GSGS | | CPGG |
| | | MA | | TYYA | | MWYS |
| | | | | GSVK | | KEYR |
| | | | | G | | Y |
| AS64182 | 56 | GQTS | 137 | CIYT | 218 | SSPH |
| | | RYLY | | GSGS | | WGGT |
| | | MG | | TGYA | | CRRW |
| | | | | DSVK | | SEYK |
| | | | | G | | Y |
| AS64183 | 57 | GHTY | 138 | SVYT | 219 | DLSG |
| | | SANC | | DDDS | | GPAG |
| | | MA | | TMYT | | CGYT |
| | | | | DSVK | | H |
| | | | | G | | |
| AS64207 | 58 | GYTY | 139 | TIVS | 220 | GGPP |
| | | SSNF | | GGGT | | TNGA |
| | | MG | | TYYA | | KWYP |
| | | | | DSVR | | LRPP |
| | | | | G | | GYNY |
| AS64276 | 59 | GYTG | 140 | QIFT | 221 | SLGP |
| | | SSRC | | GRGT | | GRGA |
| | | MA | | TGYA | | CGYN |
| | | | | DSVK | | Y |
| | | | | G | | |
| AS64336 | 60 | GRTY | 141 | HIFS | 222 | RTGW |
| | | SSCS | | DGSR | | APRC |
| | | MG | | YYAD | | AVPG |
| | | | | SVKG | | Y |
| AS64346 | 61 | GYTY | 142 | TIGT | 223 | VQSK |
| | | FMG | | GDIF | | SSNY |
| | | | | NGAA | | VLRD |
| | | | | YYVD | | ASTY |
| | | | | SVKG | | NY |
| AS64420 | 62 | GDTS | 143 | TIST | 224 | RSRY |
| | | RSVW | | AGGS | | ATY |
| | | MG | | TWYT | | |
| | | | | DSVK | | |
| | | | | G | | |
| AS64473 | 63 | GYTY | 144 | CIYT | 225 | SSPQ |
| | | RYLY | | GSGT | | WGGT |
| | | MA | | TGYA | | CRRW |
| | | | | DSVK | | SEYN |
| | | | | G | | Y |
| AS64475 | 64 | GYTW | 145 | TITI | 226 | RDTA |
| | | SRNW | | SGGS | | RTY |
| | | MG | | TWYA | | |
| | | | | DSVK | | |
| | | | | G | | |
| AS64513 | 65 | DYPY | 146 | AACT | 227 | GYYS |
| | | IDNC | | GGGS | | GSGP |
| | | MG | | TYYA | | GYLL |
| | | | | DSVK | | PWRY |
| | | | | G | | NY |
| AS64562 | 66 | GYTA | 147 | VIHT | 228 | GFRP |
| | | RRDF | | GGDT | | RGGG |
| | | MA | | TYYA | | YTGD |
| | | | | DSVK | | VLAQ |
| | | | | G | | AAAY |
| | | | | | | NY |
| AS64583 | 67 | GFTI | 148 | CTSW | 229 | KAHP |
| | | AVYT | | AGGR | | DCSG |
| | | MG | | TYTA | | DWSP |
| | | | | DSVK | | SGYE |
| | | | | G | | Y |
| AS64594 | 68 | GYTY | 149 | LIYT | 230 | RTQT |
| | | NSNY | | GGGS | | RNY |
| | | MG | | TYYA | | |
| | | | | DSVK | | |
| | | | | G | | |
| AS64605 | 69 | RYPY | 150 | RIYT | 231 | RSNS |
| | | SSIC | | GTGS | | YSYS |
| | | MG | | TWYT | | SCDY |
| | | | | DSVK | | GPLT |
| | | | | G | | RGGY |
| | | | | | | NF |
| AS64606 | 70 | GYTS | 151 | AFYL | 232 | RLDE |
| | | RSNY | | IYTR | | KMY |
| | | MG | | GGST | | |
| | | | | YYAS | | |
| | | | | SVKG | | |
| AS68121 | 71 | GYTY | 152 | TDYI | 233 | DPGS |
| | | SRNC | | RFGR | | RTDD |
| | | MG | | TYYA | | SCGT |
| | | | | DSVK | | SYNK |
| | | | | G | | GNFG |
| | | | | | | Y |
| AS68170 | 72 | GYTY | 153 | TIYT | 234 | ASDV |
| | | RSNC | | GGGR | | AVGV |
| | | MG | | NLYY | | NSCG |
| | | | | ADSV | | GRTA |
| | | | | KG | | GFDA |
| AS63964 | 73 | GYTY | 154 | AISG | 235 | EVVD |
| | | SYNN | | GRFT | | PTWG |
| | | MG | | AYAD | | SRDQ |
| | | | | SVKG | | RRYK |
| | | | | | | Y |
| AS64116 | 74 | GYIY | 155 | GIST | 236 | DRWN |
| | | SCVG | | GGGG | | SFAN |
| | | | | TVYA | | CGAW |
| | | | | DSVK | | GRYT |
| | | | | G | | Y |
| AS68270 | 75 | GYPS | 156 | AITS | 237 | ASGW |
| | | STYY | | GTGS | | IVPS |
| | | MLSM | | TSYA | | RSLT |
| | | A | | DSVK | | ANLY |
| | | | | D | | RY |
| AS68320 | 76 | GYTY | 157 | AIYR | 238 | GRAG |
| | | NTNY | | HSGN | | PWAL |
| | | MG | | TAYA | | MRPT |
| | | | | DSVK | | EFGY |
| | | | | G | | |
| AS68351 | 77 | GDTF | 158 | GISA | 239 | GAVR |
| | | RAYY | | SGGR | | LSTS |
| | | MN | | TSYA | | SVRD |
| | | | | DSVK | | SS |
| | | | | G | | |
| AS75378 | 78 | GNTR | 159 | IVYT | 240 | RSYE |
| | | STTY | | GGRD | | YTY |
| | | MG | | TYYA | | |
| | | | | ASVK | | |
| | | | | G | | |
| AS75383 | 79 | GYTF | 160 | TFNN | 241 | DRRY |
| | | SSYC | | RGVA | | GRQW |
| | | LG | | NYHD | | YQPC |
| | | | | SVKG | | EWNT |
| AS75751 | 80 | GYFY | 161 | AIDT | 242 | GFGY |
| | | NTYY | | DGRT | | MNVI |
| | | FMG | | SYAD | | QALN |
| | | | | SVKG | | GMRQ |
| | | | | | | NPDY |
| AS76422 | 81 | GYTF | 162 | TYNN | 243 | DRRD |
| | | AGNC | | FGVA | | GRRW |
| | | LG | | NYAD | | SQPC |
| | | | | SVKG | | EWNT |

TABLE 2
Anti-DLL3 human scFv CDR sequences

| | | CDR1 | | CDR2 | | CDR3 |
|---|---|---|---|---|---|---|
| Ab | ID | Sequence | ID | Sequence | ID | Sequence |
| AS56704 | 495 | VL: | 496 | VL: | 497 | VL: |
| | | RASQSV | | SASSLYS | | QQASW |
| | | SSAVA | 499 | VH: | | SPIT |
| | 498 | VH: | | YIYPSYGY | 500 | VH: |
| | | GFNISS | | TSY | | GGYYYH |
| | | SYMH | | ADSVKG | | GMDY |
| AS56788 | 501 | VL: | 502 | VL: | 503 | VL: |
| | | RASQSV | | SASSLYS | | QQHYA |
| | | SSAVA | 505 | VH: | | PSLIT |
| | 504 | VH: | | YISSYYGY | 506 | VH: |
| | | GFNIS | | TYY | | YSYYY |
| | | SSYMH | | ADSVKG | | GMDY |
| AS56788 | 501 | VL: | 502 | VL: | 503 | VL: |
| | | RASQSV | | SASS | | QQHYA |
| | | SSAVA | | LYS | | PSLIT |
| | 504 | VH: | 505 | VH: | 506 | VH: |
| | | GFNISS | | YISSYY | | YSYYY |
| | | YSMH | | GYTYY | | GMDY |
| | | | | ADSVKG | | |

Anti-DLL3 sdAb amino acid sequences were listed in Table 3. CDRs of the sdAb were underlined. Nucleic acid sequences encoding the anti-DLL3 sdAbs were shown in SEQ ID NOs: 368-448.

TABLE 3
Anti-DLL3 camel sdAb amino acid sequences

| | Camel | |
|---|---|---|
| SEQ ID | sdAb | Amino Acid Sequence |
| 274 | AS63930 | EVQLAESGGGSVQAGGSLRLSCAASGYTYSGNYMAWFRQAP |
| | | GNEREGVAVVYNIDGGGRFTTYADSVKGRFTISRGNDKNTVYL |
| | | QMNSLKPEDSGMYYCAAEVADPTWGSRDQRRYKYWGQGTQ |
| | | VTVSS |
| 275 | AS63932 | QVQLEESGGGSVQAGGSLRLSCVASGYTYGSTFMGWFRQNPG |
| | | KEREGVAVIYTGGGSTWYASSVKGRFTISQDNAKNTVYLQMN |
| | | SLKPEDTAMYYCAARYGSGNVNYWGQGTQVTVSS |
| 276 | AS63951 | QVHLMESGGGSVQAGGSLRLACETSRDIYGNNCMAWFRQAP |
| | | GKEREGVASIYPAGGRPYYADSVKGRFTISQDNAKNTVYLQMD |
| | | SLKPEDTAMYYCAARSFSIAVCATRSGITRSNFAYWGQGTQVT |
| | | VSS |
| 277 | AS63984 | QVKLVESGGGSVQAGGSLRLSCAASGYTYSSNFMGWFRQAPG |
| | | KEREGVATIVSGGGTTYYADSVRGRFTISQDNAKNTVYLQMNS |
| | | LKPEDTAMYYCAAGGPVTNAPRWYPLRPPGYNYWGQGTQVT |
| | | VSS |
| 278 | AS63987 | QVRLVESGGGSVQAGGSLRLSCEATGYRNCMAWFRQAPGKER |
| | | EGVAVIYTPSGITDYASSVKGRFTISQNNARNTQYLQMNSLKPE |
| | | DTAMYYCAADRPFVCNIANMRRSSNWGRGTQVTVSS |
| 279 | AS63997 | QVRLVESGGGSVQAGGSLRLSCAGSFSGYGVSTMAWFRQAPG |
| | | KEREGVAAITVGSGNTYYADSVKGRFTISRDNAKRTVFLQMNS |
| | | LKPEDTAMYYCAVGYLSGGSWDVPGRYNYWGQGTQVTVSS |
| 280 | AS64047 | QVHLVESGGGSVQAGGSLRLSCAASQYVYRWDLMGWFRQAP |
| | | GKEREAVAAVYTGDGITYYADSVKGRFSISQDNAKNTVYLQMN |
| | | SLKPEDTGMYFCAAGFVSGGRWNQSYRYKYWGQGTQVTVSS |
| 281 | AS64052 | QVHLMESGGGSVQAGGSLRLSCAASGYTYRSNFMGWFRQAP |
| | | GKEREGIATIHSGVATTYYADSVKGRFTISQDNAKNTVYLQMN |
| | | SLKPEDTAMYYCAAGGPPANADRWYPLRPPGYNYWGQGTQV |
| | | TVSS |
| 282 | AS64062 | QVRLVESGGGSVQVGGSLRLSCAASRSPYSSSRCMGWFRQAP |
| | | GKEREGVAALYTGGGSTSYADSVKGRFTISQDNAKNTVYLQM |
| | | NSLKPEDTAMYYCAAVVPRGGSCRLDERGYYHWGQGTQVTV |
| | | SS |
| 283 | AS64072 | QVQLVESGGGSVQAGGFLRLSCALSGYSYYINLMAWFRQAPG |
| | | KEREAVAAHGPVSGTAYYTDSVKGRFTISRDPGKNTMYLQMFS |
| | | LQPEDTALYYCAAETTMGWAHERGYRYWGQGTQVTVSS |
| 284 | AS64097 | QVHLMESGGGSVQAGGSLRLSCEASGYTYSRNCMGWFRQAP |
| | | GKEREGVAAINTGGGSTYYADSLEGRFTISQDNAKNTMYLQM |
| | | NSLKPEDTAMYYCAAGPDLGGSWCRPVERAFTYWGQGTQVT |
| | | VSS |
| 285 | AS64114 | QVQLQESGGGSVQAGGSLTLSCEASGNTYSTNYMGWFRQAPG |
| | | KEREEVAVIYTRGGHTYYVDSVRGRFTISQDNAKNTVYLQMN |
| | | SLKPEDTAMYYCAAASRHRLRLNNPRDYDYWGQGTQVTVSS |
| 286 | AS64123 | QVQLAESGGGSVQAGGSLRLSCAASGYTYTSNWLGWFRQAPG |
| | | KEREEVAIIYTGSGSTHYRSSVKGRFTISQDNAKNTVYLQMNSL |
| | | KPEDTAMYYCAARFSEYNYWGQGTQVTVSS |
| 287 | AS64130 | EVQLAESGGGSVQAGGSLRLSCAASGYTYRSNFMGWFRQAPA |
| | | KEREGVATIDSRGTITYYADSVKGRFTISQDNEKNTVYLQMNSL |
| | | KPEDTAMYYCAAGGPRTNDDRWYPLRPPGYNYWGQGTQVTV |
| | | SS |
| 288 | AS64137 | QVRLVESGGGSVQAGGSLRLSCAASGSTYSTNFMGWFRQAPG |
| | | KEREGVATLVTWVERTAYADSVKGRFTISQDRAKNTVYLQMNS |
| | | LKPEDTAMYYCAAAAASTDVRLLDPGDFAYWGQGTQVTVSS |
| 289 | AS64142 | QVHLMESGGGLVQTGGSLRLSCTASGFTFDRNAMRWYRQAPG |
| | | KEREGVSCIDWTGANIAYADSVKGRFTISRDNAKNTLYLQMNS |
| | | LKPEDTGMYYCAADTTSGYCSGFWSTSRYSWGQGTQVTVSS |
| 290 | AS64154 | QVQLKESGGGSVQAGGSLRLSCTASGYTYRYLYMGWFRQTPG |
| | | KEREGVACIYTGSGSTGYADSVKGRFTISQDNAKNTVYLQMNN |
| | | LKPEDTAMYYCAASSPRWGGTCRRWSQYNYWGQGTQVTVSS |
| 291 | AS64160 | EVQLVESGGGSVQAGGSLRLSCAASVYTSSSYCMGWFRQAPG |
| | | KEREGVAAMCFGGLVTHYADSVKGRFTISQDNAKNTVYLQMN |
| | | SLKPEDTAMYYCAADFGRDKNYLRPLLPHAYNYWGQGTQVT |
| | | VSS |
| 292 | AS64228 | QVQLKESGGGSIQAGGSLRLSCAASGVSYNRCSMGWYRQAPG |
| | | KGRELVSRIQPGGNTYYADSVKGRFTVSQDNAKNTVSLQMNS |
| | | LKPEDTAMYYCNALCWRENVNYWGQGTQVTVSS |
| 293 | AS64300 | QVHLVESGGGSVQTGGSLRLSCAVSGDIYNLMSMAWFRRAPG |
| | | KEREGVAYINTIIGNTYYTDSVKGRFTISRDNSKNTLYLQMNNL |
| | | KPEDTAMYYCAAFNYGGAWYEERSYKYWGQGTQVTVSS |
| 294 | AS64380 | EVQLVESGGGSVQAGGSLTLSCEASGNTYSSNYMGWFRQAPG |
| | | KEREEVAVIYTRGGHTYYVDSVRGRFTISQDNAKNTVYLQMN |
| | | SLKPEDTAMYYCAASSRHRLGLNNPRDYDYWGQGTQVTVSS |
| 295 | AS64395 | QVRLVESGGGSVQAGGSLRLSCAASGSTYSTNFMGWFRQAPG |
| | | KEREGVATLVTWAERTAYADSVKGRFTISQDRAKNTVYLQMN |
| | | GLKPEDTAMYYCAAAASTAVRLLDPGDFAYWGQGTQVTVSS |
| 296 | AS64443 | QVHLVESGGGSVQAGGSLRLSCAASGYTDSSVYIGWFRQAPG |
| | | KEREEVAIIYTGGESTHYRSSVKGRFTVSQDNAKNTLYLQMNS |
| | | LKPEDTAMYYCAARFPAVTYWGQGTQVTVSS |
| 297 | AS64511 | QVQLVESGGGSVQAGGSLRLSCAASRATYSTNYISWFRQAPGK |
| | | EREAVATITTGDGETAYADSVKGRFTISRDNAKNTVYLQMNSL |
| | | KPEDTAMYYCAANLRIGGDWFDGRDFRAWGQGTQVTVSS |
| 298 | AS64536 | QVKLVESGGGSVQAGGSLRLSCAASRYTDNFVYMGWFRQAPG |
| | | KEREGVALIYPGGGSTYYASSVKGRFTISQDNAKGTVHLQMNN |
| | | LKPEDTAMYYCAAKWGLGGGGLKSDTYMYWGQGTQVTVSS |
| 299 | AS64597 | QVHLVESGGGSVQAGGSLRLSCAASGYTYRVNFMGWFRQTPG |
| | | KEREGVATIDSGVGTTYYADSVKGRFTISHNNAKNTIYLQMNS |
| | | LKPEDTAMYYCAAGGPPTDGDRWYPLRPPGYNYWGQGTQVT |
| | | VSS |
| 300 | AS64617 | QVQLVESGGGSVQAGGSLRLSCAASGYTDRCSMAWYRQAPG |
| | | KERELVSRISTSGFTNYAASVKGRFTISQDNAKNTVYLQMNSLN |
| | | PGDTGMYYCAIIVGRTCSLNYWGNGILVTVSS |
| 301 | AS64634 | QVRLVESGGGSVQAGGSLRLSCAASGYSFRGDFMCMGWFRQT |
| | | PGKGREGVAVFYPGGGSTNYADSAKGRFTISQDNAKNTMYLQ |
| | | MNTLKPEDTAMYYCAARRWVSGTCYWDSDFHYWGQGTQVT |
| | | VSS |
| 302 | AS69498 | QVQLQESGGGSVQAGGSLRLSCEASGNTYSSNYMAWFRQAPG |
| | | KEREEVAVIYTRGGHTYYIDSVRGRFTISQDNAKNTVYLQMNS |
| | | LKPEDTAMYYCAASSRHRLRLSDPRDYDYWGQGTQVTVSS |
| 303 | AS69500 | QVRLVESGGGSVQAGGSLRLSCAADRYTYSSACMGWFRQAPG |
| | | KEREGVASIFTGTGGSTYYADSVKGRFTISQDNAKNTVYLQMN |
| | | SLKPEDTAIYYCAARAFQVGYCYLRTDVYNYWGQGTQVTVSS |
| 304 | AS69527 | EVQLAESGGGSVQAGGSLRLSCVASRYTFSSTCMAWFRQAPG |
| | | KEREEVAAIYTDDGSTWYADSVKGRFTISRDNAKNTVYLQMN |
| | | SLKPEDTAMYYCAARRWACPRVGSWHEFAYWGQGTQVTVSS |
| 305 | AS68280 | QVQLVESGGGSVHPGGSLRLSCAASGSTYSSNYLGWFRQAPG |
| | | KGRDWVAAISTGDGATAYADSVKGRFTISQDNAKNTVYLQMN |
| | | SLKLEDSAMYYCAAARGRFIDWTKATQYDYWGQGTQVTVSS |
| 306 | AS68355 | QMQLVESGGDSVQAGGSLRLSCAASGYTYSGVCMGWFRQAP |
| | | GKEREGVAAIDSDGSTSYADSVKGRFTISKDNAKNTLYLQMNS |
| | | LKPEDTAMYYCAAAIVGGFNAYCSGGYVLDFGAWGQGTQVT |
| | | VSS |
| 307 | AS69443 | EVQLAESGGGSVQAGGSLRLSCSASGFTFDDSDMAWYRQAPG |
| | | DGCDLVSTISSDGSTYYADSVKGRFTISQDNAKNTVYLQMHSL |
| | | KPEDTAVYYCAADFLTGFYYSDSPHPAPCSASDFGYWGQGTQV |
| | | TVSS |
| 308 | AS75376 | QVQLKESGGGSVQAGGSLRLSCAASGYTYSSHSMGWFRQAPG |
| | | KEREGVAVIYTGDGSTYYADSVKGRFTISQDNAKNTVYLQMNS |
| | | LKPEDTAMYYCAADPNPDYMLPFRPSRRSWWGQGTQVTVSS |
| 309 | AS75387 | QVHLVESGGGSVQAGGSLRLSCAASGYPYSSPCMAWFRQAPG |
| | | KEREGVLVAYTGGDIQYLTDSVKGRFTISRDNAKNTVYLQMNS |
| | | LKPEDTAMYYCAADLRLPRAGGCAYSYWGQGTQVTVSS |
| 310 | AS75695 | QVRLVESGGGSVQAGGSLRLSCVASGYTVSAYCMGWFRQVLG |
| | | KGRERIAFIDAGGATIYADPVKGRFTISKDNAKNTLYLQMNSLK |
| | | PEDTAMYYCVADRRGRVRRCEYNAWGQGTQVTVSS |
| 311 | AS76169 | QVHLMESGGGSVQAGGSLRLSCAASGYIYSSFCMGWFRQAPG |
| | | KEREVVAYIRDNIMTSYTDSVKGRFTISKDNAKRTLYLQMNGL |
| | | KPEDTGMYYCAVDRGGYANSCAVAARYDYWGRGTQVTVSS |
| 312 | AS63931 | EVQLAESGGGSVQAGGSLRLSCAGSFSGYGVSTMAWFRQAPG |
| | | KEREGVAAITVGSGNTYYADSVTGRFTISRDNAKRTVYLQMNS |
| | | LKPEDTAMYYCAAGWLSGGSWHVPGRYNYWGQGTQVTVSS |
| 313 | AS63937 | QVKLVESGGGSVQAGGSLRLSCAASGSTISSRPMAWFRQAPGK |
| | | EREGVACIHTGLGRTYYADSVKGRFTISQDNAKNTVYLQVNSL |
| | | KPEDTAMYYCAADSRRPCMVAAGYTYWGQGTQVTVSS |
| 314 | AS63948 | QVQLVESGGGSVQAGGSLRLSCTASGYTYRYLYMGWFRQTPG |
| | | KEREGVACIYTGSGSTGYADSVKGRFTISQDNAENTVYLQMNS |
| | | LKPEDTAMYYCAAASPRWGGTCRRWSEYNYWGQGTQVTVSS |
| 315 | AS63956 | QVHLVESGGGLVQAGGSLRLSCAASGFTYSNCCMRWYRQAPG |
| | | KARELVSLINSSGGTYYADSVRGRFTISKDNAKNTLYLQMNSL |
| | | KPEDTAMYYCAAYQAKYCSGPCAPPTDWGQGTQVTVSS |
| 316 | AS63965 | QVQLVESGGGSVQAGGSLRLSCVASGYSSGSCRMGWYRQAPG |
| | | KERELVSKVISDGTTVYADSVKGRFTLSQGNAKNTVYLQMSSL |
| | | LPEDTAMYYCNAWCREYPGGILNNGWGQGTQVTVSS |
| 317 | AS63993 | QVKLVESGGGLVQAGGSLRLSCTVSGFTFDDLVMAWFRQAPG |
| | | KERQLVSLVATAGNSVYADSVKGRFTLSRDNAHSTAYLQMNGL |
| | | KPEDTAMYYCAARTDSEHAFKFWGQGTQVTVSS |
| 318 | AS63999 | EVQLVESGGGSVQAGGSLRLSCAASGYTYSSNWMGWFRQAP |
| | | GKEREEVAIIYTGGISTHYRSSVKGRFTISQDNAKNTVYLQMNS |
| | | LKPEDTAMYYCAARYTDYNYWGQGTQVTVSS |
| 319 | AS64006 | QVHLVESGGGSVQAGGSLRLSCEVSGYTGDTTYIGWFRQAPG |
| | | KEREGVALIYTSGTSEYYADSVKGRFIISRDNAKNTVYLQMNSL |
| | | KPEDTAMYYCGARSRTMMYWGQGTQVTVSS |
| 320 | AS64057 | QVQLEESGGGLVQTGGSLRLSCTASGFTFDRNAMRWYRQAPG |
| | | KEREGVSCISWTGANIAYADSVKGRFTISRDNAKNTLYLQMNS |
| | | LKPEDTGMYYCAADTTSGSCSGFWSTSRYYWGQGTQVTVSS |
| 321 | AS64060 | QVKLVESGGGSVQAGGSLRLSCAASGSTYCTYRMSWFRQAPG |
| | | KEREFVAVIDSGGSTSYADSVKGRFTISRDNAKNTVYLQMNSL |
| | | KPEDTAMYYCKTDPTIGCPQTYRYNYWGQGTQVTVSS |
| 322 | AS64071 | QVHLMESGGGSVQAGGSLRLSCVASGNTYRLNSMGWFRQAP |
| | | GKEREGVAFIVMIRGTTYYGASVKGRFTISQDNAQTTVYLQMS |
| | | SLKPEDTAMYYCAASTKDQFYVFNPIGYDSWGQGTQVTVSS |
| 323 | AS64093 | QVHLVESGGGSVQAGGSLRLSCATSRYIYGNNCMAWFRQAPG |
| | | KEREGVASIYPAGGRTYYADSVKGRFTISQDNAKNTVYLQIDSL |
| | | KPEDTAMYYCAARSFSIGVCATQSGITWSNFAYWGQGTQVTVSS |
| 324 | AS64118 | QVQLAESGGGSVQAGGSLRLSCAASGYTYSACRMAWYRQAP |
| | | GKERELVSFINSAGSTYYADSVKGRFAISRDNAKTTVYLQMNA |
| | | LKAEDTAIYYCNTWDSSCRFQYWGQGTQVTVSS |
| 325 | AS64120 | QVRLVESGGGSVQAGGSLRLSCETSRYIYGNNCMAWFRQAPG |
| | | KEREGVASIYPAGGRPYYADSVKGRFTISQDNAKNTVYLQMDS |
| | | LKPEDTAMYYCAARSFSIADCATQSGITRSNFAYWGQGTQVTV |
| | | SS |
| 326 | AS64124 | QVKLVESGGGSVQTGGSLRLSCAVSTYTPSNNYMGWFRQAPG |
| | | KEREGVAAIATIGGTTRYADSVKGRFTISQDGAKNTIYLQMNGL |
| | | KPEDTAMYYCAAGRPYSLPLPLPLESGAYRYWGQGTQVTVSS |
| 327 | AS64135 | QVKLVESGGGSVQAGGSLRLSCVASTSTYCRYYMRWYRQAPG |
| | | KEREFVSAMQPDGTTSYSDSVKGRFTMSQDRANNMLYLQMNS |
| | | LRPEDTAMYYCKRDPMGGSRTPCTSAWGQGTQVTVSS |
| 328 | AS64163 | QVRLVESGGGSVQAGGSLRLSCAVSGYRYRWNCMAWFRQAP |
| | | GKEREGVAAISTGSGSTYYAGSVKGRFTISQDNAKNMYLQMNS |
| | | LKPEDTAMYYCAADPSVCPGGMWYSKEYRYWGQGTQVTVSS |
| 329 | AS64182 | QVHLMESGGGSVQAGGSLRLSCTASGQTSRYLYMGWFRQTPG |
| | | KEREGVACIYTGSGSTGYADSVKGRFTISQDNAKNTVYLQTNS |
| | | LKPEDTAMYYCAASSPHWGGTCRRWSEYKYWGQGTQVTVSS |
| 330 | AS64183 | QVHLVESGGGSVQAGGSLRLSCAASGHTYSANCMAWFRRAPG |
| | | KEREWVASVYTDDDSTMYTDSVKGRFTIFQDNAKNTVYLQM |
| | | NSLKPEDTGMYICAADLSGGPAGCGYTHWGQGTQVTVSS |
| 331 | AS64207 | EVQLVESGGGSVQAGGSLRLSCAASGYTYSSNFMGWFRQAPG |
| | | KEREGVATIVSGGGTTYYADSVRGRFTISQDNAKNTVYLQMNS |
| | | LKPEDTAMYYCAAGGPPTNGAKWYPLRPPGYNYWGQGTQVT |
| | | VSS |
| 332 | AS64276 | QVHLMESGGGSVQAGGSLSLSCVVSGYTGSSRCMAWFRQAPG |
| | | KEREAVAQIFTGRGTTGYADSVKGRFTISQDNAKNTVYLRMNS |
| | | LRPEDTAIYYCAASLGPGRGACGYNYWGQGTQVTVSS |
| 333 | AS64336 | QVQLVESGGGSVQAGGSLRLSCTTSGRTYSSCSMGWYRQAPG |
| | | KERELVSHIFSDGSRYYADSVKGRFTISQDNAKNTVYLQMNSL |
| | | KPEDTAMYYCNRRTGWAPRCAVPGYWGQGTQVTVSS |
| 334 | AS64346 | QVHLVESGGGSVQAGGSLRLSCAASGYTYFMGWFRQAPQKER |
| | | EWVATIGTGDIFNGAAYYVDSVKGRFAISQDNAKNTVYLQMN |
| | | SLKPEDTAVYVCAAVQSKSSNYVLRDASTYNYWGQGTQVTVS |
| | | S |
| 335 | AS64420 | EVQLVESGGGSVQAEGSLRLSCAASGDTSRSVWMGWARQVPG |
| | | KEREVVATISTAGGSTWYTDSVKGRFTISQDNAKNTVYLQMNS |
| | | LKPEDTAIYYCAARSRYATYWGQGTQVTVSS |
| 336 | AS64473 | QVRLVESGGGSVQAGGSLRLSCTASGYTYRYLYMAWFRQTPG |
| | | KEREGVACIYTGSGTTGYADSVKGRFTISQDNAKNTVYLQMNS |
| | | LNAEDTAMYYCAASSPQWGGTCRRWSEYNYWGQGTQVTVSS |
| 337 | AS64475 | QVQLQESGGGSVQAGGSLRLSCAASGYTWSRNWMGWFRQAP |
| | | GKEREGFATITISGGSTWYADSVKGRFTISLDNAGNTVYLQMN |
| | | SLKPEDTAMYYCAARDTARTYWGQGTQVTVSS |
| 338 | AS64513 | EVQLVESGGGSVQAGGSLRLSCVASDYPYIDNCMGWFRQGPG |
| | | KEREGVAAACTGGGSTYYADSVKGRFTISRDNAKNTVYLQMN |
| | | SLKPEDTDVYYCATGYYSGSGPGYLLPWRYNYWGQGTQVTVS |
| | | S |
| 339 | AS64562 | EVQLVESGGGSVQAGGSLRLSCAASGYTARRDFMAWFRQVPG |
| | | KEREGVAVIHTGGDTTYYADSVKGRFTISRDNAQNIMNLQMNS |
| | | LKPEDTAMYYCAAGFRPRGGGYTGDVLAQAAAYNYWGQGTQ |
| | | VTVSS |
| 340 | AS64583 | EVQLAESGGGLVQAGGSLRLSCTASGFTIAVYTMGWFRQAPGK |
| | | EREGISCTSWAGGRTYTADSVKGRFTISRDNAKNTLYLQMNSL |
| | | KPEDTAMYYCAAKAHPDCSGDWSPSGYEYWGQGTQVTVSS |
| 341 | AS64594 | QVHLVESGGGSVQAGGSLRLSCAASGYTYNSNYMGWFRQAP |
| | | GKEREGVALIYTGGGSTYYADSVKGRFTISRDNAKNTVYLQMN |
| | | SLKPEDTAMYYCSVRTQTRNYWGQGTQVTVSS |
| 342 | AS64605 | QVKLVESGGGSVQAGGSLRLSCAVSRYPYSSICMGWFRQAPGK |
| | | ESEGVARIYTGTGSTWYTDSVKGRFTIARDNAQNTVYLQMNSL |
| | | KPEDTAMYYCAARSNSYSYSSCDYGPLTRGGYNFWGQGTQVT |
| | | VSS |
| 343 | AS64606 | EVQLAESGGGSVQAGGSLRLSCAVSGYTSRSNYMGWFRQAPG |
| | | KEREGVALIYTRGGSTYYASSVKGRFTISQDSAKKTYLQMNSV |
| | | KPEDTAMYYCALRLDEKMYWGQGTQVTVSS |
| 344 | AS68121 | EVQLAESGGGSVQAGGSLRLSCDASGYTYSRNCMGWFRQAPG |
| | | KEREGVAAFYTDYIRFGRTYYADSVKGRFTIFQDNAKNTVYLQ |
| | | MNSLKPEDTAMYYCAADPGSRTDDSCGTSYNKGNFGYWGQG |
| | | TQVTVSS |
| 345 | AS68170 | QVQLVESGGGSVQAGGSLRLSCTASGYTYRSNCMGWFRQAPG |
| | | KEREGVATIYTGGGRNLYYADSVKGRFTISRDNAKNTLYLQMN |
| | | SLKPEDSARYYCAAASDVAVGVNSCGGRTAGFDAWGQGTQVT |
| | | VSS |
| 346 | AS63964 | QVRLVESGGGSVQAGGSLRLSCSASGYTYSYNNMGWFRQAPG |
| | | NEREGVAAISGGRFTAYADSVKGRFTISRDNAENTLYLQMNNL |
| | | KPEDTGMYYCAAEVVDPTWGSRDQRRYKYWGQGTQVTVSS |
| 347 | AS64116 | QVKLVESGGGSVQAGGSLRLSCAASGYIYSCVGWFRQAPGKE |
| | | REGVAGISTGGGGTVYADSVKGQFTISRDNAKNTVYLQMDSLK |
| | | PEDTAMYYCAADRWNSFANCGAWGRYTYWGQGTQVTVSS |
| 348 | AS68270 | QVQLAESGGGSVQAGGSLRLSCVASGYPSSTYYMLSMAWFRQ |
| | | APGKEREGVAAITSGTGSTSYADSVKDRFTISKDYANNTLYLHI |
| | | NNLKPEDTAMYYCAAASGWIVPSRSLTANLYRYWGQGTQVTV |
| | | SS |
| 349 | AS68320 | QVHLVESGGDSVQAGGSLRLSCAASGYTYNTNYMGWFRQAP |
| | | GKEREGVAAIYRHSGNTAYADSVKGRFTISQDYAKNTVYLQMN |
| | | SLKPEDTAMYYCAAGRAGPWALMRPTEFGYWGQGTQVTVSS |
| 350 | AS68351 | QVQLEESGGGLVQPGGSLRLSCAASGDTFRAYYMNWVRQAPG |
| | | KGFEWVSGISASGGRTSYADSVKGRFTISRDNAKNTLYLQLNSL |
| | | STEDTGMYYCVKGAVRLSTSSVRDSSWGQGTQVTVSS |
| 351 | AS75378 | QVQLEESGGGSVQAGGSLRLSCVVSGNTRSTTYMGWFRQAPG |
| | | KEREGVAIVYTGGRDTYYAASVKGRFTISQDNAKTTIYLQMNS |
| | | LEPEDTAMYYCAARSYEYTYWGRGTQVTVSS |
| 352 | AS75383 | EVQLAESGGGSVQAGGSLRLSCVASGYTFSSYCLGWFRQAPGK |
| | | QRQGVATFNNRGVANYHDSVKGRFTASVDNAKNTLLLQMNSL |
| | | EPDDTAMYYCAADRRYGRQWYQPCEWNTWGQGTQVTVSS |
| 353 | AS75751 | QVRLVESGGGSVQAGGSLRLSCVASGYFYNTYYFMGWFRQAP |
| | | GKEREGVAAIDTDGRTSYADSVKGRFTISKDNAKNTLYLQMNS |
| | | LKPEDTAMYYCAAGFGYMNVIQALNGMRQNPDYWGQGTQV |
| | | TVSS |
| 354 | AS76422 | QVKLVESGGGSVQAGGSLRLSCAASGYTFAGNCLGWFRQAPG |
| | | KGREGVVTYNNFGVANYADSVKGRFTVSQDNAKNTLLLQMN |
| | | SLEPEDTAMYYCAADRRDGRRWSQPCEWNTWGQGTQVTVSS |

Amino acid sequences of VH and VL domains of anti-DLL3 human scFvs were listed in Table 4. Nucleic acid sequences encoding VH or VL domain of anti-DLL3 human scFvs were shown in SEQ ID NOs: 511-514.

TABLE 4
Anti-DLL3 human scFv amino acid sequences

| scFv | SEQ ID | Amino Acid Sequence |
|---|---|---|
| A556704 | 507 | VL: |
| | | DIQMTQSPSSLSASVGDRVTITCRA |
| | | SQSVSSAVAWYQQKPGKAPKLLIYS |
| | | ASSLYSGVPSRFSGSRSGTDFTLTI |
| | | SSLQPEDFATYYCQQASWSPITFGQ |
| | | GTKVEIK |
| | 508 | VH: |
| | | EVQLVESGGGLVQPGGSLRLSCAAS |
| | | GFNISSSYMHWVRQAPGKGLEWVAY |
| | | IYPSYGYTSYADSVKGRFTISADTS |
| | | KNTAYLQMNSLRAEDTAVYYCARGG |
| | | YYYHGMDYWGQGTLVTVSS |
| A556788 | 509 | VL: |
| | | DIQMTQSPSSLSASVGDRVTITCRA |
| | | SQSVSSAVAWYQQKPGKAPKLLIYS |
| | | ASSLYSGVPSRFSGSRSGTDFTLTI |
| | | SSLQPEDFATYYCQQHYAPSLITFG |
| | | QGTKVEIK |
| | 510 | VH: |
| | | EVQLVESGGGLVQPGGSLRLSCAAS |
| | | GFNISSYSMHWVRQAPGKGLEWVAY |
| | | ISSYYGYTYYADSVKGRFTISADTS |
| | | KNTAYLQMNSLRAEDTAVYYCARYS |
| | | YYYGMDYWGQGTLVTVSS |

### Example 3. Generation of Monospecific Camel CARs

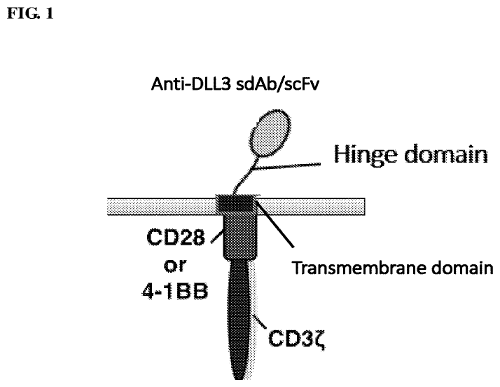

The amino acid sequences of anti-DLL3 camel sdAb fragments were provided above in Table 3 and the nucleic acid sequences of anti-DLL3 camel sdAb fragments were provided in SEQ ID NOs: 368-448. sdAb fragments of Table 3 and additional sequences were used to generate CAR constructs (SEQ ID NOs: 476-484). CAR3 scFv (SEQ ID NO: 473), which is a human anti-DLL3 scFv, was also used to generate a CAR construct as a reference (CAR3). A full length CAR contains from the N-terminus to the C-terminus: a CD8α signal peptide (SEQ ID NO: 465), a DLL3 binding domain sdAb provided in Table 3, a CD8α hinge domain (SEQ ID NO: 466), a CD8α transmembrane domain (SEQ ID NO: 467), a CD137 intracellular domain (SEQ ID NO: 468) or a CD28 intracellular domain (SEQ ID NO: 469), and a CD3ζ intracellular domain (SEQ ID NO: 470). Schematic representation of a CAR construct is shown in FIG. 1. Nucleic acid encoding the CAR fragment was then cloned into a lentiviral vector to create full length CAR construct in a single coding frame, using human EF1 alpha promoter for expression. The resulting CAR backbone vector was named “PLLV-hEF1α-DLL3”.

### Example 4. Generation of Camel Anti-DLL3 CAR-T Cells

### Preparation of Lentivirus

The lentivirus packaging plasmid mixture including pCMV-ΔR-8.47 and pMD2.G (Addgene, Cat #12259) was pre-mixed with PLLV-hEF1α-DLL3 vectors at a pre-optimized ratio (1:1:1:2) in the present of polyethylenimine before added to the HEK293 cells. The supernatants were collected after overnight incubation. The virus-containing supernatants were filtered through a 0.45 μm PES filter, and ultra-centrifuged to concentrate lentiviruses. The virus pellets were rinsed with pre-chilled DPBS. The viruses were aliquoted properly before stored at −80° C. immediately. Virus titer was determined by measurement of transduction efficiency to supT1 cell line via flow cytometric assay.

### Collection and Transduction of T Lymphocytes

Leukocytes were collected from healthy donors by apheresis. Peripheral blood mononuclear cells (PBMCs) were isolated using Ficoll-Paque™ PLUS Media according to manufacturer's protocol. Human T cells were purified from PMBCs using Pan T cell isolation kit (Miltenyi, Cat #130-096-535), following manufacturer's protocol. The purified T cells were subsequently pre-activated for 48 hours with human T cell activation/expansion kit (Miltenyi, Cat #130-091-441) according to manufacturer's protocol, in which anti-CD3/CD28 MACSiBead particles were added at a bead-to-cell ratio of 1:2. The pre-activated T cells were transduced with lentivirus stock in the presence of 7 μg/mL polybrene. The transduced cells were then transferred to the cell culture incubator for transgene expression under suitable conditions.

### Example 5. Evaluation of In Vitro Activity of Camel Anti-DLL3 CAR-T Cells

### In Vitro Cytotoxicity Assay

On day 6 post transduction, transduced T cells were harvested and co-incubated with DLL3-expressing tumor cell line SHP-77 at an effector (CAR-T) to target cell ratio of 2:1 and 5:1 for 20 hours. CAR3 CAR-T cells were used as a reference in all assays to compare assay variation and/or act as a control. Un-transduced T cells (UnT) were used as a negative control.

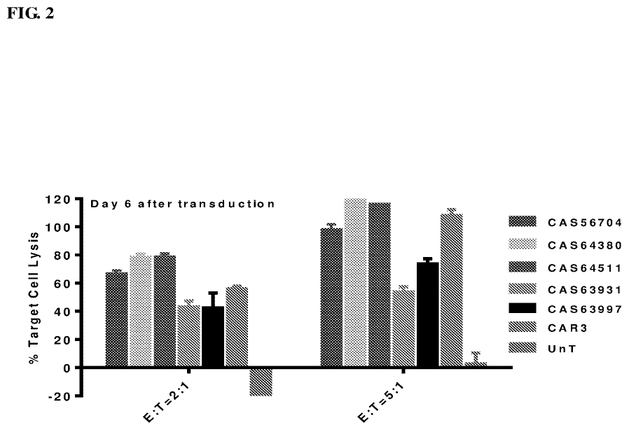

The cytotoxicity of the transduced T cells was determined by a lactate dehydrogenase (LDH) assay. Results show that CAR3 CAR-T and some anti-DLL3 CAR-Ts exhibit strong anti-tumor activities in vitro against SHP-77 cells, while UnT has no target cell killing effect (FIG. 2).

### IFN-γ and TNF-α Release Detection

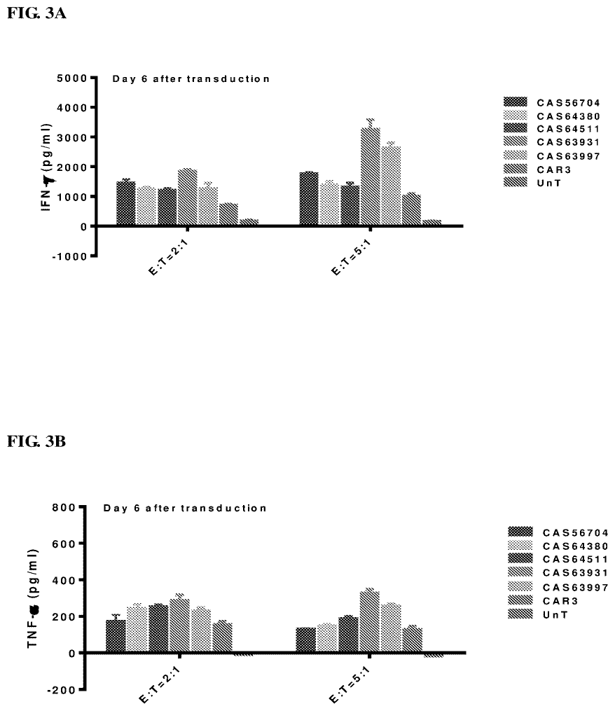

Additionally, supernatants from the in vitro cytotoxicity assay were collected to assess CAR-induced cytokine release, e.g., interferon gamma (IFN-γ) and TNF-α release. As shown in FIG. 3A and FIG. 3B, CAR3 CAR-T and some anti-DLL3 CAR-Ts were stimulated by SHP-77 to produce IFN-γ and TNF-α, whereas UnT produced little IFN-γ and TNF-α. Protocols of IFN-γ and TNF-α release detection can be referred to CISBIO's human TNF-α kits and IFN-γ kits.

### CAR-T Expansion by Long-Term Stimulation Assay

On day 0, 1×105 SHP-77 cells were plated in 24 well plates to establish a monolayer. On day 1, transduced T cells were counted and 2×105 viable CAR+ T cells were plated on top of the SHP-77 cells in fresh media in the absence of cytokines. On day 3, a new 1×105 SHP-77 cells monolayer were plated on top of the CAR-T cells. On day 4, viable CAR-T cells were counted for each well. One the same day, 2×105 CAR+ T cells from wells that expanded (had at least this amount of cells) were re-plated to establish a new monolayer as on Day 1. The process was repeated to provide 3-4 rounds of stimulation. Fold expansion after each stimulation was calculated as [viable CAR+ T cells on day 4]/2×105, the amount of CAR T cells plated on day 1 of each stimulation. To normalize for cells discarded with each new stimulation, cumulative fold expansion was determined by [(fold expansion)×(fold expansion+1) . . . ].

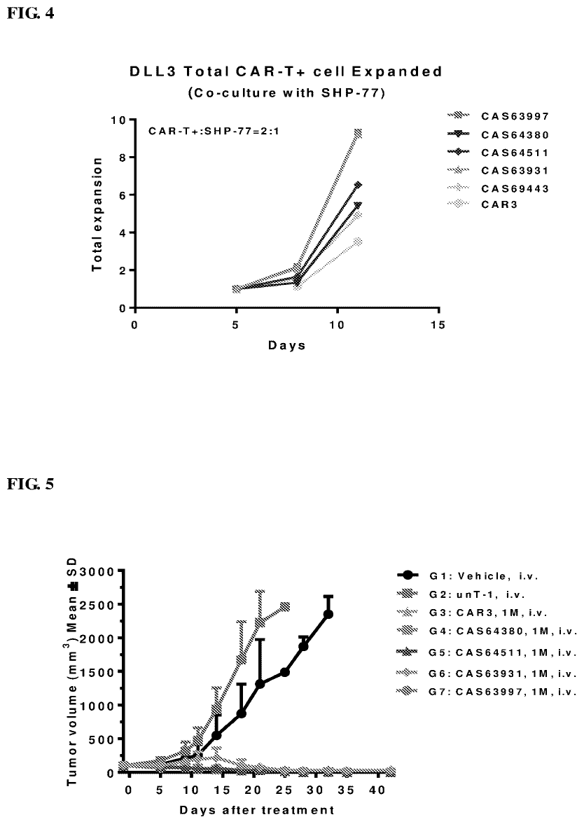

After 3 rounds of stimulation, the fold expansions of different CAR-T constructs were calculated. As it was showed in FIG. 4, most CAR-T constructs expanded more than CAR3 CAR-T with 3 rounds of stimulation by SHP-77 tumor cells.

### Example 6. In Vivo Efficacy Valuation of Camel CARs by CAR-T Cells Mediated Tumor Growth Inhibition

The anti-tumor activity of the camel CARs was evaluated in a SHP-77 tumor model. SHP-77 cells were implanted subcutaneously in NOD/SCID mice and randomized into 7 groups (4 mice per group, day 0). Group 1: vehicle (PBS only); Group 2: UnT (negative control); Group 3: CAR3; Group 4: CAS64380; Group 5: CAS64511; Group 6: CAS63931; Group 7: CAS63997. Treatment with CAR-T cells, UnT cells or vehicle (PBS only) began when tumors were palpable (100 mm3) and mice were euthanized when their tumor volumes reached about 3000 mm3. Tumor volumes were measured 2 times per week. CAR-T cells were administered intravenously with 1×106 CAR positive T cells per mouse. Mice and tumors were monitored for about 21 days after tumor cell implantation.

As shown in FIG. 5, all selected camel CARs demonstrated anti-tumor activity in this animal tumor model.

### Example 7. Humanization of Camel sdAbs

Selected camel sdAbs (SEQ ID NOs: 279, 294, 297, 312) were humanized using CDR grafting technology (see, e.g., U.S. Pat. No. 5,225,539). Briefly, the camel sdAb sequence was compared to those available in the Research Collaboratory for Structural Bioinformatics (RCSB) protein databank. A homology model of each camel sdAb was generated based on the nearest VH structures. From the model structure, residues that are in the proximity of CDRs or buried inside the molecule (i.e. with side chain solvent accessible surface area less than 15%) were identified.

Subsequently, each camel sdAb sequence was BLASTed against NCBI human germline V gene database to identify the human VH germline sequence (i.e. human acceptor) with highest identity to the sdAb (see, e.g., Foote and Winter, J. Mol. Biol. 224:487-499 (1992); Morea V. et al., Methods 20:267-279 (2000); Chothia C. et al., J. Mol. Biol. 186:651-663 (1985).) In the CDR grafting approach, CDRs of the human acceptor were replaced by those of the camel sdAbs, which produced the straight-graft sequence. Straight-graft antibody usually lost binding activity, which needed to be restored by replacing the framework residues that were critical for the activity of the antibody with non-human residues. Amino acid residues that were in the proximity of CDRs or buried inside the molecule were usually important for the activity and structure of the antibody, and therefore should be potential back-mutation sites. A series of humanized variants were designed using this method. CDR amino acid sequences of the humanized variants were shown in Table 5. Full length amino acid sequences of the humanized variants were shown in Table 6. CDRs were underlined.

TABLE 5
Anti-DLL3 humanized sdAb CDR sequences

| | | CDR1 | | CDR2 | | CDR3 |
|---|---|---|---|---|---|---|
| Ab | ID | Sequence | ID | Sequence | ID | Sequence |
| AS64380VH4 | 244 | GNTYS | 254 | VIYTR | 264 | SSRHR |
| | | SNYMG | | GGHTY | | LGLNN |
| | | | | YVDSV | | PRDYD |
| | | | | RG | | Y |
| AS64380VH5 | 245 | GNTYS | 255 | VIYTR | 265 | SSRHR |
| | | SNYMG | | GGHTY | | LGLNN |
| | | | | YVDSV | | PRDYD |
| | | | | RG | | Y |
| AS64380VH6 | 246 | GNTYS | 256 | VIYTR | 266 | SSRHR |
| | | SNYMG | | GGHTY | | LGLNN |
| | | | | YVDSV | | PRDYD |
| | | | | RG | | Y |
| AS64380VH7 | 247 | GNTYS | 257 | VIYTR | 267 | SSRHR |
| | | SNYMG | | GGHTY | | LGLNN |
| | | | | YVDSV | | PRDYD |
| | | | | RG | | Y |
| AS64511VH4 | 248 | RATYS | 258 | TITTG | 268 | NLRIG |
| | | TNYIS | | DGETA | | GDWFD |
| | | | | YADSV | | GRDFR |
| | | | | KG | | A |
| AS64511VH5 | 249 | RATYS | 259 | TITTG | 269 | NLRIG |
| | | TNYIS | | DGETA | | GDWFD |
| | | | | YADSV | | GRDFR |
| | | | | KG | | A |
| AS64511VH6 | 250 | RATYS | 260 | TITTG | 270 | NLRIG |
| | | TNYIS | | DGETA | | GDWFD |
| | | | | YADSV | | GRDFR |
| | | | | KG | | A |
| AS63997VH4 | 251 | FSGYG | 261 | AITVG | 271 | GYLSG |
| | | VSTMA | | SGNTY | | GSWDV |
| | | | | YADSV | | PGRYN |
| | | | | KG | | Y |
| AS63997VH5 | 252 | FSGYG | 262 | AITVG | 272 | GYLSG |
| | | VSTMA | | SGNTY | | GSWDV |
| | | | | YADSV | | PGRYN |
| | | | | KG | | Y |
| AS63997VH6 | 253 | FSGYG | 263 | AITVG | 273 | GYLSG |
| | | VSTMA | | SGNTY | | GSWDV |
| | | | | YADSV | | PGRYN |
| | | | | KG | | Y |

TABLE 6
Anti-DLL3 humanized sdAb amino acid sequences

| SEQ | Humanized | |
|---|---|---|
| ID | sdAb | Amino Acid Sequence |
| 355 | AS64380VH4 | EVQLVESGGGLVQPGGSLRLSCAASGNTYSSNYMGWFRQAPG |
| | | KGLEEVAVIYTRGGHTYYVDSVRGRFTISRDNAKNSLYLQMN |
| | | SLRAEDTAVYYCAASSRHRLGLNNPRDYDYWGQGTLVTVSS |
| 356 | AS64380VH5 | EVQLVESGGGLVQPGGSLRLSCAASGNTYSSNYMGWFRQAPG |
| | | KGLEEVAVIYTRGGHTYYVDSVRGRFTISQDNAKNSLYLQMN |
| | | SLRAEDTAVYYCAASSRHRLGLNNPRDYDYWGQGTLVTVSS |
| 357 | AS64380VH6 | EVQLVESGGGLVQPGGSLRLSCAASGNTYSSNYMGWFRQAPG |
| | | KGLEEVAVIYTRGGHTYYVDSVRGRFTISQDNAKNSVYLQMN |
| | | SLRAEDTAMYYCAASSRHRLGLNNPRDYDYWGQGTLVTVSS |
| 358 | AS64380VH7 | EVQLVESGGGLVQPGGSLRLSCAASGNTYSSNYMGWFRQAPG |
| | | KGREEVAVIYTRGGHTYYVDSVRGRFTISQDNAKNSVYLQMN |
| | | SLRAEDTAMYYCAASSRHRLGLNNPRDYDYWGQGTLVTVSS |
| 359 | AS64511VH4 | EVQLVESGGGLVQPGGSLRLSCAASRATYSTNYISWFRQAPG |
| | | KGLEAVATITTGDGETAYADSVKGRFTISRDNAKNSLYLQMN |
| | | SLRAEDTAVYYCAANLRIGGDWFDGRDFRAWGQGTLVTVSS |
| 360 | AS64511VH5 | EVQLVESGGGLVQPGGSLRLSCAASRATYSTNYISWFRQAPG |
| | | KGLEAVATITTGDGETAYADSVKGRFTISRDNAKNSVYLQMN |
| | | SLRAEDTAMYYCAANLRIGGDWFDGRDFRAWGQGTLVTVSS |
| 361 | AS64511VH6 | EVQLVESGGGLVQPGGSLRLSCAASRATYSTNYISWFRQAPG |
| | | KGREAVATITTGDGETAYADSVKGRFTISRDNAKNSVYLQMN |
| | | RSLAEDTAMYYCAANLRIGGDWFDGRDFRAWGQGTLVTVSS |
| 362 | AS63931VH4 | QVQLVESGGGVVQPGGSLRLSCAGSFSGYGVSTMAWFRQAPG |
| | | KGLEGVAAITVGSGNTYYADSVTGRFTISRDNSKNTLYLQMN |
| | | SLRAEDTAVYYCAAGWLSGGSWHVPGRYNYWGQGTLVTVSS |
| 363 | AS63931VH5 | QVQLVESGGGVVQPGGSLRLSCAGSFSGYGVSTMAWFRQAPG |
| | | KGLEGVAAITVGSGNTYYADSVTGRFTISRDNSKNTVYLQMN |
| | | SLRAEDTAMYYCAAGWLSGGSWHVPGRYNYWGQGTLVTVSS |
| 364 | AS63931VH6 | QVQLVESGGGVVQPGGSLRLSCAGSFSGYGVSTMAWFRQAPG |
| | | KGREGVAAITVGSGNTYYADSVTGRFTISRDNSKNTVYLQMN |
| | | SLRAEDTAMYYCAAGWLSGGSWHVPGRYNYWGQGTLVTVSS |
| 365 | AS63997VH4 | QVQLVESGGGVVQPGGSLRLSCAASFSGYGVSTMAWFRQAPG |
| | | KGLEGVAAITVGSGNTYYADSVKGRFTISRDNSKNTLYLQMN |
| | | SLRAEDTAVYYCAVGYLSGGSWDVPGRYNYWGQGTLVTVSS |
| 366 | AS63997VH5 | QVQLVESGGGVVQPGGSLRLSCAASFSGYGVSTMAWFRQAPG |
| | | KGLEGVAAITVGSGNTYYADSVKGRFTISRDNSKNTVYLQMN |
| | | SLRAEDTAMYYCAVGYLSGGSWDVPGRYNYWGQGTLVTVSS |
| 367 | AS63997VH6 | QVQLVESGGGVVQPGGSLRLSCAASFSGYGVSTMAWFRQAPG |
| | | KGREGVAAITVGSGNTYYADSVKGRFTISRDNSKNTVYLQMN |
| | | SLRAEDTAMYYCAVGYLSGGSWDVPGRYNYWGQGTLVTVSS |

The camel and humanized sdAb sequences were fused with human IgG1 hinge and Fc, resulting the chimeric and humanized HCAb sequences. The DNAs encoding these HCAbs were synthesized and inserted into pTT5 vector. HCAb expression plasmids were used to transfect HEK293 cells. Crude HCAb proteins secreted to the medium were subjected to SPR affinity measurement as follows: briefly, capturing antibody anti-human Fc pAb (GE healthcare) was immobilized on a Biacore™ CM5 chip to approximately 6,000 RU using EDC-activated amine coupling chemistry. HCAb of interest was captured for 300 seconds onto the sensorchip surface. Human DLL3 (AdipoGen, AG-40B-0151) was flowed over the sensorchip surface at a series of increasing concentrations. Association and dissociation phases were monitored. Captured antibody and antigen were removed between cycles using 10 mM Glycine-HCl, pH 2.0 buffer in order to ensure a fresh binding surface for the antigen. The resulting sensorgrams were fit globally using a 1:1 binding model in order to calculate on- and off-rates (ka and kd, respectively), as well as affinities (K_{D}).

The binding affinities of some humanized sdAbs were measured and compared those of the original camel sdAbs (Table 7). Most of the humanized antibodies retained the binding affinities of the camel sdAbs. This example demonstrated that the humanization of sdAbs using our standard protocol was successful. Most of the sdAbs retained their binding affinities after humanization.

The scFvs have comparable KD values (AS56788 and AS56704 in Table 7) when assayed with the same procedure as described above.

TABLE 7
Monovalent binding affinity of camel and
humanized antibodies as well as scFvs.

| Ligand | ka (1/Ms) | kd (1/s) | K_{D} (M) |
|---|---|---|---|
| AS64380 | 6.7E+05 | 5.6E−03 | 8.4E−09 |
| AS64380VH4 | 3.1E+05 | 4.4E−03 | 1.4E−08 |
| AS64380VH5 | 5.9E+04 | 1.5E−03 | 2.6E−08 |
| AS64380VH6 | 8.8E+04 | 1.5E−03 | 1.7E−08 |
| AS64380VH7 | 3.2E+04 | 1.5E−03 | 4.5E−08 |
| AS64511 | 1.3E+05 | 4.0E−04 | 3.0E−09 |
| AS64511VH4 | 1.6E+05 | 9.4E−04 | 6.0E−09 |
| AS64511VH5 | 1.6E+05 | 4.7E−04 | 2.9E−09 |
| AS64511VH6 | 1.4E+05 | 4.8E−04 | 3.4E−09 |
| AS63997 | 3.9E+05 | 6.4E−03 | 1.6E−08 |
| AS63997VH4 | 8.4E+05 | 1.8E−02 | 2.1E−08 |
| AS63997VH5 | 5.8E+05 | 2.1E−02 | 3.7E−08 |
| AS63997VH6 | 1.8E+06 | 6.0E−02 | 3.3E−08 |
| AS64617 | 1.9E+05 | 3.8E−03 | 2.0E−08 |
| AS69443 | 9.4E+04 | 9.0E−05 | 9.6E−10 |
| AS63931 | 3.6E+06 | 1.7E−01 | 4.7E−08 |
| AS64047 | 1.4E+05 | 9.6E−04 | 6.7E−09 |
| AS64052 | 1.0E+05 | 4.1E−04 | 4.0E−09 |
| AS64062 | 1.0E+05 | 7.1E−04 | 6.9E−09 |
| AS56788 | 1.3E+05 | 3.0E−03 | 2.3E−08 |
| AS56704 | 6.60E+04 | 5.59E−04 | 5.59E−09 |

### Example 8. Generation of Monospecific Humanized CARs

The amino acid sequences of anti-DLL3 humanized sdAbs were provided above in Table 6 and the nucleic acid sequences of anti-DLL3 humanized sdAbs were listed in SEQ ID NOs: 449-461. Humanized sdAbs in Table 6 and additional sequences were used to generate full CAR constructs (SEQ ID NOs: 485-494). A full length CAR contains from the N-terminus to the C-terminus: a CD8α signal peptide (SEQ ID NO: 465), a DLL3 binding domain (humanized sdAbs) provided in Table 6, a CD8α hinge domain (SEQ ID NO: 466), a CD8α transmembrane domain (SEQ ID NO: 467), a CD137 intracellular domain (SEQ ID NO: 468) or a CD28 intracellular domain (SEQ ID NO: 469), and a CD3ζ cytoplasmic domain (SEQ ID NO: 470). Schematic representation of a CAR construct is shown in FIG. 1. Nucleic acid encoding the CAR fragment was then cloned into a lentiviral vector to create a full length CAR construct in a single coding frame, using human EF1 alpha promoter for expression. The resulting CAR backbone vector was named “PLLV-hEF1α-DLL3”.

### Example 9. Evaluation of In Vitro Activity of Humanized Anti-DLL3 CAR-T Cells

Similar to the procedure described in Example 4, potency of humanized CARs was evaluated via CAR-T cell mediated killing of tumor cells, cytokine releasing and long-term stimulation assay.

### In Vitro Cytotoxicity Assay

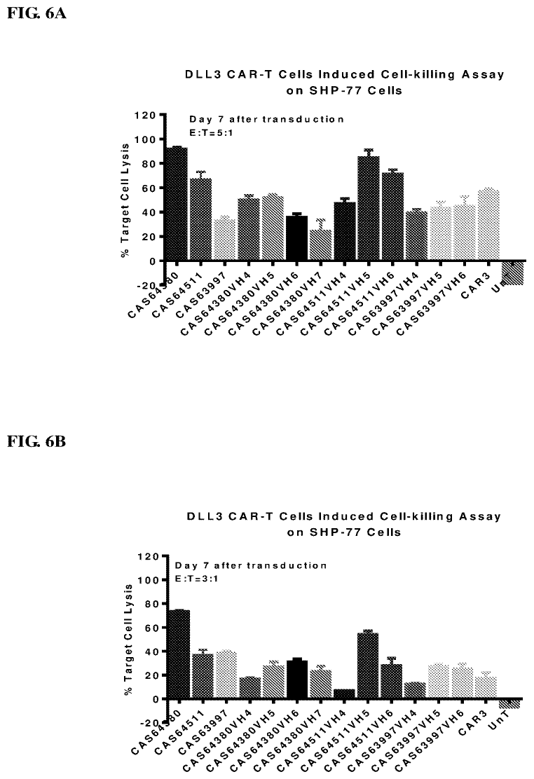

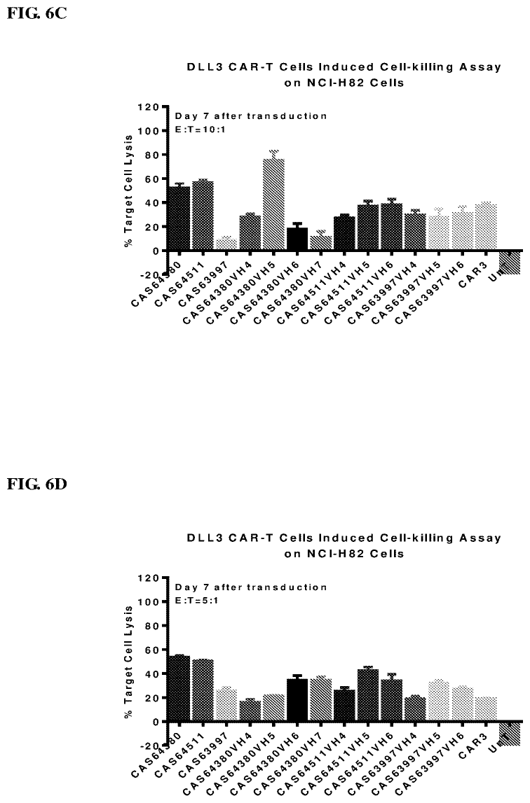

The results were shown in FIG. 6A-6D. Our humanized CAR-Ts showed superior anti-tumor efficacy in vitro.

### IFN-γ Release Assay

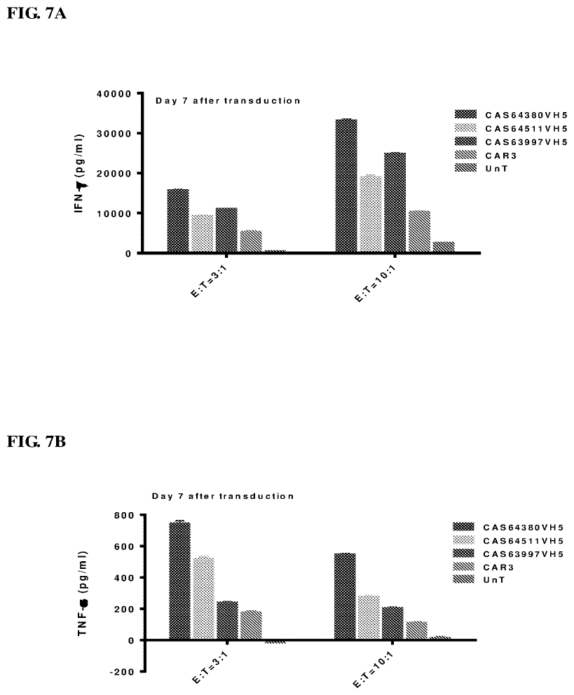

Additionally, supernatants from the in vitro cytotoxicity assay were collected to assess CAR-induced cytokine release, e.g., interferon gamma (IFN-□) and tumor necrosis factor α (TNF-α) release. As shown in FIG. 7A and FIG. 7B, CAR3 CAR-T and some anti-DLL3 CAR-Ts were stimulated by SHP-77 to produce IFN-γ and TNF-α, whereas UnT produced little IFN-γ or TNF-α.

### CART Expansion by Long-Term Stimulation Assay

On Day 0, 1×10⁵ SHP-77 cells were plated in 24 well plates to establish a monolayer. On Day 1, CAR-T cells were counted and 2×10⁵ viable CAR⁺ T cells were plated on top of the SHP-77 cells in fresh media in the absence of cytokines. On day 3, a new 1×10⁵ NCI-H82 cells monolayer were plated on top of the CAR-T cells. On day 4, viable CAR-T cells were counted for each well. On the same day, 2×10⁵ CAR⁺ T cells from wells that expanded (have at least this amount of cells) were re-plated to establish a new monolayer as on Day 1. The process was repeated for 3-4 rounds of stimulation. Fold expansion after each stimulation was calculated as [viable CAR⁺ T cells on day 4]/2×10⁵, the amount of CAR-T cells plated on day 1 of each stimulation. To normalize for cells discarded with each new stimulation, cumulative fold expansion was determined by [(fold expansion)×(fold expansion+1) . . . ].

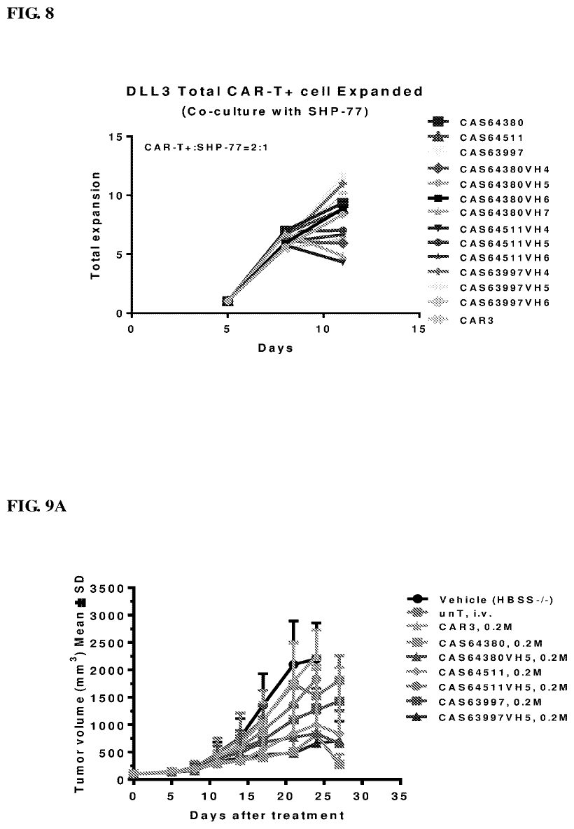

After 3 rounds of stimulation, the fold expansions of different CAR-T constructs were calculated. As it was showed in FIG. 8, most CAR-T constructs expanded more than CAR3 CAR-T with 3 rounds of stimulation by SHP-77 tumor cells.

### Example 10. In Vivo Efficacy Valuation of Humanized CARs by CAR-T Cells Mediated Tumor Growth Inhibition

The anti-tumor activity of the humanized CARs was evaluated in a SHP-77 tumor model. SHP-77 cells were implanted subcutaneously in NOD/SCID mice and randomized into 9 groups (4 mice per group, day 0): Vehicle (PBS only), UnT (negative control), CAR3, Treatment with CAR-T cells, UnT cells or vehicle (PBS only) began when tumors were palpable (100 mm3) and mice were euthanized when their tumor volumes reached about 3000 mm3. Tumor volumes were measured 2 times per week. CAR-T cells were administered intravenously with 0.2×10⁶ CAR positive T cells per mouse. Mice and tumors were monitored for about 21 days after tumor cell implantation.

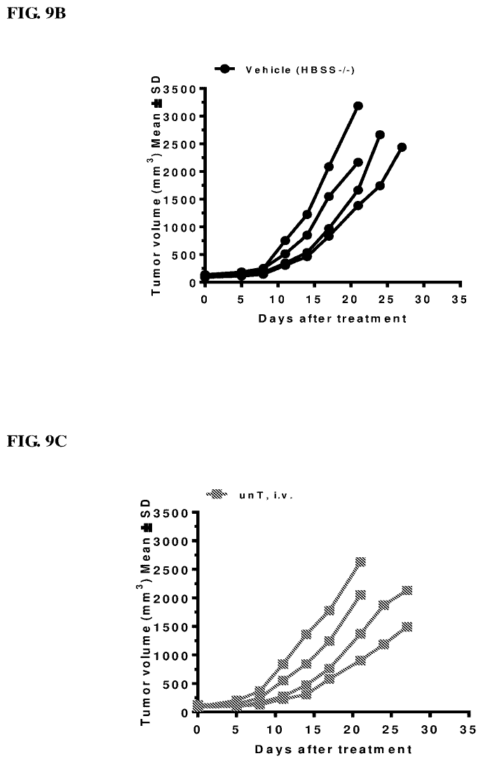

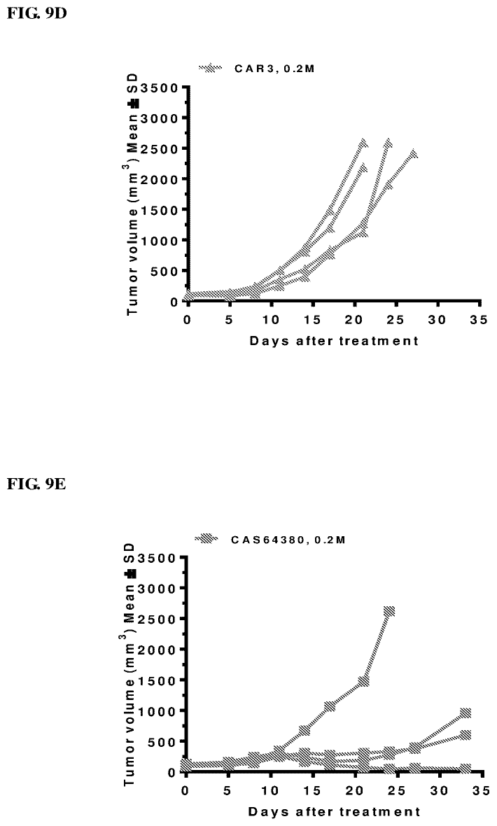

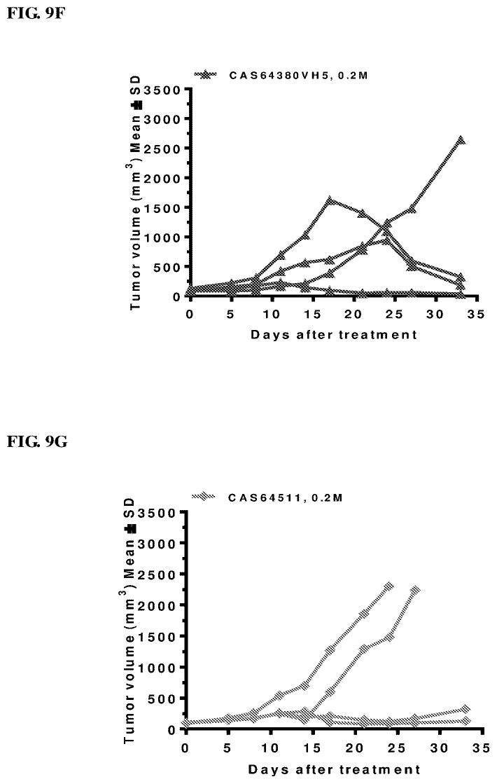

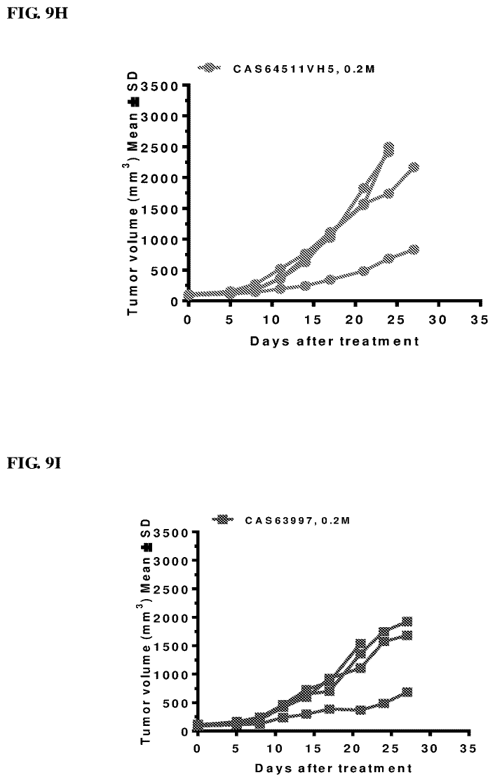

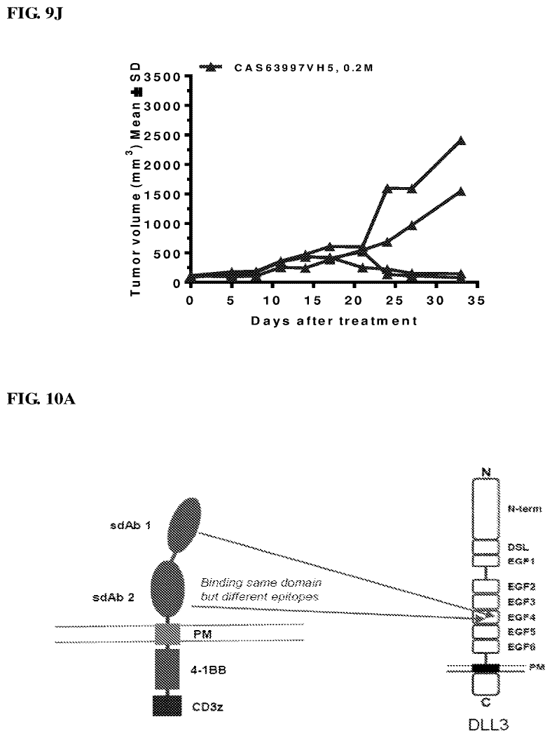

As shown in FIG. 9A-9J, compared to benchmark CAR3, CAS64380VH5 and CAS63997VH5 showed superior anti-tumor activity in this animal model.

Anti-tumor activities of these humanized CARs were not observed to correlate with their potency of in vitro cell killing.

Listed below are some amino acid sequences and nucleic acid sequences mentioned herein.

Camel sdAb Nucleic Acid Sequences
(Camel sdAb AS63930 Nucleic Acid Sequence)
SEQ ID NO: 368
GAGGTGCAACTGGCGGAGTCTGGGGGAGGATCGGT
GCAGGCTGGAGGGTCTCTGAGACTCTCCTGTGCAG
CCTCTGGATACACCTACAGTGGCAACTATATGGCC
TGGTTCCGCCAGGCTCCAGGGAACGAGCGCGAGGG
GGTCGCAGTTGTTTATAATATTGACGGTGGCGGTC
GTTTCACTACCTATGCCGACTCCGTGAAGGGCCGA
TTCACCATCTCCCGAGGCAACGACAAGAACACGGT
GTATCTGCAAATGAACAGCCTGAAACCTGAGGATA
GTGGCATGTACTACTGTGCGGCAGAGGTAGCTGAT
CCGACCTGGGGGTCGCGTGACCAAAGACGATATAA
GTACTGGGGCCAGGGGACCCAGGTCACCGTCTCCT
CA
(Camel sdAb AS63932 Nucleic Acid Sequence)
SEQ ID NO: 369
CAGGTGCAATTGGAGGAGTCTGGGGGAGGCTCGGT
GCAGGCTGGAGGGTCTCTGAGACTCTCCTGTGTAG
CCTCTGGATACACCTACGGTAGCACCTTCATGGGC
TGGTTCCGCCAGAATCCAGGGAAGGAGCGCGAGGG
GGTCGCAGTTATTTATACTGGTGGTGGTAGTACAT
GGTATGCCAGCTCCGTGAAGGGCCGATTCACCATC
TCCCAGGACAACGCCAAGAACACGGTGTATCTGCA
AATGAACAGCCTGAAACCTGAGGACACTGCCATGT
ACTACTGTGCGGCGCGTTACGGGTCGGGAAACGTT
AACTACTGGGGCCAGGGGACCCAGGTCACCGTCTC
CTCA
(Camel sdAb AS63951 Nucleic Acid Sequence)
SEQ ID NO: 370
CAGGTGCACCTGATGGAGTCTGGGGGAGGCTCGGT
GCAGGCTGGAGGGTCTCTGAGACTCGCCTGTGAAA
CCTCTAGAGACATCTACGGTAACAACTGCATGGCC
TGGTTCCGCCAGGCTCCAGGGAAGGAGCGCGAGGG
AGTCGCGTCTATTTATCCTGCTGGTGGTCGCCCGT
ACTATGCCGACTCCGTGAAGGGCCGATTCACCATC
TCCCAAGACAACGCCAAGAACACGGTGTATCTGCA
AATGGACAGCCTGAAACCTGAGGACACGGCCATGT
ACTACTGCGCGGCACGCTCTTTTTCGATAGCAGTT
TGCGCGACGCGCTCTGGTATTACCAGGTCTAATTT
TGCTTACTGGGGCCAGGGGACCCAGGTCACCGTCT
CCTCA
(Camel sdAb AS63984 Nucleic Acid Sequence)
SEQ ID NO: 371
CAGGTGAAGTTGGTGGAGTCTGGGGGAGGCTCGGT
GCAGGCTGGAGGGTCTCTGAGACTCTCCTGTGCAG
CCTCTGGATACACCTACAGTAGCAACTTCATGGGC
TGGTTCCGCCAGGCTCCAGGGAAGGAGCGCGAGGG
GGTCGCAACTATTGTTTCTGGTGGTGGTACCACAT
ACTATGCCGACTCCGTGAGGGGCCGATTCACCATC
TCCCAAGACAACGCCAAGAACACGGTGTATCTGCA
AATGAACAGCCTGAAACCTGAGGACACTGCCATGT
ACTACTGTGCGGCAGGGGGCCCCGTTACGAACGCA
CCTAGATGGTACCCCCTCCGACCTCCTGGTTATAA
CTACTGGGGCCAGGGGACCCAGGTCACCGTCTCCT
CA
(Camel sdAb AS63987 Nucleic Acid Sequence)
SEQ ID NO: 372
CAGGTGAGGTTGGTGGAGTCTGGGGGAGGCTCGGT
GCAGGCTGGAGGGTCTCTGAGACTCTCCTGTGAAG
CCACTGGATATAGAAACTGCATGGCCTGGTTCCGC
CAAGCTCCAGGGAAGGAACGCGAGGGGGTCGCAGT
TATTTATACTCCTAGTGGTATCACGGACTATGCAA
GCTCCGTGAAGGGCCGATTCACCATCTCCCAAAAC
AACGCCAGGAACACGCAGTATCTGCAAATGAACAG
CCTGAAACCTGAGGACACTGCCATGTACTACTGTG
CGGCAGATCGACCCTTTGTTTGTAATATAGCGAAT
ATGAGAAGGTCCTCCAACTGGGGCCGGGGGACCCA
GGTCACCGTCTCCTCA
(Camel sdAb AS63997 Nucleic Acid Sequence)
SEQ ID NO: 373
CAGGTGAGGTTGGTGGAGTCTGGGGGAGGCTCGGT
GCAGGCTGGAGGGTCTCTGAGACTCTCCTGTGCAG
GCTCTTTCTCTGGATACGGCGTCAGTACCATGGCC
TGGTTCCGCCAGGCTCCAGGGAAGGAGCGCGAGGG
GGTCGCAGCTATTACAGTTGGTAGTGGAAACACAT
ACTATGCCGACTCCGTGAAGGGCCGATTCACCATC
TCCCGAGACAACGCCAAGAGGACGGTGTTTTTGCA
AATGAACAGCCTGAAACCTGAGGACACTGCCATGT
ACTACTGTGCGGTCGGATACTTGTCGGGTGGTAGT
TGGGACGTTCCCGGAAGGTATAACTACTGGGGCCA
GGGGACCCAGGTCACCGTCTCCTCA
(Camel sdAb AS64047 Nucleic Acid Sequence)
SEQ ID NO: 374
CAGGTGCACCTGGTGGAGTCTGGGGGAGGCTCGGT
GCAGGCTGGAGGGTCTCTGAGACTCTCCTGTGCAG
CCTCTCAGTACGTTTACAGGTGGGACCTCATGGGC
TGGTTCCGCCAGGCTCCAGGGAAGGAGCGCGAGGC
GGTCGCTGCTGTTTATACTGGTGATGGTATTACAT
ACTATGCCGACTCCGTGAAGGGCCGATTCAGCATT
TCCCAAGACAACGCCAAGAACACGGTGTATCTGCA
AATGAACAGCCTGAAACCTGAGGACACTGGCATGT
ACTTCTGTGCGGCAGGCTTCGTCTCTGGTGGTAGA
TGGAACCAGTCATATCGTTATAAATACTGGGGCCA
GGGGACCCAGGTCACCGTCTCCTCA
(Camel sdAb AS64052 Nucleic Acid Sequence)
SEQ ID NO: 375
CAGGTGCACCTGATGGAGTCTGGGGGAGGCTCGGT
GCAGGCTGGAGGGTCTCTGAGACTCTCCTGTGCAG
CCTCTGGATACACCTACCGCAGCAACTTCATGGGC
TGGTTCCGCCAGGCTCCAGGGAAGGAGCGCGAGGG
GATCGCAACTATTCATTCTGGTGTGGCTACCACAT
ACTATGCCGACTCCGTGAAGGGCCGATTCACCATC
TCCCAAGACAACGCCAAGAACACGGTGTATCTGCA
AATGAACAGCCTGAAACCTGAGGACACTGCCATGT
ACTACTGTGCGGCAGGGGGCCCCCCTGCGAACGCT
GATAGATGGTACCCCCTCCGACCTCCTGGTTATAA
CTACTGGGGCCAGGGGACCCAGGTCACCGTCTCCT
CA
(Camel sdAb AS64062 Nucleic Acid Sequence)
SEQ ID NO: 376
CAGGTGAGGTTGGTGGAGTCTGGGGGAGGCTCGGT
GCAGGTTGGAGGGTCTCTGAGACTCTCCTGTGCAG
CCTCTAGATCTCCCTACAGTAGCAGTAGGTGCATG
GGGTGGTTCCGCCAGGCTCCAGGGAAGGAGCGCGA
GGGGGTCGCAGCTCTTTATACTGGTGGTGGTAGCA
CATCCTATGCCGACTCCGTGAAGGGCCGATTCACC
ATCTCCCAAGACAACGCCAAGAATACGGTGTATCT
GCAAATGAACAGCCTGAAACCTGAGGACACTGCCA
TGTACTACTGTGCGGCAGTTGTCCCTAGGGGTGGT
AGCTGCCGTCTTGATGAAAGAGGGTATTACCACTG
GGGCCAGGGGACCCAGGTCACCGTCTCCTCA
(Camel sdAb AS64072 Nucleic Acid Sequence)
SEQ ID NO: 377
CAGGTGCAGCTGGTGGAGTCTGGGGGAGGCTCGGT
GCAGGCCGGAGGGTTTCTGAGACTCTCCTGTGCAC
TCTCTGGATACAGCTACTATATTAACTTGATGGCG
TGGTTCCGTCAGGCTCCAGGGAAGGAGCGCGAGGC
AGTCGCAGCTCATGGTCCTGTGAGTGGGACAGCAT
ACTATACCGACTCCGTGAAGGGCCGATTCACCATC
TCCCGAGACCCCGGCAAGAACACGATGTATCTTCA
AATGTTTAGCCTGCAACCGGAGGACACTGCCCTCT
ACTACTGTGCGGCGGAAACGACTATGGGTTGGGCC
CACGAACGCGGGTATAGGTACTGGGGCCAGGGGAC
CCAGGTCACCGTCTCCTCA
(Camel sdAb AS64097 Nucleic Acid Sequence)
SEQ ID NO: 378
CAGGTGCACCTGATGGAGTCTGGGGGAGGCTCGGT
GCAGGCTGGAGGGTCTCTGAGACTCTCCTGTGAAG
CCTCTGGATACACCTACAGTCGCAACTGCATGGGC
TGGTTCCGCCAGGCTCCAGGGAAGGAGCGCGAGGG
GGTCGCAGCTATTAACACTGGTGGTGGTAGCACAT
ATTATGCCGACTCCCTTGAGGGCCGATTCACCATC
TCCCAAGACAACGCCAAGAATACTATGTATCTGCA
AATGAACAGCCTGAAACCTGAGGACACTGCCATGT
ACTACTGTGCGGCAGGTCCCGATCTCGGTGGTAGC
TGGTGTCGGCCCGTTGAGCGGGCTTTTACGTACTG
GGGCCAGGGGACCCAGGTCACCGTCTCCTCA
(Camel sdAb AS64114 Nucleic Acid Sequence)
SEQ ID NO: 379
CAGGTGCAACTGCAGGAGTCTGGGGGGGGCTCGGT
GCAGGCTGGAGGGTCTCTGACACTCTCCTGTGAAG
CCTCTGGAAACACCTACAGTACTAATTACATGGGC
TGGTTCCGCCAGGCTCCAGGGAAGGAGCGCGAAGA
GGTCGCGGTTATTTACACTCGTGGTGGTCACACAT
ACTATGTCGACTCCGTGAGGGGCCGATTCACCATC
TCCCAAGACAACGCCAAGAACACGGTGTATCTGCA
AATGAACAGCCTGAAACCTGAGGACACTGCCATGT
ACTACTGTGCGGCAGCTTCACGACATAGACTCCGT
TTAAATAACCCACGGGACTATGACTACTGGGGCCA
GGGGACCCAGGTCACCGTCTCCTCA
(Camel sdAb AS64123 Nucleic Acid Sequence)
SEQ ID NO: 380
CAGGTGCAACTGGCGGAGTCTGGGGGAGGCTCGGT
GCAGGCTGGAGGGTCTCTGAGACTCTCCTGTGCAG
CCTCTGGATACACTTATACGAGCAACTGGCTGGGC
TGGTTCCGCCAGGCTCCAGGGAAGGAGCGCGAGGA
GGTCGCAATTATTTATACTGGTAGTGGTAGTACAC
ACTATCGCAGCTCCGTGAAGGGCCGATTCACCATC
TCCCAAGACAACGCCAAGAACACGGTGTATCTGCA
AATGAACAGCCTGAAACCTGAGGACACTGCCATGT
ACTACTGTGCGGCGCGTTTCTCAGAGTATAATTAC
TGGGGCCAGGGGACCCAGGTCACCGTCTCCTCA
(Camel sdAb AS64130 Nucleic Acid Sequence)
SEQ ID NO: 381
GAGGTGCAACTGGCGGAGTCTGGGGGAGGCTCGGT
GCAGGCTGGAGGGTCTCTGAGACTCTCCTGTGCAG
CCTCTGGATACACCTACCGTAGCAACTTCATGGGC
TGGTTCCGCCAGGCTCCGGCGAAGGAGCGCGAGGG
GGTCGCAACTATTGATTCTCGTGGTACTATCACAT
ACTATGCCGACTCCGTGAAGGGCCGATTCACCATC
TCCCAAGACAACGAGAAGAACACGGTGTATCTGCA
AATGAACAGCCTGAAACCTGAGGACACTGCCATGT
ACTACTGTGCGGCAGGGGGCCCCCGCACGAACGAT
GATAGATGGTACCCCCTCCGACCTCCTGGTTATAA
CTACTGGGGCCAGGGGACCCAGGTCACCGTCTCCT
CA
(Camel sdAb AS64137 Nucleic Acid Sequence)
SEQ ID NO: 382
CAGGTGAGGTTAGTGGAGTCTGGGGGAGGCTCGGT
GCAGGCTGGAGGGTCTCTGAGACTCTCCTGTGCTG
CCTCTGGATCCACCTACAGTACAAACTTCATGGGC
TGGTTCCGCCAGGCTCCAGGGAAGGAGCGCGAGGG
GGTCGCAACGCTGGTTACTTGGGTTGAACGCACAG
CCTATGCCGACTCCGTGAAGGGCCGATTCACCATC
TCCCAAGACCGCGCCAAGAACACGGTGTATCTACA
AATGAACAGCCTGAAACCTGAGGACACTGCCATGT
ACTACTGTGCGGCAGCCGCCGCTTCCACTGATGTA
CGTCTCCTCGACCCGGGGGACTTTGCTTACTGGGG
CCAGGGGACCCAGGTCACCGTCTCCTCA
(Camel sdAb AS64142 Nucleic Acid Sequence)
SEQ ID NO: 383
CAGGTGCACCTGATGGAGTCTGGGGGAGGCTTGGT
GCAGACAGGGGGGTCTCTGAGACTCTCCTGTACAG
CCTCTGGATTCACTTTTGATCGTAATGCCATGCGC
TGGTACCGCCAGGCTCCAGGGAAGGAGCGCGAGGG
GGTCTCATGTATTGATTGGACGGGTGCAAATATTG
CCTATGCAGACTCCGTGAAGGGCCGATTCACCATC
TCCAGAGACAACGCCAAGAACACGCTGTATCTGCA
AATGAACAGCCTGAAACCTGAGGACACGGGCATGT
ATTACTGTGCGGCAGATACGACGTCGGGGTATTGT
TCAGGCTTTTGGTCTACGAGCCGGTACTCATGGGG
CCAGGGGACCCAGGTCACCGTCTCCTCA
(Camel sdAb AS64154 Nucleic Acid Sequence)
SEQ ID NO: 384
CAGGTGCAATTGAAGGAGTCTGGGGGAGGCTCGGT
GCAGGCTGGAGGGTCTCTGAGACTCTCCTGTACAG
CCTCTGGATACACCTACAGATACCTCTACATGGGC
TGGTTCCGCCAGACTCCAGGGAAGGAGCGCGAGGG
GGTCGCATGTATTTATACTGGTAGTGGTAGCACAG
GGTATGCCGACTCCGTGAAGGGCCGATTCACCATC
TCCCAAGACAACGCCAAGAACACGGTGTATCTGCA
AATGAACAACCTGAAACCTGAGGACACTGCCATGT
ACTACTGTGCGGCAAGTTCGCCCCGGTGGGGCGGT
ACCTGTCGACGCTGGTCTCAGTATAACTACTGGGG
CCAGGGGACCCAGGTCACCGTCTCCTCA
(Camel sdAb AS64160 Nucleic Acid Sequence)
SEQ ID NO: 385
GAGGTGCAGCTGGTGGAGTCTGGGGGAGGCTCGGT
GCAGGCTGGAGGGTCTCTGAGACTCTCCTGTGCGG
CCTCTGTATACACCAGCAGTAGCTACTGCATGGGC
TGGTTCCGCCAGGCTCCAGGGAAGGAGCGCGAGGG
GGTCGCAGCTATGTGTTTTGGTGGTCTTGTCACAC
ACTATGCCGACTCCGTGAAGGGCCGATTCACCATC
TCCCAAGACAATGCCAAGAACACGGTGTATCTGCA
AATGAACAGCCTGAAACCTGAGGACACTGCCATGT
ACTACTGTGCGGCAGATTTCGGCAGGGATAAAAAC
TATTTACGACCGTTACTGCCCCATGCATATAACTA
CTGGGGCCAAGGGACCCAGGTCACCGTCTCCTCA
(Camel sdAb AS64228 Nucleic Acid Sequence)
SEQ ID NO: 386
CAGGTGCAATTGAAGGAGTCTGGGGGAGGTTCGAT
CCAGGCTGGAGGGTCTCTGAGACTCTCCTGTGCAG
CCTCTGGAGTCTCCTACAATAGGTGCAGTATGGGC
TGGTACCGCCAGGCTCCAGGGAAGGGGCGCGAGTT
GGTCTCACGTATTCAGCCGGGTGGTAATACATACT
ATGCAGACTCCGTGAAGGGCCGATTCACCGTCTCC
CAAGACAACGCCAAGAACACAGTATCTCTACAAAT
GAACAGCCTGAAACCTGAGGACACGGCCATGTATT
ACTGTAACGCACTGTGCTGGCGGGAGAATGTTAAC
TACTGGGGCCAGGGGACCCAGGTCACCGTCTCCTC
A
(Camel sdAb AS64300 Nucleic Acid Sequence)
SEQ ID NO: 387
CAGGTGCACCTGGTGGAGTCTGGGGGAGGCTCGGT
GCAGACTGGAGGATCTCTGAGACTCTCCTGTGCAG
TCTCTGGAGACATCTATAACCTCATGTCGATGGCC
TGGTTCCGCCGGGCTCCAGGGAAGGAGCGCGAGGG
GGTCGCATATATTAATACTATTATTGGTAACACAT
ACTATACTGACTCCGTGAAGGGCCGATTCACCATC
TCCCGCGATAACTCCAAGAACACTTTGTATCTGCA
AATGAACAACCTGAAACCTGAGGACACAGCCATGT
ACTACTGTGCGGCGTTCAATTACGGAGGTGCCTGG
TACGAGGAACGCAGCTATAAATACTGGGGCCAGGG
GACCCAGGTCACCGTCTCCTCA
(Camel sdAb AS64380 Nucleic Acid Sequence)
SEQ ID NO: 388
GAGGTGCAGCTGGTGGAGTCTGGGGGGGGCTCGGT
GCAGGCTGGAGGGTCTCTGACACTCTCCTGTGAAG
CCTCTGGAAACACCTACAGTAGTAATTACATGGGC
TGGTTCCGCCAGGCTCCAGGGAAGGAGCGCGAGGA
GGTCGCGGTTATTTACACTCGTGGTGGTCACACAT
ACTATGTCGACTCCGTGAGGGGCCGATTCACCATC
TCCCAAGACAACGCCAAGAACACGGTGTATCTGCA
AATGAACAGCCTGAAACCTGAGGACACTGCCATGT
ACTACTGTGCGGCGTCTTCACGACATAGACTCGGT
TTAAATAACCCACGGGACTATGACTACTGGGGCCA
GGGGACCCAGGTCACCGTCTCCTCA
(Camel sdAb AS64395 Nucleic Acid Sequence)
SEQ ID NO: 389
CAGGTGAGGTTGGTGGAGTCTGGGGGAGGCTCGGT
GCAGGCTGGAGGGTCTCTGAGACTCTCCTGTGCTG
CCTCTGGATCCACCTACAGTACAAACTTCATGGGC
TGGTTCCGCCAGGCTCCAGGGAAGGAGCGCGAGGG
GGTCGCAACGCTTGTTACTTGGGCTGAACGCACAG
CCTATGCCGACTCCGTGAAGGGCCGATTCACCATC
TCCCAAGACCGCGCCAAGAACACGGTGTATCTACA
AATGAACGGCCTGAAACCTGAGGACACTGCCATGT
ACTACTGTGCGGCAGCCGCTTCCACTGCTGTACGT
CTCCTCGACCCGGGGGACTTTGCTTACTGGGGCCA
GGGGACCCAGGTCACCGTCTCCTCA
(Camel sdAb AS64443 Nucleic Acid Sequence)
SEQ ID NO: 390
CAGGTGCACCTGGTGGAGTCTGGGGGAGGCTCGGT
GCAGGCTGGAGGGTCTCTGAGACTCTCCTGTGCAG
CCTCTGGATATACCGACAGTAGCGTCTACATAGGC
TGGTTCCGCCAGGCTCCAGGGAAGGAGCGCGAGGA
GGTCGCGATTATTTATACTGGTGGTGAAAGCACAC
ACTATCGCAGCTCCGTGAAGGGCCGATTCACCGTC
TCCCAAGACAACGCCAAGAACACGCTGTATCTGCA
AATGAACAGCCTGAAACCTGAGGACACGGCCATGT
ATTACTGTGCAGCACGATTCCCAGCTGTTACCTAC
TGGGGCCAGGGGACCCAGGTCACCGTCTCCTCA
(Camel sdAb AS64511 Nucleic Acid Sequence)
SEQ ID NO: 391
CAGGTGCAGCTGGTGGAGTCTGGGGGAGGCTCGGT
GCAGGCTGGAGGGTCTCTGAGACTCTCCTGTGCAG
CCTCTAGAGCCACCTACAGTACCAACTACATAAGC
TGGTTCCGCCAGGCTCCAGGGAAGGAGCGCGAGGC
GGTCGCAACAATTACTACTGGTGATGGTGAGACAG
CGTATGCCGACTCCGTGAAGGGCCGATTCACCATC
TCCCGAGACAACGCCAAGAACACGGTCTATCTGCA
AATGAACAGCCTGAAACCTGAGGACACTGCCATGT
ACTACTGTGCGGCGAACTTGCGAATCGGTGGCGAC
TGGTTCGACGGACGCGATTTTCGTGCCTGGGGCCA
GGGGACCCAGGTCACCGTCTCCTCA
(Camel sdAb AS64536 Nucleic Acid Sequence)
SEQ ID NO: 392
CAGGTGAAGTTGGTGGAGTCTGGGGGAGGCTCGGT
GCAGGCTGGAGGGTCTCTGAGACTCTCCTGTGCAG
CCTCTAGATACACCGACAATTTCGTGTACATGGGC
TGGTTCCGCCAGGCTCCAGGGAAGGAGCGCGAGGG
GGTCGCACTGATTTATCCTGGTGGTGGTAGCACCT
ACTATGCCTCCTCCGTGAAGGGCCGATTCACCATC
TCCCAAGACAACGCCAAGGGCACGGTGCATCTGCA
AATGAACAACCTGAAACCTGAGGACACTGCCATGT
ACTACTGTGCGGCAAAATGGGGGCTGGGCGGGGGG
GGCCTGAAATCAGATACGTATATGTACTGGGGCCA
GGGGACCCAGGTCACCGTCTCCTCA
(Camel sdAb AS64597 Nucleic Acid Sequence)
SEQ ID NO: 393
CAGGTGCACCTGGTGGAGTCTGGGGGAGGCTCGGT
GCAGGCTGGAGGGTCTCTGAGACTCTCCTGTGCAG
CCTCTGGATACACCTACCGTGTCAACTTCATGGGC
TGGTTCCGCCAGACTCCAGGGAAGGAGCGCGAGGG
GGTCGCAACTATTGATTCTGGTGTGGGTACCACAT
ACTATGCCGACTCCGTGAAGGGCCGATTCACCATC
TCCCATAACAACGCCAAGAACACGATTTATCTGCA
AATGAACAGCCTGAAACCTGAGGACACTGCCATGT
ACTACTGTGCGGCAGGGGGCCCCCCTACGGACGGT
GATAGATGGTACCCCCTCCGACCTCCTGGTTATAA
CTATTGGGGCCAGGGGACCCAGGTCACCGTCTCCT
CA
(Camel sdAb AS64617 Nucleic Acid Sequence)
SEQ ID NO: 394
CAGGTGCAGCTGGTGGAGTCTGGGGGAGGCTCGGT
GCAGGCTGGAGGGTCTCTGAGACTCTCCTGTGCAG
CCTCTGGATACACTGATAGATGCAGCATGGCCTGG
TACCGCCAGGCTCCAGGGAAGGAGCGCGAGTTGGT
CTCGCGTATTAGCACGAGCGGTTTCACAAACTACG
CAGCCTCCGTGAAGGGCCGATTCACCATCTCCCAA
GACAACGCCAAGAACACGGTGTATCTGCAAATGAA
CAGCCTGAACCCCGGGGACACGGGCATGTATTACT
GTGCCATAATCGTAGGACGTACTTGTAGTTTGAAC
TACTGGGGCAACGGCATCCTGGTCACCGTCTCCTC
A
(Camel sdAb AS64634 Nucleic Acid Sequence)
SEQ ID NO: 395
CAGGTGAGGTTGGTGGAGTCTGGGGGAGGCTCGGT
GCAGGCTGGAGGGTCTCTGAGACTCTCCTGTGCAG
CCTCCGGATACAGTTTTAGAGGCGACTTTATGTGT
ATGGGCTGGTTCCGCCAGACTCCAGGGAAGGGGCG
CGAGGGGGTCGCAGTTTTTTATCCTGGTGGCGGCA
GCACAAACTATGCCGACTCCGCGAAGGGCCGATTC
ACCATCTCCCAAGACAACGCCAAGAACACGATGTA
TCTGCAAATGAACACCCTGAAACCTGAGGACACTG
CCATGTACTACTGTGCGGCTCGACGGTGGGTCAGT
GGTACCTGCTACTGGGATAGTGACTTTCATTACTG
GGGCCAGGGGACCCAGGTCACCGTCTCCTCA
(Camel sdAb AS69498 Nucleic Acid Sequence)
SEQ ID NO: 396
CAGATGCAGCTGGTGGAGTCTGGGGGAGGCTCGGT
GCAGGCTGGAGGGTCTCTGAGACTCTCCTGTGCAG
CCTCTGGAAACACCTACAGTACCAATTACATGGCG
TGGTTCCGCCAGGCTCCAGGGAAGGAGCGCGAGGA
GGTCGCGGTTATTTACACTCGTGGTGGTCACACCT
ACTATATCGACTCCGTGAGGGGCCGATTCACCATC
TCCCAAGACAACGCCAAGAACACGGTGTATCTGCA
AATAAACAGCCTGAAACCTGAGGACACTGCCATGT
ACTACTGTGCGGCGTCTTCACGAATTAGACTCCAT
TGAATCGACCCACGGGACTATCACGACTGGGGCCA
GGGGACCCAGGTCACCGTCTCCTCA
(Camel sdAb AS69500 Nucleic Acid Sequence)
SEQ ID NO: 397
CAGGTGAGGTTAGTGGAGTCTGGGGGAGGCTCGGT
GCAGGCTGGAGGGTCTCTGAGACTCTCCTGTGCAG
CCGACAGATACACCTACAGTAGCGCCTGCATGGGC
TGGTTCCGCCAGGCTCCAGGGAAGGAGCGCGAGGG
GGTCGCCTCTATTTTTACTGGTACTGGTGGTAGCA
CATACTATGCCGACTCCGTGAAGGGCCGATTCACC
ATCTCCCAAGACAACGCCAAGAACACGGTGTATCT
GCAAATGAACAGCCTGAAACCTGAGGACACTGCCA
TATACTACTGTGCGGCAAGGGCCTTCCAGGTCGGT
TACTGCTACCTGCGAACCGATGTGTATAACTACTG
GGGCCAGGGGACCCAGGTCACCGTCTCCTCA
(Camel sdAb AS69527 Nucleic Acid Sequence)
SEQ ID NO: 398
GAGGTGCAGCTGGCGGAGTCTGGGGGAGGCTCGGTC
CAGGCTGGAGGGTCTCTGAGACTCTCCTGTGTAGC
CTCTAGATACACCTTCAGTAGCACCTGCATGGCCT
GGTTCCGCCAGGCTCCAGGGAAGGAGCGCGAGGAG
GTCGCAGCTATTTATACTGATGATGGTAGCACATG
GTATGCCGACTCCGTGAAGGGCCGATTCACCATCT
CCAGAGACAACGCCAAGAACACGGTGTATCTGCAA
ATGAACAGCCTGAAACCTGAGGACACTGCTATGTA
CTACTGTGCGGCACGTAGGTGGGCGTGCCCCAGGG
TTGGTAGCTGGCATGAGTTCGCCTACTGGGGCCAG
GGGACCCAGGTCACCGTCTCCTCA
(Camel sdAb AS68280 Nucleic Acid Sequence)
SEQ ID NO: 399
CAGGTGCAACTGGTGGAGTCTGGGGGAGGCTCGGT
GCACCCTGGAGGGTCTCTGAGACTCTCCTGTGCAG
CCTCTGGATCCACCTACAGTTCCAACTACCTCGGC
TGGTTCCGCCAGGCTCCAGGAAAGGGGCGCGACTG
GGTTGCGGCTATTAGCACTGGTGACGGTGCCACAG
CCTATGCCGACTCCGTGAAGGGCCGATTCACCATC
TCCCAAGACAACGCCAAGAACACGGTGTATCTGCA
AATGAACAGCTTGAAACTTGAGGACAGTGCCATGT
ACTACTGTGCGGCGGCTCGCGGCAGATTTATCGAT
TGGACAAAGGCAACCCAGTATGACTACTGGGGCCA
GGGGACCCAGGTCACCGTCTCCTCA
(Camel sdAb AS68355 Nucleic Acid Sequence)
SEQ ID NO: 400
CAGATGCAGCTGGTGGAGTCTGGGGGAGACTCGGT
GCAGGCTGGAGGGTCTCTGAGACTCTCCTGTGCAG
CCTCTGGATACACCTACAGCGGCGTCTGCATGGGC
TGGTTCCGCCAGGCTCCAGGGAAGGAGCGCGAGGG
GGTCGCAGCTATTGATAGTGATGGTAGCACAAGCT
ACGCAGACTCCGTGAAGGGCCGATTCACCATCTCC
AAAGACAACGCCAAGAACACTCTGTATCTGCAAAT
GAACAGCCTGAAACCTGAGGACACTGCCATGTACT
ACTGTGCGGCAGCCATTGTCGGGGGTTTTAATGCA
TATTGTAGTGGTGGTTATGTTCTGGACTTTGGTGC
CTGGGGCCAGGGGACCCAGGTCACCGTCTCCTCA
(Camel sdAb AS69443 Nucleic Acid Sequence)
SEQ ID NO: 401
GAGGTGCAGCTGGCGGAGTCTGGGGGAGGCTCGGT
GCAGGCTGGAGGGTCTCTGAGACTCTCCTGTTCAG
CCTCTGGTTTCACTTTTGATGATTCTGACATGGCC
TGGTACCGCCAGGCTCCAGGGGATGGGTGCGACTT
GGTCTCAACTATTAGTAGTGATGGTAGCACATACT
ATGCAGACTCCGTGAAGGGCCGATTCACCATCTCC
CAAGACAACGCCAAGAACACGGTGTATCTGCAAAT
GCACAGCCTGAAACCTGAGGACACGGCCGTGTATT
ACTGTGCGGCAGATTTCCTCACCGGCTTTTACTAT
AGCGACTCCCCCCATCCGGCCCCTTGTTCTGCATC
CGACTTTGGTTACTGGGGCCAGGGGACCCAGGTCA
CCGTCTCCTCA
(Camel sdAb AS75376 Nucleic Acid Sequence)
SEQ ID NO: 402
CAGGTGCAATTGAAGGAGTCTGGGGGAGGCTCGGT
GCAGGCTGGAGGGTCTCTGAGACTCTCCTGTGCAG
CCTCTGGATACACCTACAGTAGCCACTCCATGGGC
TGGTTCCGCCAGGCTCCAGGGAAGGAGCGCGAGGG
GGTCGCAGTTATTTATACTGGTGATGGTAGCACAT
ACTATGCCGACTCCGTGAAGGGCCGATTCACCATC
TCCCAAGACAACGCCAAGAACACGGTGTATCTGCA
AATGAACAGCCTGAAACCTGAGGACACTGCCATGT
ACTACTGTGCGGCAGATCCGAACCCCGATTATATG
CTTCCGTTTCGGCCGTCCCGTAGGTCGTGGTGGGG
CCAGGGGACCCAGGTCACCGTCTCCTCA
(Camel sdAb AS75387 Nucleic Acid Sequence)
SEQ ID NO: 403
CAGGTGCACCTGGTGGAGTCTGGGGGAGGCTCGGT
GCAGGCTGGAGGGTCTCTGAGACTCTCCTGTGCAG
CCTCTGGATACCCCTACAGTAGCCCCTGCATGGCC
TGGTTTCGCCAGGCTCCAGGGAAGGAGCGCGAGGG
GGTTTTAGTTGCTTATACTGGTGGGGACATTCAAT
ACCTTACCGACTCCGTGAAGGGCCGATTCACCATC
TCCCGAGACAACGCCAAGAACACGGTGTATCTGCA
AATGAACAGCCTGAAACCTGAGGACACTGCCATGT
ACTACTGTGCGGCGGATCTGCGATTACCTCGTGCC
GGCGGTTGTGCGTATAGCTACTGGGGCCAGGGGAC
CCAGGTCACCGTCTCCTCA
(Camel sdAb AS75695 Nucleic Acid Sequence)
SEQ ID NO: 404
CAGGTGAGGTTGGTGGAGTCTGGGGGAGGCTCGGT
GCAGGCTGGAGGGTCTCTGAGACTCTCCTGTGTAG
CCTCTGGATACACCGTCAGTGCCTACTGTATGGGC
TGGTTCCGCCAGGTTCTAGGGAAGGGGCGCGAGAG
GATCGCATTTATCGATGCCGGGGGTGCTACGATTT
ACGCAGACCCCGTGAAGGGCCGATTCACCATCTCC
AAAGACAACGCCAAGAACACTCTGTATCTGCAAAT
GAACAGCCTGAAACCTGAGGACACTGCCATGTACT
ACTGTGTTGCAGATCGCCGGGGGCGGGTACGTCGG
TGCGAGTATAACGCCTGGGGCCAGGGGACCCAGGT
CACCGTCTCCTCA
(Camel sdAb AS76169 Nucleic Acid Sequence)
SEQ ID NO: 405
CAGGTGCACCTGATGGAGTCTGGGGGAGGCTCGGT
GCAGGCTGGAGGGTCTCTGAGACTCTCCTGTGCAG
CCTCTGGATACATTTACAGTAGCTTCTGCATGGGC
TGGTTCCGCCAGGCTCCAGGGAAGGAGCGCGAGGT
GGTCGCGTATATTCGCGATAATATTATGACAAGTT
ACACAGACTCCGTGAAGGGCCGATTCACCATCTCC
AAAGACAACGCCAAGAGAACTCTGTATCTACAAAT
GAACGGCCTGAAACCTGAAGATACTGGCATGTACT
ACTGTGCGGTAGACCGGGGGGGATACGCTAATAGT
TGCGCGGTAGCGGCCCGGTATGATTACTGGGGCCG
GGGGACCCAGGTCACCGTCTCCTCA
(Camel sdAb AS63931 Nucleic Acid Sequence)
SEQ ID NO: 406
GAGGTGCAGCTGGCGGAGTCTGGGGGAGGCTCGGT
GCAGGCTGGGGGGTCTCTGAGACTCTCCTGTGCAG
GCTCTTTCTCTGGATACGGCGTCAGTACCATGGCC
TGGTTCCGCCAGGCTCCAGGGAAGGAGCGCGAGGG
GGTCGCAGCTATTACTGTTGGTAGTGGAAACACAT
ACTATGCCGACTCCGTGACGGGCCGATTCACCATC
TCCCGAGACAACGCCAAGAGGACGGTGTATTTGCA
AATGAACAGCCTGAAACCTGAGGACACTGCCATGT
ACTACTGTGCGGCCGGATGGTTGTCGGGTGGTAGT
TGGCACGTTCCCGGCAGGTATAACTACTGGGGCCA
GGGGACCCAGGTCACCGTCTCCTCA
(Camel sdAb AS63937 Nucleic Acid Sequence)
SEQ ID NO: 407
CAGGTGAAGTTAGTGGAGTCTGGGGGAGGCTCGGT
GCAGGCTGGAGGGTCTCTGAGACTCTCCTGTGCAG
CCTCTGGATCCACCATCAGTAGTCGCCCGATGGCC
TGGTTCCGCCAGGCTCCAGGGAAGGAGCGCGAGGG
GGTCGCGTGTATACATACTGGTCTTGGTAGAACAT
ACTATGCCGACTCCGTGAAGGGCCGATTCACCATC
TCCCAAGACAACGCCAAGAACACGGTGTATCTGCA
AGTGAACAGCCTGAAACCTGAGGACACTGCCATGT
ACTACTGTGCGGCAGACTCGCGGCGGCCGTGTATG
GTAGCCGCAGGGTATACCTACTGGGGCCAGGGGAC
CCAGGTCACCGTCTCCTCA
(Camel sdAb AS63948 Nucleic Acid Sequence)
SEQ ID NO: 408
CAGGTGCAGCTGGTGGAGTCTGGGGGAGGCTCGGT
GCAGGCTGGAGGGTCTCTGAGACTCTCCTGTACAG
CCTCTGGATACACCTATAGATACCTCTACATGGGC
TGGTTCCGCCAGACTCCAGGGAAGGAGCGCGAGGG
GGTCGCATGTATTTATACTGGTAGTGGTAGCACAG
GGTATGCCGACTCCGTGAAGGGCCGATTCACCATC
TCCCAAGACAACGCCGAGAACACGGTGTATCTGCA
AATGAACAGCCTGAAACCTGAGGACACTGCCATGT
ACTACTGTGCGGCAGCTTCGCCCCGGTGGGGTGGT
ACCTGTCGACGGTGGTCCGAGTATAACTACTGGGG
CCAGGGGACCCAGGTCACCGTCTCCTCA
(Camel sdAb AS63956 Nucleic Acid Sequence)
SEQ ID NO: 409
CAGGTGCACCTGGTGGAGTCTGGGGGAGGCTTGGT
GCAGGCTGGAGGGTCTCTGAGACTCTCCTGTGCAG
CCTCCGGATTCACCTACAGTAACTGCTGCATGAGG
TGGTACCGCCAGGCTCCAGGGAAGGCGCGCGAGTT
GGTCTCATTAATTAATAGTAGTGGTGGCACATACT
ATGCAGACTCTGTGAGGGGCCGATTCACCATCTCC
AAAGACAACGCCAAGAACACGCTGTATCTGCAAAT
GAACAGCCTGAAACCTGAGGACACGGCCATGTATT
ACTGTGCGGCTTACCAAGCCAAGTACTGTTCAGGC
CCTTGCGCCCCCCCAACTGACTGGGGCCAGGGGAC
CCAGGTCACCGTCTCCTCA
(Camel sdAb AS63965 Nucleic Acid Sequence)
SEQ ID NO: 410
CAGGTGCAGCTGGTGGAGTCTGGGGGAGGCTCGGT
GCAGGCTGGAGGGTCTCTGAGACTCTCCTGTGTAG
CCTCTGGATACAGCAGCGGTAGTTGTCGCATGGGC
TGGTACCGCCAGGCTCCAGGGAAGGAGCGCGAGTT
GGTTTCAAAGGTTATTAGTGATGGTACTACAGTCT
ATGCAGACTCCGTGAAGGGCCGATTCACCCTCTCC
CAAGGAAACGCCAAGAACACGGTGTATCTGCAAAT
GAGTAGCCTGTTACCTGAGGACACGGCCATGTATT
ACTGTAATGCATGGTGTAGGGAGTATCCCGGGGGG
ATCCTGAATAACGGCTGGGGCCAGGGGACCCAGGT
CACCGTCTCCTCA
(Camel sdAb AS63993 Nucleic Acid Sequence)
SEQ ID NO: 411
CAGGTGAAGTTGGTGGAGTCTGGGGGGGGCTTGGT
GCAGGCAGGGGGGTCTCTGAGACTCTCCTGTACAG
TTTCTGGATTCACTTTCGATGACCTCGTCATGGCC
TGGTTCCGCCAGGCTCCAGGGAAGGAGCGCCAACT
TGTCTCGTTGGTTGCGACTGCTGGTAATAGCGTCT
ATGCAGACTCCGTGAAGGGCCGATTCACACTCTCC
AGAGACAACGCCCACAGCACGGCGTATCTGCAAAT
GAACGGCCTGAAACCTGAGGACACGGCCATGTATT
ACTGTGCGGCACGTACCGATTCTGAGCATGCGTTT
AAGTTCTGGGGTCAGGGGACCCAGGTCACCGTCTC
CTCA
(Camel sdAb AS63999 Nucleic Acid Sequence)
SEQ ID NO: 412
GAGGTGCAACTGGTGGAGTCTGGGGGAGGCTCGGT
GCAGGCTGGAGGGTCTCTGAGACTCTCCTGTGCAG
CCTCTGGATACACTTACAGTAGCAACTGGATGGGC
TGGTTCCGCCAGGCTCCAGGGAAGGAGCGCGAGGA
GGTCGCAATTATTTATACTGGTGGTATTAGTACAC
ACTATCGCAGCTCCGTGAAGGGCCGATTCACCATC
TCCCAAGACAACGCCAAGAACACGGTGTATCTGCA
AATGAACAGCCTGAAACCTGAGGACACTGCCATGT
ACTACTGTGCGGCGCGTTATACAGACTATAACTAC
TGGGGCCAGGGGACCCAGGTCACCGTCTCCTCA
(Camel sdAb AS64006 Nucleic Acid Sequence)
SEQ ID NO: 413
CAGGTGCACCTGGTGGAGTCTGGGGGAGGCTCGGT
GCAGGCTGGAGGGTCTCTGAGACTCTCCTGTGAAG
TCTCTGGATACACCGGCGATACGACTTACATAGGC
TGGTTCCGCCAGGCTCCAGGGAAGGAGCGCGAGGG
GGTCGCACTTATTTATACTAGTGGTACTAGCGAGT
ACTACGCCGACTCCGTGAAGGGCCGATTCATCATC
TCCCGAGACAACGCCAAGAACACGGTGTATTTACA
AATGAACAGCCTGAAACCTGAGGACACTGCCATGT
ACTACTGTGGCGCACGGAGCCGCACGATGATGTAC
TGGGGCCAGGGGACCCAGGTCACCGTCTCCTCA
(Camel sdAb AS64057 Nucleic Acid Sequence)
SEQ ID NO: 414
CAGGTGCAATTGGAGGAGTCTGGGGGAGGCTTGGT
GCAGACAGGGGGGTCTCTGAGACTCTCCTGTACAG
CCTCTGGATTCACTTTTGATCGTAATGCCATGCGC
TGGTACCGCCAGGCTCCAGGGAAGGAGCGCGAGGG
GGTCTCATGTATTAGTTGGACGGGTGCAAATATTG
CCTATGCAGACTCCGTGAAGGGCCGATTCACCATC
TCCAGAGACAACGCCAAGAACACGCTGTATCTGCA
AATGAACAGCCTGAAACCTGAGGACACGGGCATGT
ATTACTGTGCGGCAGATACGACGTCGGGGTCTTGT
TCAGGCTTTTGGTCTACGAGCCGGTACTACTGGGG
CCAGGGGACCCAGGTCACCGTCTCCTCA
(Camel sdAb AS64060 Nucleic Acid Sequence)
SEQ ID NO: 415
CAGGTGAAGTTAGTGGAGTCTGGGGGAGGCTCGGT
GCAGGCTGGAGGGTCTCTGAGACTCTCCTGTGCAG
CCTCTGGATCCACCTACTGTACCTACCGTATGAGC
TGGTTCCGCCAGGCTCCAGGGAAGGAGCGCGAGTT
CGTCGCAGTTATTGATAGTGGCGGTAGCACAAGCT
ACGCAGACTCCGTGAAGGGCCGATTCACCATCTCC
CGAGACAACGCCAAGAACACGGTGTATCTGCAAAT
GAACAGCCTGAAACCTGAGGACACGGCCATGTATT
ACTGTAAAACAGATCCAACCATCGGCTGCCCCCAG
ACATATAGGTATAACTACTGGGGCCAGGGGACCCA
GGTCACCGTCTCCTCA
(Camel sdAb AS64071 Nucleic Acid Sequence)
SEQ ID NO: 416
CAGGTGCACCTGATGGAGTCTGGGGGAGGCTCGGT
GCAGGCTGGAGGGTCTCTGAGACTCTCCTGTGTAG
CCTCTGGAAACACTTACAGGCTCAACTCTATGGGC
TGGTTCCGCCAGGCTCCAGGGAAGGAGCGCGAGGG
GGTCGCATTTATTGTTATGATTAGAGGTACCACAT
ACTATGGCGCCTCCGTAAAGGGCCGATTCACCATC
TCCCAAGACAACGCCCAGACCACGGTGTATCTGCA
AATGAGCAGCCTGAAACCGGAGGACACTGCCATGT
ACTACTGTGCGGCATCCACTAAGGACCAGTTTTAT
GTATTTAATCCTATTGGGTATGACTCTTGGGGCCA
GGGGACCCAGGTCACCGTCTCCTCA
(Camel sdAb AS64093 Nucleic Acid Sequence)
SEQ ID NO: 417
CAGGTGCACCTGGTGGAGTCTGGGGGAGGCTCGGT
GCAGGCTGGAGGGTCTCTGAGACTCTCCTGTGCAA
CCTCTAGATACATCTACGGTAACAACTGCATGGCC
TGGTTCCGCCAGGCTCCAGGGAAGGAGCGCGAGGG
AGTCGCGTCTATTTATCCTGCTGGTGGTCGCACGT
ACTATGCCGACTCCGTGAAGGGCCGATTCACCATC
TCCCAAGACAACGCCAAGAACACGGTGTATCTGCA
AATTGACAGCCTGAAACCTGAGGACACGGCCATGT
ACTACTGCGCGGCACGCTCTTTTTCGATAGGAGTT
TGCGCGACGCAGTCTGGTATTACCTGGTCTAATTT
TGCTTACTGGGGCCAGGGGACCCAGGTCACCGTCT
CCTCA
(Camel sdAb AS64118 Nucleic Acid Sequence)
SEQ ID NO: 418
CAGGTGCAACTGGCGGAGTCTGGGGGAGGCTCGGT
GCAGGCTGGAGGGTCTCTGAGACTCTCCTGTGCAG
CCTCTGGATACACCTACAGTGCCTGTAGAATGGCC
TGGTACCGCCAGGCTCCCGGGAAGGAGCGCGAGTT
GGTTTCATTTATTAATAGTGCTGGTAGCACATACT
ATGCCGACTCCGTGAAGGGCCGATTCGCCATCTCC
CGAGACAACGCCAAGACAACGGTGTATCTACAAAT
GAACGCCCTGAAAGCTGAGGACACGGCCATATATT
ACTGTAACACATGGGATAGTAGCTGCCGCTTTCAG
TACTGGGGCCAGGGGACCCAGGTCACCGTCTCCTC
A
(Camel sdAb AS64120 Nucleic Acid Sequence)
SEQ ID NO: 419
CAGGTGAGGTTGGTGGAGTCTGGGGGAGGCTCGGT
GCAGGCTGGAGGGTCTCTGAGACTCTCCTGTGAAA
CCTCTAGATACATCTACGGTAACAACTGCATGGCC
TGGTTCCGCCAGGCTCCAGGGAAGGAGCGCGAGGG
AGTCGCGTCTATTTATCCTGCTGGTGGTCGCCCGT
ACTATGCCGACTCCGTGAAGGGCCGATTCACCATC
TCCCAAAGACAACGCCAAGAACACGGTGTATCTGC
AAAATGGACAGCCCTGAAAACCTGAGGACACGGCC
CATGTACTACTGCGCGGGCACGCTTCTTTTTTCGA
TAGCAAATTGCGCCAACGCAATCCTGGTATTAACC
AGGGCCAAATTTTGGCTTACGGGGGCCAGGGGACC
CAGGTCACCCTTCTCCTCA
(Camel sdAb AS64124 Nucleic Acid Sequence)
SEQ ID NO: 420
CAGGTGAAGTTGGTGGAGTCTGGGGGAGGCTCGGT
GCAGACTGGAGGGTCTCTGAGACTCTCCTGTGCAG
TCTCTACGTACACCCCCAGTAACAACTACATGGGC
TGGTTCCGCCAGGCTCCAGGGAAGGAGCGCGAGGG
CGTCGCGGCTATCGCTACTATTGGTGGTACCACAC
GTTATGCCGACTCCGTGAAGGGCCGATTCACCATC
TCCCAAGACGGCGCCAAGAACACGATATATCTGCA
AATGAACGGCCTGAAACCGGAGGACACTGCCATGT
ACTACTGTGCGGCCGGGCGGCCATACTCATTACCC
TTACCCTTACCCTTGGAAAGCGGTGCGTATCGCTA
CTGGGGCCAGGGGACCCAGGTCACCGTCTCCTCA
(Camel sdAb AS64135 Nucleic Acid Sequence)
SEQ ID NO: 421
CAGGTGAAGTTGGTGGAGTCTGGGGGAGGCTCGGT
GCAGGCTGGAGGGTCTCTGAGACTCTCCTGTGTAG
CCTCTACATCAACCTACTGTAGGTACTACATGCGC
TGGTACCGGCAGGCTCCAGGGAAAGAGCGCGAGTT
CGTCTCAGCGATGCAACCCGATGGTACGACAAGCT
ACTCAGACTCCGTGAAGGGCCGATTCACCATGTCC
CAAGACAGAGCCAACAATATGTTGTATCTGCAAAT
GAACAGCCTGAGGCCTGAGGACACGGCCATGTATT
ACTGTAAAAGAGATCCAATGGGGGGTTCAAGGACC
CCGTGCACCTCCGCCTGGGGCCAGGGGACCCAGGT
CACCGTCTCCTCA
(Camel sdAb AS64163 Nucleic Acid Sequence)
SEQ ID NO: 422
CAGGTGAGGTTGGTGGAGTCTGGGGGAGGCTCGGT
GCAGGCTGGAGGGTCTCTGAGACTCTCCTGTGCAG
TCTCTGGATACAGATATAGATGGAACTGCATGGCC
TGGTTCCGCCAGGCTCCAGGGAAGGAGCGCGAGGG
GGTCGCAGCTATTTCTACTGGAAGCGGAAGCACAT
ACTATGCCGGCTCCGTGAAGGGCCGATTCACCATC
TCCCAAGACAACGCCAAGAACATGTATCTGCAAAT
GAACAGCCTGAAACCTGAGGACACTGCCATGTACT
ACTGTGCGGCAGATCCTTCGGTTTGCCCCGGTGGT
ATGTGGTACTCCAAAGAGTATAGGTACTGGGGCCA
GGGGACCCAGGTCACCGTCTCCTCA
(Camel sdAb AS64182 Nucleic Acid Sequence)
SEQ ID NO: 423
CAGGTGCACCTGATGGAGTCTGGGGGAGGCTCGGT
GCAGGCTGGAGGGTCTCTGAGACTCTCCTGCACAG
CCTCTGGACAGACCTCCAGATACCTCTACATGGGC
TGGTTTCGCCAGACTCCAGGGAAGGAGCGCGAGGG
GGTCGCATGTATTTATACTGGTAGTGGTAGCACAG
GGTATGCCGACTCCGTGAAGGGCCGATTCACCATC
TCCCAAGACAACGCCAAGAACACGGTGTATCTGCA
AACGAATAGCCTGAAACCTGAGGACACTGCCATGT
ACTACTGTGCGGCAAGTTCGCCCCATTGGGGCGGT
ACCTGTCGACGCTGGTCCGAGTATAAGTACTGGGG
CCAGGGGACCCAGGTCACCGTCTCCTCA
(Camel sdAb AS64183 Nucleic Acid Sequence)
SEQ ID NO: 424
CAGGTGCACCTGGTGGAGTCTGGGGGAGGCTCGGT
GCAGGCTGGAGGGTCTCTGAGACTCTCCTGTGCAG
CCTCTGGACACACCTACAGTGCCAACTGCATGGCC
TGGTTCCGCCGGGCCCCAGGGAAGGAGCGCGAGTG
GGTCGCGTCGGTTTATACTGATGATGATAGCACAA
TGTATACCGACTCCGTGAAGGGCCGATTCACCATC
TTCCAAGACAACGCCAAGAACACGGTGTATCTGCA
AATGAACAGCCTGAAACCTGAGGACACTGGCATGT
ACATCTGTGCGGCAGATTTAAGCGGAGGACCGGCC
GGTTGTGGGTATACCCACTGGGGCCAGGGGACCCA
GGTCACCGTCTCCTCA
(Camel sdAb AS64207 Nucleic Acid Sequence)
SEQ ID NO: 425
GAGGTGCAGCTGGTGGAGTCTGGGGGAGGCTCGGT
GCAGGCTGGAGGGTCTCTGAGACTCTCCTGTGCAG
CCTCTGGATACACCTACAGTAGCAACTTCATGGGC
TGGTTCCGCCAGGCTCCAGGGAAGGAGCGCGAGGG
GGTCGCAACTATTGTTTCTGGTGGTGGTACCACAT
ACTATGCCGACTCCGTGAGGGGCCGATTCACCATC
TCCCAAGACAACGCCAAGAACACGGTGTATCTGCA
AATGAACAGCCTGAAACCTGAGGACACTGCCATGT
ACTACTGTGCGGCAGGGGGCCCCCCTACGAACGGT
GCTAAGTGGTACCCCCTCCGACCTCCTGGTTATAA
CTACTGGGGCCAGGGGACCCAGGTCACCGTCTCCT
CA
(Camel sdAb AS64276 Nucleic Acid Sequence)
SEQ ID NO: 426
CAGGTGCACCTGATGGAGTCTGGGGGAGGCTCGGT
GCAGGCTGGAGGGTCTCTGAGCCTCTCCTGTGTAG
TCTCTGGATACACCGGCAGTAGCCGCTGTATGGCC
TGGTTCCGCCAGGCTCCAGGGAAGGAGCGCGAGGC
GGTCGCACAAATTTTTACTGGTCGTGGTACCACAG
GCTATGCCGACTCCGTGAAGGGCCGATTCACTATT
TCCCAAGACAACGCCAAGAACACGGTGTATCTGCG
AATGAACAGTCTGAGACCTGAGGACACTGCCATTT
ACTACTGTGCGGCGAGTCTCGGCCCGGGACGCGGA
GCCTGTGGGTATAACTACTGGGGCCAGGGGACCCA
GGTCACCGTCTCCTCA
(Camel sdAb AS64336 Nucleic Acid Sequence)
SEQ ID NO: 427
CAGGTGCAGCTGGTGGAGTCTGGGGGAGGCTCGGT
GCAGGCTGGAGGGTCTCTAAGACTCTCCTGTACAA
CCTCTGGACGCACCTACAGTAGCTGCAGCATGGGC
TGGTACCGCCAGGCTCCAGGGAAGGAGCGCGAGTT
GGTCTCACATATTTTTAGTGATGGTAGCAGATACT
ATGCAGACTCCGTGAAGGGCCGATTCACCATCTCC
CAAGACAACGCCAAGAACACGGTGTATCTGCAAAT
GAACAGCCTGAAACCTGAGGACACGGCCATGTATT
ACTGTAACCGCCGTACGGGTTGGGCACCAAGGTGC
GCTGTTCCCGGTTACTGGGGCCAGGGGACCCAGGT
CACCGTCTCCTCA
(Camel sdAb AS64346 Nucleic Acid Sequence)
SEQ ID NO: 428
CAGGTGCACCTGGTGGAGTCTGGGGGAGGCTCGGT
GCAGGCTGGAGGGTCTCTGAGACTCTCCTGTGCAG
CCTCTGGATACACCTATTTCATGGGCTGGTTCCGC
CAGGCTCCACAGAAGGAGCGCGAATGGGTCGCGAC
TATTGGTACTGGTGATATTTTCAATGGCGCTGCTT
ACTATGTCGACTCCGTGAAGGGCCGATTCGCCATC
TCCCAAGACAACGCCAAGAACACGGTGTATCTGCA
AATGAACAGCCTGAAACCTGAAGACACTGCCGTGT
ACGTCTGTGCGGCAGTTCAATCGAAATCCTCAAAC
TACGTGTTGAGAGACGCATCTACCTACAACTACTG
GGGCCAGGGGACCCAGGTCACCGTCTCCTCA
(Camel sdAb AS64420 Nucleic Acid Sequence)
SEQ ID NO: 429
GAGGTGCAGCTGGTGGAGTCTGGGGGAGGCTCGGT
GCAGGCTGAAGGGTCTCTGAGACTCTCCTGTGCAG
CCTCTGGAGACACCAGTAGAAGCGTCTGGATGGGC
TGGGCCCGCCAGGTTCCAGGGAAAGAGCGCGAGGT
GGTCGCAACCATTAGTACTGCCGGTGGTAGTACAT
GGTATACCGACTCCGTGAAGGGCCGATTCACCATC
TCCCAAGACAACGCCAAGAACACGGTGTACCTGCA
AATGAACAGCCTGAAACCTGAGGACACTGCCATAT
ACTATTGTGCGGCCAGAAGCAGATATGCTACCTAC
TGGGGCCAGGGGACCCAGGTCACCGTCTCCTCA
(Camel sdAb AS64473 Nucleic Acid Sequence)
SEQ ID NO: 430
CAGGTGAGGTTGGTGGAGTCTGGGGGAGGCTCGGT
GCAGGCTGGAGGGTCTCTGAGACTCTCCTGTACAG
CCTCTGGATACACCTACAGATACCTCTACATGGCC
TGGTTCCGCCAGACTCCAGGGAAGGAGCGCGAGGG
GGTCGCATGTATTTATACTGGTAGTGGTACCACAG
GGTATGCCGACTCCGTGAAGGGCCGATTCACCATC
TCCCAAGACAACGCCAAGAATACGGTGTATCTGCA
AATGAACAGCCTGAACGCTGAGGACACTGCCATGT
ACTACTGTGCGGCAAGTTCGCCCCAGTGGGGCGGT
ACCTGTCGACGCTGGTCCGAGTATAACTACTGGGG
CCAGGGGACCCAGGTCACCGTCTCCTCA
(Camel sdAb AS64475 Nucleic Acid Sequence)
SEQ ID NO: 431
CAGGTGCAACTGCAGGAGTCTGGGGGAGGCTCGGT
GCAGGCTGGAGGGTCTCTGAGACTCTCCTGTGCAG
CCTCTGGATACACCTGGAGTCGCAACTGGATGGGC
TGGTTCCGCCAGGCTCCAGGGAAGGAGCGCGAGGG
GTTCGCAACTATTACAATTAGTGGTGGTAGCACAT
GGTATGCCGACTCCGTGAAGGGCCGATTCACCATC
TCCCTAGACAACGCCGGGAACACGGTGTATCTGCA
AATGAACAGCCTGAAACCTGAGGACACTGCCATGT
ACTACTGTGCGGCGCGGGATACCGCGCGGACCTAC
TGGGGCCAGGGTACCCAGGTCACCGTCTCCTCA
(Camel sdAb AS64513 Nucleic Acid Sequence)
SEQ ID NO: 432
GAGGTGCAGCTGGTGGAATCTGGGGGAGGCTCGGT
GCAGGCTGGAGGGTCTCTGAGACTCTCCTGTGTAG
CCTCTGACTACCCCTACATAGACAACTGCATGGGC
TGGTTCCGCCAGGGTCCAGGGAAGGAGCGCGAGGG
GGTCGCAGCTGCGTGTACTGGTGGTGGTAGCACAT
ATTATGCCGACTCCGTGAAGGGCCGATTCACCATC
TCCCGAGACAACGCCAAGAACACGGTGTATCTGCA
AATGAACAGCCTGAAACCTGAGGACACTGACGTGT
ACTACTGTGCGACAGGCTACTATAGCGGCTCTGGT
CCGGGGTATTTACTCCCATGGAGGTATAACTACTG
GGGCCAGGGGACCCAGGTCACCGTCTCCTCA
(Camel sdAb AS64562 Nucleic Acid Sequence)
SEQ ID NO: 433
GAGGTGCAACTGGTGGAATCTGGGGGAGGCTCGGT
GCAGGCTGGAGGGTCTCTGAGACTCTCCTGTGCAG
CCTCTGGATACACCGCTAGGCGCGACTTCATGGCC
TGGTTCCGCCAGGTTCCAGGGAAGGAGCGCGAGGG
GGTCGCAGTCATTCATACTGGTGGTGACACCACAT
ACTATGCCGACTCCGTGAAGGGCCGATTCACCATC
TCCCGCGACAACGCCCAGAACATAATGAATCTGCA
AATGAACAGCCTTAAACCTGAGGACACTGCCATGT
ACTACTGTGCGGCAGGTTTCCGTCCGCGTGGTGGA
GGATACACGGGTGACGTCTTGGCCCAGGCTGCGGC
ATACAACTACTGGGGCCAGGGGACCCAGGTCACCG
TCTCCTCA
(Camel sdAb AS64583 Nucleic Acid Sequence)
SEQ ID NO: 434
GAGGTGCAGCTGGCGGAGTCTGGGGGAGGCTTGGT
GCAGGCAGGGGGGTCTCTGAGACTCTCCTGTACAG
CCTCTGGATTCACTATTGCTGTTTATACCATGGGC
TGGTTCCGCCAGGCTCCAGGGAAGGAGCGCGAGGG
AATCTCATGTACTAGCTGGGCTGGTGGTCGCACAT
ACACTGCAGACTCCGTGAAGGGCCGATTCACCATC
TCCAGAGACAACGCCAAGAACACGCTGTATCTGCA
AATGAACAGCCTGAAACCTGAGGACACGGCCATGT
ATTACTGTGCGGCAAAGGCACATCCCGACTGTTCA
GGGGATTGGTCCCCATCTGGGTATGAATACTGGGG
CCAGGGGACCCAGGTCACCGTCTCCTCA
(Camel sdAb AS64594 Nucleic Acid Sequence)
SEQ ID NO: 435
CAGGTGCACCTGGTGGAGTCTGGGGGAGGCTCGGT
GCAGGCTGGAGGGTCTCTGAGACTCTCCTGTGCAG
CCTCTGGATACACCTACAATAGCAACTACATGGGC
TGGTTCCGCCAGGCTCCAGGGAAGGAGCGCGAGGG
GGTCGCACTTATTTATACTGGTGGTGGTAGCACAT
ATTATGCCGACTCCGTGAAGGGCCGATTCACCATC
TCCCGAGACAACGCCAAAAACACGGTGTATCTGCA
AATGAACAGCCTGAAACCTGAGGACACTGCCATGT
ACTACTGTTCGGTAAGAACGCAGACGCGTAACTAC
TGGGGCCAGGGGACCCAGGTCACCGTCTCCTCA
(Camel sdAb AS64605 Nucleic Acid Sequence)
SEQ ID NO: 436
CAGGTGAAGTTGGTGGAGTCTGGGGGAGGCTCGGT
GCAGGCTGGAGGGTCTCTGAGACTCTCCTGTGCAG
TTTCTAGATATCCCTACAGCAGCATCTGCATGGGC
TGGTTCCGCCAGGCTCCAGGGAAGGAGAGCGAGGG
TGTCGCACGTATTTATACTGGTACTGGTAGTACAT
GGTATACCGACTCCGTGAAGGGCCGATTCACCATC
GCCCGAGACAACGCCCAGAACACGGTGTATCTGCA
AATGAACAGCCTGAAACCTGAGGACACTGCCATGT
ACTACTGTGCGGCGCGTAGCAATTCATATTCATAT
TCAAGTTGTGATTACGGCCCCCTCACGAGGGGGGG
GTATAACTTCTGGGGCCAGGGGACCCAGGTCACCG
TCTCCTCA
(Camel sdAb AS64606 Nucleic Acid Sequence)
SEQ ID NO: 437
GAGGTGCAGCTGGCGGAGTCTGGGGGAGGATCGGT
GCAGGCTGGAGGGTCTCTGAGACTCTCCTGTGCAG
TCTCTGGATACACCAGCCGTAGCAATTACATGGGC
TGGTTCCGCCAGGCTCCAGGGAAGGAGCGCGAGGG
GGTCGCGCTAATTTATACTCGTGGTGGTAGCACAT
ACTATGCCTCCTCCGTGAAGGGCCGGTTCACCATC
TCCCAAGACAGTGCCAAGAAAACGTATCTGCAAAT
GAACAGTGTGAAACCGGAGGACACTGCCATGTACT
ACTGTGCTTTGCGCCTTGATGAGAAGATGTACTGG
GGCCAGGGGACCCAGGTCACCGTCTCCTCA
(Camel sdAb AS68121 Nucleic Acid Sequence)
SEQ ID NO: 438
GAGGTGCAGCTGGCGGAGTCTGGGGGAGGCTCGGT
GCAGGCTGGAGGGTCTCTGAGACTCTCCTGTGATG
CCTCTGGATACACCTACAGCCGCAACTGCATGGGC
TGGTTCCGCCAGGCTCCAGGGAAGGAGCGCGAGGG
GGTCGCAGCGTTCTATACTGATTATATTCGTTTTG
GGCGCACATATTATGCCGACTCCGTGAAGGGCCGA
TTCACCATCTTCCAAGACAACGCCAAGAACACGGT
GTATCTGCAAATGAACAGCCTGAAACCTGAGGACA
CTGCCATGTACTACTGTGCGGCAGATCCTGGGAGT
CGTACAGACGATAGTTGTGGTACCTCATACAACAA
AGGGAATTTTGGTTACTGGGGCCAGGGGACCCAGG
TCACCGTCTCCTCA
(Camel sdAb AS68170 Nucleic Acid Sequence)
SEQ ID NO: 439
CAGGTGCAGCTGGTGGAGTCTGGGGGAGGCTCGGT
GCAGGCTGGAGGGTCGCTGAGACTCTCCTGTACAG
CCTCTGGATACACCTACAGAAGCAACTGTATGGGC
TGGTTCCGCCAGGCTCCAGGGAAGGAGCGCGAGGG
GGTCGCAACAATCTATACTGGTGGTGGTCGTAATC
TATACTATGCCGACTCCGTGAAGGGCCGATTCACC
ATCTCCCGAGACAACGCCAAGAACACCCTGTACCT
GCAAATGAACAGCCTGAAGCCTGAGGACTCTGCCA
GGTACTACTGTGCGGCCGCGAGTGACGTGGCAGTT
GGTGTTAATTCCTGCGGGGGAAGGACTGCGGGGTT
TGACGCCTGGGGCCAGGGGACCCAGGTCACCGTCT
CCTCA
(Camel sdAb AS63964 Nucleic Acid Sequence)
SEQ ID NO: 440
CAGGTGAGGTTAGTGGAGTCTGGGGGAGGATCGGT
ACAGGCTGGAGGGTCTCTGAGACTCTCCTGCTCAG
CCTCTGGATACACCTACAGTTACAACAATATGGGC
TGGTTCCGCCAGGCTCCAGGGAACGAGCGCGAGGG
GGTCGCAGCTATTAGTGGTGGTCGTTTCACCGCCT
ATGCCGACTCCGTGAAGGGCCGATTCACCATCTCC
CGAGACAACGCCGAGAACACGCTGTATCTGCAAAT
GAACAACCTGAAACCTGAGGACACTGGGATGTACT
ACTGTGCGGCAGAGGTAGTTGATCCGACCTGGGGG
TCGCGTGACCAAAGACGATATAAGTACTGGGGCCA
GGGGACCCAGGTCACCGTCTCCTCA
(Camel sdAb AS64116 Nucleic Acid Sequence)
SEQ ID NO: 441
CAGGTGAAGTTGGTGGAGTCTGGGGGAGGCTCGGT
GCAGGCTGGAGGGTCTCTGAGACTCTCGTGTGCAG
CCTCTGGATACATCTACAGTTGCGTGGGCTGGTTC
CGCCAGGCTCCAGGGAAGGAGCGCGAGGGGGTCGC
AGGTATTAGTACTGGTGGTGGTGGCACAGTCTATG
CCGACTCCGTGAAGGGCCAATTCACCATCTCCCGA
GACAACGCCAAGAACACGGTGTACCTGCAAATGGA
CAGCCTGAAACCTGAGGACACTGCCATGTACTACT
GTGCGGCAGATCGATGGAATTCATTCGCTAATTGC
GGTGCCTGGGGAAGGTATACCTACTGGGGCCAGGG
GACCCAGGTCACCGTCTCCTCA
(Camel sdAb AS68270 Nucleic Acid Sequence)
SEQ ID NO: 442
CAGGTGCAACTGGCGGAGTCTGGGGGAGGCTCGGT
GCAGGCTGGAGGGTCTCTGAGACTCTCCTGTGTAG
CCTCTGGATACCCCTCTTCCACCTACTACATGCTC
AGCATGGCGTGGTTCCGCCAGGCTCCAGGGAAGGA
GCGCGAGGGGGTCGCCGCTATTACTAGCGGTACTG
GGAGCACAAGCTACGCAGACTCCGTGAAGGACCGA
TTCACCATCTCCAAAGACTACGCCAACAACACTCT
GTATCTGCACATAAACAACCTGAAACCTGAGGACA
CTGCCATGTACTACTGTGCGGCAGCCTCAGGTTGG
ATCGTTCCTAGTAGGTCCCTGACCGCCAACCTATA
TAGGTATTGGGGCCAGGGGACCCAGGTCACCGTCT
CCTCA
(Camel sdAb AS68320 Nucleic Acid Sequence)
SEQ ID NO: 443
CAGGTGCACCTGGTGGAGTCTGGGGGAGACTCGGT
GCAGGCTGGAGGGTCCCTGAGACTCTCCTGTGCAG
CCTCTGGATACACCTACAATACCAACTACATGGGC
TGGTTCCGCCAGGCTCCAGGGAAGGAGCGCGAGGG
GGTCGCAGCTATTTATAGACATAGTGGTAACACAG
CCTATGCCGACTCCGTGAAGGGCCGATTCACCATC
TCCCAAGACTACGCCAAGAACACCGTGTATCTGCA
AATGAACAGCCTGAAACCTGAGGACACTGCCATGT
ACTACTGTGCGGCAGGGCGCGCTGGTCCCTGGGCC
CTGATGCGCCCGACTGAGTTTGGTTACTGGGGCCA
GGGGACCCAGGTCACCGTCTCCTCA
(Camel sdAb AS68351 Nucleic Acid Sequence)
SEQ ID NO: 444
CAGGTGCAATTGGAGGAGTCTGGGGGAGGCTTGGT
GCAGCCTGGGGGGTCTCTGAGACTCTCCTGTGCGG
CCTCTGGAGACACATTTCGTGCCTATTACATGAAC
TGGGTCCGCCAGGCTCCAGGGAAGGGATTCGAGTG
GGTCTCAGGTATTAGCGCCAGTGGCGGCCGTACGT
CATACGCAGACTCCGTGAAGGGCCGATTCACCATC
TCCAGAGACAACGCCAAAAACACGCTGTATCTGCA
ATTGAACAGCCTGAGCACTGAGGACACGGGCATGT
ATTATTGTGTAAAGGGAGCTGTCCGTCTCTCGACA
TCGTCAGTACGGGATTCGTCCTGGGGCCAGGGGAC
CCAGGTCACCGTCTCCTCA
(Camel sdAb AS75378 Nucleic Acid Sequence)
SEQ ID NO: 445
CAGGTGCAATTGGAGGAGTCTGGGGGAGGCTCGGT
GCAGGCTGGAGGGTCTCTGAGACTCTCCTGTGTAG
TCTCTGGAAACACCCGCAGTACCACGTACATGGGC
TGGTTCCGCCAGGCTCCAGGGAAGGAGCGCGAGGG
GGTCGCAATAGTTTATACTGGTGGTCGTGACACAT
ACTATGCCGCCTCCGTGAAGGGCCGATTCACCATC
TCCCAAGACAACGCCAAGACAACGATCTATCTGCA
AATGAACAGTCTGGAACCTGAGGACACTGCCATGT
ACTACTGTGCGGCACGCTCATATGAGTATACCTAC
TGGGGTCGGGGGACCCAGGTCACCGTCTCCTCA
(Camel sdAb AS75383 Nucleic Acid Sequence)
SEQ ID NO: 446
GAGGTGCAACTGGCGGAGTCTGGGGGAGGCTCGGT
GCAGGCTGGAGGGTCTCTGAGACTCTCCTGTGTAG
CCTCTGGATACACCTTCAGTAGCTACTGCTTGGGC
TGGTTCCGCCAGGCTCCAGGGAAGCAGCGCCAGGG
GGTCGCAACGTTTAATAATAGAGGTGTCGCAAACT
ACCACGATTCCGTGAAGGGCCGATTCACCGCCTCC
GTAGACAACGCCAAGAACACTCTGCTTCTGCAAAT
GAACAGCCTGGAACCTGACGACACGGCCATGTACT
ACTGTGCGGCGGATCGCCGGTACGGTCGTCAGTGG
TATCAGCCTTGCGAGTGGAACACCTGGGGCCAGGG
GACCCAGGTCACCGTCTCCTCA
(Camel sdAb AS75751 Nucleic Acid Sequence)
SEQ ID NO: 447
CAGGTGAGGTTGGTGGAGTCTGGGGGAGGCTCGGT
GCAGGCTGGAGGGTCTCTGAGACTCTCCTGTGTAG
CCTCTGGATACTTCTACAATACCTACTACTTTATG
GGCTGGTTCCGCCAGGCTCCAGGGAAGGAGCGCGA
GGGGGTCGCAGCTATTGATACTGATGGTAGAACAA
GTTACGCAGACTCCGTGAAGGGCCGATTCACCATC
TCCAAAGACAACGCCAAGAACACTCTGTATCTGCA
AATGAACAGCCTGAAACCTGAGGACACTGCCATGT
ACTACTGTGCGGCAGGTTTTGGCTATATGAATGTT
ATTCAGGCTCTTAATGGCATGAGACAGAATCCCGA
CTACTGGGGCCAGGGGACCCAGGTCACCGTCTCCT
CA
(Camel sdAb AS76422 Nucleic Acid Sequence)
SEQ ID NO: 448
CAGGTGAAGTTGGTGGAGTCTGGGGGAGGCTCGGT
GCAGGCTGGAGGGTCTCTGAGACTCTCCTGTGCAG
CCTCTGGATACACCTTCGCTGGCAACTGCTTGGGC
TGGTTTCGCCAGGCTCCAGGGAAGGGGCGCGAGGG
GGTCGTAACGTACAATAACTTCGGTGTCGCCAACT
ACGCCGATTCCGTGAAGGGCCGATTCACCGTCTCC
CAAGACAACGCCAAGAACACTCTGCTTCTGCAAAT
GAACAGCCTGGAACCTGAGGACACTGCCATGTACT
ACTGTGCGGCGGACCGCCGGGACGGTCGTCGCTGG
TCTCAGCCTTGCGAGTGGAATACCTGGGGCCAGGG
GACCCAGGTCACCGTCTCCTCA
Humanized Camel sdAb Nucleic Acid Sequences
(Humanized sdAb AS64380VH4 Nucleic Acid
Sequence)
SEQ ID NO: 449
GAGGTGCAGCTGGTGGAGAGCGGAGGAGGACTGGT
GCAGCCAGGAGGCAGCCTGAGGCTGTCCTGCGCAG
CATCTGGAAACACCTACAGCTCCAATTATATGGGA
TGGTTCAGGCAGGCACCTGGCAAGGGACTGGAGGA
GGTGGCCGTGATCTACACCAGGGGAGGACACACAT
ACTATGTGGACTCCGTGCGGGGACGGTTCACCATC
AGCAGGGATAACGCCAAGAACAGCCTGTATCTGCA
GATGAACTCTCTGAGAGCCGAGGACACAGCCGTGT
ACTATTGTGCAGCATCTAGCAGGCACAGGCTGGGC
CTGAACAATCCAAGGGACTACGATTATTGGGGCCA
GGGCACCCTGGTGACAGTGTCCTCT
(Humanized sdAb AS64380VH5 Nucleic Acid Sequence)
SEQ ID NO: 450
GAGGTGCAGCTGGTGGAGAGCGGAGGAGGACTGGT
GCAGCCAGGAGGCAGCCTGAGGCTGTCCTGCGCAG
CATCTGGAAACACCTACAGCTCCAATTATATGGGA
TGGTTCAGGCAGGCACCTGGCAAGGGCCTGGAGGA
GGTGGCCGTGATCTACACCAGAGGCGGCCACACAT
ACTATGTGGACTCCGTGCGGGGACGGTTCACCATC
AGCCAGGATAACGCCAAGAACAGCCTGTATCTGCA
GATGAACTCTCTGAGGGCCGAGGACACAGCCGTGT
ACTATTGTGCAGCATCTAGCAGGCACAGGCTGGGC
CTGAACAATCCAAGGGACTACGATTATTGGGGCCA
GGGCACCCTGGTGACAGTGTCCTCT
(Humanized sdAb AS64380VH6 Nucleic Acid Sequence)
SEQ ID NO: 451
GAGGTGCAGCTGGTGGAGAGCGGAGGAGGACTGGT
GCAGCCAGGAGGCAGCCTGAGGCTGTCCTGCGCAG
CATCTGGAAACACCTACAGCTCCAATTATATGGGA
TGGTTCAGGCAGGCACCTGGCAAGGGCCTGGAGGA
GGTGGCCGTGATCTACACCAGAGGCGGCCACACAT
ACTATGTGGACTCCGTGCGGGGACGGTTCACCATC
AGCCAGGATAACGCCAAGAACAGCGTGTATCTGCA
GATGAACTCTCTGAGGGCCGAGGACACAGCCATGT
ACTATTGTGCAGCATCTAGCAGGCACAGGCTGGGC
CTGAACAATCCAAGGGACTACGATTATTGGGGCCA
GGGCACCCTGGTGACAGTGTCCTCT
(Humanized sdAb AS64380VH7 Nucleic
Acid Sequence)
SEQ ID NO: 452
GAGGTGCAGCTGGTGGAGAGCGGAGGAGGACTGGT
GCAGCCAGGAGGCAGCCTGAGGCTGTCCTGCGCAG
CATCTGGAAACACCTACAGCTCCAATTATATGGGA
TGGTTCAGGCAGGCACCTGGCAAGGGAAGAGAGGA
GGTGGCCGTGATCTACACCAGGGGAGGACACACAT
ACTATGTGGACTCCGTGCGGGGACGGTTCACCATC
AGCCAGGATAACGCCAAGAACAGCGTGTATCTGCA
GATGAACTCTCTGAGGGCCGAGGACACAGCCATGT
ACTATTGTGCAGCATCTAGCAGGCACAGGCTGGGC
CTGAACAATCCAAGGGACTACGATTATTGGGGCCA
GGGCACCCTGGTGACAGTGTCCTCT
(Humanized sdAb AS64511VH4 Nucleic
Acid Sequence)
SEQ ID NO: 453
GAGGTGCAGCTGGTGGAGTCCGGAGGAGGACTGGT
GCAGCCAGGAGGCTCTCTGAGGCTGAGCTGCGCAG
CATCCAGAGCAACCTACTCTACAAACTATATCAGC
TGGTTCAGGCAGGCACCTGGCAAGGGACTGGAGGC
AGTGGCAACCATCACCACAGGCGATGGCGAGACAG
CCTACGCCGACTCTGTGAAGGGCAGGTTTACCATC
TCCCGCGATAACGCCAAGAACAGCCTGTATCTGCA
GATGAACAGCCTGAGGGCCGAGGACACAGCCGTGT
ACTATTGTGCAGCAAATCTGAGGATCGGAGGCGAC
TGGTTCGATGGAAGGGACTTTAGAGCATGGGGACA
GGGAACCCTGGTGACAGTGAGCTCC
(Humanized sdAb AS64511VH5 Nucleic
Acid Sequence)
SEQ ID NO: 454
GAGGTGCAGCTGGTGGAGTCCGGAGGAGGACTGGT
GCAGCCAGGAGGCTCTCTGAGGCTGAGCTGCGCAG
CATCCAGAGCAACCTACTCTACAAACTATATCAGC
TGGTTCAGGCAGGCACCTGGCAAGGGACTGGAGGC
AGTGGCAACCATCACCACAGGCGATGGCGAGACAG
CCTACGCCGACTCTGTGAAGGGCAGGTTTACCATC
TCCCGCGATAACGCCAAGAACAGCGTGTATCTGCA
GATGAACAGCCTGAGGGCCGAGGACACAGCCATGT
ACTATTGTGCAGCAAATCTGAGGATCGGAGGCGAC
TGGTTCGATGGAAGGGACTTTAGAGCATGGGGACA
GGGAACCCTGGTGACAGTGAGCTCC
(Humanized sdAb AS64511VH6 Nucleic
Acid Sequence)
SEQ ID NO: 455
GAGGTGCAGCTGGTGGAGTCCGGAGGAGGACTGGT
GCAGCCAGGAGGCTCTCTGAGGCTGAGCTGCGCAG
CATCCAGAGCAACCTACTCTACAAACTATATCAGC
TGGTTCAGGCAGGCACCTGGCAAGGGAAGGGAGGC
AGTGGCCACCATCACCACAGGCGATGGCGAGACAG
CCTACGCCGACTCTGTGAAGGGCAGGTTTACCATC
TCCCGCGATAACGCCAAGAACAGCGTGTATCTGCA
GATGAACAGCCTGCGGGCCGAGGACACAGCCATGT
ACTATTGTGCAGCAAATCTGAGGATCGGAGGCGAC
TGGTTCGATGGAAGGGACTTTAGAGCATGGGGACA
GGGAACCCTGGTGACAGTGAGCTCC
(Humanized sdAb AS63931VH4 Nucleic
Acid Sequence)
SEQ ID NO: 456
CAGGTGCAGCTGGTGGAGAGCGGAGGAGGAGTGGT
GCAGCCAGGAGGCAGCCTGAGGCTGTCCTGCGCAG
GCTCTTTCAGCGGATACGGCGTGTCCACCATGGCA
TGGTTTAGGCAGGCACCTGGCAAGGGACTGGAGGG
AGTGGCAGCAATCACCGTGGGATCCGGAAACACAT
ACTATGCCGACTCTGTGACCGGCCGGTTCACAATC
TCTAGAGATAACAGCAAGAATACCCTGTATCTGCA
GATGAACAGCCTGCGGGCCGAGGACACAGCCGTGT
ACTATTGTGCAGCAGGATGGCTGTCCGGAGGATCT
TGGCACGTGCCCGGCAGGTACAACTATTGGGGCCA
GGGCACCCTGGTGACAGTGAGCTCC
(Humanized sdAb AS63931VH5 Nucleic
Acid Sequence)
SEQ ID NO: 457
CAGGTGCAGCTGGTGGAGAGCGGAGGAGGAGTGGT
GCAGCCAGGAGGCAGCCTGAGGCTGTCCTGCGCAG
GCTCTTTCAGCGGATACGGCGTGTCCACCATGGCA
TGGTTTAGGCAGGCACCTGGCAAGGGACTGGAGGG
AGTGGCAGCAATCACCGTGGGATCCGGAAACACAT
ACTATGCCGACTCTGTGACCGGCCGGTTCACAATC
TCTAGAGATAACAGCAAGAATACCGTGTATCTGCA
GATGAACAGCCTGCGGGCCGAGGACACAGCCATGT
ACTATTGTGCAGCAGGATGGCTGTCCGGAGGATCT
TGGCACGTGCCCGGCAGGTACAACTATTGGGGCCA
GGGCACCCTGGTGACAGTGAGCTCC
(Humanized sdAb AS63931VH6 Nucleic
Acid Sequence)
SEQ ID NO: 458
CAGGTGCAGCTGGTGGAGAGCGGAGGAGGAGTGGT
GCAGCCAGGAGGCAGCCTGAGGCTGTCCTGCGCAG
GCTCTTTCAGCGGATACGGCGTGTCCACCATGGCC
TGGTTTAGGCAGGCACCTGGCAAGGGAAGGGAGGG
AGTGGCAGCAATCACCGTGGGATCCGGAAACACAT
ACTATGCCGACTCTGTGACCGGCCGGTTCACAATC
TCTAGAGATAACAGCAAGAATACCGTGTATCTGCA
GATGAACAGCCTGCGGGCCGAGGACACAGCCATGT
ACTATTGTGCAGCAGGATGGCTGTCCGGAGGATCT
TGGCACGTGCCCGGCAGGTACAACTATTGGGGCCA
GGGCACCCTGGTGACAGTGAGCTCC
(Humanized sdAb AS63997VH4 Nucleic
Acid Sequence)
SEQ ID NO: 459
CAGGTGCAGCTGGTGGAGAGCGGAGGAGGAGTGGT
GCAGCCAGGAGGCAGCCTGAGGCTGTCCTGCGCAG
CCTCTTTCAGCGGATACGGCGTGTCCACCATGGCA
TGGTTTAGGCAGGCACCTGGCAAGGGACTGGAGGG
AGTGGCAGCAATCACCGTGGGATCCGGAAACACAT
ACTATGCCGACTCTGTGAAGGGCCGGTTCACCATC
TCTAGAGATAACAGCAAGAATACACTGTACCTGCA
GATGAACAGCCTGCGGGCCGAGGACACAGCCGTGT
ACTATTGTGCCGTGGGCTATCTGTCCGGAGGATCT
TGGGATGTGCCAGGAAGGTACAACTATTGGGGCCA
GGGCACCCTGGTGACAGTGAGCTCC
(Humanized sdAb AS63997VH5 Nucleic
Acid Sequence)
SEQ ID NO: 460
CAGGTGCAGCTGGTGGAGAGCGGAGGAGGAGTGGT
GCAGCCAGGAGGCAGCCTGAGGCTGTCCTGCGCAG
CCTCTTTCAGCGGATACGGCGTGTCCACCATGGCA
TGGTTTAGGCAGGCACCTGGCAAGGGACTGGAGGG
AGTGGCAGCAATCACCGTGGGATCCGGAAACACAT
ACTATGCCGACTCTGTGAAGGGCCGGTTCACCATC
TCTAGAGATAACAGCAAGAATACAGTGTACCTGCA
GATGAACAGCCTGCGGGCCGAGGACACAGCCATGT
ACTATTGTGCCGTGGGCTATCTGTCCGGAGGATCT
TGGGATGTGCCAGGAAGGTACAACTATTGGGGCCA
GGGCACCCTGGTGACAGTGAGCTCC
(Humanized sdAb AS63997VH6 Nucleic
Acid Sequence)
SEQ ID NO: 461
CAGGTGCAGCTGGTGGAGAGCGGAGGAGGAGTGGT
GCAGCCAGGAGGCAGCCTGAGGCTGTCCTGCGCAG
CCTCTTTCAGCGGATACGGCGTGTCCACCATGGCC
TGGTTTAGGCAGGCACCTGGCAAGGGAAGGGAGGG
AGTGGCAGCAATCACCGTGGGATCCGGAAACACAT
ACTATGCCGACTCTGTGAAGGGCCGGTTCACCATC
TCTAGAGATAACAGCAAGAATACAGTGTACCTGCA
GATGAACAGCCTGCGGGCCGAGGACACAGCCATGT
ACTATTGTGCCGTGGGCTATCTGTCCGGAGGATCT
TGGGATGTGCCAGGAAGGTACAACTATTGGGGCCA
GGGCACCCTGGTGACAGTGAGCTCC
(Linker amino acid sequence)
SEQ ID NO: 462
GGGGSGGGGSGGGGS
(Linker amino acid sequence)
SEQ ID NO: 463
GGGGSGGGGS
(Linker amino acid sequence)
SEQ ID NO: 464
GGGGS
(CD8α signal peptide amino acid sequence)
SEQ ID NO: 465
MALPVTALLLPLALLLHAARP
(CD8α hinge amino acid sequence)
SEQ ID NO: 466
TTTPAPRPPTPAPTIASQPLSLRPEACRPAAGGAV
HTRGLDFACDIYIWAPLAGTCGVLLLSLVITLYC
(CD8α transmembrane domain amino
acid sequence)
SEQ ID NO: 467
IYIWAPLAGTCGVLLLSLVITLYC
(4-1BB intracellular domain amino
acid sequence)
SEQ ID NO: 468
KRGRKKLLYIFKQPFMRPVQTTQEEDGCSCRFPEE
EEGGCEL
(CD28 intracellular domain amino
acid sequence)
SEQ ID NO: 469
RSKRSRLLHSDYMNMTPRRPGPTRKHYQPYAPPRDFAAYRS
(CD3ζ intracellular domain amino acid
sequence)
SEQ ID NO: 470
RVKFSRSADAPAYKQGQNQLYNELNLGRREEYDVL
DKRRGRDPEMGGKPRRKNPQEGLYNELQKDKMAEA
YSEIGMKGERRRGKGHDGLYQGLSTATKDTYDALH
MQALPPR
(F2A element amino acid sequence)
SEQ ID NO: 471
GSGVKQTLNFDLLKLAGDVESNPGP
(P2A element amino acid sequence)
SEQ ID NO: 472
GSGATNFSLLKQAGDVEENPGP
(CAR3 anti-DLL3 scFv amino acid sequence)
SEQ ID NO: 473
MALPVTALLLPLALLLHAARPAIQLTQSPSSLSAS
VGDRVTITCRASENIYYNLAWYQQKPGKAPKWYTA
NSLEDVPSRFSGSGSGTDFTLTISSLQPEDFATYF
CKQAYDVPPTFGGGTKLEIKGGGGSGGGGSGGGGS
QVQLVQSGAEVKKPGASVKVSCKASGYTFTRYWIF
IWIRQAPGQGLEWMGYINPTVYTEFNQNFKDRVTM
TRDTSTSTVYMELSSLRSEDTAVYYCARGGSNFFD
YWQGTTVTVSS
(CD28 transmembrane domain amino acid
sequence)
SEQ ID NO: 474
FWVLVVVGGVLACYSLLVTVAFIIFWV
(CD28 hinge)
SEQ ID NO: 475
IEVMYPPPYLDNEKSNGTIIHVKGKHLCPSPLFPG
PSKP
Camel anti-DLL3 CAR sequences
(CAS63997)
SEQ ID NO: 476
MALPVTALLLPLALLLHAARPQVRLVESGGGSVQA
GGSLRLSCAGSFSGYGVSTMAWFRQAPGKEREGVA
AITVGSGNTYYADSVKGRFTISRDNAKRTVFLQMN
SLKPEDTAMYYCAVGYLSGGSWDVPGRYNYWGQGT
QVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS64380)
SEQ ID NO: 477
MALPVTALLLPLALLLHAARPEVQLVESGGGSVQA
GGSLTLSCEASGNTYSSNYMGWFRQAPGKEREEVA
VIYTRGGHTYYVDSVRGRFTISQDNAKNTVYLQMN
SLKPEDTAMYYCAASSRHRLGLNNPRDYDYWGQGT
QVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS64511)
SEQ ID NO: 478
MALPVTALLLPLALLLHAARPQVQLVESGGGSVQA
GGSLRLSCAASRATYSTNYISWFRQAPGKEREAVA
TITTGDGETAYADSVKGRFTISRDNAKNTVYLQMN
SLKPEDTAMYYCAANLRIGGDWFDGRDFRAWGQGT
QVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS64617)
SEQ ID NO: 479
MALPVTALLLPLALLLHAARPQVQLVESGGGSVQA
GGSLRLSCAASGYTDRCSMAWYRQAPGKERELVSR
ISTSGFTNYAASVKGRFTISQDNAKNTVYLQMNSL
NPGDTGMYYCAIIVGRTCSLNYWGNGILVTVSSTT
TPAPRPPTPAPTIASQPLSLRPEACRPAAGGAVHT
RGLDFACDIYIWAPLAGTCGVLLLSLVITLYCKRG
RKKLLYIFKQPFMRPVQTTQEEDGCSCRFPEEEEG
GCELRVKFSRSADAPAYKQGQNQLYNELNLGRREE
YDVLDKRRGRDPEMGGKPRRKNPQEGLYNELQKDK
MAEAYSEIGMKGERRRGKGHDGLYQGLSTATKDTY
DALHMQALPPR
(CAS69443)
SEQ ID NO: 480
MALPVTALLLPLALLLHAARPEVQLAESGGGSVQA
GGSLRLSCSASGFTFDDSDMAWYRQAPGDGCDLVS
TISSDGSTYYADSVKGRFTISQDNAKNTVYLQMHS
LKPEDTAVYYCAADFLTGFYYSDSPHPAPCSASDF
GYWGQGTQVTVSSTTTPAPRPPTPAPTIASQPLSL
RPEACRPAAGGAVHTRGLDFACDIYIWAPLAGTCG
VLLLSLVITLYCKRGRKKLLYIFKQPFMRPVQTTQ
EEDGCSCRFPEEEEGGCELRVKFSRSADAPAYKQG
QNQLYNELNLGRREEYDVLDKRRGRDPEMGGKPRR
KNPQEGLYNELQKDKMAEAYSEIGMKGERRRGKGH
DGLYQGLSTATKDTYDALHMQALPPR
(CAS63931)
SEQ ID NO: 481
MALPVTALLLPLALLLHAARPEVQLAESGGGSVQA
GGSLRLSCAGSFSGYGVSTMAWFRQAPGKEREGVA
AITVGSGNTYYADSVTGRFTISRDNAKRTVYLQMN
SLKPEDTAMYYCAAGWLSGGSWHVPGRYNYWGQGT
QVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS64047)
SEQ ID NO: 482
MALPVTALLLPLALLLHAARPQVHLVESGGGSVQA
GGSLRLSCAASQYVYRWDLMGWFRQAPGKEREAVA
AVYTGDGITYYADSVKGRFSISQDNAKNTVYLQMN
SLKPEDTGMYFCAAGFVSGGRWNQSYRYKYWGQGT
QVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS64052)
SEQ ID NO: 483
MALPVTALLLPLALLLHAARPQVHLMESGGGSVQAG
GSLRLSCAASGYTYRSNFMGWFRQAPGKEREGIAT
IHSGVATTYYADSVKGRFTISQDNAKNTVYLQMNS
LKPEDTAMYYCAAGGPPANADRWYPLRPPGYNYWG
QGTQVTVSSTTTPAPRPPTPAPTIASQPLSLRPEA
CRPAAGGAVHTRGLDFACDIYIWAPLAGTCGVLLL
SLVITLYCKRGRKKLLYIFKQPFMRPVQTTQEEDG
CSCRFPEEEEGGCELRVKFSRSADAPAYKQGQNQL
YNELNLGRREEYDVLDKRRGRDPEMGGKPRRKNPQ
EGLYNELQKDKMAEAYSEIGMKGERRRGKGHDGLY
QGLSTATKDTYDALHMQALPPR
(CAS64062)
SEQ ID NO: 484
MALPVTALLLPLALLLHAARPQVRLVESGGGSVQV
GGSLRLSCAASRSPYSSSRCMGWFRQAPGKEREGV
AALYTGGGSTSYADSVKGRFTISQDNAKNTVYLQM
NSLKPEDTAMYYCAAVVPRGGSCRLDERGYYHWGQ
GTQVTVSSTTTPAPRPPTPAPTIASQPLSLRPEAC
RPAAGGAVHTRGLDFACDIYIWAPLAGTCGVLLLS
LVITLYCKRGRKKLLYIFKQPFMRPVQTTQEEDGC
SCRFPEEEEGGCELRVKFSRSADAPAYKQGQNQLY
NELNLGRREEYDVLDKRRGRDPEMGGKPRRKNPQE
GLYNELQKDKMAEAYSEIGMKGERRRGKGHDGLYQ
GLSTATKDTYDALHMQALPPR
Humanized anti-DLL3 CAR sequences
(CAS64380VH4)
SEQ ID NO: 485
MALPVTALLLPLALLLHAARPEVQLVESGGGLVQP
GGSLRLSCAASGNTYSSNYMGWFRQAPGKGLEEVA
VIYTRGGHTYYVDSVRGRFTISRDNAKNSLYLQMN
SLRAEDTAVYYCAASSRHRLGLNNPRDYDYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS64380VH5)
SEQ ID NO: 486
MALPVTALLLPLALLLHAARPEVQLVESGGGLVQP
GGSLRLSCAASGNTYSSNYMGWFRQAPGKGLEEVA
VIYTRGGHTYYVDSVRGRFTISQDNAKNSLYLQMN
SLRAEDTAVYYCAASSRHRLGLNNPRDYDYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS64380VH6)
SEQ ID NO: 487
MALPVTALLLPLALLLHAARPEVQLVESGGGLVQP
GGSLRLSCAASGNTYSSNYMGWFRQAPGKGLEEVA
VIYTRGGHTYYVDSVRGRFTISQDNAKNSVYLQMN
SLRAEDTAMYYCAASSRHRLGLNNPRDYDYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS64380VH7)
SEQ ID NO: 488
MALPVTALLLPLALLLHAARPEVQLVESGGGLVQP
GGSLRLSCAASGNTYSSNYMGWFRQAPGKGREEVA
VIYTRGGHTYYVDSVRGRFTISQDNAKNSVYLQMN
SLRAEDTAMYYCAASSRHRLGLNNPRDYDYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS64511VH4)
SEQ ID NO: 489
MALPVTALLLPLALLLHAARPEVQLVESGGGLVQP
GGSLRLSCAASRATYSTNYISWFRQAPGKOLEAVA
TITTGDGETAYADSVKGRETISRDNAKNSLYLQMN
SLRAEDTAVYYCAANLRIGGDWFDGRDFRAWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS64511VH5)
SEQ ID NO: 490
MALPVTALLLPLALLLHAARPEVQLVESGGGLVQP
GGSLRLSCAASRATYSTNYISWFRQAPGKOLEAVA
TITTGDGETAYADSVKGRETISRDNAKNSVYLQMN
SLRAEDTAMYYCAANLRIGGDWFDGRDFRAWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS64511VH6)
SEQ ID NO: 491
MALPVTALLLPLALLLHAARPEVQLVESGGGLVQP
GGSLRLSCAASRATYSTNYISWFRQAPGKGREAVA
TITTGDGETAYADSVKGRFTISRDNAKNSVYLQMN
SLRAEDTAMYYCAANLRIGGDWFDGRDFRAWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS63997VH4)
SEQ ID NO: 492
MALPVTALLLPLALLLHAARPQVQLVESGGGVVQP
GGSLRLSCAASFSGYGVSTMAWFRQAPGKOLEGVA
AITVGSGNTYYADSVKGRETISRDNSKNTLYLQMN
SLRAEDTAVYYCAVGYLSGGSWDVPGRYNYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS63997VH5)
SEQ ID NO: 493
MALPVTALLLPLALLLHAARPQVQLVESGGGVVQP
GGSLRLSCAASFSGYGVSTMAWFRQAPGKOLEGVA
AITVGSGNTYYADSVKGRETISRDNSKNTVYLQMN
SLRAEDTAMYYCAVGYLSGGSWDVPGRYNYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(CAS63997VH6)
SEQ ID NO: 494
MALPVTALLLPLALLLHAARPQVQLVESGGGVVQP
GGSLRLSCAASFSGYGVSTMAWFRQAPGKGREGVA
AITVGSGNTYYADSVKGRFTISRDNSKNTVYLQMN
SLRAEDTAMYYCAVGYLSGGSWDVPGRYNYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
Anti-DLL3 Human scFv VL and VH Domain
Nucleic Acid Sequences
(Nucleic Acid Sequence for VL domain
of anti-DLL3 human scFv AS56704)
SEQ ID NO: 511
GACATCCAGATGACCCAGAGCCCGAGCAGCCTGAG
CGCGAGCGTTGGTGACCGTGTTACCATTACCTGCC
GTGCGAGCCAGAGCGTTAGCAGCGCGGTGGCGTGG
TACCAGCAAAAGCCGGGTAAAGCGCCGAAGCTGCT
GATCTATAGCGCGAGCAGCCTGTATAGCGGCGTTC
CGAGCCGTTTCAGCGGTAGCCGTAGCGGCACCGAC
TTTACCCTGACCATTAGCAGCCTGCAGCCGGAAGA
TTTCGCAACTTATTACTGTCAGCAAGCTTCTTGGT
CTCCGATCACGTTCGGACAGGGCACCAAAGTTGAG
ATTAAA
(Nucleic Acid Sequence for VH domain
of anti-DLL3 human scFv AS56704)
SEQ ID NO: 512
GAGGTTCAACTGGTGGAGAGCGGTGGTGGTCTGGT
TCAGCCGGGTGGTAGCCTGCGTCTGAGCTGCGCAG
CTTCTGGCTTCAACATCTCTTCTTCTTATATGCAC
TGGGTGCGTCAGGCGCCGGGTAAAGGCCTGGAATG
GGTTGCATATATTTATCCTTCTTATGGCTATACTT
CTTATGCCGATAGCGTCAAGGGCCGTTTCACCATC
AGCGCGGATACCAGCAAAAACACCGCATACCTGCA
AATGAACAGCCTGCGTGCGGAAGATACCGCCGTCT
ATTATTGTGCTCGCGGTGGTTACTACTACCATGGT
ATGGACTACTGGGGTCAAGGCACCCTGGTTACCGT
GAGCAGC
(Nucleic Acid Sequence for VL domain
of anti-DLL3 human scFv AS56788)
SEQ ID NO: 513
GACATCCAGATGACCCAGAGCCCGAGCAGCCTGAG
CGCGAGCGTTGGTGACCGTGTTACCATTACCTGCC
GTGCGAGCCAGAGCGTTAGCAGCGCGGTGGCGTGG
TACCAGCAAAAGCCGGGTAAAGCGCCGAAGCTGCT
GATCTATAGCGCGAGCAGCCTGTATAGCGGCGTTC
CGAGCCGTTTCAGCGGTAGCCGTAGCGGCACCGAC
TTTACCCTGACCATTAGCAGCCTGCAGCCGGAAGA
TTTCGCAACTTATTACTGTCAGCAACATTACGCTC
CGTCTCTGATCACGTTCGGACAGGGCACCAAAGTT
GAGATTAAA
(Nucleic Acid Sequence for VH domain
of anti-DLL3 human scFv AS56788)
SEQ ID NO: 514
GAGGTTCAACTGGTGGAGAGCGGTGGTGGTCTGGT
TCAGCCGGGTGGTAGCCTGCGTCTGAGCTGCGCAG
CTTCTGGCTTCAACATCTCTTCTTATTCTATGCAC
TGGGTGCGTCAGGCGCCGGGTAAAGGCCTGGAATG
GGTTGCATATATTTCTTCTTATTATGGCTATACTT
ATTATGCCGATAGCGTCAAGGGCCGTTTCACCATC
AGCGCGGATACCAGCAAAAACACCGCATACCTGCA
AATGAACAGCCTGCGTGCGGAAGATACCGCCGTCT
ATTATTGTGCTCGCTACTCTTACTACTACGGTATG
GACTACTGGGGTCAAGGCACCCTGGTTACCGTGAG
CAGC
Human anti-DLL3 scFv CAR sequences
(CAS56704)
SEQ ID NO: 515
MALPVTALLLPLALLLHAARPDIQMTQSPSSLSAS
VGDRVTITCRASQSVSSAVAWYQQKPGKAPKLLIY
SASSLYSGVPSRFSGSRSGTDFTLTISSLQPEDFA
TYYCQQASWSPITFGQGTKVEIKGGGGSGGGGSGG
GGSEVQLVESGGGLVQPGGSLRLSCAASGFNISSS
YMHWVRQAPGKGLEWVAYIYPSYGYTSYADSVKGR
FTISADTSKNTAYLQMNSLRAEDTAVYYCARGGYY
YHGMDYWGQGTLVTVSSTTTPAPRPPTPAPTIASQ
PLSLRPEACRPAAGGAVHTRGLDFACDIYIWAPLA
GTCGVLLLSLVITLYCKRGRKKLLYIFKQPFMRPV
QTTQEEDGCSCRFPEEEEGGCELRVKFSRSADAPA
YKQGQNQLYNELNLGRREEYDVLDKRRGRDPEMGG
KPRRKNPQEGLYNELQKDKMAEAYSEIGMKGERRR
GKGHDGLYQGLSTATKDTYDALHMQALPPR
(CAS56788)
SEQ ID NO: 516
MALPVTALLLPLALLLHAARPDIQMTQSPSSLSAS
VGDRVTITCRASQSVSSAVAWYQQKPGKAPKLLIY
SASSLYSGVPSRFSGSRSGTDFTLTISSLQPEDFA
TYYCQQHYAPSLITFGQGTKVEIKGGGGSGGGGSG
GGGSEVQLVESGGGLVQPGGSLRLSCAASGFNISS
YSMHWVRQAPGKGLEWVAYISSYYGYTYYADSVKG
RFTISADTSKNTAYLQMNSLRAEDTAVYYCARYSY
YYGMDYWGQGTLVTVSSTTTPAPRPPTPAPTIASQ
PLSLRPEACRPAAGGAVHTRGLDFACDIYIWAPLA
GTCGVLLLSLVITLYCKRGRKKLLYIFKQPFMRPV
QTTQEEDGCSCRFPEEEEGGCELRVKFSRSADAPA
YKQGQNQLYNELNLGRREEYDVLDKRRGRDPEMGG
KPRRKNPQEGLYNELQKDKMAEAYSEIGMKGERRR
GKGHDGLYQGLSTATKDTYDALHMQALPPR
Anti-DLL3 benchmark CAR
(1H2.1 amino acid sequence)
SEQ ID NO: 517
MALPVTALLLPLALLLHAARPQVQLQESGPGLVKP
SETLSLTCTVSGDSISSYYWTWIRQPPGKGLEWIG
YIYYSGTTNYNPSLKSRVTISVDTSKSQFSLKLSS
VTAADTAVYYCASIAVRGFFFDYWGQGTLVTVSSG
GGGSGGGGSGGGGSEIVLTQSPGTLSLSPGERATL
SCRASQSVSSSYLAWYQQKPGQAPRLLIYGASTRA
TGIPDRFSGSGSGTDFTLTISRLEPEDFAVYYCQQ
YGTSPLTFGGGTKVEIKRAAALDNEKSNGTIIHVK
GKHLCPSPLFPGPSKPFWVLVVVGGVLACYSLLVT
VAFIIFWVRSKRSRLLHSDYMNMTPRRPGPTRKHY
QPYAPPRDFAAYRSRVKFSRSADAPAYQQGQNQLY
NELNLGRREEYDVLDKRRGRDPEMGGKPRRKNPQE
GLYNELQKDKMAEAYSEIGMKGERRRGKGHDGLYQ
GLSTATKDTYDALHMQALPPR

### Example 11. Evaluation of In Vitro Activity of Humanized Anti-DLL3 Tandem CAR-T Cells

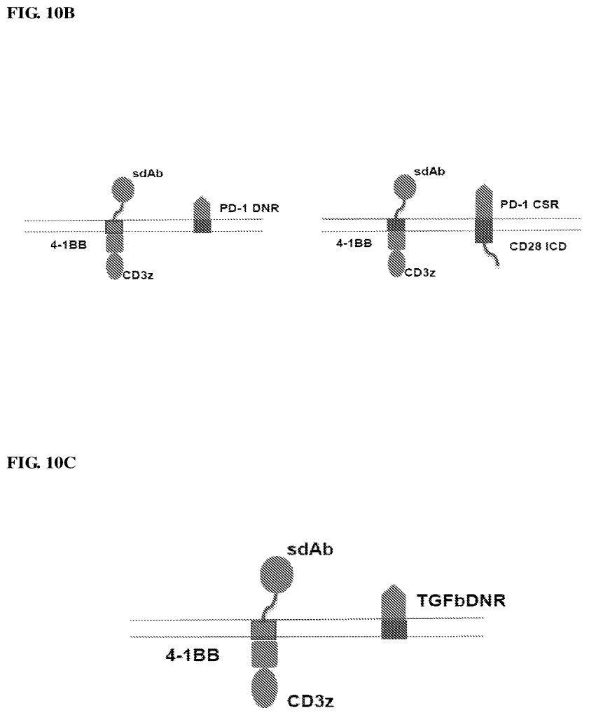

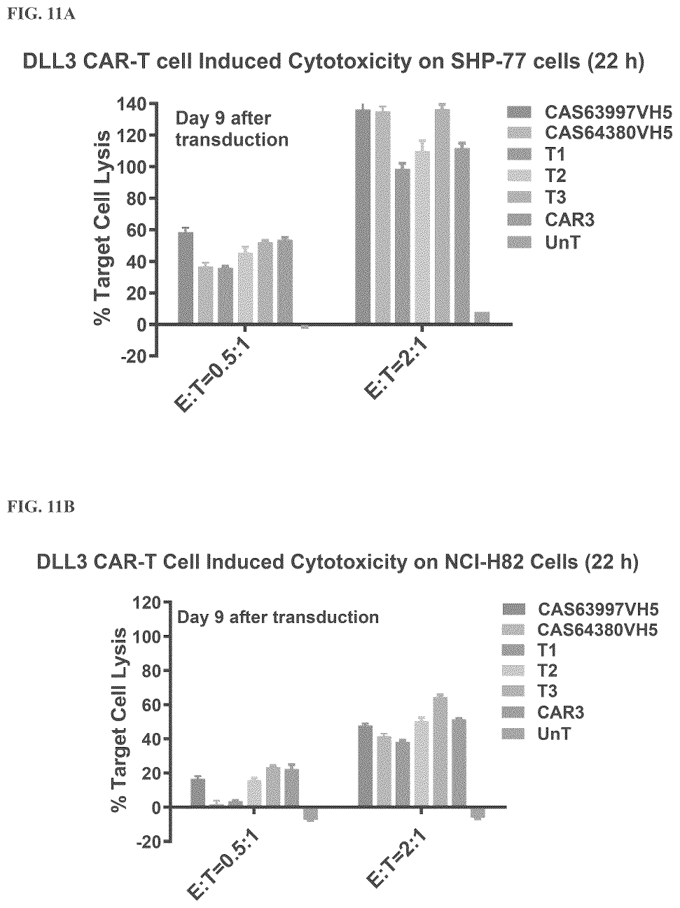

To improve anti-tumor efficacy of CAR-Ts, we constructed three tandem CARs (T1, T2 and T3). The amino acid sequences of the tandem CARs were provided in SEQ ID: 518-520. The amino acid sequences of anti-DLL3 humanized sdAb fragments were provided in SEQ ID NO: 356 (AS64380VH5) and SEQ ID NO: 366 (AS63997VH5). 1H2.1 (SEQ ID NO: 517, e.g, see, WO2019200007), which is an anti-DLL3 CAR, was also used to generate a CAR construct as a reference. A full length CAR contains from the N-terminus to the C-terminus: a CD8α signal peptide (SEQ ID NO: 465), DLL3 binding domain sdAbs provided in SEQ ID NO: 356 (AS64380VH5) and SEQ ID NO: 366 (AS63997VH5), a CD8α hinge domain (SEQ ID NO: 466), a CD8α transmembrane domain (SEQ ID NO: 467), a CD137 intracellular domain (SEQ ID NO: 468) or a CD28 intracellular domain (SEQ ID NO: 469), and a CD3ζ intracellular domain (SEQ ID NO: 470). Schematic representation of a CAR construct is shown in FIG. 10A. For T1, both of sdAb 1 and sdAb 2 were AS64380VH5. For T2, both of sdAb 1 and sdAb 2 were AS63997VH5. For T3, sdAb 1 and sdAb 2 were AS63997VH5 and AS64380VH5, respectively. Nucleic acid encoding the CAR fragment was then cloned into a lentiviral vector to create full length CAR construct in a single coding frame, using human EF1 alpha promoter for expression.

(T1 amino acid sequence)
SEQ ID NO: 518
MALPVTALLLPLALLLHAARPEVQLVESGGGLVQP
GGSLRLSCAASGNTYSSNYMGWFRQAPGKGLEEVA
VIYTRGGHTYYVDSVRGRFTISQDNAKNSLYLQMN
SLRAEDTAVYYCAASSRHRLGLNNPRDYDYWGQGT
LVTVSSGGGGSGGGGSGGGGSEVQLVESGGGLVQP
GGSLRLSCAASGNTYSSNYMGWFRQAPGKGLEEVA
VIYTRGGHTYYVDSVRGRFTISQDNAKNSLYLQMN
SLRAEDTAVYYCAASSRHRLGLNNPRDYDYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(T2 amino acid sequence)
SEQ ID NO: 519
MALPVTALLLPLALLLHAARPQVQLVESGGGVVQP
GGSLRLSCAASFSGYGVSTMAWFRQAPGKGLEGVA
AITVGSGNTYYADSVKGRFTISRDNSKNTVYLQMN
SLRAEDTAMYYCAVGYLSGGSWDVPGRYNYWGQGT
LVTVSSGGGGSGGGGSGGGGSQVQLVESGGGVVQP
GGSLRLSCAASFSGYGVSTMAWFRQAPGKGLEGVA
AITVGSGNTYYADSVKGRFTISRDNSKNTVYLQMN
SLRAEDTAMYYCAVGYLSGGSWDVPGRYNYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR
(T3 amino acid sequence)
SEQ ID NO: 520
MALPVTALLLPLALLLHAARPQVQLVESGGGVVQP
GGSLRLSCAASFSGYGVSTMAWFRQAPGKGLEGVA
AITVGSGNTYYADSVKGRFTISRDNSKNTVYLQMN
SLRAEDTAMYYCAVGYLSGGSWDVPGRYNYWGQGT
LVTVSSGGGGSGGGGSGGGGSEVQLVESGGGLVQP
GGSLRLSCAASGNTYSSNYMGWFRQAPGKGLEEVA
VIYTRGGHTYYVDSVRGRFTISQDNAKNSLYLQMN
SLRAEDTAVYYCAASSRHRLGLNNPRDYDYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPR

### In Vitro Cytotoxicity Assay

On day 9 post transduction, transduced T cells were harvested and co-incubated with DLL3-expressing tumor cell lines (SHP-77 with DLL3 high expression, NCI-H82 with DLL3 medium expression and NCI-H2171 with DLL3 low expression) and DLL3 negative expressing cell lines (NCI-H460 and HEK293) at an effector (CAR-T) to target cell ratio of 0.5:1 and 2:1 for 22 hours. CAR3 CAR-T cells were used as a reference in all assays to compare assay variation and/or act as a control. Un-transduced T cells (UnT) were used as a negative control.

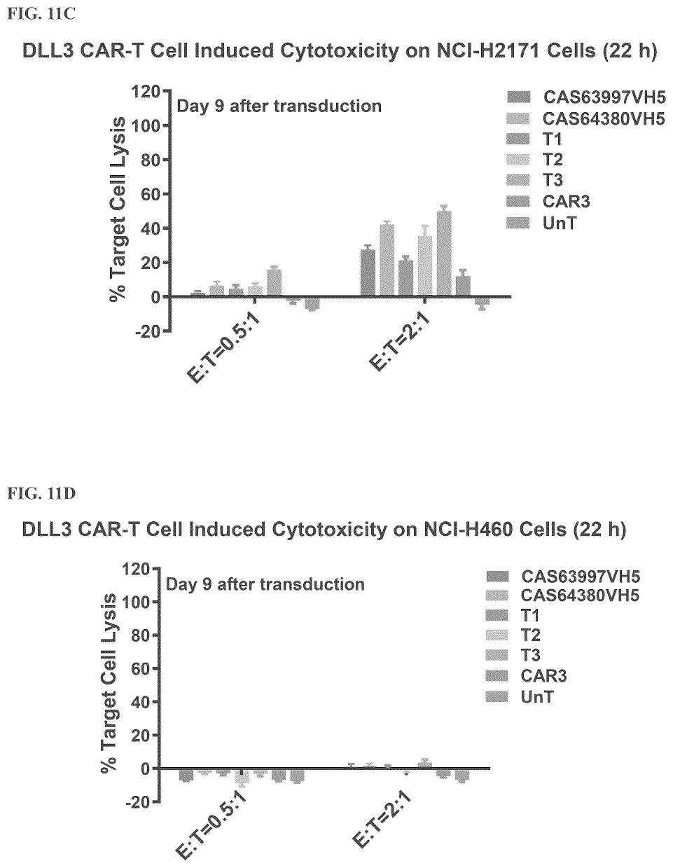

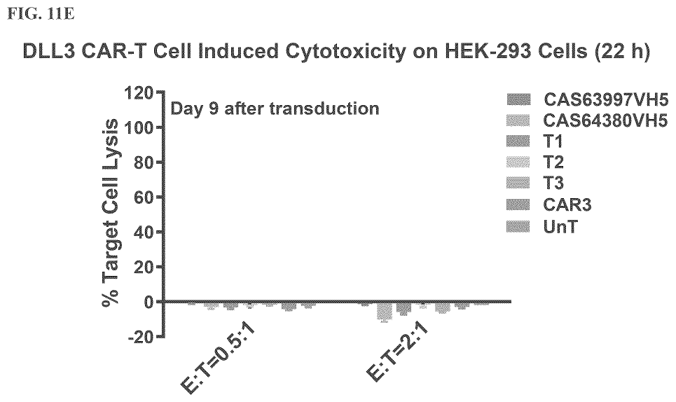

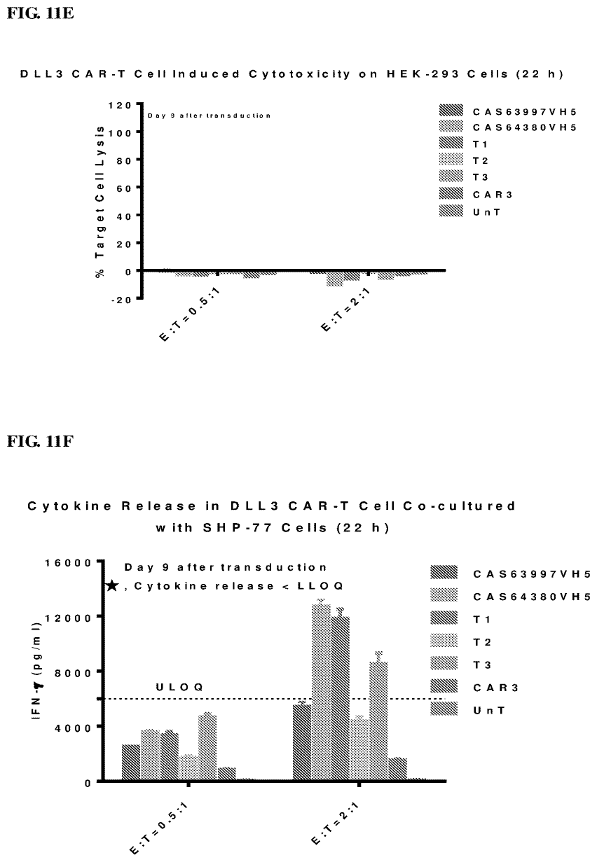

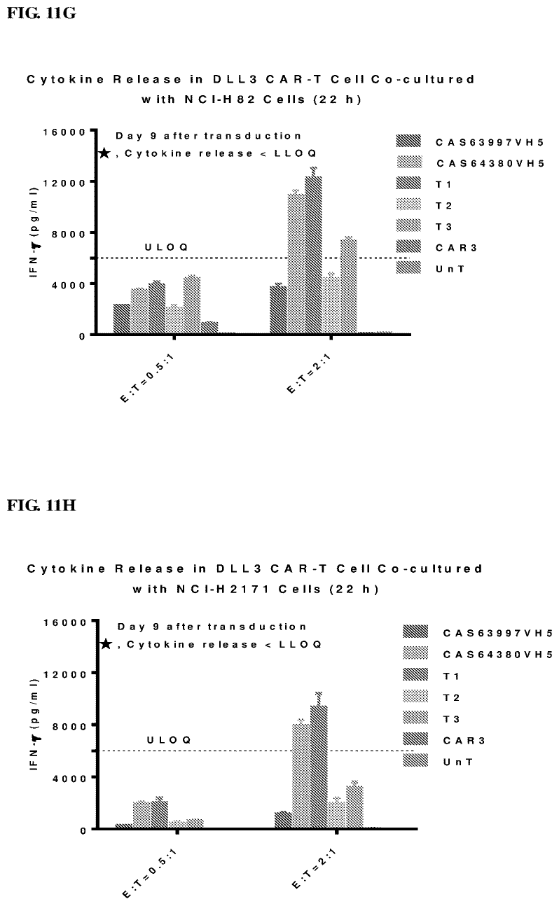

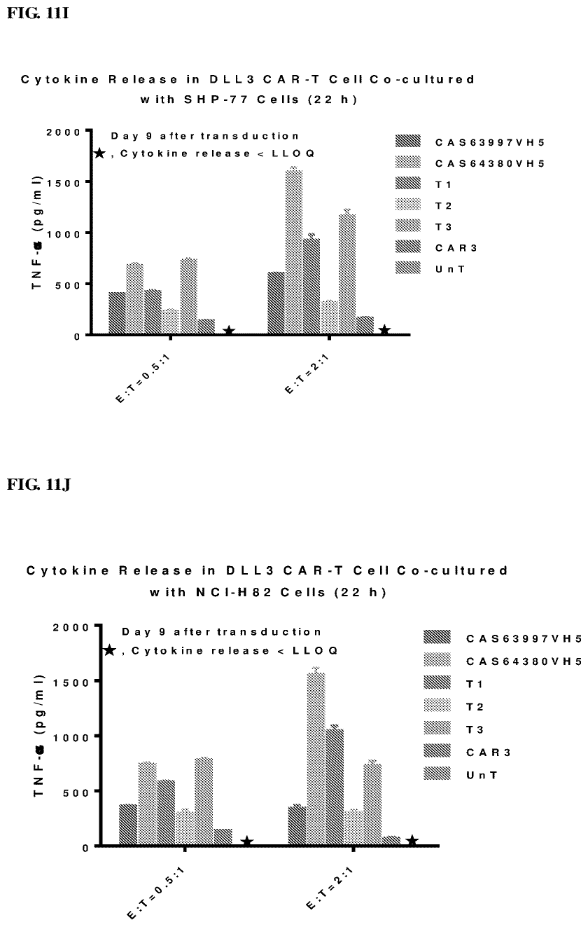

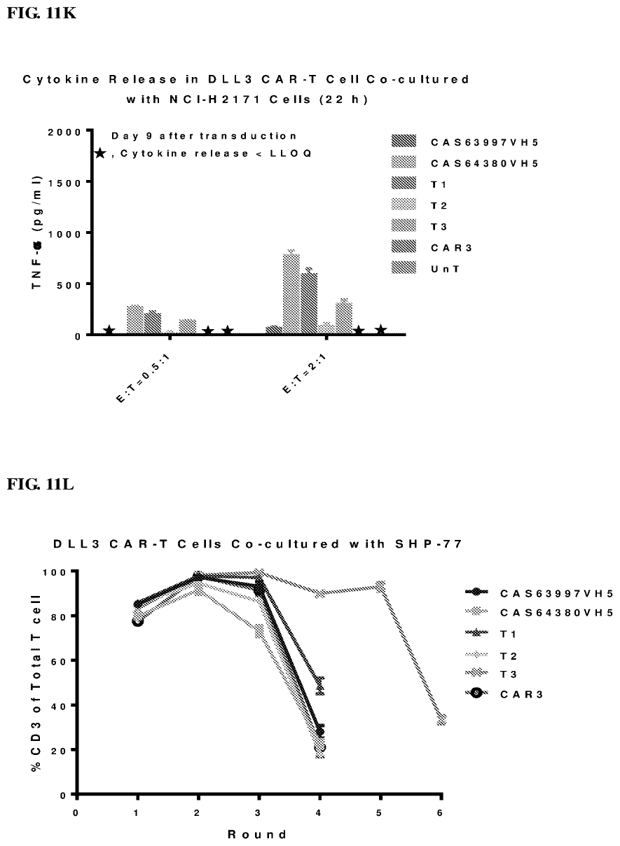

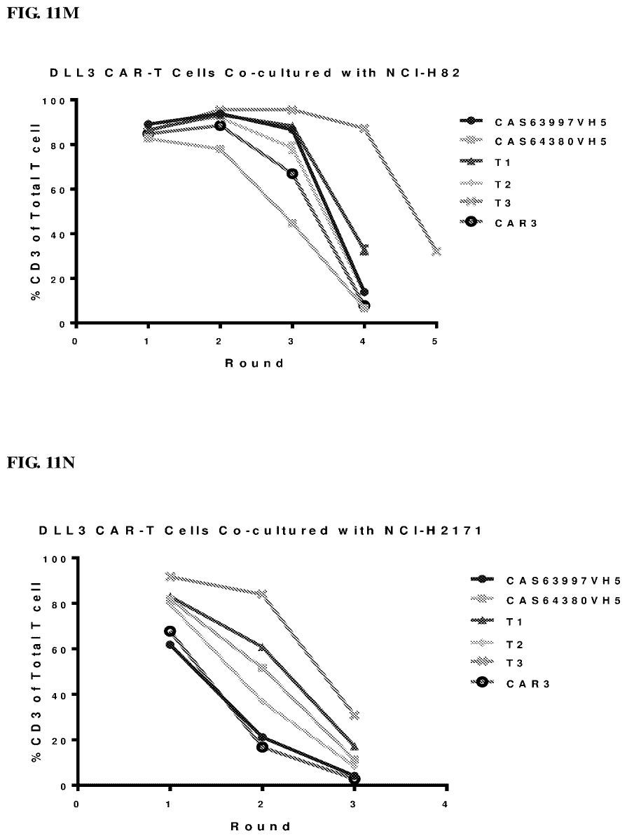

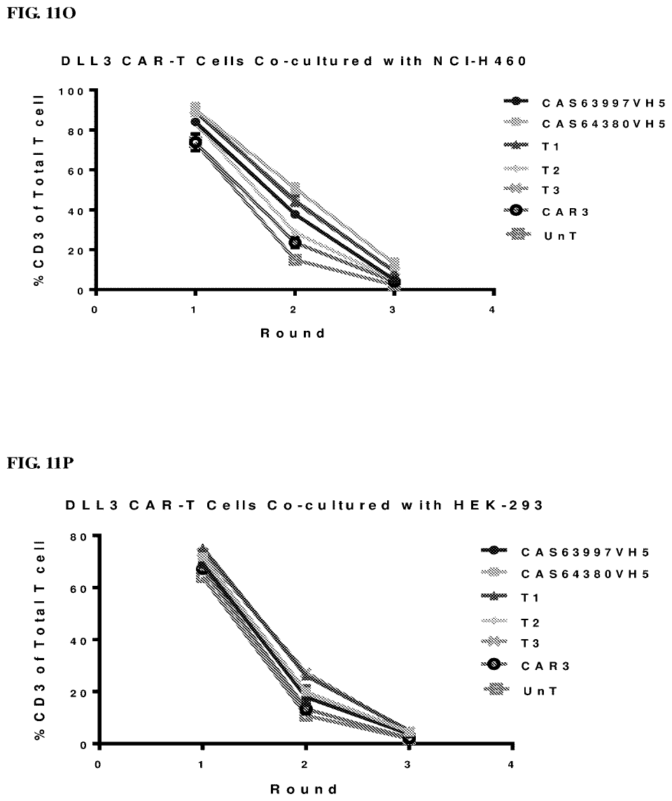

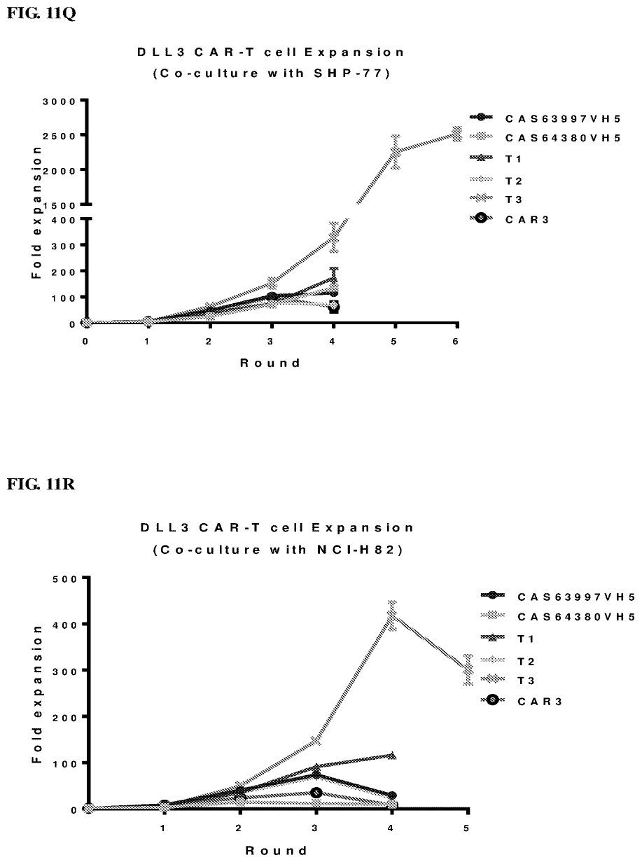

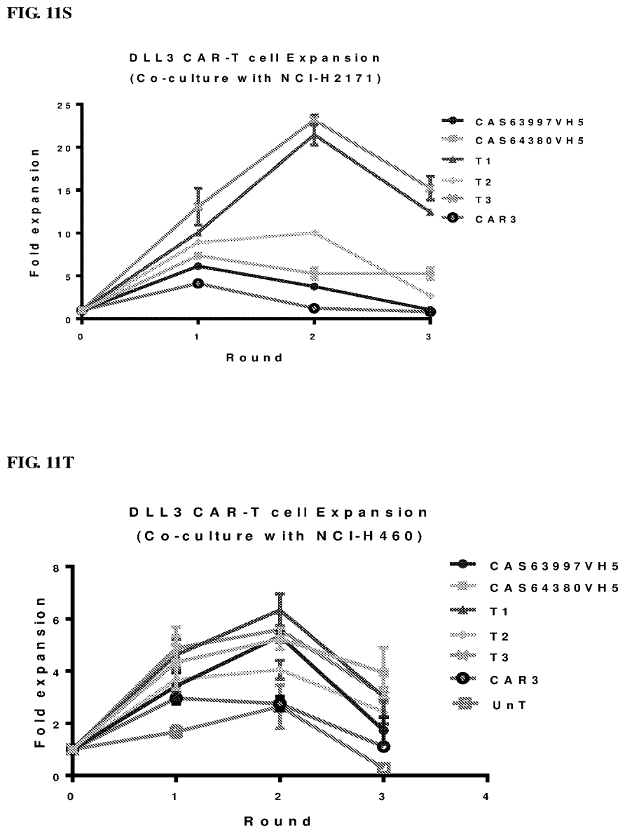

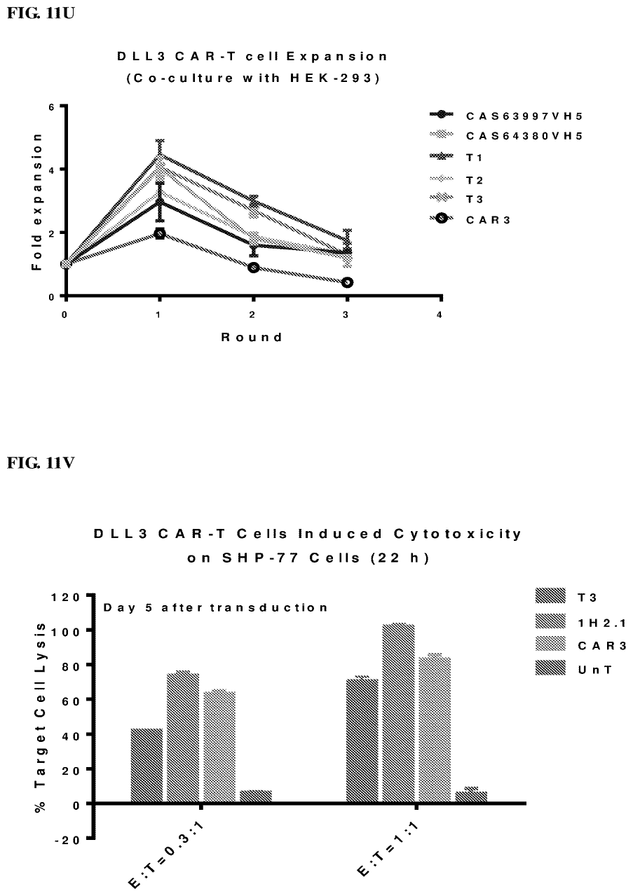

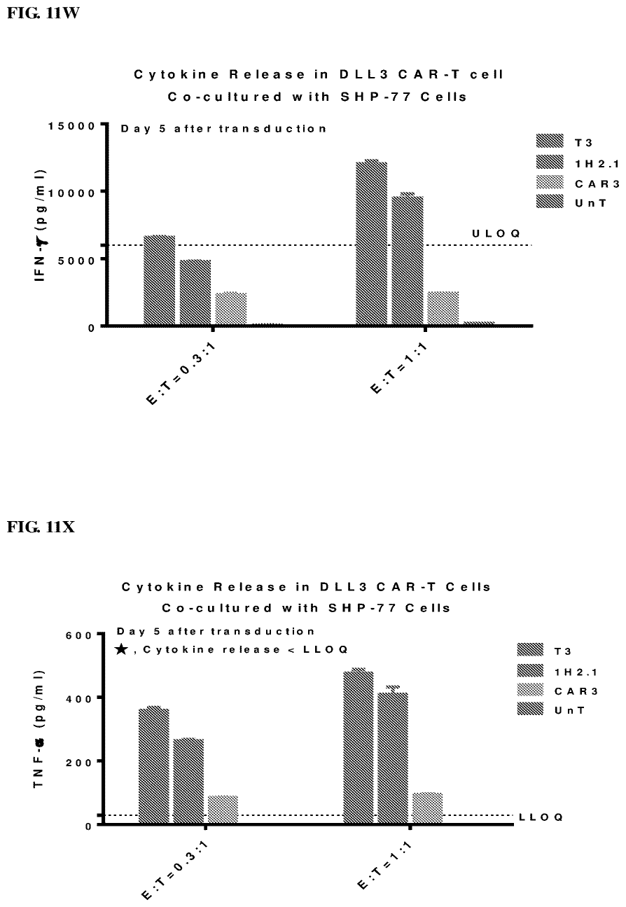

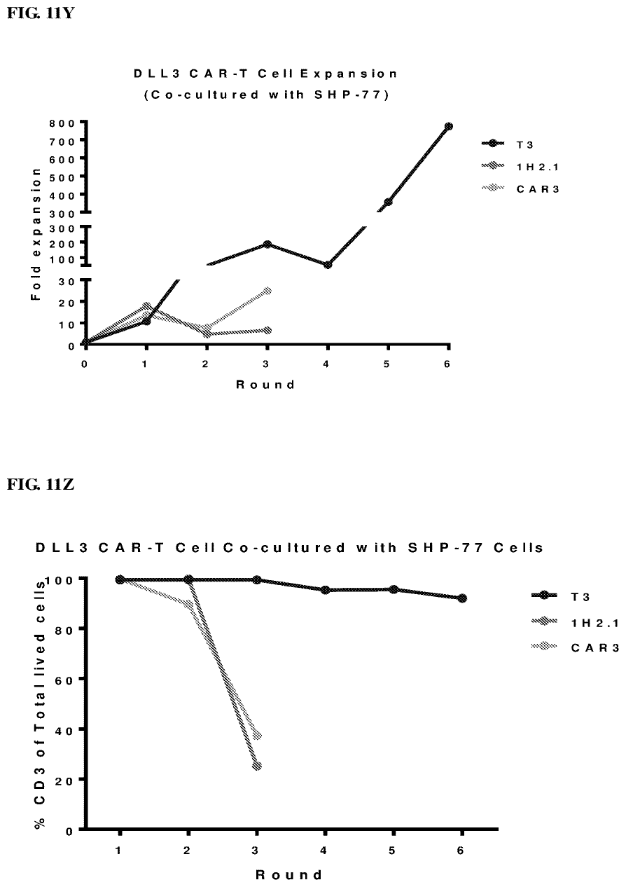

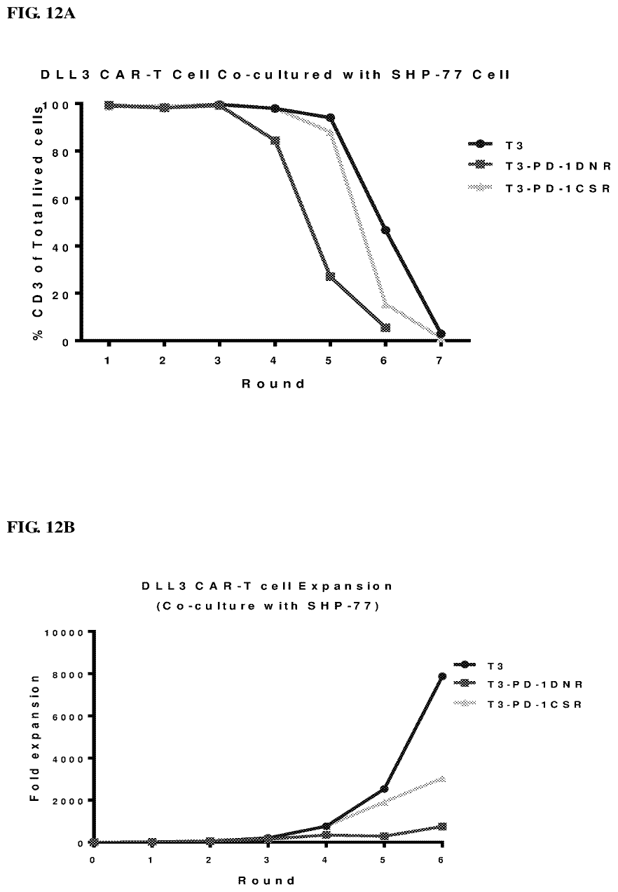

The cytotoxicity of the transduced T cells was determined by a lactate dehydrogenase (LDH) assay. Results show that CAR3 CAR-T and some anti-DLL3 tandem CAR-Ts exhibit strong anti-tumor activities in vitro against SHP-77 cells, while UnT has no target cell killing effect (FIG. 11A-C) and DLL3 negative expression cells (NCI-H460 and HEK293) did not induce cytotoxicity (FIG. 11D-E).Besides CAR3, We also compared in vitro cytotoxicity of SHP-77 cells of T3 and 1H2.1. Result shows that T3 had a comparable or less potent cell killing activity in short-term stimulation (FIG. 11V).

### IFN-γ and TNF-α Release Detection

Additionally, supernatants from the in vitro cytotoxicity assay were collected to assess CAR-induced cytokine release, e.g., interferon gamma (IFN-γ) and TNF-α release. As shown in FIG. 11F-K, CAR3 CAR-T and some anti-DLL3 tandem CAR-Ts were stimulated by DLL3 expressing cell lines to produce IFN-γ and TNF-α, whereas UnT produced little IFN-γ and TNF-α. DLL3 negative expressing cell lines did not reduce specific release of IFN-γ and TNF-α (data not shown). Protocols of IFN-γ and TNF-α release detection can be referred to CISBIO's human TNF-α kits and IFN-γ kits.

Compared with 1H2.1, T3 released more IFN-γ and TNF-α (after co-incubation for 22 hours) (FIG. 11W-X).

### Tandem CAR-T Cytotoxicity and Expansion by Long-Term Stimulation Assay

The DLL3 CAR-T cells were evaluated by repetitive antigen stimulation assay. Upon repetitive stimulation by the SCLC cell lines and control cell lines, the tandem CAR-T cell T3 showed more potent cytotoxicity to SCLC cells, especially to SHP-77 and NCI-H82 cells (FIG. 11L-P). In addition to cytotoxicity activity, tandem CAR-T cell T3 also showed higher proliferation capacity than other CARTs, especially when stimulated by SHP-77 and NCI-H82 cells (FIG. 11Q-U).

Besides CAR3, We also compared in vitro cytotoxicity of SHP-77 cells of T3 and 1H2.1. Result shows that T3 had a superior cytotoxicity and expansion in long-term stimulation (FIG. 11Y-Z).

The repetitive stimulation were carried out as follows.

Round 1: CAR-T cells and 3×105 target cells (e.g, SHP-77) were added to a 24-well plate at an effector to target cell ratio of 1:5, and co-incubated in a carbon dioxide incubator in 37° C., 5% CO2 for 3 days. 200 μL of the cell culture supernatant was pipetted for cytokine detection, and the co-incubated cells were harvested to assess % CD3 and CAR positive rate by flow cytometry;

Round 2: Based on the CAR-T positive rate of the harvested cells in Round 1, the harvested cells were continued to co-incubate with the same volume of fresh target cells (SHP-77) at an effector to target cell ratio of 1:2 for another 3 days. 200 μL of the cell culture supernatant was pipetted for cytokine detection, and the co-incubated cells were harvested to assess % CD3 and CAR positive rate by flow cytometry;

Based on the CAR-T positive rate of each previous Round, Round 3 and next Rounds were performed in a similar manner to that of Round 2.

### Example 12. Evaluation of In Vitro Activity of Humanized Anti-DLL3 PD-1 DNR or CSR Armed CAR-T Cells

To improve persistence of CAR-Ts, we constructed PD-1 Dominant Negative Receptor (PD-IDNR) or PD-1 Chimeric Switch Receptor (PD-1CSR) armored DLL3 CARs. The amino acid sequences of the two CARs were provided in SEQ ID: 521-522. PD-IDNR and PD-1CSR sequences were linked to T3 C terminal via P2A. The amino acid sequences of PD-IDNR and PD-1CRS were provided in SEQ ID: 523-524. Schematic representation of the CAR construct were shown in FIG. 10B.

(T3-PD-1DNR amino acid sequence)
SEQ ID NO: 521
MALPVTALLLPLALLLHAARPQVQLVESGGGVVQP
GGSLRLSCAASFSGYGVSTMAWFRQAPGKGLEGVA
AITVGSGNTYYADSVKGRFTISRDNSKNTVYLQMN
SLRAEDTAMYYCAVGYLSGGSWDVPGRYNYWGQGT
LVTVSSGGGGSGGGGSGGGGSEVQLVESGGGLVQP
GGSLRLSCAASGNTYSSNYMGWFRQAPGKGLEEVA
VIYTRGGHTYYVDSVRGRFTISQDNAKNSLYLQMN
SLRAEDTAVYYCAASSRHRLGLNNPRDYDYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPRGSGATNFSLLKQAGDV
EENPGPMQIPQAPWPVVWAVLQLGWRPGWFLDSPD
RPWNPPTFSPALLVVTEGDNATFTCSFSNTSESFV
LNWYRMSPSNQTDKLAAFPEDRSQPGQDCRFRVTQ
LPNGRDFHMSVVRARRNDSGTYLCGAISLAPKAQI
KESLRAELRVTERRAEVPTAHPSPSPRPAGQAAAP
TTTPAPRPPTPAPTIASQPLSLRPEACRPAAGGAV
HTRGLDFACDIYIWAPLAGTCGVLLLSLVITLYCN
HRRIQ
(T3-PD-1CSR amino acid sequence)
SEQ ID NO: 522
MALPVTALLLPLALLLHAARPQVQLVESGGGVVQP
GGSLRLSCAASFSGYGVSTMAWFRQAPGKGLEGVA
AITVGSGNTYYADSVKGRFTISRDNSKNTVYLQMN
SLRAEDTAMYYCAVGYLSGGSWDVPGRYNYWGQGT
LVTVSSGGGGSGGGGSGGGGSEVQLVESGGGLVQP
GGSLRLSCAASGNTYSSNYMGWFRQAPGKGLEEVA
VIYTRGGHTYYVDSVRGRFTISQDNAKNSLYLQMN
SLRAEDTAVYYCAASSRHRLGLNNPRDYDYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPRGSGATNFSLLKQAGDV
EENPGPMQIPQAPWPVVWAVLQLGWRPGWFLDSPD
RPWNPPTFSPALLVVTEGDNATFTCSFSNTSESFV
LNWYRMSPSNQTDKLAAFPEDRSQPGQDCRFRVTQ
LPNGRDFHMSVVRARRNDSGTYLCGAISLAPKAQI
KESLRAELRVTERRAEVPTAHCPSPLFPGPSKPFW
VLVVVGGVLACYSLLVTVAFIIFWVRSKRSRLLHS
DYMNMTPRRPGPTRKHYQPYAPPRDFAAYRS
(PD-1DNR amino acid sequence)
SEQ ID NO: 523
MQIPQAPWPVVWAVLQLGWRPGWFLDSPDRPWNPP
TFSPALLVVTEGDNATFTCSFSNTSESFVLNWYRM
SPSNQTDKLAAFPEDRSQPGQDCRFRVTQLPNGRD
FHMSVVRARRNDSGTYLCGAISLAPKAQIKESLRA
ELRVTERRAEVPTAHPSPSPRPAGQAAAPTTTPAP
RPPTPAPTIASQPLSLRPEACRPAAGGAVHTRGLD
FACDIYIWAPLAGTCGVLLLSLVITLYCNHRRIQ
(PD-1CSR amino acid sequence)
SEQ ID NO: 524
MQIPQAPWPVVWAVLQLGWRPGWFLDSPDRPWNPP
TFSPALLVVTEGDNATFTCSFSNTSESFVLNWYRM
SPSNQTDKLAAFPEDRSQPGQDCRFRVTQLPNGRD
FHMSVVRARRNDSGTYLCGAISLAPKAQIKESLRA
ELRVTERRAEVPTAHCPSPLFPGPSKPFWVLVVVG
GVLACYSLLVTVAHIFWVRSKRSRLLHSDYMNMTP
RRPGPTRKHYQPYAPPRDFAAYRS

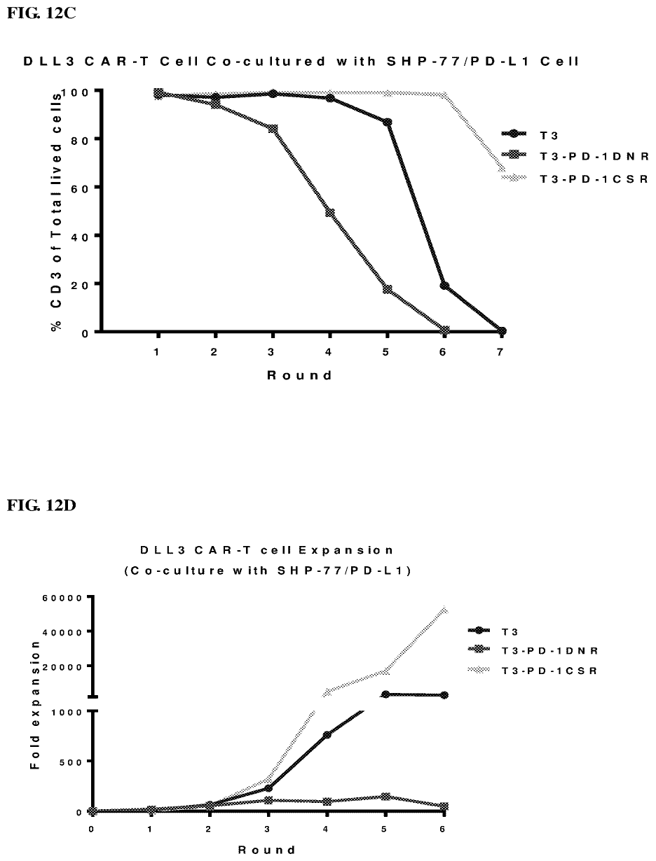

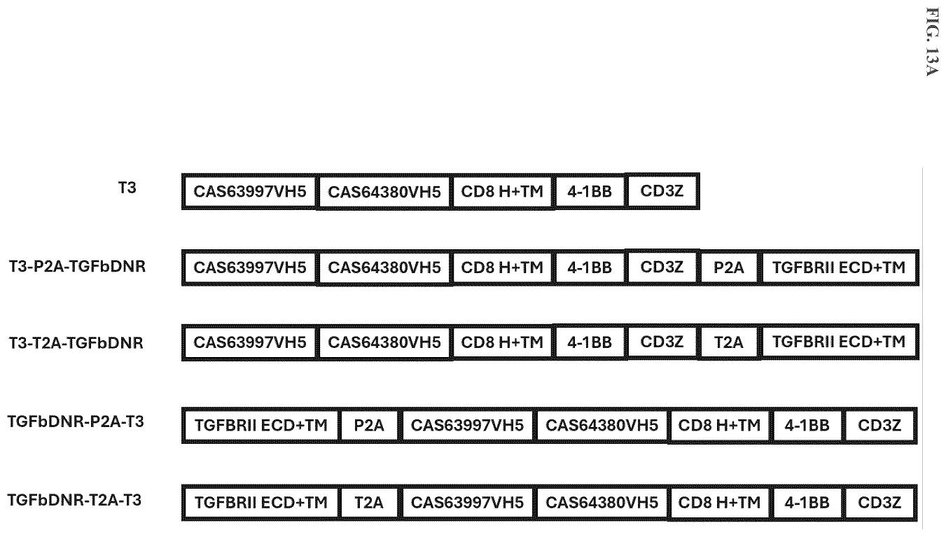

The PD-IDNR or PD-1CSR armored DLL3 CAR-T cells were evaluated by repetitive antigen stimulation assay. Upon repetitive stimulation by the SHP-77 cells, the armored CAR-T cells did not increase cytotoxicity potency and did not improve expansion capability compared with conventional CARTs (FIG. 12A-B). Upon repetitive stimulation by the SHP-77/PD-L1 cells (overexpressing human PD-L1 in SHP-77 cells), the PD-1 CSR armored CAR-T cells showed a superior cytotoxicity potency and expansion capability (FIG. 12C-D).

### Example 13. TGF-β-DNR Enhances the Anti-Tumor Efficacy of DLL3 CAR-T Cells Construction of TGF-β-DNR Armored DLL3 CAR-T Cells

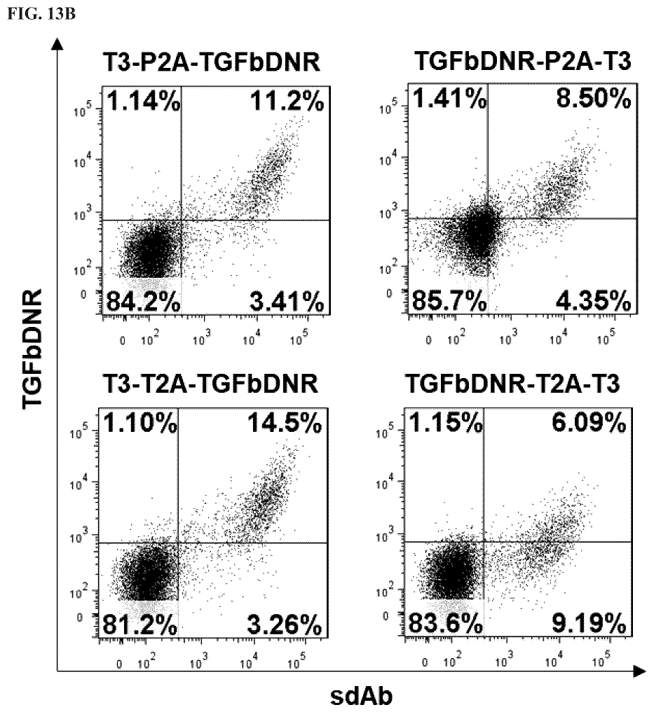

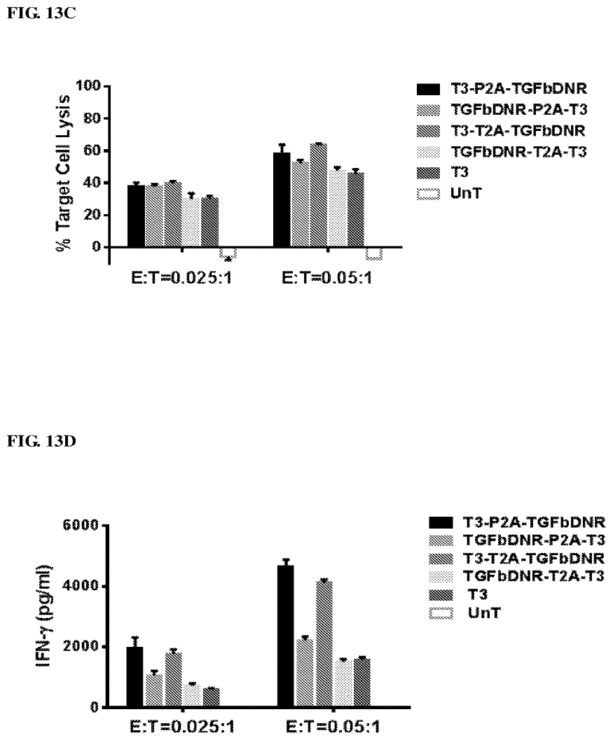

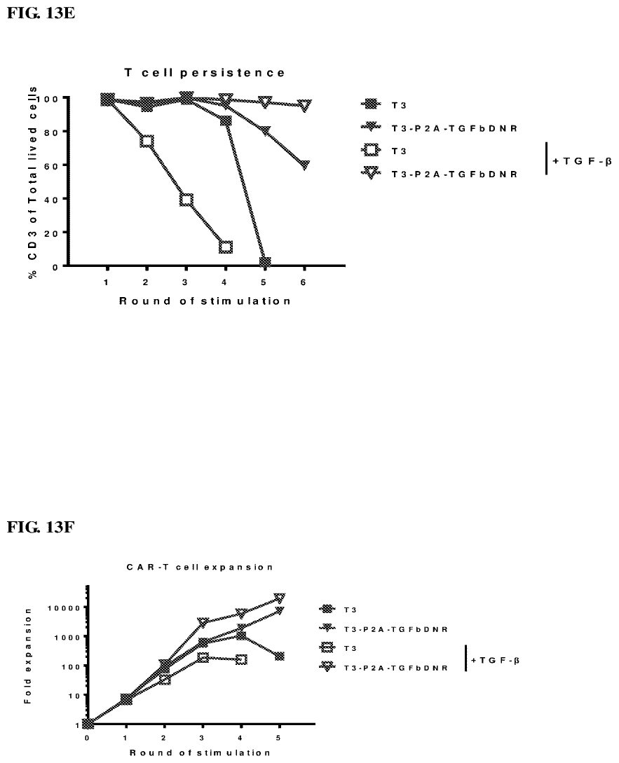

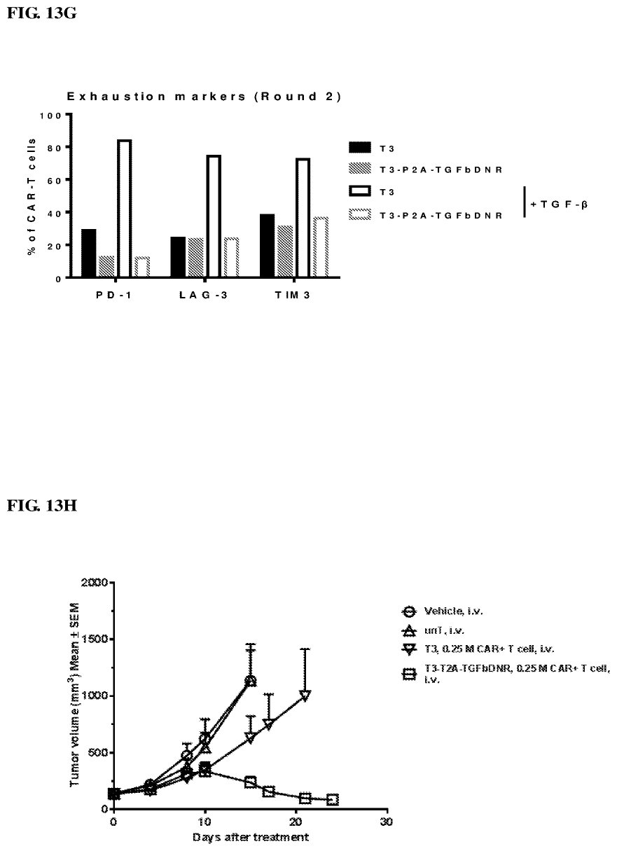

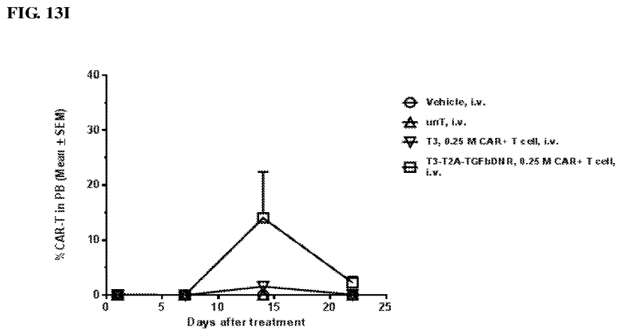

To improve the antitumor performance of the DLL3 CAR-T cells in tumor microenvironment, a TGF-β-DNR sequence was incorporated into the DLL3 CAR as shown in FIG. 13A. The TGF-β-DNR is a truncated version of TGF□RII, which consist of the extracellular and transmembrane domain of the TGFBRII. The constructs T3-P2A-TGF-β-DNR, T3-T2A-TGF-β-DNR comprise a T3-BBZ sequence in the N terminal, a P2A or T2A peptide as indicated, a TGF-β-DNR in the C terminal; the constructs TGF-β-DNR-P2A-T3 and TGF-β-DNR-T2A-T3 comprise a TGF-β-DNR sequence in the N terminal, a P2A or T2A peptide as indicated, and a T3-BBZ sequence in the C terminal. The detailed sequences of the TGF-β-DNR and armored DLL3 CARs were provided in SEQ ID: 525-529.

(T3-P2A-TGF-β-DNR amino acid sequence)
SEQ ID NO: 525
MALPVTALLLPLALLLHAARPQVQLVESGGGVVQP
GGSLRLSCAASFSGYGVSTMAWFRQAPGKGLEGVA
AITVGSGNTYYADSVKGRFTISRDNSKNTVYLQMN
SLRAEDTAMYYCAVGYLSGGSWDVPGRYNYWGQGT
LVTVSSGGGGSGGGGSGGGGSEVQLVESGGGLVQP
GGSLRLSCAASGNTYSSNYMGWFRQAPGKGLEEVA
VIYTRGGHTYYVDSVRGRFTISQDNAKNSLYLQMN
SLRAEDTAVYYCAASSRHRLGLNNPRDYDYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPRGSGATNFSLLKQAGDV
EENPGPMGRGLLRGLWPLHIVLWTRIASTIPPHVQ
KSVNNDMIVTDNNGAVKFPQLCKFCDVRFSTCDNQ
KSCMSNCSITSICEKPQEVCVAVWRKNDENITLET
VCHDPKLPYHDFILEDAASPKCIMKEKKKPGETFF
MCSCSSDECNDNIIFSEEYNTSNPDLLLVIFQVTG
ISLLPPLGVAISVIIIFYCYRVNRQQKLSS
(TGF-β-DNR-P2A-T3 amino acid sequence)
SEQ ID NO: 526
MGRGLLRGLWPLHIVLWTRIASTIPPHVQKSVNND
MIVTDNNGAVKFPQLCKFCDVRFSTCDNQKSCMSN
CSITSICEKPQEVCVAVWRKNDENITLETVCHDPK
LPYHDFILEDAASPKCIMKEKKKPGETFFMCSCSS
DECNDNIIFSEEYNTSNPDLLLVIFQVTGISLLPP
LGVAISVIIIFYCYRVNRQQKLSSGSGATNFSLLK
QAGDVEENPGPMALPVTALLLPLALLLHAARPQVQ
LVESGGGVVQPGGSLRLSCAASFSGYGVSTMAWFR
QAPGKGLEGVAAITVGSGNTYYADSVKGRFTISRD
NSKNTVYLQMNSLRAEDTAMYYCAVGYLSGGSWDV
PGRYNYWGQGTLVTVSSGGGGSGGGGSGGGGSEVQ
LVESGGGLVQPGGSLRLSCAASGNTYSSNYMGWFR
QAPGKGLEEVAVIYTRGGHTYYVDSVRGRFTISQD
NAKNSLYLQMNSLRAEDTAVYYCAASSRHRLGLNN
PRDYDYWGQGTLVTVSSTTTPAPRPPTPAPTIASQ
PLSLRPEACRPAAGGAVHTRGLDFACDIYIWAPLA
GTCGVLLLSLVITLYCKRGRKKLLYIFKQPFMRPV
QTTQEEDGCSCRFPEEEEGGCELRVKFSRSADAPA
YKQGQNQLYNELNLGRREEYDVLDKRRGRDPEMGG
KPRRKNPQEGLYNELQKDKMAEAYSEIGMKGERRR
GKGHDGLYQGLSTATKDTYDALHMQALPPR
(T3-T2A-TGF-β-DNR amino acid sequence)
SEQ ID NO: 527
MALPVTALLLPLALLLHAARPQVQLVESGGGVVQP
GGSLRLSCAASFSGYGVSTMAWFRQAPGKGLEGVA
AITVGSGNTYYADSVKGRFTISRDNSKNTVYLQMN
SLRAEDTAMYYCAVGYLSGGSWDVPGRYNYWGQGT
LVTVSSGGGGSGGGGSGGGGSEVQLVESGGGLVQP
GGSLRLSCAASGNTYSSNYMGWFRQAPGKGLEEVA
VIYTRGGHTYYVDSVRGRFTISQDNAKNSLYLQMN
SLRAEDTAVYYCAASSRHRLGLNNPRDYDYWGQGT
LVTVSSTTTPAPRPPTPAPTIASQPLSLRPEACRP
AAGGAVHTRGLDFACDIYIWAPLAGTCGVLLLSLV
ITLYCKRGRKKLLYIFKQPFMRPVQTTQEEDGCSC
RFPEEEEGGCELRVKFSRSADAPAYKQGQNQLYNE
LNLGRREEYDVLDKRRGRDPEMGGKPRRKNPQEGL
YNELQKDKMAEAYSEIGMKGERRRGKGHDGLYQGL
STATKDTYDALHMQALPPRGSGEGRGSLLTCGDVE
ENPGPMGRGLLRGLWPLHIVLWTRIASTIPPHVQK
SVNNDMIVTDNNGAVKFPQLCKFCDVRFSTCDNQK
SCMSNCSITSICEKPQEVCVAVWRKNDENITLETV
CHDPKLPYHDFILEDAASPKCIMKEKKKPGETFFM
CSCSSDECNDNIIFSEEYNTSNPDLLLVIFQVTGI
SLLPPLGVAISVIIIFYCYRVNRQQKLSS
(TGF-β-DNR-T2A-T3 amino acid sequence)
SEQ ID NO: 528
MGRGLLRGLWPLHIVLWTRIASTIPPHVQKSVNND
MIVTDNNGAVKFPQLCKFCDVRFSTCDNQKSCMSN
CSITSICEKPQEVCVAVWRKNDENITLETVCHDPK
LPYHDFILEDAASPKCIMKEKKKPGETFFMCSCSS
DECNDNIIFSEEYNTSNPDLLLVIFQVTGISLLPP
LGVAISVIIIFYCYRVNRQQKLSSGSGEGRGSLLT
CGDVEENPGPMALPVTALLLPLALLLHAARPQVQL
VESGGGVVQPGGSLRLSCAASFSGYGVSTMAWFRQ
APGKGLEGVAAITVGSGNTYYADSVKGRFTISRDN
SKNTVYLQMNSLRAEDTAMYYCAVGYLSGGSWDVP
GRYNYWGQGTLVTVSSGGGGSGGGGSGGGGSEVQL
VESGGGLVQPGGSLRLSCAASGNTYSSNYMGWFRQ
APGKGLEEVAVIYTRGGHTYYVDSVRGRFTISQDN
AKNSLYLQMNSLRAEDTAVYYCAASSRHRLGLNNP
RDYDYWGQGTLVTVSSTTTPAPRPPTPAPTIASQP
LSLRPEACRPAAGGAVHTRGLDFACDIYIWAPLAG
TCGVLLLSLVITLYCKRGRKKLLYIFKQPFMRPVQ
TTQEEDGCSCRFPEEEEGGCELRVKFSRSADAPAY
KQGQNQLYNELNLGRREEYDVLDKRRGRDPEMGGK
PRRKNPQEGLYNELQKDKMAEAYSEIGMKGERRRG
KGHDGLYQGLSTATKDTYDALHMQALPPR
(TGF-β-DNR amino acid sequence)
SEQ ID NO: 529
MGRGLLRGLWPLHIVLWTRIASTIPPHVQKSVNND
MIVTDNNGAVKFPQLCKFCDVRFSTCDNQKSCMSN
CSITSICEKPQEVCVAVWRKNDENITLETVCHDPK
LPYHDFILEDAASPKCIMKEKKKPGETFFMCSCSS
DECNDNIIFSEEYNTSNPDLLLVIFQVTGISLLPP
LGVAISVIIIFYCYRVNRQQKLSS

All the constructs were packaged into lentiviruses based on the second generation lentiviral system. Then primary T cells isolated from the PBMC of healthy donors were transduced with the lentiviruses. 4 days after transduction, the positive ratios of sdAb and TGF-β-DNR were detected by FACS (FIG. 13B). Results showed that the sdAb positive ratios of T3-P2A-TGF-β-DNR, T3-T2A-TGF-β-DNR, TGF-β-DNR-P2A-T3 and TGF-β-DNR-T2A-T3 CAR-T cells were comparable. However, the positive ratio of TGF-β-DNR were higher in constructs T3-P2A-TGF-β-DNR and T3-T2A-TGF-β-DNR than in TGF-β-DNR-P2A-T3 and TGF-β-DNR-T2A-T3. These results indicated the expression of TGF-β-DNR was higher when it conjugates to the C terminal of CAR.

### In Vitro Cytotoxicity Assays

Then the cytotoxicity of these CAR-T cells was evaluated by LDH or IFN-γ releasing assays. 5 days after transduction, the CAR-T cells were adjusted to same sdAb positive ratios by untransduced T cells (UnT). Then the CAR-T cells or UnT cells were co-incubated with SHP77 for 48 h in the presence of 5 ng/ml TGF-β, and the LDH and IFN-γ releasing were measured (FIG. 13C-D). The results showed that the TGF-β-DNR armored CAR-T cells induced more specific lysis of target cell than the unarmored CAR-T cells. Accordingly, the TGF-β-DNR armored CAR-T cells showed a higher capacity of IFN-γ release upon antigen activation. In consistent with the TGF-β-DNR expression levels of the different CAR-T cells, the T3-P2A-TGF-β-DNR, T3-T2A-TGF-β-DNR showed higher levels of IFN-γ secretion than TGF-β-DNR-P2A-T3 and TGF-β-DNR-T2A-T3 CAR-T cells. Collectively these results demonstrated that the TGF-β-DNR was able to enhance the cytotoxicity of DLL3 CAR-T cells against the DLL3 positive SCLC cells.

### Long-Term Stimulation Assay

To determine whether the TGF-β-DNR could resist the inhibitory effects of TGF-β on CAR-T cells, a long-term stimulation assay was performed. Briefly, the T3-P2A-TGF-β-DNR and T3 CAR-T cells were repetitively challenged with SHP77 cells every 3 days in the presence or absence of 5 ng/ml of TGF-β. At the end of each round stimulation, the percentages of T cells in total viable cells were analyzed by FACS, and the expansion of CAR-T cells were calculated. As shown in FIG. 13E and FIG. 13F, the persistence and expansion of T3 CAR-T cells was inhibited by TGF-β. In contrast, the persistence and expansion of T3-P2A-TGF-β-DNR was well maintained even in the presence of TGF-β. T cell exhaustion markers were analyzed by FACS after 2 rounds stimulation with SHP77 cells. As shown in FIG. 13G, treatment with TGF-β upregulated the expression of exhaustion markers in T3 cells, but not in T3-P2A-TGF-β-DNR CAR-T cells. Collectively, these results demonstrated that TGF-β-DNR protects DLL3 CAR-T cells from the inhibition by TGF-β. Because the expression level of TGF-β is usually elevated in the microenvironment of solid tumors, our results indicate that the addition of TGF-β-DNR improves the anti-tumor efficacy of DLL3 CAR-T cells in solid tumors.

### In Vivo Anti-Tumor Efficacy Study

To further investigate whether the TGF-β-DNR could enhance the anti-tumor efficacy of DLL3 CAR-T cells in vivo, the T3-T2A-TGF-β-DNR CAR-T cells or the parental CAR-T cells were evaluated in xenograft model. Briefly, 1×10⁷ SHP77 cells were subcutaneously implanted into NCG mice. After 7˜10 days, when tumor volume reached 100˜200 mm³, 2.5×10⁵ CAR-T cells were injected intravenously into the mice. Then tumor volume was measured twice a week, and percentage of CAR-T in peripheral blood was measured once a week. As shown in FIG. 13H, at a suboptimal dose, the T3-T2A-TGF-β-DNR CAR-T cells could potently suppress tumor growth, while the parental T3 CAR-T could not. As shown in FIG. 13I, the percentage of T3-T2A-TGF-β-DNR CAR-T in peripheral blood leukocytes was higher than T3. Collectively, these results demonstrate that the TGF-β-DNR could enhance the anti-tumor efficacy of DLL3 CAR-T cells in vivo.

1. An engineered immune cell expressing a chimeric antigen receptor (CAR) and a dominant negative receptor (DNR);
wherein the CAR specifically binds to DLL3, the CAR comprising:
a) a first single domain antibody (sdAb) moiety comprising a CDR1 comprising the amino acid sequence of SEQ ID NO: 6; a CDR2 comprising the amino acid sequence of SEQ ID NO: 87; and
a CDR3 comprising the amino acid sequence of SEQ ID NO: 168; and
b) a second single domain antibody (sdAb) moiety comprising a CDR1 comprising the amino acid sequence of SEQ ID NO: 21; a CDR2 comprising the amino acid sequence of SEQ ID NO: 102;
and a CDR3 comprising the amino acid sequence of SEQ ID NO: 183, and
wherein the DNR is a TGF-β DNR having the amino acid sequence of SEQ ID NO: 529.

2. (canceled)

3. The engineered immune cell of claim 1, wherein the first sdAb moiety comprises the amino acid sequence of SEQ ID NO: 366 and the second sdAb moiety comprises the amino acid sequence of SEQ ID NO: 356.

4. The engineered immune cell of claim 1, wherein the CAR comprises, from N-terminus to C-terminus, a signal peptide, a DLL3 binding domain, a hinge domain, a transmembrane domain, and an intracellular signaling domain.

5. The engineered immune cell of claim 4, wherein the intracellular signaling domain is derived from CD3ζ, FcRγ, FcRβ, CD3γ, CD3δ, CD3ε, CD5, CD22, CD79a, CD79b, or CD66d.

6. The engineered immune cell of claim 4, wherein the intracellular signaling domain further comprises an intracellular co-stimulatory sequence.

7. The engineered immune cell of claim 6, wherein the intracellular co-stimulatory sequence is derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof.

8. A nucleic acid molecule encoding a chimeric antigen receptor (CAR) and a dominant negative receptor (DNR);
wherein the CAR specifically binds to DLL3, the CAR comprising:
a) a first single domain antibody (sdAb) moiety comprising a CDR1 comprising the amino acid sequence of SEQ ID NO: 6; a CDR2 comprising the amino acid sequence of SEQ ID NO: 87; and
a CDR3 comprising the amino acid sequence of SEQ ID NO: 168; and
b) a second single domain antibody (sdAb) moiety comprising a CDR1 comprising the amino acid sequence of SEQ ID NO: 21; a CDR2 comprising the amino acid sequence of SEQ ID NO: 102;
and a CDR3 comprising the amino acid sequence of SEQ ID NO: 183, and
wherein the DNR is a TGF-β DNR having the amino acid sequence of SEQ ID NO: 529.

9. (canceled)

10. The nucleic acid molecule of claim 8, wherein the first sdAb moiety comprises the amino acid sequence of SEQ ID NO: 366 and the second sdAb moiety comprises the amino acid sequence of SEQ ID NO: 356.

11. The nucleic acid molecule of claim 8, wherein the nucleic acid molecule further comprises a polynucleotide sequence encoding a 2A self-cleaving peptide linking the CAR and the TGF-β DNR.

12. The nucleic acid molecule of claim 11, wherein the 2A self-cleaving peptide is a T2A peptide or a P2A peptide.

13. The nucleic acid molecule of claim 8, wherein the nucleic acid molecule encodes a peptide having at least about 95% sequence identity to an amino acid sequence of SEQ ID NOs: 525-528.

14. The nucleic acid molecule of claim 13, wherein the nucleic acid molecule encodes a peptide having at least about 95% sequence identity to an amino acid sequence of SEQ ID NOs: 527.

15. An expression vector comprising the nucleic acid molecule of claim 8.

16. A cell comprising the nucleic acid molecule of claim 8.

17. The engineered immune cell of claim 1, wherein the engineered immune cell is selected from the group consisting of a cytotoxic T cell, a helper T cell, a natural killer T cell, a γδ T cell and a Nature Killer cell.

18. A pharmaceutical composition comprising the engineered immune cell of claim 1 and a physiologically acceptable excipient.

19. A method for treating a DLL3 associated disorder in a subject, the method comprising administering to the subject in need thereof a therapeutically effective amount of the pharmaceutical composition of claim 18.

20. The method of claim 19, wherein the DLL3 associated disorder is a cancer selected from the group consisting of lung cancer, melanoma, breast cancer, prostate cancer, colon cancer, renal cell carcinoma, ovarian cancer, neuroblastoma, rhabdomyosarcoma, leukemia and lymphoma.

21. The method of claim 19, wherein the DLL3 associated disorder is small cell lung cancer.
